(19) United States
(12) Patent Application Publication
(10) Pub. No.: US 2026/0117300 A1
(43) Pub. Date: Apr. 30, 2026

(54) DESIGN METHOD, MANUFACTURING METHOD, DESIGN DEVICE, DESIGN PROGRAM, AND RECORDING MEDIUM FOR PRIMER FOR AMPLICON METHYLATION SEQUENCE ANALYSIS

(71) Applicant: FUJIFILM Corporation, Tokyo (JP)

(72) Inventors: Naoko YAMAGUCHI, Ashigarakami-gun (JP); Yukina MURAO, Ashigarakami-gun (JP); Masaya NAGASE, Ashigarakami-gun (JP)

(73) Assignee: FUJIFILM Corporation, Tokyo (JP)

(21) Appl. No.: 19/004,078

(22) Filed: Dec. 27, 2024

Related U.S. Application Data
(63) Continuation of application No. PCT/JP2023/021016, filed on Jun. 6, 2023.

(30) Foreign Application Priority Data
Aug. 31, 2022 (JP) 2022-137785

(51) Int. Cl. C12Q 1/6876; C12Q 1/6811; C12Q 1/6851

(52) U.S. Cl. CPC C12Q 1/6876; C12Q 1/6811; C12Q 1/6851; C12Q 2600/154; C12Q 2600/16

(57) ABSTRACT

An object of the present invention is to provide a design method, a manufacturing method, a design device, a design program, and a recording medium of a primer for amplicon methylation sequence analysis, which can improve a design success rate of the primer while suppressing the formation rate of a primer dimer extremely low.

The present invention is a primer design method for amplicon methylation sequence analysis, including a primer sequence determination step of selecting one or more primer candidate sequence pairs related to a predetermined target site from one or more primer candidate sequences, calculating a local alignment score between predetermined primer sequences, and adopting and determining a primer candidate sequence pair having a score equal to or less than a predetermined threshold value as a forward primer sequence and a reverse primer sequence for amplifying a region including the predetermined target site.

### CROSS-REFERENCE TO RELATED APPLICATIONS

This application is a Continuation of PCT International Application No. PCT/JP2023/021016 filed on Jun. 6, 2023, which claims priority under 35 U.S.C. § 119 (a) to Japanese Patent Application No. 2022-137785 filed on Aug. 31, 2022. The above applications are hereby expressly incorporated by reference, in their entirety, into the present application.

### REFERENCE TO ELECTRONIC SEQUENCE LISTING

The application contains a Sequence Listing which has been submitted electronically in XML format and is hereby incorporated by reference in its entirety. Said .XML copy, created on May 30, 2023, is named “22F0085201_220830.xml” and is 893,680 bytes in size. The sequence listing contained in this .XML file is part of the specification and is hereby incorporated by reference herein in its entirety.

### BACKGROUND OF THE INVENTION

### 1. Field of the Invention

The present invention relates to a design method, a manufacturing method, a design device, a design program, and a recording medium for a primer for amplicon methylation sequence analysis. Particularly, the present invention relates to a primer design method for designing a primer for simultaneously amplifying a plurality of amplification target regions including a plurality of target sites in deoxyribonucleic acid (DNA) treated with bisulfite or an enzyme by a multiplex polymerase chain reaction (PCR) and a manufacturing method, a design device, a design program, and a recording medium for the primer.

### 2. Description of the Related Art

DNA methylation is known as one of the epigenetic mechanisms, which is a gene expression control mechanism that is not involved in changes in DNA base sequence. Mammalian DNA methylation occurs mainly at the 5-position carbon atom of cytosine (C) in a CG sequence on DNA.

Gene promoter regions have a lot of regions called CpG islands where the CG sequence appear with high frequency. It is known that many CG sequences in these regions are unmethylated initially, but they are methylated due to diseases, development, differentiation, inflammation, aging, and the like and suppress gene expression. For example, it is known that in cancer cells, many of cancer suppressor gene groups are inactivated due to the acceleration of methylation of the CpG islands in a gene promoter region.

As described above, DNA methylation is highly involved in the control of gene expression. Therefore, the information on DNA methylation is considered to be useful for clarification of the mechanism of a disease such as cancer, evaluation of the differentiation status of various cells, and the like and is drawing attention in various fields such as diagnosis, treatment, drug discovery, and regenerative medicine, and research and development are actively carried out for the DNA methylation. For example, the DNA methylation status of a specific region is measured and analyzed to make an attempt to investigate whether or not different types of cells have drug resistance in developing drugs, an attempt to evaluate the presence or absence of cancer cells or malignancy (progress) of cancer cells based on the ratio between normal cells and abnormal cells, and an attempt to evaluate the differentiation status of stem cells and use the evaluation result for quality control of the stem cells.

As one of the methods of analyzing the DNA methylation status, there is a method using a bisulfite (hydrogen sulfite) reaction.

For example, cytosine (C) in a CG sequence related to a certain disease is picked up and adopted as a target site (measurement site). In FIG. 13A, [1] to [4] are methylation sites, and among these sites, [2] and [4] are set as target sites A and B (FIG. 13A shows only one strand).

Subsequently, a template DNA is treated with bisulfite (hydrogen sulfite). In a case where cytosine (C) in the CG sequence is methylated on the template DNA, cytosine (C) remains as it is after the treatment (see the methylation sites [3] and [4] in FIG. 13A). On the other hand, in a case where cytosine (C) in the CG sequence is unmethylated on the template DNA, cytosine (C) is deaminated and converted into uracil (U) (see methylation sites [1] and [2] in FIG. 13A).

Recently, instead of the bisulfite treatment, a method has been used which is a method of performing base conversion similar to the aforementioned reaction by using, for example, an enzyme such as NEB Next Enzymatic Methyl-seq Kit manufactured by New England Biolabs.

Then, for sequence analysis, the bisulfite-treated DNA is amplified using a polymerase chain reaction (PCR). The amplified DNA, that is, the PCR amplification product is subjected to sequence analysis using a capillary sequencer or a next generation sequencer (NGS).

In a case where the bisulfite-treated DNA is amplified using PCR, cytosine (C) remains as it is, (see the methylation sites [3] and [4] in FIG. 13A), whereas uracil (U) is replaced with thymine (T) and amplified (see the methylation sites [1] and [2] in FIG. 13A).

For example, utilizing the difference between cytosine (C) and thymine (T) caused in the sequence of the PCR amplification product makes it possible to ascertain the methylation status of a predetermined target site in DNA before the bisulfite treatment (template DNA), that is, to detect whether or not DNA of a predetermined target site selected from one cell is methylated. More specifically, based on whether a base in a predetermined target site of a PCR amplification product is cytosine (C) or thymine (T), it is possible to ascertain whether cytosine (C) in the predetermined target site of a template DNA is methylated or unmethylated. As shown in FIG. 13A, the base in a target site A of the PCR amplification product is thymine (T), which tells that cytosine (C) in the target site A of the template DNA is unmethylated. On the other hand, the base of the PCR amplification product of a target site B is cytosine (C), which tells that cytosine (C) in the target site B of the template DNA is methylated.

In addition, utilizing the difference between cytosine (C) and thymine (T) caused in the sequence of the PCR amplification product makes it possible to detect the methylation status (frequency) of bisulfite-untreated DNA (template DNA) of a specific target site derived from a plurality of cells, that is, to detect whether or not the DNA of a specific target site derived from a plurality of cells is methylated, and also makes it possible to ascertain the proportion of cells in which DNA methylation has occurred in a specific target site based on the detection result. In a case where there is a plurality of specific target sites, by detecting whether or not DNA methylation has occurred in each of the specific target sites, it is possible to detect the proportion of cells in which DNA methylation has occurred for each of the target sites based on the detection result. More specifically, based on whether the base in the specific target sites which occurs in the sequence of the PCR amplification product is cytosine (C) or thymine (T), it is possible to ascertain the DNA methylation status (frequency) of the specific target sites derived from a plurality of cells. The DNA methylation status (frequency) of the specific target sites can be obtained by calculating Methylation degree=C/(C+T) based on the number of cytosine (C) and thymine (T) generated in each target site (measurement site). In a case where there is a plurality of specific target sites, the proportion of cells in which DNA methylation has occurred can be ascertained for each of the specific target sites.

For example, as shown in FIG. 13B, in a case where a plurality of cells (cells C1 to C3 in FIG. 13B) is used to evaluate methylation status (frequency) of target sites (measurement sites) A and B derived from the plurality of cells, the number of cytosine (C) generated in the target site A is 2 and the number of thymine (T) is 1. Accordingly, the methylation degree is calculated to be 2/(2+1)=0.67. Therefore, the DNA methylation status (frequency) in the target site A of FIG. 13B is 0.67 which is a methylation degree derived from 3 cells and can be ascertained as the proportion of cells where DNA methylation has occurred. Meanwhile, the number of cytosine (C) and the number of thymine (T) generated in the target site B is 3 and 0 respectively. Therefore, the methylation degree is calculated to be 3/(3+0)=1. Accordingly, the DNA methylation status (frequency) in the target site B of FIG. 13B is 1 which is a methylation degree derived from 3 cells and can be ascertained as the proportion of cells where DNA methylation has occurred.

Likewise, the methylation status (frequency) of the target site A shown in FIG. 13A can be detected as a methylation degree of 0 Derived from one cell, and the methylation status (frequency) of the target site B can be detected as a methylation degree of 1 derived from one cell.

For the amplification of the bisulfite-treated DNA, sometimes multiplex PCR capable of simultaneously amplifying two or more amplification target regions on DNA by the same reaction is used.

In order to ascertain the DNA methylation status of a predetermined target site or the DNA methylation status (frequency) of a specific target site derived from a plurality of cells by using multiplex PCR, as shown in FIG. 13C (FIG. 13C shows only one strand), it is necessary to use a primer pair (a forward primer and a reverse primer) for amplifying one or more amplification target regions each including two or more target sites. Specifically, as shown in FIG. 13A, it is necessary to use a primer pair for amplifying an amplification target region (amplification region) including the target site A and a primer pair for amplifying an amplification target region (amplification region) including the target site B.

In designing primers for bisulfite-treated DNA, in addition to the conditions considered in the usual primer design (that is, the design of a primer for bisulfite-untreated DNA), the following conditions should also be considered.

First, there is a premise that whether or not DNA methylation will occur is unpredictable unlike in the base sequence. That is, some bases are not sure whether they will be thymine (T) or cytosine (C) after the bisulfite treatment. Therefore, in the primer design for analyzing the DNA methylation status, in order to prevent the amplification efficiency of the primer from changing depending on the methylation status of the periphery of the target site, it is necessary that the primer have no CG sequences in a binding site as far as possible or that the position of CG sequences in the primer be limited to reduce the influence thereof even though the primer includes CG sequences.

In the two strands of DNA, many cytosines (C) on DNA are converted into thymines (T) by the bisulfite treatment. Therefore, in the DNA sequence of each strand, the region configured with three bases other than cytosine (C) increases after the bisulfite treatment. Accordingly, it is also necessary to consider that a primer capable of specifically binding to the region composed of three bases should be designed.

In addition, due to the conversion of many cytosines (C) on DNA into thymines (T), the double-stranded DNA loses the complementarity. Therefore, in a case where both strands of DNA need to be amplified and analyzed, it is necessary to design a primer pair (a forward primer and a reverse primer) for amplifying one or more amplification target regions each including a target site of each strand, that is, two sets of primer pair.

Therefore, compared to designing general primers, designing primers for bisulfite-treated DNA having the aforementioned unique circumstances is more difficult because the design conditions are different.

There are many primer design software, and most of them are for designing general primers, such as Primer-BLAST. Therefore, these software are incapable of setting conditions considering the cytosine that undergoes base conversion by the bisulfite treatment. That is, because the general primer design software does not take into account at all the unique circumstances involved in designing primers for bisulfite-treated DNA as described above, it is impossible to design primers for bisulfite-treated DNA with these software.

Furthermore, in a case where multiplex PCR is used for the amplification of the bisulfite-treated DNA, because a plurality of amplification target regions including each of the target sites related to the analysis of methylation degree is simultaneously amplified, it is necessary to consider designing a primer suppressing the formation of primer dimers.

Therefore, in a case where a bisulfite reaction or multiplex PCR is used for measuring the methylation degree of DNA of a predetermined site, unfortunately, designing a primer for multiplex PCR used for the analysis (that is, a primer for bisulfite amplicon sequence analysis) is more complicated compared to designing a primer for bisulfite-treated DNA and is time consuming.

As described above, most of the primer design software relates to general primer design software, and few software relates to the design of a primer for bisulfite-treated DNA. In addition, the primer design software for designing a primer for amplifying the bisulfite-treated DNA by multiplex PCR (that is, a primer for bisulfite amplicon sequence analysis) is fewer. Examples of the few available software include software described in WO2022/113835A proposed by the present inventors.

### SUMMARY OF THE INVENTION

In the bisulfite amplicon sequence analysis, generally, 5 to 1,000 target sites are preset as measurement targets, but it is desirable to output primer sequences at as many target sites as possible. That is, a high primer design success rate (the number of target sites for which the primer can be designed/total number of target sites [%]) is required.

The software described in WO2022/113835A can improve the primer design success rate as compared with the primer design software targeted for DNA subjected to the bisulfite treatment in the related art, but further improvement of the primer design success rate is required. In addition, even in a case where the primer design success rate can be improved, there is a possibility that a problem of a high probability of occurrence of primer dimers and a deterioration in the accuracy of the primer may occur.

In addition, in a case of designing a primer, the user selects the design success rate according to the state of each DNA sample and the content of the research, and thus does not only necessarily desire a primer having a high design success rate. However, there is an object that it takes time, effort, and cost to perform a plurality of primer designs according to the design success rate.

The present invention has been made to address the above object, and an object thereof is to provide a design method, a manufacturing method, a design device, a design program, and a recording medium for a primer for bisulfite amplicon sequence analysis (more specifically, a primer for amplicon methylation sequence analysis) which can further improve a design success rate of the primer.

In addition, an object of the present invention is to provide a design method, a manufacturing method, a design device, a design program, and recording medium for a primer for bisulfite amplicon sequence analysis (more specifically, a primer for amplicon methylation sequence analysis) which enable easy realization of primer design according to the design success rate at which a user desires.

[1] A primer design method for amplicon methylation sequence analysis according to the present invention is a method for designing a primer for amplicon methylation sequence analysis, the method utilizing a bisulfite reaction or an enzyme reaction and a multiplex PCR for measuring a methylation degree of at least one double-stranded genomic DNA and being used for simultaneously amplifying a plurality of regions each including two or more target sites where the methylation degree is measured, the design method comprising:
a complementary strand generation step of generating a complementary strand with respect to a template strand of the DNA;
a partial sequence cutting step of selecting one target site from the two or more target sites and, from each of the strands, cutting out one or more partial sequences having a predetermined length from a base sequence located on a 5′ terminal side of the selected target site;
a primer candidate sequence selection step of selecting the one or more cut-out partial sequences as one or more primer candidate sequences;
a primer sequence determination step of adopting and determining a forward primer sequence and a reverse primer sequence for amplifying a region including the selected predetermined target site from the one or more primer candidate sequences; and
a repeating step of repeating the partial sequence cutting step, the primer candidate sequence selection step, and the primer sequence determination step until all of the two or more target sites are selected in the partial sequence cutting step,
in which (I) in a case where one or more primer sequences of a different target site have not yet been determined, the primer sequence determination step includes [1] selecting one or more primer candidate sequence pairs related to the predetermined target site from the one or more primer candidate sequences, [2] selecting one primer candidate sequence pair from the one or more primer candidate sequence pairs of the predetermined target site, and calculating a local alignment score between sequences of the selected primer candidate sequence pair, and [3] adopting and determining the primer candidate sequence pair for which the local alignment score being less than a predetermined threshold value is calculated as the forward primer sequence and the reverse primer sequence for amplifying the region including the predetermined target site, (II) in a case where one or more primer sequences of the different target site have already been determined, the primer sequence determination step includes [1] selecting one or more primer candidate sequence pairs related to the predetermined target site from the one or more primer candidate sequences, [2] selecting one primer candidate sequence pair from the one or more primer candidate sequence pairs of the predetermined target site, and calculating a local alignment score between each of candidate sequences of the selected primer candidate sequence pair and each of the already determined primer sequences of the different target site, and a local alignment score between the sequences of the selected primer candidate sequence pair, and [3] detecting a maximum value from all the calculated local alignment scores, and adopting and determining a primer candidate sequence pair for which a local alignment score having the maximum value being equal to or less than a predetermined threshold value is calculated, as the forward primer sequence and the reverse primer sequence for amplifying the region including the predetermined target site,
in the step [3] of the (I) and the (II), in a case where the primer candidate sequence pair is not adopted as the forward primer sequence and the reverse primer sequence for amplifying the region including the predetermined target site, one different pair is selected from the one or more primer candidate sequence pairs selected in the step [1] of the (I) and the (II), and the steps [2] and [3] are repeated until at least one primer candidate sequence pair is adopted,
in a case where <1> a complementary base pair is set to “X” per pair, <2> a non-complementary base pair is set to “Y” per pair, and <3> a case where there is insertion or deletion is set to “Z” per one insertion or deletion between the primer candidate sequences, the local alignment score is calculated using “X” of 1, “Y” of −4 to −2, and “Z” of −6 to −3, and
the predetermined threshold value is 1 to 4.

[2] The primer design method for amplicon methylation sequence analysis according to [1], in which in the primer sequence determination step, (I) in the case where the number of the target sites is two or more and one or more primer sequences of a different target site have not yet been determined, in the step [2], all pairs are selected from the one or more primer candidate sequence pairs of the predetermined target site, and for each pair, a local alignment score between sequences of the selected primer candidate sequence pair is calculated, and in the step [3], one or more primer candidate sequence pairs for which the local alignment score being equal to or less than the predetermined threshold value is calculated are selected, and a primer candidate sequence pair having a smallest value of the maximum value of the local alignment score is further detected from all the selected pairs, and is adopted and determined as the forward primer sequence and the reverse primer sequence for amplifying the region including the predetermined target site, and (II) in the case where one or more primer sequences of the different target site have already been determined, in the step [2], all pairs are selected from the one or more primer candidate sequence pairs of the predetermined target site, and for each pair, a local alignment score between each of candidate sequences of the selected primer candidate sequence pair and each of the already determined primer sequences of the different target site, and a local alignment score between the sequences of the selected primer candidate sequence pair are calculated, and in the step [3], for each pair, a maximum value is detected from all the calculated local alignment scores, a primer candidate sequence pair for which a local alignment score having the maximum value being equal to or less than a predetermined threshold value is calculated is selected, and a primer candidate sequence pair having a smallest value of the maximum value of the local alignment score is further detected from all the selected pairs, and is adopted and determined as the forward primer sequence and the reverse primer sequence for amplifying the region including the predetermined target site.

[3] The primer design method for amplicon methylation sequence analysis according to [1] or [2], the design method further comprising:
a base sequence data acquisition step of acquiring base sequence data of the double-stranded genomic DNA;
a target site information acquisition step of acquiring the two or more target sites and position information of the target sites; and
a base conversion step of converting “C” which is methylatable in the double-stranded genomic DNA into “Y” and converting the other “C” into “T” in the base sequence data,
wherein in the complementary strand generation step, a complementary strand is generated for each template strand of the double-stranded genomic DNA after the base conversion,
in the partial sequence cutting step, one target site is selected from the two or more target sites, and from each of the strands, one or more partial sequences having a predetermined length are cut out from a base sequence located on a 5′ terminal side of the “Y” obtained by conversion of the selected target site or “R” complementary to the “Y”, based on the position information of the selected target site,
in the primer candidate sequence selection step, a partial sequence satisfying a predetermined selection condition is selected from the one or more partial sequences cut out from each of the strands, as the primer candidate sequence,
the methylatable “C” is “C” in a CG sequence, and
the predetermined selection condition includes (1) a Tm value is within a predetermined range, (2) the number of YG sequences or CR sequences included in the partial sequence is equal to or less than predetermined number, and (3) an upper limit of the number of binding sites with a sequence outside a related region on the double-stranded genomic DNA after the base conversion is equal to or less than a predetermined number that is equal to or more than 1,
[provided that “C”, “G”, “Y”, and “R” are base codes established by IUPAC, “C” represents cytosine, “G” represents guanine, “Y” represents thymine or cytosine, and “R” represents adenine or guanine].

[4] The primer design method according to [3], in which the methylatable “C” further includes “C” in a CHG sequence, and the predetermined selection condition further includes (4) the number of YHG sequences or CDR sequences included in the partial sequence is equal to or less than a predetermined number,
[provided that “C”, “G”, “Y”, “H”, “R”, and “D” are base codes established by IUPAC, “C” represents cytosine, “G” represents guanine, “Y” represents thymine or cytosine, “H” represents adenine, cytosine, or thymine, “D” represents thymine, guanine, or adenine, and “R” represents adenine or guanine].

[5] The primer design method according to [3] or [4], in which the methylatable “C” further includes “C” in a CHH sequence, and the predetermined selection condition further includes (5) the number of YHH sequences or DDR sequences included in the partial sequence is equal to or less than a predetermined number,
[provided that, “Y”, “H”, “R”, and “D” are base notations determined by IUPAC, “Y” represents thymine or cytosine, “H” represents adenine, cytosine, or thymine, “D” represents thymine, guanine, or adenine, and “R” represents adenine or guanine].

[6] The primer design method according to any one of [3] to [5], in which in the primer candidate sequence selection step, the double-stranded genomic DNA after the base conversion is divided into a first template strand and a second template strand, a complementary strand of the first template strand is a first complementary strand, a complementary strand of the second template strand is a second complementary strand, and
the primer candidate sequence selection step is a step of selecting a partial sequence satisfying a predetermined selection condition as a forward primer candidate sequence of the first template strand from one or more partial sequences cut out from the first template strand, selecting a partial sequence satisfying the predetermined selection condition as a reverse primer candidate sequence of the first template strand from one or more partial sequences cut out from the first complementary strand, selecting a partial sequence satisfying the predetermined selection condition as a forward primer candidate sequence of the second template strand from one or more partial sequences cut out from the second template strand, and selecting a partial sequence satisfying the predetermined selection condition as a reverse primer candidate sequence of the second template strand from one or more partial sequences cut out from the second complementary strand.

[7] The primer design method according to any one of [3] to [6], in which the primer sequence determination step is a step of calculating a length of a PCR amplification product predicted to be amplified by PCR for all combinations of the one or more forward primer candidate sequences of the first template strand and the one or more reverse primer candidate sequences of the first template strand selected in the primer candidate sequence selection step, adopting a combination of primer candidate sequences for which the calculated length of the PCR amplification product is within a predetermined range as a forward primer sequence and a reverse primer sequence of the first template strand for amplifying a region including the target site selected in the partial sequence cutting step, calculating a length of a PCR amplification product predicted to be amplified by PCR for all combinations of the one or more forward primer candidate sequences of the second template strand and the one or more reverse primer candidate sequences of the second template strand selected in the primer candidate sequence selection step, and adopting and determining a combination of primer candidate sequences for which the calculated length of the PCR amplification product is within a predetermined range as a forward primer sequence and a reverse primer sequence of the second template strand for amplifying the region including the target site selected in the partial sequence cutting step.

[8] The primer design method according to any one of [1] to [7], in which in advance, a correspondence relationship between at least the number of the target sites, the predetermined threshold value, and a primer design success rate is measured using the primer design method for amplicon methylation sequence analysis according to any one of [1] to [7], and the correspondence relationship is stored in a storage unit,
in a case where a user sets at least the primer design success rate desired by the user and the number of the target sites via an input unit and gives an instruction to execute primer design, the predetermined threshold value corresponding to the primer design success rate and the number of the target sites, which are equal to or greater than set values and have a small difference, is read out from the correspondence relationship stored in the storage unit, and
a primer sequence for amplifying a region including the predetermined target site is adopted and determined from the one or more primer candidate sequences based on the read-out predetermined threshold value.

[9] A manufacturing method for a primer for amplicon methylation sequence analysis in the present invention comprising:
a primer design step according to any one of [1] to [8]; and
a synthesis step of synthesizing a primer based on a primer sequence designed in the primer design step,
in which the primer design step is performed by the primer design method for amplicon methylation sequence analysis described above.

[10] A primer design device for amplicon methylation sequence analysis in the present invention is a device for designing a primer for amplicon methylation sequence analysis, the device utilizing a bisulfite reaction or an enzyme reaction and a multiplex PCR for measuring a methylation degree of at least one double-stranded DNA and being used for simultaneously amplifying a plurality of regions each including two or more target sites where the methylation degree is measured, the design device comprising:
a complementary strand generation unit that generates a complementary strand with respect to a template strand of the DNA;
a partial sequence cutting unit that selects one target site from the two or more target sites and, from each of the strands, cuts out one or more partial sequences having a predetermined length from a base sequence located on a 5′ terminal side of the selected target site;
a primer candidate sequence selection unit that selects the one or more cut-out partial sequences as one or more primer candidate sequences;
a primer sequence determination unit that adopts and determines a forward primer sequence and a reverse primer sequence for amplifying a region including the selected predetermined target site from the one or more primer candidate sequences; and
a control unit that performs control configured to repeat each processing in the partial sequence cutting unit, the primer candidate sequence selection unit, and the primer sequence determination unit until all of the two or more target sites are selected in the partial sequence cutting unit,
in which (I) in a case where one or more primer sequences of a different target site have not yet been determined, the primer sequence determination unit performs the following steps, [1] selecting one or more primer candidate sequence pairs related to the predetermined target site from the one or more primer candidate sequences, [2] selecting one primer candidate sequence pair from the one or more primer candidate sequence pairs of the predetermined target site, and calculating a local alignment score between sequences of the selected primer candidate sequence pair, and [3] adopting and determining the primer candidate sequence pair for which the local alignment score being equal to or less than a predetermined threshold value is calculated as the forward primer sequence and the reverse primer sequence for amplifying the region including the predetermined target site, (II) in a case where one or more primer sequences of the different target site have already been determined, the primer sequence determination unit performs the following steps, [1] selecting one or more primer candidate sequence pairs related to the predetermined target site from the one or more primer candidate sequences, [2] selecting one primer candidate sequence pair from the one or more primer candidate sequence pairs of the predetermined target site, and for each pair, calculating a local alignment score between each of candidate sequences of the selected primer candidate sequence pair and each of the already determined primer sequences of the different target site, and a local alignment score between the sequences of the selected primer candidate sequence pair, and [3] detecting a maximum value from all the calculated local alignment scores, and adopting and determining a primer candidate sequence pair for which a local alignment score having the maximum value being equal to or less than a predetermined threshold value is calculated, as the forward primer sequence and the reverse primer sequence for amplifying the region including the predetermined target site,
in the step [3] of the (I) and the (II), in a case where the primer candidate sequence pair is not adopted as the forward primer sequence and the reverse primer sequence for amplifying the region including the predetermined target site, one different pair is selected from the one or more primer candidate sequence pairs selected in the step [1] of the (I) and the (II), and the steps [2] and [3] are repeated until at least one primer candidate sequence pair is adopted,
in a case where <1> a complementary base pair is set to “X” per pair, <2> a non-complementary base pair is set to “Y” per pair, and <3> a case where there is insertion or deletion is set to “Z” per one insertion or deletion between the primer candidate sequences, the local alignment score is calculated using “X” of 1, “Y” of −4 to −2, and “Z” of −6 to −3, and
the predetermined threshold value is 1 to 4.

[11] The primer design device for amplicon methylation sequence analysis according to [10], in which in the primer sequence determination unit, (I) in the case where one or more primer sequences of a different target site have not yet been determined, in the step [2], all pairs are selected from the one or more primer candidate sequence pairs of the predetermined target site, and for each pair, a local alignment score between sequences of the selected primer candidate sequence pair is calculated, and in the step [3], one or more primer candidate sequence pairs for which the local alignment score being equal to or less than the predetermined threshold value is calculated are selected, and a primer candidate sequence pair having a smallest value of the maximum value of the local alignment score is further detected from all the selected pairs, and is adopted and determined as the forward primer sequence and the reverse primer sequence for amplifying the region including the predetermined target site, and (II) in the case where one or more primer sequences of a different target site have already been determined, in the step [2], all pairs are selected from the one or more primer candidate sequence pairs of the predetermined target site, and for each pair, a local alignment score between each of candidate sequences of the selected primer candidate sequence pair and each of the already determined primer sequences of the different target site, and a local alignment score between the sequences of the selected primer candidate sequence pair are calculated, and in the step [3], for each pair, a maximum value is detected from all the calculated local alignment scores, a primer candidate sequence pair for which a local alignment score having the maximum value being equal to or less than a predetermined threshold value is calculated is selected, and a primer candidate sequence pair having a smallest value of the maximum value of the local alignment score is further detected from all the selected pairs, and is adopted and determined as the forward primer sequence and the reverse primer sequence for amplifying the region including the predetermined target site.

[12] The primer design device for amplicon methylation sequence analysis according to or [11], the design device further comprising:
a base sequence data acquisition unit that acquires base sequence data of the double-stranded genomic DNA;
a target site information acquisition unit that acquires the two or more target sites and position information of the target sites; and
a base conversion unit that converts “C” which is methylatable in the double-stranded genomic DNA into “Y” and converts the other “C” into “T” in the base sequence data,
in which in the complementary strand generation unit, a complementary strand is generated for each template strand of the double-stranded genomic DNA after the base conversion,
in the partial sequence cutting unit, one target site is selected from the two or more target sites, and from each of the strands, one or more partial sequences having a predetermined length are cut out from a base sequence located on a 5′ terminal side of the “Y” obtained by conversion of the selected target site or “R” complementary to the “Y”, based on the position information of the selected target site,
in the primer candidate sequence selection unit, a partial sequence satisfying a predetermined selection condition is selected from the one or more partial sequences cut out from each of the strands, as the primer candidate sequence,
the methylatable “C” is “C” in a CG sequence, and
the predetermined selection condition includes (1) Tm is within a predetermined range, (2) the number of YG sequences or CR sequences included in the partial sequence is equal to or less than predetermined number, and (3) an upper limit of the number of binding sites with a sequence outside a related region on the double-stranded genomic DNA after the base conversion is equal to or less than a predetermined number that is equal to or more than 1,
[provided that “C”, “G”, “Y”, and “R” are base codes established by IUPAC, “C” represents cytosine, “G” represents guanine, “Y” represents thymine or cytosine, and “R” represents adenine or guanine].

[13] The primer design device according to [12], in which the methylatable “C” further includes “C” in a CHG sequence, and
the predetermined selection condition further includes (4) the number of YHG sequences or CDR sequences included in the partial sequence is equal to or less than a predetermined number,
[provided that “C”, “G”, “Y”, “H”, “R”, and “D” are base codes established by IUPAC, “C” represents cytosine, “G” represents guanine, “Y” represents thymine or cytosine, “H” represents adenine, cytosine, or thymine, “D” represents thymine, guanine, or adenine, and “R” represents adenine or guanine].

[14] The design device for a primer according to or [13], in which the methylatable “C” further includes “C” in a CHH sequence, and
the predetermined selection condition further includes (5) the number of YHH sequences or DDR sequences included in the partial sequence is equal to or less than a predetermined number,
[provided that “C”, “G”, “Y”, “H”, “R”, and “D” are base codes established by IUPAC, “C” represents cytosine, “G” represents guanine, “Y” represents thymine or cytosine, “H” represents adenine, cytosine, or thymine, “D” represents thymine, guanine, or adenine, and “R” represents adenine or guanine].

[15] The primer design device according to any one of to [14], in which in the primer candidate sequence selection unit, the double-stranded genomic DNA after the base conversion is divided into a first template strand and a second template strand, a complementary strand of the first template strand is a first complementary strand, a complementary strand of the second template strand is a second complementary strand, and
the primer candidate sequence selection unit is a unit that selects a partial sequence satisfying a predetermined selection condition as a forward primer candidate sequence of the first template strand from one or more partial sequences cut out from the first template strand, selects a partial sequence satisfying the predetermined selection condition as a reverse primer candidate sequence of the first template strand from one or more partial sequences cut out from the first complementary strand, selects a partial sequence satisfying the predetermined selection condition as a forward primer candidate sequence of the second template strand from one or more partial sequences cut out from the second template strand, and selects a partial sequence satisfying the predetermined selection condition as a reverse primer candidate sequence of the second template strand from one or more partial sequences cut out from the second complementary strand.

[16] The primer design device according to [15], in which the primer sequence determination unit is a unit that calculates a length of a PCR amplification product predicted to be amplified by PCR for all combinations of the one or more forward primer candidate sequences of the first template strand and the one or more reverse primer candidate sequences of the first template strand selected in the primer candidate sequence selection unit, adopts a combination of primer candidate sequences for which the calculated length of the PCR amplification product is within a predetermined range as a forward primer sequence and a reverse primer sequence of the first template strand for amplifying a region including the target site selected in the partial sequence cutting unit, calculates a length of a PCR amplification product predicted to be amplified by PCR for all combinations of the one or more forward primer candidate sequences of the second template strand and the one or more reverse primer candidate sequences of the second template strand selected in the primer candidate sequence selection unit, and adopts and determines a combination of primer candidate sequences for which the calculated length of the PCR amplification product is within a predetermined range as a forward primer sequence and a reverse primer sequence of the second template strand for amplifying the region including a target site selected in the partial sequence cutting unit.

[17] The primer design device for amplicon methylation sequence analysis according to [10], further comprising:
a storage unit that measures a correspondence relationship between at least the number of the target sites, the predetermined threshold value, and a primer design success rate in advance using the primer design device according to [10], and stores the correspondence relationship; and
an input unit through which a user inputs an instruction,
in which, in the primer sequence determination unit, in a case where the user sets at least the primer design success rate desired by the user and the number of the target sites via the input unit and gives an instruction to execute primer design, the predetermined threshold value corresponding to the primer design success rate and the number of the target sites, which are equal to or greater than set values and have a small difference, is read out from the correspondence relationship stored in the storage unit, and a primer sequence for amplifying a region including the predetermined target site is adopted and determined from the one or more primer candidate sequences based on the read-out predetermined threshold value.

The primer design device for amplicon methylation sequence analysis according to any one of [12] to [17] further comprises a communication interface, in which the design device is capable of being connected to a server via an external communication network by the communication interface and is capable of operating at least one unit selected from the group consisting of the base sequence data acquisition unit, the target site information acquisition unit, the base conversion unit, the complementary strand generation unit, the partial sequence cutting unit, the primer candidate sequence selection unit, and the primer sequence determination unit by programs in the server.

The design program for a primer for amplicon methylation sequence analysis according to any one of [1] to [8] of the present invention is a program that can execute the above-described primer design method on a computer.

The recording medium readable by the computer described in in the present invention is a recording medium on which the design program of the primer for amplicon methylation sequence analysis described above is recorded.

According to the present invention, it is possible to further improve the design success rate of a primer for bisulfite amplicon sequencing analysis (more specifically, a primer for amplicon methylation sequence analysis) as compared with the related art, and it is also possible to suppress the probability of occurrence of primer dimers low. In addition, a primer based on the design of the present invention can be obtained. As a result, many target sites can be amplified and measured.

According to the present invention, it is possible to design a primer for bisulfite amplicon sequence analysis (more specifically, a primer for amplicon methylation sequence analysis) according to a desired design success rate of the user, more easily and in a short time. In addition, a primer based on the design can be obtained.

### BRIEF DESCRIPTION OF THE DRAWINGS

FIG. 1 is a block diagram conceptually showing an example of the configuration of a primer design device according to a first embodiment of the present invention.

FIG. 2 is a flowchart showing an example of a primer design method according to the first embodiment performed by the primer design device shown in FIG. 1.

FIG. 3A is a schematic view for illustrating a base sequence data acquisition step of the primer design method shown in FIG. 2.

FIG. 3B is a schematic view for illustrating a base conversion step of the primer design method shown in FIG. 2.

FIG. 3C is a schematic view for illustrating a complementary strand generation step of the primer design method shown in FIG. 2.

FIG. 3D is a schematic view for illustrating a partial sequence cutting step of the primer design method shown in FIG. 2.

FIG. 4 is a flowchart showing an example of the operation of a partial sequence cutting unit 28, a primer candidate sequence selection unit 30, and a primer sequence determination unit 32.

FIG. 5A is a view for illustrating the condition (3) “the upper limit of the number of binding sites with a sequence outside the related region on the double-stranded genomic DNA after base conversion is equal to or less than a predetermined number that 1 or more”.

FIG. 5B is a view for illustrating the condition (3) “the upper limit of the number of binding sites with a sequence outside the related region on the double-stranded genomic DNA after base conversion is equal to or less than a predetermined number that 1 or more”.

FIG. 6A is a diagram for describing a combination of sequence comparisons related to local alignment score calculation.

FIG. 6B is a diagram for describing a combination of sequence comparisons related to the local alignment score calculation.

FIG. 7A is a diagram for describing a combination of sequence comparisons related to local alignment score calculation.

FIG. 7B is a diagram for describing a combination of sequence comparisons related to local alignment score calculation.

FIG. 8 is a diagram for describing a method of calculating a local alignment score and a determination method based on a threshold value.

FIG. 9 is a diagram showing a correspondence relationship between the number of target sites, a threshold value, and a primer design success rate, which is stored in a storage unit of the primer design device according to Modification Example 2 of Example 1 of the present invention.

FIG. 10 is a block diagram conceptually showing an example of the configuration of a primer design device according to a second embodiment of the present invention.

FIG. 11 is a block diagram conceptually showing an example of the connection between the primer design device according to the second embodiment of the present invention and an external server.

FIG. 12A is a graph showing the primer design success rate of Examples 1 to 4 and Comparative Examples 2 to 4.

FIG. 12B is a graph showing the primer dimer formation rate of Examples 1 to 4 and Comparative Examples 2 to 4.

FIG. 13A is a schematic view for illustrating an example of a method of analyzing the methylation status of DNA using a bisulfite reaction.

FIG. 13B is a schematic view for illustrating an example of a method of analyzing methylation status (frequency) of DNA using a bisulfite reaction.

FIG. 13C is a view for illustrating a target site (measurement site) and an amplification target region.

### DESCRIPTION OF THE PREFERRED EMBODIMENTS

Hereinafter, based on public embodiments shown in the accompanying drawings, a design method, a manufacturing method, a design device, a design program and a recording medium for a primer for a bisulfite amplicon sequence (a primer for amplicon methylation sequence analysis) according to embodiments of the present invention will be specifically described.

### EXPLANATION OF TERMS

In the present specification, “primer for bisulfite amplicon sequence analysis” means a primer for analysis that is for simultaneously amplifying a plurality of amplification target regions each including a plurality of target sites in bisulfite-treated DNA by multiplex PCR.

“Primer for bisulfite amplicon methylation sequence analysis” means a primer for analysis that is for simultaneously amplifying a plurality of amplification target regions each including a plurality of target sites in bisulfite-treated or enzyme-treated DNA by multiplex PCR.

“Amplification target region” means a region to be amplified by a primer pair.

“Methylation site” means a methylatable site.

“Target site” is a “methylation site” which refers to a site (measurement site) for measuring a methylation degree.

The “primer candidate sequence” means any one of a forward candidate primer sequence or a reverse candidate primer sequence, unless otherwise specified.

The “primer candidate sequence pair” means one combination of a forward candidate primer sequence and a reverse candidate primer sequence.

The “primer sequence” means any of a forward primer sequence or a reverse primer sequence, unless otherwise specified.

The “primer sequence pair” means one combination of a forward primer sequence and a reverse primer sequence.

The base sequences such as “GC sequence” and “YG sequence” all mean sequences read from the 5′ terminal side.

A range described using “to” is regarded as including both sides of “to”. For example, a range described as “A to B” includes A and B.

### First Embodiment

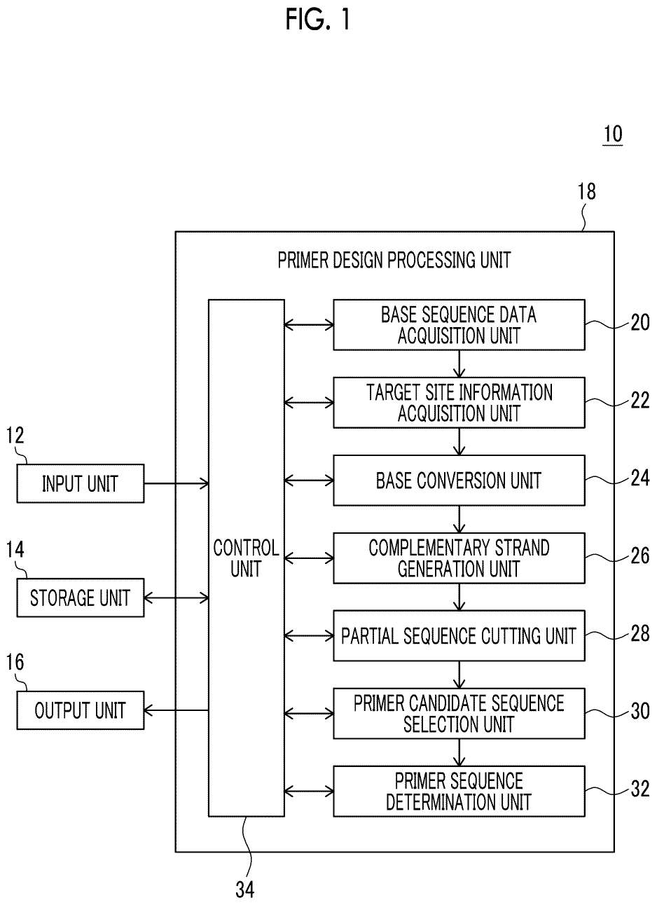

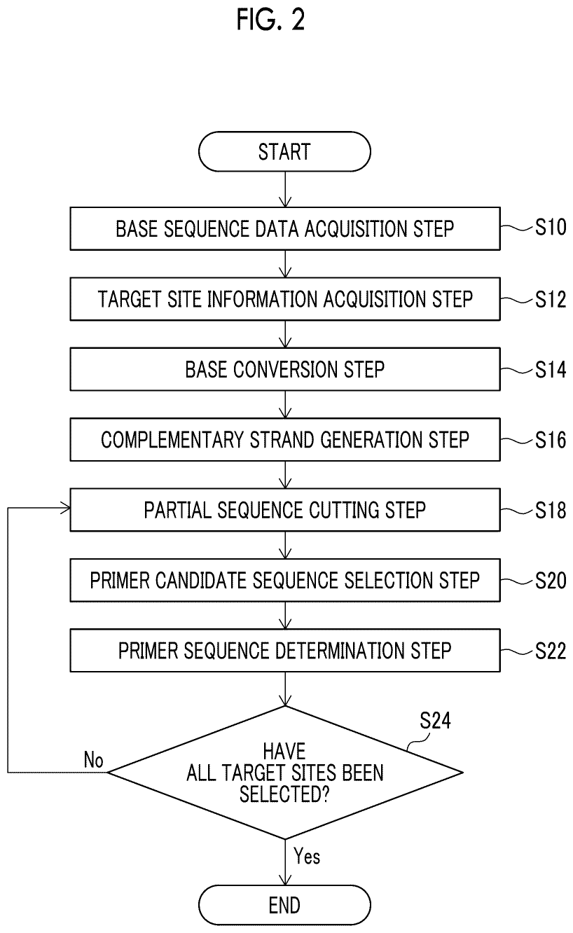

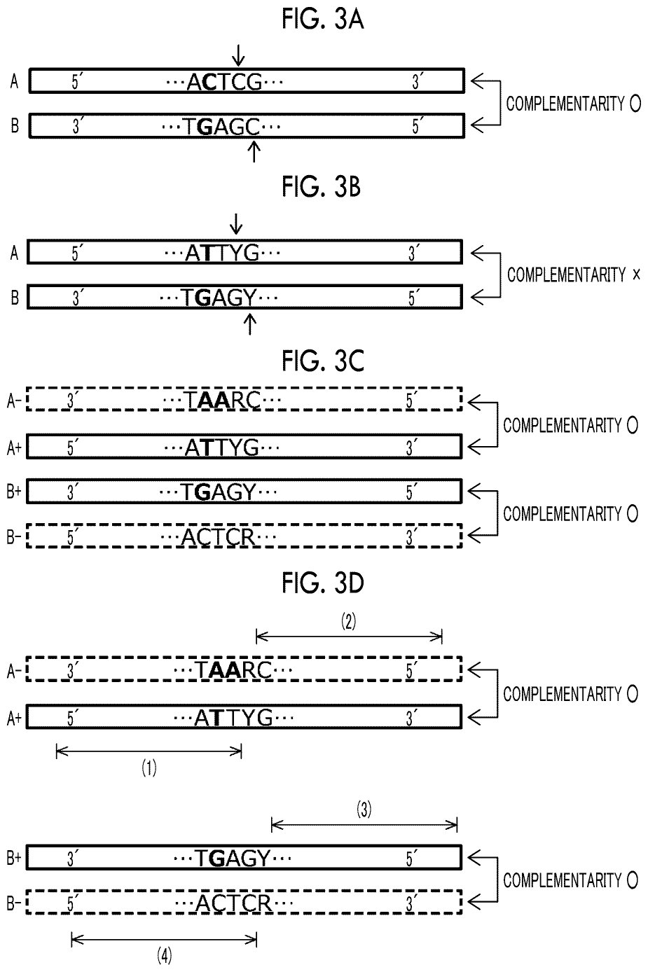

FIG. 1 is a block diagram conceptually showing an example a primer design device according to a first embodiment of the present invention. FIG. 2 is a flowchart showing an example of a primer design method performed by the primer design device shown in FIG. 1. FIGS. 3A to 3D are schematic views for illustrating each step of the primer design method.

As shown in FIG. 1, a primer design device 10 comprises an input unit 12, a storage unit 14, an output unit 16, and a primer design processing unit 18. The input unit 12, the storage unit 14, the output unit 16, and the primer design processing unit 18 are connected to each other.

The input unit 12 is a unit that acquires information input by the user, various setting instructions, selection instructions, input instructions, creation instructions, and the like, and is configured with, for example, an input device such as a keyboard and a mouse.

The storage unit 14 stores an operation program of the primer design device, and can also temporarily store information and data necessary for executing primer design processing. As the storage unit 14, for example, it is possible to use recording media such as a hard disc drive (HDD), a solid state drive (SSD), a flexible disc (FD), a magneto-optical (MO) disc, a magnetic tape (MT), a random access memory (RAM), a compact disc (CD), a digital versatile disc (DVD), a secure digital (SD) card, a universal serial bus (USB) memory, and the like.

The output unit 16 is a unit that outputs DNA base sequence information, instructions, design conditions, primer sequence information designed by the primer design processing unit 18, and the like which are input from the input unit 12, and is configured with, for example, display units, such as a liquid crystal display (LCD), organic light-emitting diodes (OLED), flat panel displays, individual displays, and cathode ray tubes (CRT), various types of printers, and the like.

The primer design processing unit 18 is a unit that performs a series of processing for primer design.

The primer design processing unit 18 comprises a base sequence data acquisition unit 20, a target site information acquisition unit 22, a base conversion unit 24, a complementary strand generation unit 26, a partial sequence cutting unit 28, a primer candidate sequence selection unit 30, a primer sequence determination unit 32, and a control unit 34.

The primer design processing unit 18 can be configured with a processor including a central processing unit (CPU) or the like, a computer, and the like.

As shown in FIG. 2, a primer design method includes a base sequence data acquisition step S10, a target site information acquisition step S12, a base conversion step S14, a complementary strand generation step S16, a partial sequence cutting step S18, a primer candidate sequence selection step S20, a primer sequence determination step S22, and a repetition step of repeating the partial sequence cutting step S18, the primer candidate sequence selection step S20, and the primer sequence determination step S22 until all target sites are detected by a determination step S24.

### (Base Sequence Data Acquisition Unit)

The base sequence data acquisition unit 20 shown in FIG. 1 is a unit that performs the base sequence data acquisition step S10 shown in FIG. 2, and acquires the data of the double-stranded DNA sequence (reference sequence) of the genome of biological species, for which a primer is to be designed, via the input unit 12. In a case where the data of the reference sequence is stored in the storage unit 14 in advance, the data may be acquired from the storage unit 14.

It is preferable that the data of the double-stranded genomic DNA sequence to be acquired be the data of the complete sequence of the genome of the biological species for which a primer is to be designed.

In order to explain the primer design method of the present embodiment, the double-stranded DNA of the double-stranded DNA sequence data acquired in this step will be called template DNA which will be referred to as a strand A and a strand B, respectively (see FIG. 3A).

The base sequence data acquisition unit 20 is configured with a computer and functions to acquire the data of the double-stranded DNA sequence of the genome described above.

### (Target Site Information Acquisition Unit)

The target site information acquisition unit 22 shown in FIG. 1 is a unit that performs the target site information acquisition step S12 shown in FIG. 2, and can acquire one or more target sites included in the double-stranded genomic DNA acquired by the base sequence data acquisition unit 20 and the position information of the target sites via the input unit 12. In a case where the target sites and the position information thereof are stored in the storage unit 14 in advance, the target sites and the position information thereof may be acquired from the storage unit 14.

“Target site” is a site related to a predetermined biological phenomenon, is cytosine (C) of a CG sequence which is methylatable cytosine (C), and is a site for measuring a methylation degree.

The number of target sites to be selected is not particularly limited as long as it is 2 or more. From the viewpoint of markedly obtaining the desired effect of the present invention, it is preferable to select 5 to 1,000 sites.

The position of each target site can be indicated by a chromosome, a genomic coordinate, or the like.

The target site information acquisition unit 22 is configured with a computer and functions to acquire two or more target sites included in the aforementioned double-stranded genomic DNA and position information thereof.

### (Base Conversion Unit)

The base conversion unit 24 is a unit that performs the base conversion step S14 shown in FIG. 2. As shown in FIGS. 3A and 3B, the base conversion unit 24 converts cytosine (C) of a CG sequence on the template DNA acquired from the base sequence data acquisition unit 20 into “Y” (see the bases indicated by the arrows in FIGS. 3A and 3B) and converts cytosine (C) of other sequences into thymine (T). Cytosine (C) in a CG sequence of DNA is likely to be methylated or unmethylated. Therefore, cytosine (C) is converted into “Y” having both the possibility of being converted into thymine (T) and the possibility of remaining as cytosine (C).

Note that this conversion processing is computer simulation that reproduces the generation of DNA amplified by PCR after a bisulfite treatment.

The base conversion unit 24 is configured with a computer and functions to convert cytosine (C) of the CG sequence on the aforementioned template DNA into “Y” and cytosine (C) of other sequences into thymine (T).

As described above, due to the bisulfite treatment, the DNA double strands lose the complementarity thereof. This is because the bisulfite treatment induces the conversion of the cytosine (C) of the CG base pair having complementarity into the thymine (T), which removes the complementarity of the base pair (see the bolded bases in FIGS. 3A and 3B). With one set of primers, it is impossible to equally amplify both strands of the amplification target region on the bisulfite-treated DNA having lost the complementarity in this way. Therefore, in a case where the methylation status of double-stranded DNA is to be analyzed, a primer pair (a forward primer and a reverse primer) for amplifying an amplification target region including each target site of each strand needs to be prepared for each target site. That is, it is necessary to design a primer pair related to the amplification target region including the target site of the strand A after base conversion in FIG. 3B and a primer pair related to the amplification target region including the target site of the strand B after base conversion, respectively.

Provided that, as will be explained in Modification Example 5 that will be described later, in a case where the user wants to analyze only the strand A or strand B, or in a case where it will be fine if either the strand A or the strand B can be analyzed, it is not necessary to design two sets of primer pair.

### (Complementary Strand Generation Unit)

The complementary strand generation unit 26 is a unit that performs the complementary strand generation step S16 shown in FIG. 2, and generates a complementary strand for each of two DNA strands after the base conversion processing.

In order to illustrate the primer design method of the present embodiment, the strand A after base conversion and the strand B after base conversion will be called a first template strand (strand A+) and a second template strand (strand B+) respectively, and a complementary strand of the first template strand and a complementary strand of the second template strand will be called a first complementary strand (strand A−) and a second complementary strand (strand B−) respectively (see FIG. 3C).

As shown in FIG. 3C, a sequence complementary to the base sequence of the strand A+ is generated to prepare a complementary strand A−, and a sequence complementary to the base sequence of the strand B+ is generated to prepare a complementary strand B−. The base complementary to “Y” is denoted by “R” having both the possibility of being adenine (A) and the possibility of being guanine (G).

The complementary strand generation unit 26 is configured with a computer and functions to generate the aforementioned complementary strand for each of the two strands of DNA after base conversion processing.

As a result, the first template strand (strand A+) is configured with three bases of thymine (T), adenine (A), and guanine (G) excluding “Y” (that is, a methylation site), the first complementary strand (strand A−) is configured with three bases of thymine (T), adenine (A), and cytosine (C) excluding “R” (a methylation site), and the first template strand (strand A+) and the first complementary strand (strand A−) can have complementarity.

Likewise, the second template strand (strand B+) is configured with three bases of thymine (T), adenine (A), and guanine (G) excluding “Y” (a methylation site), the second complementary strand (strand B−) is configured with three bases of thymine (T), adenine (A), and cytosine (C) excluding “R” (a methylation site), and the second template strand (strand B+) and the second complementary strand (strand B−) can have complementarity.

### (Partial Sequence Cutting Unit)

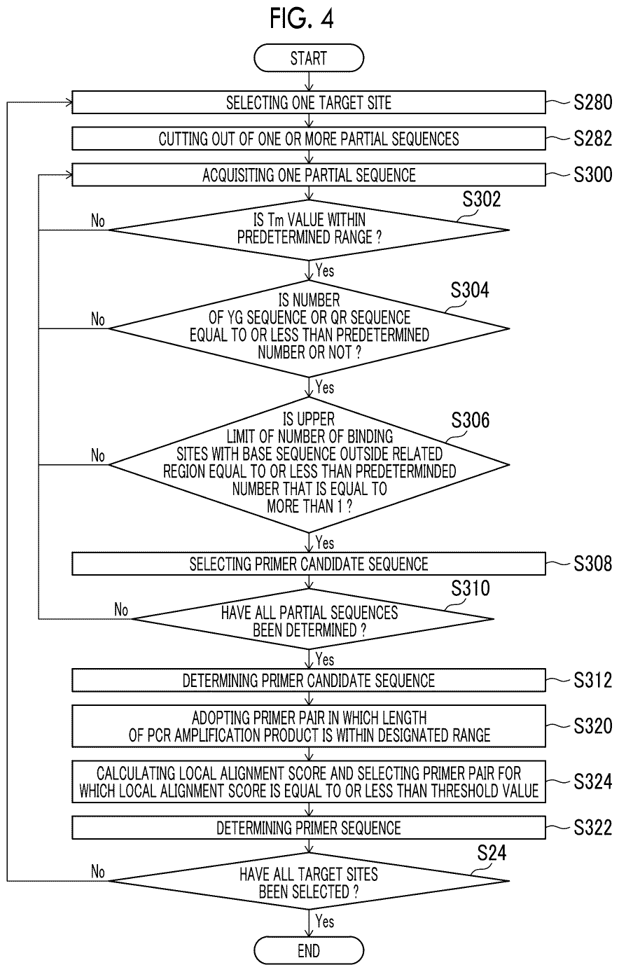

The partial sequence cutting unit 28 is a unit that performs the partial sequence cutting step S18 shown in FIG. 2. As shown in the flowchart of FIG. 4, the partial sequence cutting unit 28 selects one target site from two or more target sites acquired by the target site information acquisition unit 22 (step S280), detects “Y” of the selected target site or “R” (that is, a base which is in a methylation site in the target site) complementary to “Y” from the DNA sequence of each strand based on the position information of the selected target site, and cuts partial sequences as much as possible from the partial sequences having a predetermined length from the base sequences ((1) to (4) in FIG. 3D) positioned on 5′ terminal side of the detected “Y” or “R” (step S282) to obtain one or more partial sequences.

FIG. 4 is a flowchart showing an example of the operation of a partial sequence cutting unit 28, a primer candidate sequence selection unit 30, and a primer sequence determination unit 32.

The partial sequence cutting unit 28 is configured with a computer and functions to cut partial sequences as much as possible from partial sequences having a predetermined length from “Y” of the selected target site or “R” complementary to “Y” from the DNA sequence of each strand based on the position information of the selected target site described above to obtain one or more partial sequences.

The length of one or more partial sequences to be cut out is not particularly limited. From the viewpoint of processing efficiency and markedly obtaining the desired effect of the present invention, it is preferable that the length of one or more partial sequences to be cut out be equal to maximum length of PCR amplification product that the user desires-minimum length of primer-length (one base) of target site.

The length of the PCR amplification product is not particularly limited as long as it is in a known range, that is, 70 to several kilo base pairs. It is preferable to consider a PCR success rate, the sequencing ability of a DNA sequencer, and the like.

The length of the primer is not particularly limited as long as it is in a known range, that is, 15 to 45 bases. It is preferable to consider the specificity of the primer and the primer dimer forming properties.

For example, in a case where the maximum length of the PCR product set by the user is 300 bases and the minimum length of the primer is 20 bases, a predetermined length x to be cut out is calculated by x=300−20−1 (length of the target site), which is equal to 279. Therefore, first, 279 bases on 5′ terminal side of each target site are cut out. As shown in FIG. 3D, a target site of each strand, that is, 279 bases on 5′ terminal side of “Y” of the strand A+, “R” of the strand A−, “Y” of the strand B+, and “R” of the strand B− ((1) to (4) in FIG. 3D) are cut out from each strand.

Subsequently, by cutting partial sequences from the 279 bases in the length of the primer (equal to or less than a predetermined length consisting of 20 or more bases) as much as possible, it is possible to obtain one or more partial sequences.

The numerical value or numerical range of the length of the PCR amplification product and the length of the primer are set by the user via the input unit 12. In a case where these conditions are stored in the storage unit 14 in advance, these conditions can be set by being acquired from the storage unit 14.

### (Primer Candidate Sequence Selection Unit)

The primer candidate sequence selection unit 30 is a unit that performs the primer candidate sequence selection step S20 shown in FIG. 2, and selects partial sequences satisfying all the predetermined selection conditions (1) to (3) as primer candidate sequences from one or more partial sequences of each strand cut out by the partial sequence cutting unit 28.

Specifically, a partial sequence that satisfies the predetermined selection conditions is selected as a forward primer candidate sequence of the first template strand (strand A+) among one or more partial sequences cut out from the first template strand (strand A+) (that is, one or more partial sequences cut out from (1) in FIG. 3D), a partial sequence that satisfies the predetermined selection conditions is selected as a reverse primer candidate sequence of the first template strand (strand A+) among one or more partial sequences cut out from the first complementary strand (strand A−) (that is, one or more partial sequences cut out from (2) in FIG. 3D), a partial sequence that satisfies the predetermined selection conditions is selected as a forward primer candidate sequence of the second template strand (strand B+) among one or more partial sequences cut out from the second template strand (strand B+) (that is, one or more partial sequences cut out from (3) in FIG. 3D), and a partial sequence that satisfies the predetermined selection conditions is selected as a reverse primer candidate sequence of the second template strand (strand B+) among one or more partial sequences cut out from the second complementary strand (strand B−) (that is, one or more partial sequences cut out from (4) in FIG. 3D).

The primer candidate sequence selection unit 30 is configured with a computer and functions to select partial sequences that satisfy all the predetermined selection conditions (1) to (3) as primer candidate sequences from one or more partial sequences of each strand described above.

“Predetermined selection conditions” of the primer candidate sequences are conditions (1) to (3) described below. The user can preset the numerical value and numerical range of the predetermined selection conditions via the input unit 12.
(1) The Tm value is within a predetermined range
(2) The number of YG sequences or CR sequences included in a partial sequence is equal to or less than a predetermined number
(3) An upper limit of the number of binding sites with a base sequence outside a related region on the template strand DNA (double-stranded genomic DNA) after base conversion is equal to or less than a predetermined number that is equal to or more than 1

The range of “Tm value” related to the condition (1) is not particularly limited as long as it is in a known numerical range, that is, 45° C. to 70° C. It is preferable to consider the thermal cycle conditions of PCR, the ease of PCR amplification (the temperature range in which amplification can easily proceed by the PCR enzyme used), and the specificity of PCR amplification. The Tm value can be calculated by, for example, the nearest neighbor base pair method.

The number of “YG sequences or CR sequences included in a partial sequence” related to the condition (2) is not particularly limited. From the viewpoint of markedly obtaining the desired effect of the present invention, the number of YG sequences or CR sequences is preferably 2 or less, more preferably 1 or less, and particularly preferably 0.

In a case where the above condition is satisfied, the influence of the binding of the primer to cytosine (C) of the CG sequence in the primer binding site can be reduced.

“Sequence outside the related region on the template strand DNA (double-stranded genomic DNA) after base conversion” related to the condition (3) described above refers to the base sequence excluding the sequence at the position on the template strand DNA after base conversion, the position corresponding to the partial sequence, and a base sequence complementary to the sequence (the template strand DNA sequence after base conversion) excluding the partial sequence.

“Upper limit of the number of binding sites with the sequence outside the related region on the template strand DNA after base conversion” is not particularly limited. From the viewpoint of markedly obtaining the desired effect of the present invention, the upper limit of the number of such binding sites is preferably 5 or less, and particularly preferably 2 or less.

In a case where the above condition is satisfied, the influence of binding of the primer to the outside of the related region on the bisulfite-treated DNA can be reduced.

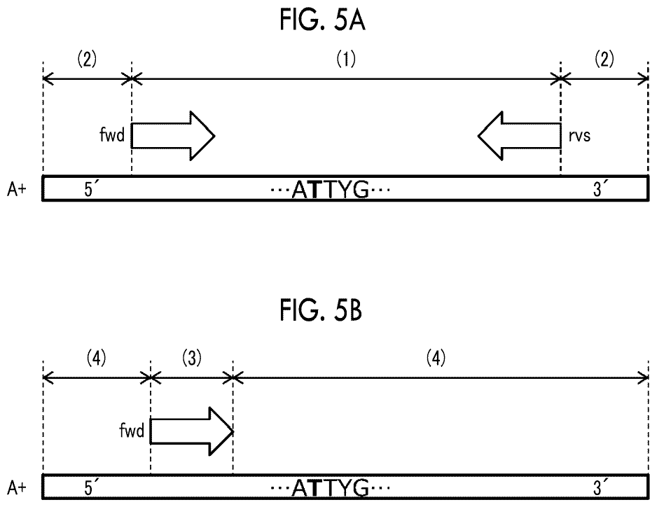

In a case where the number of heating cycles in PCR is set to n, and a primer pair (a forward primer and a reverse primer) binds to DNA as shown in FIG. 5A, PCR amplification products are generated in the order of 2ⁿ. In contrast, in a case where either the forward primer or the reverse primer binds to DNA as shown in FIG. 5B, PCR amplification products are generated in the order of 2n (FIG. 5B shows a case where the forward primer binds to DNA).

Therefore, in a case where PCR is performed using a general number of heating cycles (n is about 20 to 40), and a primer pair binds to a DNA sequence outside the amplification target region, unfortunately, non-specific products are generated in large amounts. However, in a case where either the forward primer or the reverse primer binds to the DNA sequence outside the related region, the amounts of generated non-specific products are not that large, which does not cause a special problem. Accordingly, in the related art, the problem of non-specific products being generated in a case where either the forward primer or the reverse primer binds to the DNA sequence outside the related region has not been especially considered. In FIG. 5A, (1) is the DNA sequence of the amplification target region, and (2) is the DNA sequence outside the amplification target region. Furthermore, in FIG. 5B, (3) is the DNA sequence of the related region of a partial sequence, and (4) is the DNA sequence outside the related region.

As described above, it is possible to increase the primer design success rate by performing determination under conditions created by adding the condition (3), which allows each primer to bind to DNA outside the target region within a predetermined range, to the condition of the related art in which determination is performed in designing a primer.

The processing of selecting partial sequences satisfying predetermined selection conditions as primer candidate sequences among one or more partial sequences cut out from each strand will be described using the flowchart in FIG. 4.

First, the primer candidate sequence selection unit 30 acquires one partial sequence from one or more partial sequences cut out from the first template strand (strand A+) (step S300) and determines whether or not the Tm value of the partial sequence is within a predetermined range (step S302).

In a case where the Tm value is not within a predetermined range, the primer candidate sequence selection unit 30 acquires another partial sequence (step S300). In a case where the Tm value is within a predetermined range, the primer candidate sequence selection unit 30 determines whether or not the number of YG sequences or CR sequences included in the partial sequence is equal to or less than a predetermined number. (Step S304).

In a case where the number of YG sequences or CR sequences included in the partial sequence is not equal to or less than a predetermined number, the primer candidate sequence selection unit 30 acquires another partial sequence (step S300). In a case where the number of YG sequences or CR sequences included in the partial sequence is equal to or less than a predetermined number, the primer candidate sequence selection unit 30 determines whether or not the upper limit of the number of binding sites with the base sequence outside the related region on the template strand DNA after base conversion is equal to or less than a predetermined number which is 1 or more (step S306).

In a case where the upper limit of the number of binding sites between the base sequence outside the related region on the template strand DNA after base conversion and the partial sequence is not equal to or less than “a predetermined number which is 1 or more”, the primer candidate sequence selection unit 30 acquires another partial sequence (step S300). In a case where the upper limit of the number of binding sites with the sequence outside the related region on the template strand DNA after base conversion is equal to or less than a predetermined number which is 1 or more, the primer candidate sequence selection unit 30 selects the partial sequence as a primer candidate sequence (step S308) and determines whether or not all the partial sequences cut out from the first template strand (strand A+) have been subjected to determination (step S310).

In a case where not all the partial sequences cut out from the first template strand (strand A+) have been subjected to determination, the primer candidate sequence selection unit 30 acquires another partial sequence (step S300). In a case where all the partial sequences have been subjected to determination, the primer candidate sequence selection unit 30 determines one or more selected primer candidate sequences as forward primer candidate sequences of the first template strand (strand A+) (step S312).

One or more partial sequences cut out from the first complementary strand (strand A−), one or more partial sequences cut out from the second template strand (strand B+), and one or more partial sequences cut out from the second complementary strand (strand B−) are subjected to the same determination (steps S300 to S310), and reverse primer candidate sequences of the first template strand (strand A+), forward primer candidate sequences of the second template strand (strand B+), and reverse primer candidate sequences of the second template strand (strand B+) are determined (step S312).

### (Primer Sequence Determination Unit)

The primer sequence determination unit 32 is a unit that performs the primer sequence determination step S22 shown in FIG. 2. In (I) a case where one or more primer sequences of a different target site have not yet been determined and (II) a case where one or more primer sequences of the different target site have already been determined, the primer sequence determination unit 32 creates a combination (pair) of predetermined sequences from one or more primer candidate sequences determined by the primer candidate sequence selection unit 30, that is, from one or more forward primer candidate sequences of the first template strand (strand A+), one or more reverse primer candidate sequences of the first template strand (strand A+), one or more forward primer candidate sequences of the second template strand (strand B+), and one or more reverse primer candidate sequences of the second template strand (strand B+), calculates a local alignment score between the sequences of each combination, adopts and determines a forward primer sequence and a reverse primer sequence for amplifying a region including the predetermined target site selected in the partial sequence cutting unit 28 in each strand (strand A+ or strand B+) based on whether or not the value of the local alignment score exceeds a predetermined threshold value. Hereinafter, a primer sequence determination method performed in each chain will be described.

(I) In a case where one or more primer sequences of a different target site have not yet been determined, [1] one or more primer candidate sequence pairs related to a predetermined target site are selected from one or more primer candidate sequences of the first template strand (strand A+), [2] one pair is selected from the one or more primer candidate sequence pairs of the predetermined target site, and a local alignment score between the sequences of the selected primer candidate sequence pair is calculated, and [3] the primer candidate sequence pair for which the local alignment score being equal to or less than the predetermined threshold value is calculated is adopted and determined as a forward primer sequence and a reverse primer sequence for amplifying a region including the predetermined target site in the first template strand (strand A+).

Here, in a case where the score of the primer candidate sequence pair selected in [2] is higher than the threshold value and the primer sequence pair (the forward primer sequence and the reverse primer sequence) cannot be determined, one different pair is selected from the primer candidate sequence pairs selected in [1], the steps [2] and [3] are performed, and such steps are repeated until at least one primer sequence pair is determined. In a case where at least one primer sequence pair can be determined, it is not necessary to always perform the step of calculating the score of all the primer candidate sequence pairs selected in [1] and the like, and the process may return to the partial sequence cutting step S18 to select another target site (step S280 of FIG. 4) and determine the primer sequence of the other target site. The method has an effect of reducing a calculation cost and saving time and effort.

In addition, in a case where the scores of all the primer candidate sequence pairs selected in [1] are higher than the threshold value and the primer sequence pairs cannot be adopted and determined as a primer sequence pair, the process returns to the partial sequence cutting step S18, another target site is selected (step S280 of FIG. 4), and the primer sequence of the other target site is determined.

(II) In a case where one or more primer sequences of the different target site have already been determined, one or more primer candidate sequence pairs related to the predetermined target site are selected from the one or more primer candidate sequences of the first template strand (strand A+), [2] one pair is selected from the one or more primer candidate sequence pairs of the predetermined target site, and a local alignment score between each of the candidate sequences of the selected primer candidate sequence pair and each of the already determined primer sequences of the different target site, and a local alignment score between the sequences of the selected primer candidate sequence pair are calculated, and [3] a maximum value (that is, a score of a pair which is most likely to form a primer dimer) from all the calculated local alignment scores, and a primer candidate sequence pair for which a local alignment score having the maximum value being equal to or less than a predetermined threshold value is calculated is adopted and determined as the forward primer sequence and the reverse primer sequence for amplifying the region including the predetermined target site in the first template strand (strand A+).

Here, in a case where the maximum value of the score calculated for the primer candidate sequence pair selected in [2] is higher than the threshold value and the primer sequence pair (the forward primer sequence and the reverse primer sequence) cannot be determined, one different pair is selected from the primer candidate sequence pairs selected in [1], the steps [2] and [3] are performed, and such steps are repeated until at least one primer sequence pair is determined. In a case where at least one primer sequence pair can be determined, it is not necessary to always perform the step of calculating the score of all the primer candidate sequence pairs selected in [1] and the like, and the process may return to the partial sequence cutting step S18 to select another target site (step S280 of FIG. 4) and determine the primer sequence of the other target site. The method has an effect of reducing a calculation cost and saving time and effort.

In addition, in a case where the maximum value of the scores calculated for all the primer candidate sequence pairs selected in [1] are higher than the threshold value and the primer sequence pairs cannot be adopted and determined as a primer sequence pair, the process returns to the partial sequence cutting step S18, another target site is selected (step S280 of FIG. 4), and the primer sequence of the other target site is determined.

Here, in a case where, <1> a complementary base pair is set to “X” per pair, <2> a non-complementary base pair is set to “Y” per pair, and <3> a case where there is insertion or deletion is set to “Z” per one insertion or deletion between the primer candidate sequences or between the primer candidate sequence and the already determined primer sequence, the local alignment score is calculated using “X” of 1, “Y” of −4 to −2, and “Z” of −6 to −3. In addition, the predetermined threshold value is 1 to 4.

The present inventors have focused on the fact that, in the related art, parameters (for example, a complementary score of 1, a non-complementary score of −1, and a gap/deletion score of −2), a threshold value (0), and a method of comparing sequences (for example, brute force of candidate sequences) used for general score calculation have been used, and have conducted intensive studies on a method of calculating a local alignment score, a combination or order of sequence comparisons related to score calculation, a predetermined threshold value for selecting a determination of a primer sequence, and the like that are not particularly examined, and have found that, according to the method, it is possible to obtain a high primer design success rate while suppressing a formation rate of primer dimer extremely low with a small calculation cost. In particular, the more the number of target sites is, specifically, in a case of performing primer design in which the number of target sites is 50 or more, the desired effect of the present invention can be significantly acquired.

In a case where the above-described effect is acquired by a method in the related art, there is an object that it is necessary to improve score calculation by using a method with a high calculation cost, such as chemical energy calculation or deep learning, and in a case where there are many target sites, it is not possible to perform calculation in a practical time. However, since in the present method, the above-described effect can be obtained “within the range of score calculation by simple addition”, the method has an effect that design can be performed in a practical time (about several days in the case of a general computer) with a small calculation cost in a case where the number of target sites is about several thousand.

The primer sequence determination unit 32 is configured with a computer and functions to adopt and determine a forward primer sequence and a reverse primer sequence from one or more primer candidate sequences described above.

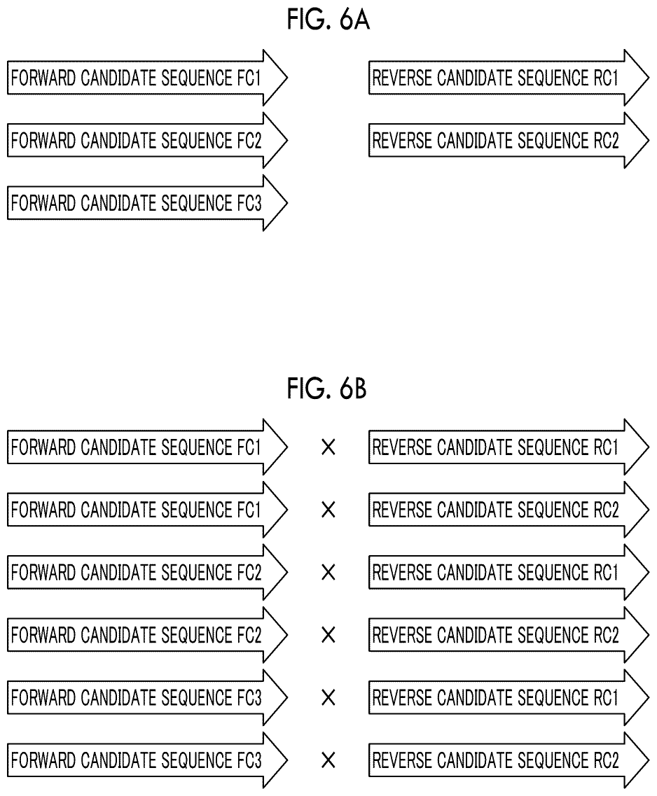

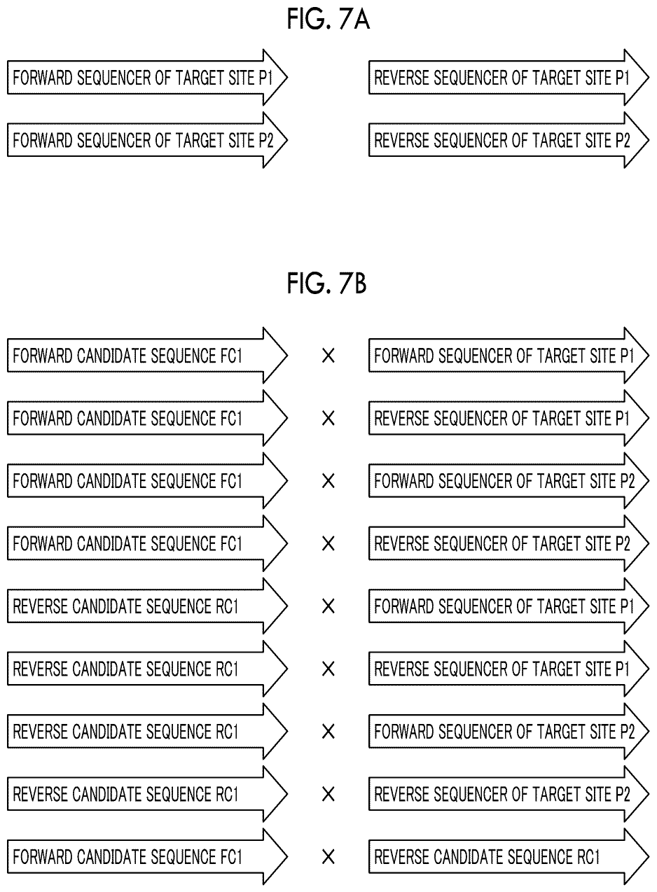

Here, a method of comparing sequences (combination of sequences) and an order thereof, which are related to calculation of the local alignment score, will be described more specifically with reference to FIGS. 6 and 7.

First, a primer sequence determination method in (I) a case where one or more primer sequences of a different target site have not yet been determined will be described.

In the step [1], first, all primer pairs (a combination of a forward primer and a reverse primer) that can be prepared are acquired from one or more forward primer candidate sequences of the first template strand (strand A+) and one or more reverse primer candidate sequences of the first template strand (strand A+), and a length of a PCR amplification product expected to be amplified by PCR is calculated for each of the primer pairs. Next, it is determined whether or not the calculated length of the PCR amplification product is within a predetermined numerical range, and in a case where the calculated length of the PCR amplification product is within the predetermined numerical range, the primer pair (that is, the combination of the forward primer candidate sequence of the first template strand and the reverse primer candidate sequence of the first template strand) for which the length of the PCR amplification product is calculated is adopted as one or more primer candidate sequence pairs for amplifying a region including the target site selected in the partial sequence cutting unit 28 (partial sequence cutting step), that is, one or more pairs of the forward primer candidate sequence of the first template strand and the reverse primer candidate sequence of the first template strand (step S320 of FIG. 4).

“Predetermined numerical range” for determining the calculated length of the PCR amplification product is a range including the length of the PCR amplification product that the user desires. As described above, the predetermined numerical range is not particularly limited as long as it is a known range, that is, 70 to several kilo base pairs. It is preferable to consider a PCR success rate, the sequencing ability of a DNA sequencer, and the like.

FIG. 6A shows primer candidate sequences (three forward primer candidate sequences and two reverse primer candidate sequences) selected in the primer candidate sequence selection unit 30 (primer candidate sequence selection step). FIG. 6B shows one or more primer candidate sequences pairs related to the predetermined target site selected in the step [1] (that is, in this case, it is determined that the length of the PCR amplification product expected to be amplified by PCR of all pairs of the three forward primer candidate sequences and the two reverse primer candidate sequences is within a predetermined range).

Next, in the step [2], a “forward candidate sequence FC1” and a “reverse candidate sequence RC1” are selected as one pair from the primer candidate sequence pairs (6 pairs) shown in FIG. 6B, and the local alignment score between the sequences of the pair is calculated.

Next, in a case where the value of the calculated local alignment score is equal to or less than a predetermined threshold value in the step [3], the pair of the “forward candidate sequence FC1” and the “reverse candidate sequence RC1” selected in the step [2] are adopted and determined as the forward primer sequence and the reverse primer sequence for amplifying the region including the selected predetermined target site (step S324 and step S322 of FIG. 4).

Next, a primer sequence determination method in (II) a case where one or more primer sequences of the different target site have already been determined will be described.

In the step [1], first, in the same manner as in the step (I)-[1], one or more pairs of forward primer candidate sequences and reverse primer candidate sequence of the first template strand for amplifying the region including the target site selected in the partial sequence cutting unit 28 (partial sequence cutting step) (step S320 in FIG. 4). FIG. 6A shows primer candidate sequences (three forward primer candidate sequences and two reverse primer candidate sequences) selected in the primer candidate selection step. FIG. 6B shows one or more primer candidate sequences pairs related to the six predetermined target site selected in the [1] (that is, in this case, it is determined that the length of the PCR amplification product expected to be amplified by PCR of all pairs of the three forward primer candidate sequences and the two reverse primer candidate sequences is within a predetermined range). FIG. 7A shows already determined primer sequence pairs related to different target sites P1 and P2.

Next, in the step [2], a “forward candidate sequence FC1” and a “reverse candidate sequence RC1” are selected as one pair from the primer candidate sequence pairs shown in FIG. 6B, and a local alignment score between each of the candidate sequences and each of the already determined primer sequences of the different target site, and a local alignment score between the selected candidate sequence and the primer candidate sequence forming a pair with the selected candidate sequence are calculated. That is, as shown in FIG. 7B, a local alignment score between the “forward candidate sequence FC1” and the “forward sequence of target site P1”, the “reverse sequence of target site P1”, the “forward sequence of target site P2”, or the “reverse sequence of target site P2”, a local alignment score between the “reverse candidate sequence RC1” and the “forward sequence of target site P1”, the “reverse sequence of target site P1”, the “forward sequence of target site P2”, or the “reverse sequence of target site P2”, and a local alignment score between the pair of the “forward candidate sequence FC1” and the “reverse candidate sequence RC1” are calculated. That is, nine local alignment scores are calculated.

Next, in the step [3], maximum value is detected from the calculated nine local alignment scores, and the primer candidate sequence pair for which the local alignment score having the maximum value being equal to or less than the predetermined threshold value is calculated is adopted and determined as the forward primer sequence and the reverse primer sequence for amplifying the region including the selected predetermined target site (step S324 and step S322 of FIG. 4).

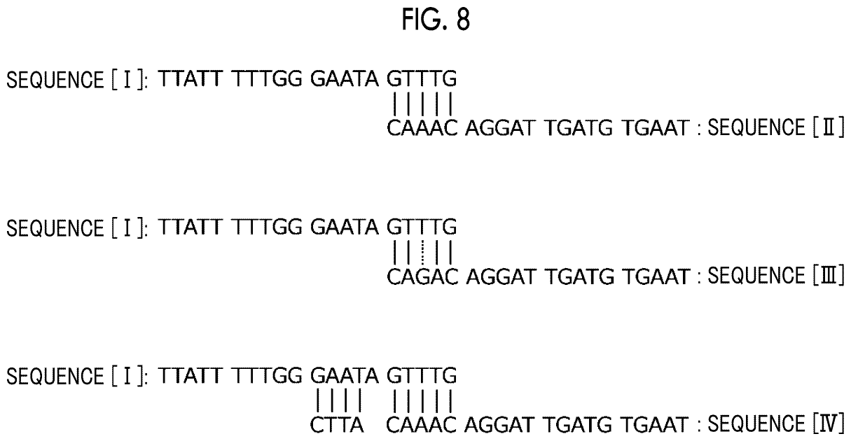

Here, with reference to the example of the local alignment shown in FIG. 8, a method of calculating a local alignment score and a determination method based on a threshold value will be described more specifically. FIG. 8 shows, from the top, (1) local alignment of the sequence [I] and the sequence [II], (2) local alignment of the sequence [I] and the sequence [III], and (3) local alignment of the sequence [I] and the sequence [IV], which are related to determination whether or not the primer candidate sequence is adopted as the primer sequence. In the figure, in a case where a base between sequences forms a complementary pair, “|” is attached, in a case where a non-complementary pair is formed, “:” is attached, “−” is attached to a gap, and nothing is attached to a deletion.

In the calculation of the local alignment score, <1> a complementary base pair is set to “X”=1 per pair, <2> a non-complementary base pair is set to “Y”=−3 per pair, and <3> a case where there is insertion or deletion is set to “Z”=−6 per one insertion or deletion between the sequences, and the threshold value is set to 4.

Since there are five complementary pairs between the sequence [I] and the sequence [II] in (1), the score is 1×5−3×0−6×0=5 (upper part of FIG. 8). However, since this score exceeds the threshold value of 4, the primer candidate sequence [I] is not adopted.

Since there are four complementary pairs and one non-complementary pair between the sequence [I] and the sequence [III] in (2), the score is 1×4−3×1−6×0=1. Since this score is equal to or less than the threshold value of 4, the primer candidate sequence [I] can be adopted.

Since there are nine complementary pairs and one deletion between the sequence [I] and the sequence [IV] in (3), the score is 1×9−3×0−6×1=3. Since this score is equal to or less than the threshold value of 4, the primer candidate sequence [I] can be adopted.

In the step [2], it is assumed that all the pairs (6 pairs) are selected from the primer candidate sequence pairs shown in FIG. 6B, and the maximum value of the local alignment scores calculated for each pair is as shown in Table 1. Here, in a case where the predetermined threshold value is set to 3, the candidate primer pair adopted and determined as the forward primer sequence and the reverse primer sequence for amplifying the region including the predetermined target site is only four pairs having a maximum value of 3 or less among all six pairs. That is, the four primer candidate sequence pairs which are the forward sequence and the reverse sequence of the first template strand (strand A+) are adopted and determined as the primer sequences.

TABLE 1

| Forward candidate | Reverse candidate | Maximum value | Adopt/Not |
|---|---|---|---|
| sequence | sequence | of score | adopt |
| Forward candidate | Reverse candidate | 3 | ◯ |
| sequence FC1 | sequence RC1 | | |
| Forward candidate | Reverse candidate | 6 | X |
| sequence FC1 | sequence RC2 | | |
| Forward candidate | Reverse candidate | 2 | ◯ |
| sequence FC2 | sequence RC1 | | |
| Forward candidate | Reverse candidate | 1 | ◯ |
| sequence FC2 | sequence RC2 | | |
| Forward candidate | Reverse candidate | 4 | X |
| sequence FC3 | sequence RC1 | | |
| Forward candidate | Reverse candidate | 2 | ◯ |
| sequence FC3 | sequence RC2 | | |

Similarly, first, in (I) a case where one or more primer sequences of a different target site have not yet been determined and (II) a case where one or more primer sequences of the different target site have already been determined, a combination (pair) of predetermined sequences is created from one or more forward primer candidate sequences of the second template strand (strand B+), and one or more reverse primer candidate sequences of the second template strand (strand B+), a local alignment score between the sequences of each combination is calculated, and a forward primer sequence and a reverse primer sequence for amplifying a region including the predetermined target site selected in the partial sequence cutting unit 28 in strand B+ are adopted and determined based on whether or not the value of the local alignment score exceeds a predetermined threshold value (step S322).

Once the determination of whether or not the length of a PCR amplification product is within a predetermined range is completed for all primer pairs, whether or not all target sites have been selected in the partial sequence cutting unit 28 (partial sequence cutting step) is determined (step S24).

In a case where not all the target sites have been selected, the processing returns to the partial sequence cutting step S18 to select other target sites (step S280). In a case where all the target sites have been selected, the processing ends.

### (Control Unit)

The control unit 34 is a unit that is connected not only to the portions in the primer design processing unit 18 but also to the input unit 12, the storage unit 14, and the output unit 16 directly or indirectly, controls each unit of the primer design device 10 based on the user's instruction from the input unit 12 or based on a predetermined operation program stored in the storage unit 14, and designs a primer. The control unit 34 is configured with, for example, a central processing unit (CPU) of a computer or the like.

The control unit 34 controls the primer candidate sequence selection unit 30, such that the determination operation (steps S300 to S308) is repeated until the determination of whether or not all the partial sequences satisfy a predetermined selection standard is completed in the primer candidate sequence selection unit 30 (step S310).

The control unit 34 controls the primer sequence determination unit 32, such that the determination operation (step S320) is repeated until the determination of whether or not the length of a PCR amplification product is within a predetermined range is completed for all the produced primer pairs in the primer sequence determination unit 32.

The control unit 34 controls the primer sequence determination unit 32 to repeat selecting one different pair from the primer candidate sequence pairs selected in [1] of (I) and (II) and performing the steps of [2] and [3] of (I) and (II) until at least one primer sequence pair related to a predetermined target site is determined, and in a case where the primer sequence pair related to the predetermined target site is not determined, to select one different target site in the partial sequence cutting step (steps S18, S280 to S282) and to perform the primer candidate sequence selection step (steps S20, S300 to S312) and the primer sequence determination step (steps S22, S320 to S322).

The control unit 34 controls the partial sequence cutting unit 28, the primer candidate sequence selection unit 30, and the primer sequence determination unit 32, such that the repetition step of repeating the partial sequence cutting step (steps S18 and S280 to S282), the primer candidate sequence selection step (step S20 and S300 to S312), and the primer sequence determination step (steps S22 and S320 to S322) is carried out until all the target sites acquired by the target site information acquisition unit 22 are detected in the partial sequence cutting unit 28 (step S24).

With the primer design device 10 according to the first embodiment of the present invention, it is possible to design a primer for amplicon methylation sequence analysis with an excellent design success rate. In addition, a primer based on the design can be obtained. As a result, it is possible to design a primer for more target sites and measure the methylation degree.

### Modification Example 1

Next, a primer design device according to Modification Example 1 of the first embodiment of the present invention will be described. Regarding the primer design device according to Modification Example 1, the same processing as that of the first embodiment will not be described.

In the first embodiment, in the determination of the primer sequence, the number of primer candidate sequence pairs for calculating the local alignment score and the number of forward primer sequences and reverse primer sequences for amplifying a region including a predetermined target site are not particularly limited. The present invention is not limited thereto, and the score can be calculated for all the pairs, and only one primer sequence pair for amplifying the region including each target site can be selected.

In Modification Example 1, the primer sequence determination unit 32 can also perform the following steps.

(I) In the case where one or more primer sequences of a different target site have not yet been determined, in the step [2], all pairs are selected from the one or more primer candidate sequence pairs of the predetermined target site, and for each pair, a local alignment score between sequences of the selected primer candidate sequence pair is calculated, and in the step [3], one or more primer candidate sequence pairs for which the local alignment score being equal to or less than the predetermined threshold value is calculated are selected, and a primer candidate sequence pair having a smallest value of the maximum value of the local alignment score is further detected from all the selected pairs, and is adopted and determined as the forward primer sequence and the reverse primer sequence for amplifying the region including the predetermined target site.

(II) In the case where one or more primer sequences of a different target site have already been determined, in the step [2], all pairs are selected from the one or more primer candidate sequence pairs of the predetermined target site, and for each pair, a local alignment score between each of candidate sequences of the selected primer candidate sequence pair and each of the already determined primer sequences of the different target site, and a local alignment score between the sequences of the selected primer candidate sequence pair are calculated, and in the step [3], for each pair, a maximum value is detected from all the calculated local alignment scores, a primer candidate sequence pair for which a local alignment score having the maximum value being equal to or less than a predetermined threshold value is calculated is selected, and a primer candidate sequence pair having a smallest value of the maximum value of the local alignment score is further detected from all the selected pairs, and is adopted and determined as the forward primer sequence and the reverse primer sequence for amplifying the region including the predetermined target site.

By performing such steps, an effect is obtained that a pair having the lowest primer dimer formation rate and the highest primer design success rate can be determined as a primer sequence from one or more primer sequence pairs capable of amplifying a region including a predetermined target site.

For example, in the step [2], it is assumed that all the pairs are selected from the primer candidate sequence pairs shown in FIG. 6B, and the maximum value of the local alignment scores calculated for each pair is as shown in Table 1. In a case where the predetermined threshold value is set to 3, four pairs having the maximum value of 3 or less are selected as pair the maximum value of the score equal to or less than the predetermined threshold value, and further, from all the selected pairs, the primer candidate sequence pair having the smallest value of the maximum value of the local alignment score is the primer candidate sequence pair of the “forward primer candidate sequence FC2” and the “reverse primer candidate sequence RC2”, thereby this pair is adopted and determined as the forward primer sequence and the reverse primer sequence for amplifying the region including the predetermined target site.

TABLE 2

| | | Maximum | Select/ | Adopt/ |
|---|---|---|---|---|
| Forward candidate | Reverse candidate | value of | Not | Not |
| sequence | sequence | score | select | adopt |
| Forward candidate | Reverse candidate | 3 | ◯ | X |
| sequence FC1 | sequence RC1 | | | |
| Forward candidate | Reverse candidate | 6 | X | X |
| sequence FC1 | sequence RC2 | | | |
| Forward candidate | Reverse candidate | 2 | ◯ | X |
| sequence FC2 | sequence RC1 | | | |
| Forward candidate | Reverse candidate | 1 | ◯ | ◯ |
| sequence FC2 | sequence RC2 | | | |
| Forward candidate | Reverse candidate | 4 | X | X |
| sequence FC3 | sequence RC1 | | | |
| Forward candidate | Reverse candidate | 2 | ◯ | X |
| sequence FC3 | sequence RC2 | | | |

### Modification Example 2

Next, a primer design device according to Modification Example 2 of the first embodiment of the present invention will be described. Regarding the primer design device according to Modification Example 2, the same processing as that of the first embodiment will not be described.

In the first embodiment, the user designs the primer without setting the primer design rate. The present invention is not limited thereto, and the primer design can also be performed based on a primer design success rate desired by the user, which is set in advance.

In advance, a correspondence relationship between at least a predetermined threshold value, the number of target sites (measurement sites), and the primer design success rate is measured using the primer design device (method) described in the first embodiment and each modification example, and the correspondence relationship is stored in the storage unit 14. Here, the “predetermined threshold value” is not particularly limited as long as it is 1 to 4.

In a case where all of X, Y, and Z are integers and the number of target sites is less than 1,000, it is preferable to create a correspondence relationship for all of the threshold values 1, 2, 3, and 4. In a case where the number of target sites is 1,000 or more, it is preferable to create a correspondence relationship for at least two or more threshold values. In a case where X, Y, and Z include non-integer values and the number of target sites is less than 1,000, it is preferable to create at least 5 or more correspondence relationships. In a case where the number of target sites is 1,000 or more, it is preferable to create a correspondence relationship for at least two or more threshold values.

In a case where via the input unit 12, the user sets at least the desired primer design success rate and the number of target sites and inputs a command to execute the primer design, the primer sequence determination unit 32 reads out the predetermined threshold value corresponding to the primer design success rate and the number of target sites, which are equal to or more than the set values of the primer design success rate and the number of target sites and have a small difference therebetween, from the correspondence relationships stored in the storage unit 14, and determines the primer sequence based on the predetermined threshold value.

With such primer design device of Modification Example 2, the user can easily design a primer sequence with a small cost burden according to the background or circumstances at the time of primer design, such as a case where the amount of the sample is small, or a case where the user wants to attempt primer design with a desired primer design success rate or a plurality of primer design success rates. In addition, a primer based on the design can be obtained.

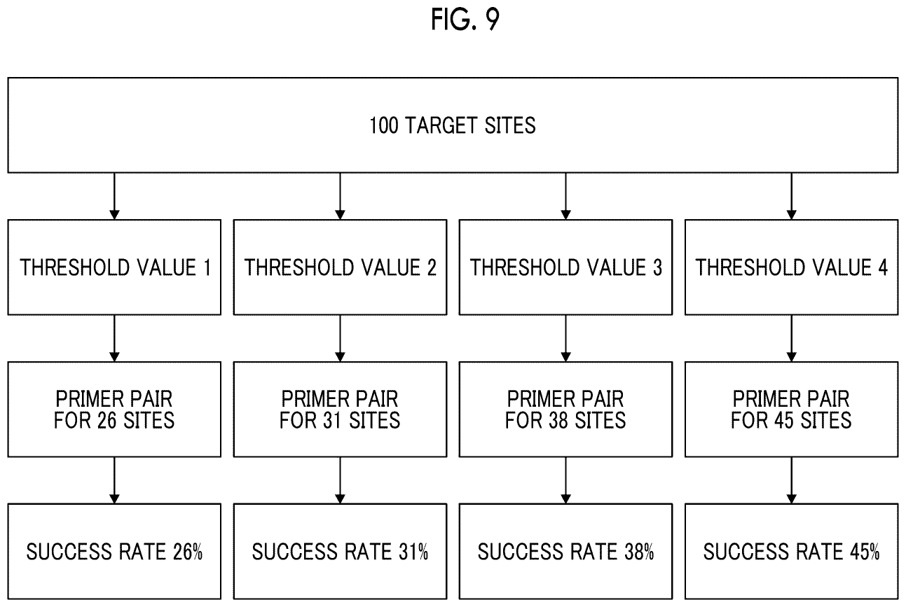

A method of selecting a threshold value used in a case of determining a primer sequence will be specifically described with reference to FIG. 9. FIG. 9 shows primer design success rates measured in a case where primers related to 100 target sites are designed in advance using the primer design device (method) described in the first embodiment and each modification example, and the threshold value for determining a local alignment score of each pair, are set to an integer of 1 to 4, that is, a correspondence relationship between the predetermined threshold value, the number of target sites (measurement sites), and the primer design success rate. This correspondence relationship is stored in the storage unit 17.

For example, in a case where the user desires a primer design success rate of 30% or more, the user sets at least the primer design success rate to 30% and the number of target sites to 100 and inputs an instruction to execute the primer design via the input unit 12. The number of target sites satisfying the condition: 100 and the threshold value of 2, corresponding to a primer design success rate of 31% which is greater than and closest to a primer design success rate desired by the user: 30%, are read out from the correspondence relationship in the storage unit 14 to the primer sequence determination unit 32, the primer sequence determination step is performed based on the threshold value 2 in the correspondence relationship, and primer sequence pairs for 31 sites are acquired.

### Modification Example 3

Next, a primer design device according to Modification Example 3 of the first embodiment of the present invention will be described. Regarding the primer design device according to Modification Example 3, the same processing as that of the first embodiment will not be described.

In the first embodiment, the methylatable cytosine (C) is limited to cytosines (C) in the CG sequence, and cytosine (C) picked up from such cytosines (C) is adopted as a target site. However, the methylatable cytosine (C) is not limited thereto and may also include cytosines (C) in a CHG sequence, and cytosine (C) picked up from such cytosines may be adopted as a target site.

In Modification Example 3, the target site information acquisition unit 22 additionally acquires two or more target sites included in the double-stranded genomic DNA acquired by the base sequence data acquisition unit 20 and the position information of the target sites via the input unit 12.

The base conversion unit 24 also converts cytosine (C) of a CHG sequence on the template DNA acquired from the base sequence data acquisition unit 20 into “Y”, and converts cytosine (C) of other sequences (that is, sequences other than a CG sequence and a CHG sequence) into thymine (T).

The primer candidate sequence selection unit 30 additionally selects partial sequences satisfying all the predetermined selection conditions (1) to (4) including the following condition (4) as primer candidate sequences, among one or more partial sequences of each strand cut out by the partial sequence cutting unit 28.

(4) The number of YHG sequences or CDR sequences included in a partial sequence is equal to or less than a predetermined number.

The number of “YHG sequences or CDR sequences included in a partial sequence” related to the condition (4) is not particularly limited. From the viewpoint of markedly obtaining the desired effect of the present invention, the number of YHG sequences or CDR sequences is preferably 2 or less, more preferably 1 or less, and particularly preferably 0.

In a case where the above condition is satisfied, the influence of the binding of the primer to cytosine (C) of the CHG sequence in the primer binding site can be reduced.

With the primer design device of Modification Example 3 according to the first embodiment of the present invention, it is possible to easily and rapidly design a primer for amplicon methylation sequence analysis that is also applicable to a CHG sequence. In addition, a primer based on the design can be obtained. As a result, the analysis related to these sequences can be performed, which makes it possible to more specifically analyze the DNA methylation status (methylation degree).

Modification Example 3 can be combined with Modification Example 1 or 2 described above.

### Modification Example 4

Next, a primer design device according to Modification Example 4 of the first embodiment of the present invention will be described. Regarding the primer design device according to Modification Example 4, the same processing as that of the first embodiment will not be described.

In the first embodiment, the methylatable cytosine (C) is limited to cytosines (C) in the CG sequence, and cytosine (C) picked up from such cytosines (C) is adopted as a target site. However, the methylatable cytosine (C) is not limited thereto and may also include cytosines (C) in a CHH sequence, and cytosine (C) picked up from such cytosines may be adopted as a target site.

In Modification Example 4, the target site information acquisition unit 22 additionally acquires two or more target sites included in the double-stranded genomic DNA acquired by the base sequence data acquisition unit 20 and the position information of the target sites via the input unit 12.

The base conversion unit 24 also converts cytosine (C) of a CHH sequence on the template DNA acquired from the base sequence data acquisition unit 20 into “Y”, and converts cytosine (C) of other sequences (that is, sequences other than a CG sequence and a CHH sequence) into thymine (T).

The primer candidate sequence selection unit 30 additionally selects partial sequences satisfying all the predetermined selection conditions (1) to (3) and (5) including the following condition (5) as primer candidate sequences, among one or more partial sequences of each strand cut out by the partial sequence cutting unit 28.

(5) The number of YHH sequences or DDR sequences included in a partial sequence is equal to or less than a predetermined number.

The number of “YHH sequences or DDR sequences included in a partial sequence” related to the condition (5) is not particularly limited. From the viewpoint of markedly obtaining the desired effect of the present invention, the number of YHH sequences or DDR sequences is preferably 2 or less, more preferably 1 or less, and particularly preferably 0.

In a case where the above condition is satisfied, the influence of the binding of the primer to cytosine (C) of the CHH sequence in the primer binding site can be reduced.

With the primer design device of Modification Example 4 according to the first embodiment of the present invention, it is possible to easily and rapidly design a primer for amplicon methylation sequence analysis that is also applicable to a CHH sequence. In addition, a primer based on the design can be obtained. As a result, the analysis related to these sequences can be performed, which makes it possible to more specifically analyze the DNA methylation status (methylation degree).

Modification Example 4 can be combined with Modification Example 1 or 2 described above. In addition, Modification Example 4 can be combined with Modification Example 3 described above. That is, the methylatable cytosine (C) may include both the cytosine (C) in a CHG sequence and the cytosine (C) in a CHH sequence, and cytosine (C) picked up from the above cytosines may be adopted as a target site.

In this case, the primer candidate sequence selection unit 30 additionally selects partial sequences satisfying all the selection conditions (1) to (5) as primer candidate sequences, among one or more partial sequences cut out by the partial sequence cutting unit 28.

### Modification Example 5

Next, a primer design device according to Modification Example 5 of the first embodiment of the present invention will be described. For the primer design device according to Modification Example 4, the same configuration as that in the first embodiment will be denoted by the same reference numeral, and the same processing as that in the first embodiment will not be described.

In the first embodiment, in order to amplify and analyze both strands of DNA, a device and a method for designing two sets of primers are described. However, the present invention is not limited thereto, and in a case where either of two DNA strands is to be analyzed, one set of primers may be designed. That is, although primers are designed based on the strand A and the strand B in FIG. 3B, primers may be designed based on only the strand A.

Furthermore, in a case where a DNA methylation maintenance mechanism is considered to be working, only one set of primers may be designed, because in a case where C in the CG sequence of one DNA strand is methylated, C in the CG sequence of the other strand is extremely highly likely to be methylated, and in a case where C in the CG sequence of one DNA strand is unmethylated, C in the CG sequence of the other strand is extremely highly likely to be unmethylated. When one set of primers cannot be designed based on one strand in this case, the primers may be designed based on the other strand.

In a case where only one set of primers is to be designed as described above, the complementary strand generation unit 26 produces only a complementary strand A-having a base sequence complementary to the base sequence of the strand A+ shown in FIG. 3C.

Then, the partial sequence cutting unit 28 selects one target site from one target site from two or more target sites acquired in the target site information acquisition unit 22 (step S280), detects “Y” of the selected target site or “R” (that is, a base which is in a methylation site in the target site) complementary to “Y” from the DNA sequences of the strand A+ and the strand A-based on the position information of the selected target site, cuts partial sequences as much as possible from partial sequences having a predetermined length from the base sequences ((1) and (2) in FIG. 3D) positioned on 5′ terminal side of the detected “Y” or “R” (step S282) to obtain one or more partial sequences.

The primer candidate sequence selection unit 30 is a unit that performs the primer candidate sequence selection step S20 shown in FIG. 2, and selects partial sequences satisfying all the predetermined selection conditions (1) to (3) as primer candidate sequences from one or more partial sequences of each strand cut out by the partial sequence cutting unit 28.

Among one or more partial sequences cut out from the first template strand (strand A+) (that is, one or more partial sequences cut out from (1) in FIG. 3D), a partial sequence that satisfies predetermined selection conditions is selected as a forward primer candidate sequence of the first template strand (strand A+). Among one or more partial sequences cut out from the first complementary strand (strand A−), a partial sequence that satisfies predetermined selection conditions is selected as a reverse primer candidate sequence of the first template strand (strand A+).

In (I) a case where one or more primer sequences of a different target site have not yet been determined and (II) a case where one or more primer sequences of the different target site have already been determined, in the primer sequence determination unit 32, a combination (pair) of predetermined sequences is created from one or more forward primer candidate sequences of the first template strand (strand A+), and one or more reverse primer candidate sequences of the first template strand (strand A+), a local alignment score between the sequences of each combination is calculated, and a forward primer sequence and a reverse primer sequence for amplifying a region including the predetermined target site selected in the partial sequence cutting unit 28 in strand A+ are adopted and determined based on whether or not the value of the local alignment score exceeds a predetermined threshold value.

Note that Modification Example 5 can be combined with at least one of Modification Examples 1 to 4 described above.

### Second Embodiment

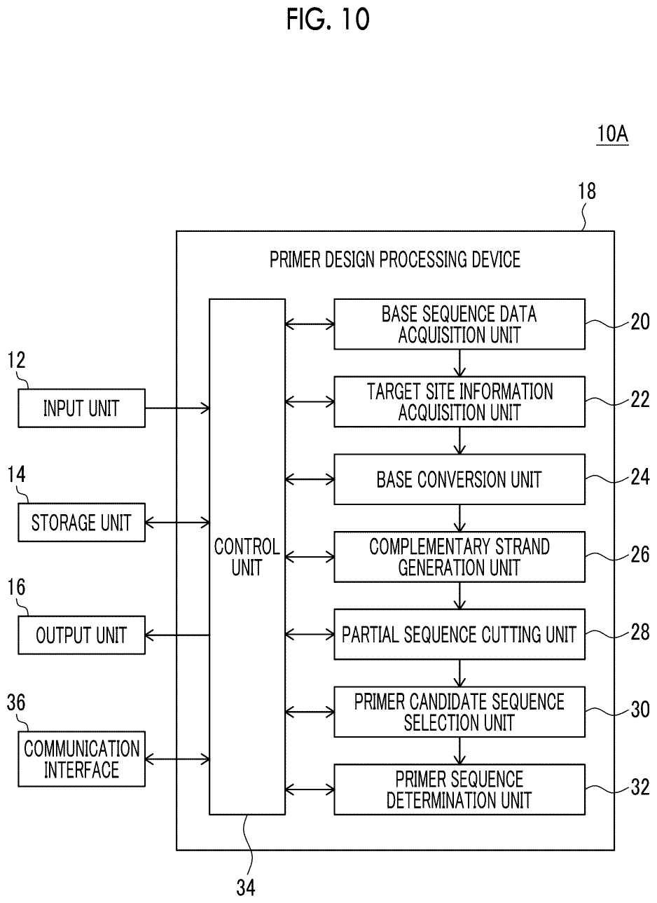

FIG. 10 is a block diagram conceptually showing an example a primer design device according to a second embodiment of the present invention. The primer design device 10 of the first embodiment can also comprise a communication interface (communication device).

A primer design device 10A of the second embodiment shown in FIG. 10 has the same configuration as the primer design device 10 of the first embodiment shown in FIG. 1 except that the primer design device 10A has a communication interface 36. Therefore, the same configuration requirements are denoted by the same reference numerals and will not be described.

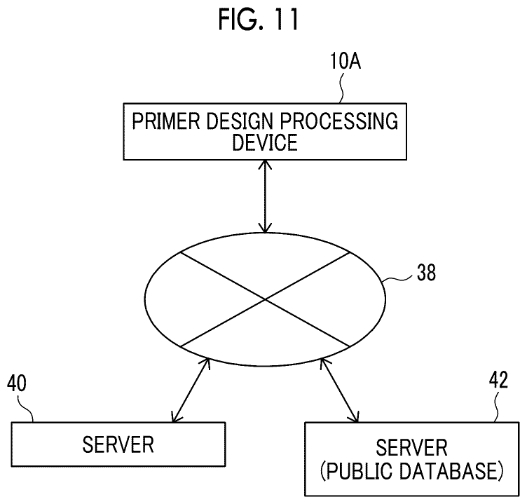

As shown in FIG. 11, via a communication network 38 such as the internet, the primer design device 10A can be connected to a search server 42 comprising a public database installed on the outside of the device.

The device 10A of the present embodiment can operate at least one of the base sequence data acquisition unit 20, the target site information acquisition unit 22, the base conversion unit 24, the complementary strand generation unit 26, the partial sequence cutting unit 28, the primer candidate sequence selection unit 30, or the primer sequence determination unit 32 via the communication interface 36 according to the program located at the site of an external server 40. In this a case, a primer design device 10A of the present embodiment may not include the units operated according to the program in the external server.

For example, based on the instructions from the control unit 34, the communication interface 36 can acquire a DNA base sequence including genes and genomes from a public database via the communication network 38 and store the database in the storage unit 14. Examples of the public database include GenBank of the National Center for Biotechnology Information (NCBI) of the United States, ENA of the European Molecular Biology Laboratory (EMBL), and DDBJ of National Institute of Genetics, and the like.

The base sequence acquired from the public database may be a partial sequence of the base sequence of the genomic DNA of biological species for which a primer is to be designed. The base sequence is preferably a complete sequence.

For example, the communication interface 36 can perform a sequence homology search using a public search server 42 via the communication network 38 based on an instruction of the control unit 34, and perform local alignment search or the like of the primer sequence determination unit 32. Examples of the public search server 42 include BLAST of the National Center for Biotechnology Information (NCBI) of the United States and the like.

### Third Embodiment

A third embodiment is a method of manufacturing a primer by synthesizing a primer based on the primer sequence designed by the primer design device and the primer design method according to the first and second embodiments.

The primer design method is as shown in the first and second embodiments.

Known methods can be used as the primer synthesis method. Examples thereof include a method of chemically synthesizing a primer from terminal bases with a DNA synthesizer or an RNA synthesizer by using deoxyribonucleoside triphosphate (dNTP) or the like as a material. Commercially available products can be used as the synthesizer.

In the device according to an embodiment of the present invention, each configuration requirements included in the device may be configured with the dedicated hardware or may be configured with a programmed computer.

The method according to an embodiment of the present invention can be performed by, for example, a program for causing a computer to execute each step of the method. In addition, a computer-readable recording medium on which the program is recorded can be provided.

Although the present invention has been described in detail above, the present invention is not limited to the embodiment described above, and it is needless to say that various improvements or changes may be made without departing from the gist of the present invention.

### EXAMPLES

### Example 1 and Comparative Example 1

Based on the base sequence data of reference genome GRCh37 (GenBank assembly accession: GCA_000001405.1, RefSeq assembly accession: GCF_000001405.13), randomly selected 100 measurement sites (target sites) shown in Table 1, and the position information on the target sites, a primer for multiplex PCR producing a PCR amplification product having a length of 70 bp to 120 bp was designed using the primer design device of the first embodiment. The primer was designed such that the primer had a length of 20 to 35 bases (mer), and that only C in a CG sequence can be methylated. In addition, the conditions for determining the partial sequence were set as follows.

Condition (1): The Tm value is in a range of 55° C. to 65° C.

Condition (2): The number of YG sequences or CR sequences included in a partial sequence is 0.

Condition (3): The upper limit of the number of binding sites with the sequence outside the related region is 2.

In the calculation of the local alignment score, in Example 1, <1> a complementary base pair is set to “X”=1 per pair, <2> a non-complementary base pair is set to “Y”=−3 per pair, and <3> a case where there is insertion or deletion is set to “Z”=−6 per one insertion or deletion between the sequences, and the threshold value is set to 1.

On the other hand, in Comparative Example 1, <1> a complementary base pair is set to “X”=1 per pair, <2> a non-complementary base pair is set to “Y”=−1 per pair, and <3> a case where there is insertion or deletion is set to “Z”=−2 per one insertion or deletion between the sequences, and the threshold value is set to 0.

Table 3 shows whether the primer for each measurement site of Example 1 and Comparative Example 1 is successfully designed or failed to be designed and shows the primer design success rate calculated from the results of the success or failure of the primer design. In addition, Table 4 shows the primers that could be designed in Example 1, and Table 5 shows the primers that could be designed in Comparative Example 1. The first pair in which the maximum value of the local alignment score was equal to or less than the threshold value was adopted as each of the primer pairs.

As shown in Table 3, the primer design success rate was 62% in Example 1 and 4% in Comparative Example 1. From this result, it was confirmed that the primer design success rate was increased by setting the threshold value for the maximum value of the local alignment score in the primer sequence determination step within a predetermined range.

TABLE 3

| | | | Success or failure | | | | | Success or failure | |
|---|---|---|---|---|---|---|---|---|---|
| | | | of design | | | | | of design | |
| Measurement site | | | | Comparative | Measurement site | | | | Comparative |
| ID | Chromosome | Coordinate | Example 1 | Example 1 | ID | Chromosome | Coordinate | Example 1 | Example 1 |
| 1 | 6 | 29870056 | — | — | 51 | 19 | 54196147 | — | — |
| 2 | 7 | 4389129 | X | — | 52 | 3 | 128199781 | — | — |
| 3 | 14 | 105391263 | X | — | 53 | 1 | 46112691 | — | — |
| 4 | 15 | 26302108 | — | — | 54 | 5 | 147714437 | X | — |
| 5 | 8 | 19313167 | X | X | 55 | 1 | 3721794 | X | — |
| 6 | 7 | 27561178 | X | — | 56 | 7 | 18534872 | X | — |
| 7 | 7 | 151553782 | — | — | 57 | 8 | 72518106 | X | — |
| 8 | 11 | 1862477 | X | — | 58 | 1 | 12268883 | X | — |
| 9 | 6 | 84221752 | — | — | 59 | 2 | 99526035 | X | — |
| 10 | 12 | 114677042 | X | — | 60 | 14 | 101513595 | — | — |
| 11 | 6 | 29589729 | — | — | 61 | 6 | 64734868 | — | — |
| 12 | 13 | 49076914 | X | — | 62 | 17 | 40322138 | — | — |
| 13 | 7 | 76109396 | X | — | 63 | 5 | 138861855 | X | — |
| 14 | 3 | 128186859 | X | — | 64 | 3 | 178984973 | X | — |
| 15 | 12 | 34756440 | X | — | 65 | 3 | 100148679 | X | — |
| 16 | 10 | 115991467 | X | — | 66 | 11 | 20152992 | X | — |
| 17 | 14 | 107095027 | — | — | 67 | 14 | 23624363 | — | — |
| 18 | 10 | 130268585 | X | — | 68 | 6 | 2623483 | — | — |
| 19 | 6 | 31515526 | — | — | 69 | 1 | 44344466 | X | — |
| 20 | 7 | 2414948 | — | — | 70 | 6 | 39849807 | X | — |
| 21 | 6 | 32030188 | — | — | 71 | 12 | 51180192 | X | — |
| 22 | 13 | 113992654 | X | — | 72 | 17 | 43651976 | X | — |
| 23 | 10 | 132099067 | X | — | 73 | 10 | 104832357 | X | — |
| 24 | 6 | 168618157 | X | — | 74 | 1 | 220876396 | X | — |
| 25 | 1 | 161916064 | — | — | 75 | 12 | 63238340 | — | — |
| 26 | 11 | 45354409 | X | X | 76 | X | 146312617 | X | — |
| 27 | 16 | 70516599 | X | — | 77 | 14 | 76734327 | X | — |
| 28 | 2 | 233096291 | X | — | 78 | 13 | 19847419 | — | — |
| 29 | 1 | 8601318 | X | — | 79 | 8 | 7004738 | — | — |
| 30 | 3 | 57125501 | X | — | 80 | 4 | 106768095 | — | — |
| 31 | 9 | 116298900 | X | — | 81 | 12 | 67278182 | X | — |
| 32 | 9 | 97317179 | X | — | 82 | 7 | 157374793 | X | — |
| 33 | 9 | 117692954 | — | — | 83 | 1 | 8427556 | X | — |
| 34 | 14 | 104742172 | — | — | 84 | 7 | 121437819 | X | — |
| 35 | 17 | 78058778 | X | — | 85 | 5 | 79222121 | — | — |
| 36 | 16 | 11482317 | X | — | 86 | 1 | 212662017 | X | — |
| 37 | 11 | 44291407 | X | X | 87 | 10 | 3805441 | X | X |
| 38 | 13 | 39564046 | X | — | 88 | 14 | 75886161 | X | — |
| 39 | 14 | 104824020 | — | — | 89 | 17 | 39781108 | — | — |
| 40 | 2 | 112822975 | — | — | 90 | 13 | 113506845 | — | — |
| 41 | 15 | 32162729 | — | — | 91 | 2 | 175436504 | — | — |
| 42 | 2 | 187826872 | X | — | 92 | 9 | 130323725 | X | — |
| 43 | 10 | 131460030 | X | — | 93 | 1 | 19717337 | X | — |
| 44 | 19 | 19106904 | — | — | 94 | 13 | 96454018 | X | — |
| 45 | 14 | 56856095 | — | — | 95 | 1 | 206644843 | X | — |
| 46 | 7 | 156755824 | — | — | 96 | 6 | 30034500 | — | — |
| 47 | 3 | 65652312 | X | — | 97 | 11 | 130116833 | X | — |
| 48 | 8 | 122680033 | — | — | 98 | 17 | 76220898 | — | — |
| 49 | 5 | 140090404 | X | — | 99 | 2 | 113931518 | X | — |
| 50 | 12 | 116715986 | — | — | 100 | 1 | 243603467 | X | — |
| | | | | | | | Design | 62% | 4% |
| | | | | | | | access | | |
| | | | | | | | rate | | |

Primer designed in Example 1

| | Forward primer | | | Reverse primer | | |
|---|---|---|---|---|---|---|
| Mea- | | Base | | | Base | |
| sure- | | se- | | | se- | |
| ment | | quence | Se- | | quence | Se- |
| site | | (5′ → | quence | | (5′ → | quence |
| ID | Name | 3′) | number | Name | 3′) | number |
| 2 | 2F1 | TTTTTT | 1 | 2R1 | AATCCC | 63 |
| | | TTATAG | | | ACTTAC | |
| | | TTTTTG | | | AAAAAA | |
| | | GTAGTG | | | CA | |
| | | A | | | | |
| 3 | 3F1 | TGTAGA | 2 | 3R1 | TTAATA | 64 |
| | | GAGGAG | | | TCTATC | |
| | | GAGGTG | | | CTAATT | |
| | | AG | | | CCAACC | |
| 5 | 5F1 | GGTTTG | 3 | 5R1 | TCACAA | 65 |
| | | AAATGT | | | TCAAAA | |
| | | TATTTT | | | CATTTC | |
| | | TAATAA | | | TAAA | |
| | | G | | | | |
| 6 | 6F1 | TAGTTG | 4 | 6R1 | AAAAAC | 66 |
| | | TTGATT | | | CAATAC | |
| | | TGATAG | | | TAACCT | |
| | | GAGGTA | | | AATCC | |
| | | G | | | | |
| 8 | 8F1 | GGTTAA | 5 | 8R1 | CAAATA | 67 |
| | | GAAGGA | | | TAAAAA | |
| | | GGATAT | | | ATAATC | |
| | | AGAGA | | | CCCA | |
| 10 | 10F1 | TTGTTT | 6 | 10R1 | CAAACA | 68 |
| | | TTTGTT | | | AAATTT | |
| | | GTGTGG | | | ACAACC | |
| | | AA | | | CA | |
| 12 | 12F1 | GGTTAT | 7 | 12R1 | CCTCAC | 69 |
| | | TTTTTA | | | CCACTT | |
| | | AATGGA | | | CTCCTA | |
| | | TAGTGA | | | CA | |
| 13 | 13F1 | TTTTTA | 8 | 13R1 | CTCAAA | 70 |
| | | AGGTGT | | | ATCCCA | |
| | | TAGGGG | | | ACCTCA | |
| | | AA | | | AAA | |
| 14 | 14F1 | GGAGTT | 9 | 14R1 | TCCCCA | 71 |
| | | TTTTAT | | | CTAACT | |
| | | GAAGGG | | | CCCCAA | |
| | | AG | | | AA | |
| 15 | 15F1 | GTGTGG | 10 | 15R1 | ACCCAA | 72 |
| | | AAGGAA | | | AATCTA | |
| | | AAAAAA | | | CAAAAC | |
| | | AG | | | CC | |
| 16 | 16F1 | TTGTTT | 11 | 16R1 | TTACCA | 73 |
| | | GTTTGT | | | ATATTC | |
| | | TATTTT | | | TCATTA | |
| | | TTTATT | | | ATTTAA | |
| | | AG | | | TATAA | |
| 18 | 18F1 | GGGGTA | 12 | 18R1 | TCCCCA | 74 |
| | | GAGTAT | | | CTAATA | |
| | | AGGTTA | | | CTTCCT | |
| | | GTTGA | | | TAC | |
| 22 | 22F1 | TGGTAG | 13 | 22R1 | TCTAAT | 75 |
| | | GTGTTT | | | CCCAAT | |
| | | TGGGTT | | | TCAATT | |
| | | GA | | | AAAA | |
| 23 | 23F1 | GTTTGT | 14 | 23R1 | AAAAAT | 76 |
| | | ATGGAT | | | AAACCT | |
| | | TATTAG | | | TACAAA | |
| | | GTTGA | | | CTACAC | |
| | | | | | A | |
| 24 | 24F1 | GGATTT | 15 | 24R1 | TCCAAA | 77 |
| | | TTTTTT | | | TCTCCT | |
| | | AGTTTT | | | ACTAAT | |
| | | TTAAAT | | | AAAAAC | |
| | | AAG | | | | |
| 26 | 26F1 | TTATTA | 16 | 26R1 | CCACTA | 78 |
| | | TTTATT | | | CACAAA | |
| | | TTTTGG | | | TAAAAA | |
| | | GTGAA | | | AATAAA | |
| 27 | 27F1 | GGAGGT | 17 | 27R1 | AAACAT | 79 |
| | | TAGTTT | | | AAAAAA | |
| | | GGTTAT | | | TCTAAT | |
| | | AGGTAG | | | CTATTC | |
| | | | | | AAA | |
| 28 | 28F1 | TTTTGG | 18 | 28R1 | ATTCAC | 80 |
| | | TTTTAA | | | TACTAT | |
| | | AAGAGA | | | CTAATA | |
| | | GAAA | | | AAACCC | |
| | | | | | A | |
| 29 | 29F1 | TTAGGG | 19 | 29R1 | TTTTTT | 81 |
| | | TTATAT | | | TCTCTT | |
| | | TTTAAT | | | TTTCCC | |
| | | ATGTAG | | | AA | |
| | | AAAA | | | | |
| 30 | 30F1 | TTATAT | 20 | 30R1 | AATTAC | 82 |
| | | TTTTAA | | | CCAAAC | |
| | | GTGGTA | | | AATATT | |
| | | AGAGGA | | | AATACC | |
| | | G | | | | |
| 31 | 31F1 | GGTTTT | 21 | 31R1 | CCAATA | 83 |
| | | TGTTGT | | | ACATTA | |
| | | GTTGGG | | | AAACAA | |
| | | AG | | | CCA | |
| 32 | 32F1 | TTTTTA | 22 | 32R1 | TCTCCT | 84 |
| | | TATTTA | | | AACCTC | |
| | | TATATA | | | AATATA | |
| | | AGTGTT | | | TAAAAC | |
| | | AGAAAT | | | A | |
| | | GA | | | | |
| 35 | 35F1 | TAAGGG | 23 | 35R1 | AAACTC | 85 |
| | | TTTATT | | | TACCCC | |
| | | AATTTT | | | ACCAAA | |
| | | TTTAAT | | | CA | |
| | | GAA | | | | |
| 36 | 36F1 | TTATGG | 24 | 36R1 | CTCCTT | 86 |
| | | TTGGGG | | | CTTCCA | |
| | | AAATTG | | | TACTAA | |
| | | AG | | | TAACC | |
| 37 | 37F1 | AGATTG | 25 | 37R1 | CCCACA | 87 |
| | | GGGTTA | | | AAAAAC | |
| | | GGATGA | | | CCTAAA | |
| | | GA | | | AAAC | |
| 38 | 38F1 | GTTTTT | 26 | 38R1 | AAATTA | 88 |
| | | TTGGTA | | | TTCAAA | |
| | | ATATAA | | | AAATAA | |
| | | GGTATA | | | TTATAA | |
| | | GAG | | | TAATAA | |
| | | | | | TATAC | |
| 42 | 42F1 | TTTTGT | 27 | 42R1 | AAAAAA | 89 |
| | | AGTTTT | | | ATCCCT | |
| | | GAGAGG | | | CAATAC | |
| | | TGA | | | AAC | |
| 43 | 43F1 | AAATTA | 28 | 43R1 | CTAAAA | 90 |
| | | TTAGTA | | | TTTCCA | |
| | | AATTAA | | | ATTTTA | |
| | | AAATAT | | | AATCC | |
| | | TAAAAT | | | | |
| | | AAAA | | | | |
| 47 | 47F1 | TTGAGA | 29 | 47R1 | TTATTT | 91 |
| | | AGTTTT | | | CCTAAA | |
| | | GAAGGG | | | ACTTAT | |
| | | AA | | | AAATTT | |
| | | | | | ATAAAA | |
| 49 | 49F1 | TTGTTT | 30 | 49R1 | AATTAT | 92 |
| | | TTAAAA | | | ATTTCA | |
| | | AAATTA | | | AACCTT | |
| | | AAAAGA | | | ATCTTA | |
| | | G | | | AAAC | |
| 54 | 54F1 | TGAGAT | 31 | 54R1 | CTTACA | 93 |
| | | GATTAA | | | CACCTA | |
| | | ATGAAG | | | AACTAA | |
| | | ATTAAA | | | TTACCA | |
| 55 | 55F1 | GTTTGT | 32 | 55R1 | CCTACT | 94 |
| | | TGTTTT | | | AATCTT | |
| | | GTAGAA | | | ACTCAA | |
| | | AAATAA | | | CAAACA | |
| 56 | 56F1 | TAGTTT | 33 | 56R1 | ATTAAT | 95 |
| | | GAGAAA | | | TCTAAA | |
| | | TAGGTA | | | ATAATA | |
| | | ATAAAA | | | CTAAAA | |
| | | ATAG | | | ACTTTT | |
| | | | | | AC | |
| 57 | 57F1 | TTATTT | 34 | 57R1 | CCTTTC | 96 |
| | | TTGTTA | | | ATTTAA | |
| | | ATTTAA | | | AATATT | |
| | | GTAAGG | | | TCCAA | |
| | | TAGA | | | | |
| 58 | 58F1 | GGGTTT | 35 | 58R1 | CCCTCA | 97 |
| | | TTTATT | | | ACCTCC | |
| | | TTGGAA | | | TAAATA | |
| | | TTAAG | | | CA | |
| 59 | 59F1 | TTGTGG | 36 | 59R1 | AAAAAT | 98 |
| | | GTGTAA | | | ACCATT | |
| | | ATAAAT | | | TACCTA | |
| | | TGA | | | ACCA | |
| 63 | 63F1 | TTGGTT | 37 | 63R1 | AACATC | 99 |
| | | GTTTTG | | | TCATTT | |
| | | GATGAT | | | TCAAAC | |
| | | GA | | | TACAC | |
| 64 | 64F1 | GTTTAA | 38 | 64R1 | CATACC | 100 |
| | | GGTTTA | | | ATTAAC | |
| | | TAAGAA | | | TAAAAA | |
| | | GAGGAA | | | ACCC | |
| 65 | 65F1 | TTGGGG | 39 | 65R1 | TCCATA | 101 |
| | | TTATAG | | | ACAATC | |
| | | TTGGAG | | | ACTCAC | |
| | | AG | | | TAAA | |
| 66 | 66F1 | TGTTTT | 40 | 66R1 | AACTTA | 102 |
| | | AGAAAG | | | AACCCA | |
| | | AAAAGA | | | AAACTT | |
| | | AAAAGA | | | TAAAAC | |
| | | | | | TACA | |
| 69 | 69F1 | GGAAAT | 41 | 69R1 | CCTTCC | 103 |
| | | ATTGAT | | | ACCAAA | |
| | | TTTTGA | | | TATTCA | |
| | | TAGAAG | | | AA | |
| 70 | 70F1 | TAGGAT | 42 | 70R1 | TCCTTC | 104 |
| | | GGTAGG | | | ACATAC | |
| | | GTTGGG | | | CAAAAA | |
| | | AG | | | AA | |
| 71 | 71F1 | AAAATT | 43 | 71R1 | TCCTTA | 105 |
| | | AATGAA | | | AAAAAA | |
| | | TTGTTA | | | AACCTA | |
| | | AAGTTT | | | CAAA | |
| | | AAG | | | | |
| 72 | 72F1 | TTTTTT | 44 | 72R1 | CCATTT | 106 |
| | | GAGATT | | | AATATA | |
| | | TGTTAA | | | AATCAC | |
| | | GAAAG | | | ATAACC | |
| | | | | | A | |
| 73 | 73F1 | TTGATT | 45 | 73R1 | ACTAAC | 107 |
| | | TTGTTT | | | CACCCT | |
| | | TGGAGT | | | CTCCTA | |
| | | GA | | | CA | |
| 74 | 74F1 | AATTTT | 46 | 74R1 | AACAAA | 108 |
| | | GAATTT | | | ACACTT | |
| | | TATTTG | | | AATCTC | |
| | | TAAAGT | | | CTACA | |
| | | AGA | | | | |
| 76 | 76F1 | TTGTTT | 47 | 76R1 | TTCTTA | 109 |
| | | ATTTTT | | | AAATAA | |
| | | AGTGGA | | | AACACT | |
| | | TTGAG | | | ACACAC | |
| | | | | | A | |
| 77 | 77F1 | ATGTTG | 48 | 77R1 | CACAAC | 110 |
| | | GTAGAG | | | AAACTA | |
| | | TGGGGT | | | ATTAAC | |
| | | TGA | | | CAAA | |
| 81 | 81F1 | GAGGAT | 49 | 81R1 | ACAATT | 111 |
| | | TTGTAA | | | CTCTTT | |
| | | TTGGTA | | | CCTTTA | |
| | | TAGAAG | | | AAATAA | |
| 82 | 82F1 | GTGGGT | 50 | 82R1 | AAATAC | 112 |
| | | GATTTG | | | CCTCCT | |
| | | ATGGGT | | | ATTATT | |
| | | GA | | | TTAAAA | |
| | | | | | C | |
| 83 | 83F1 | ATGTTT | 51 | 83R1 | AATAAT | 113 |
| | | TGAAGG | | | CAAAAA | |
| | | AGGGTT | | | TAATTT | |
| | | GA | | | ATTAAA | |
| | | | | | TATTAA | |
| | | | | | ATAC | |
| 84 | 84F1 | TTGGTG | 52 | 84R1 | CACAAA | 114 |
| | | ATTGTT | | | AACATC | |
| | | GAAAAT | | | TCTCTA | |
| | | GA | | | TATACA | |
| | | | | | A | |
| 86 | 86F1 | ATAAAT | 53 | 86R1 | CATCAC | 115 |
| | | TAAAGA | | | ACCCTT | |
| | | GTTAAG | | | ACTAAT | |
| | | TATTAG | | | TACC | |
| | | AAATGA | | | | |
| 87 | 87F1 | TTTTTT | 54 | 87R1 | CTTTCC | 116 |
| | | TTGTTT | | | AACCTA | |
| | | AATAAA | | | AAAAAT | |
| | | GGTGA | | | AACA | |
| 88 | 88F1 | TGTATG | 55 | 88R1 | ATCTCA | 117 |
| | | TTTTAG | | | AAAAAT | |
| | | TATTTT | | | AAATTT | |
| | | GTTTTA | | | CCAA | |
| | | AGTTAG | | | | |
| 92 | 92F1 | TGGGTG | 56 | 92R1 | CAAAAT | 118 |
| | | TTAAGT | | | ATAAAA | |
| | | TAGTTT | | | ATCAAA | |
| | | AATAAA | | | TCCC | |
| | | G | | | | |
| 93 | 93F1 | TTATAG | 57 | 93R1 | TCAAAT | 119 |
| | | GGAAGG | | | AACACC | |
| | | GTAGGG | | | TAAATA | |
| | | AG | | | ATAATC | |
| | | | | | C | |
| 94 | 94F1 | TGAATT | 58 | 94R1 | ACTCCA | 120 |
| | | TTAGTA | | | AACTTC | |
| | | TTGGTG | | | CCCAAC | |
| | | TATATG | | | AA | |
| | | AG | | | | |
| 95 | 95F1 | AGAGTT | 59 | 95R1 | ACAACT | 121 |
| | | AAGTTA | | | CAAAAC | |
| | | GATGTG | | | TCTAAA | |
| | | TTATAA | | | ATAATA | |
| | | TTAGAG | | | AAC | |
| 97 | 97F1 | ATTTAG | 60 | 97R1 | TTTATA | 122 |
| | | GAATGT | | | CAATCA | |
| | | AGATTA | | | CAAACA | |
| | | AAGTGA | | | TACCA | |
| | | A | | | | |
| 99 | 99F1 | GTGTGT | 61 | 99R1 | CTTAAC | 123 |
| | | GTTGTG | | | CTAAAC | |
| | | GTGAGG | | | TCCCCA | |
| | | AG | | | AA | |
| 100 | 100F1 | GAGTTA | 62 | 100R1 | ATTTAA | 124 |
| | | GTGTTT | | | ACCTCA | |
| | | TTATTA | | | TAACCC | |
| | | TAGGAG | | | TATAAA | |
| | | AGA | | | | |

TABLE 5
Primer designed in Comparative Example 1

| | Forward primer | | | Reverse primer | | |
|---|---|---|---|---|---|---|
| Mea- | | Base | | | Base | |
| sure- | | se- | | | se- | |
| ment | | quence | Se- | | quence | Se- |
| site | | (5′ → | quence | | (5′ → | quence |
| ID | Name | 3′) | number | Name | 3′) | number |
| 5 | 5F2 | GTTTGA | 125 | 5R2 | TCTAAA | 129 |
| | | AATGTT | | | ACTATT | |
| | | ATTTTT | | | AATATC | |
| | | AATAAG | | | TCTAAA | |
| | | G | | | AAACTA | |
| | | | | | AA | |
| 26 | 26F2 | TGTATA | 126 | 26R2 | AACAAA | 130 |
| | | GATGGG | | | AAAAAC | |
| | | GAAATA | | | ACTAAT | |
| | | GAGG | | | AAAACT | |
| | | | | | AAA | |
| 37 | 37F2 | TTGGGG | 127 | 37R2 | CCACAA | 131 |
| | | TTAGGA | | | AAAACC | |
| | | TGAGAG | | | CTAAAA | |
| | | AA | | | AACTAA | |
| | | | | | A | |
| 87 | 87F2 | ATAAAG | 128 | 87R2 | TTTCCA | 132 |
| | | GTGAAG | | | ACCTAA | |
| | | GGTGTG | | | AAAATA | |
| | | GG | | | ACAA | |

### Examples 1 to 4 and Comparative Examples 2 to 4

Based on the base sequence data of reference genome GRCh37 (GenBank assembly accession: GCA_000001405.1, RefSeq assembly accession: GCF_000001405.13), randomly selected 100 measurement sites (target sites) shown in Table 1, and the position information on the target sites, a primer sequence for multiplex PCR producing a PCR amplification product having a length of 70 bp to 120 bp was designed using the primer design device of the first embodiment. The primer was designed such that the primer had a length of 20 to 35 bases (mer), and that only C in a CG sequence can be methylated. In addition, the conditions for selecting the partial sequence were set as follows.

Condition (1): The Tm value is in a range of 55° C. to 65° C.

Condition (2): The number of YG sequences or CR sequences included in a partial sequence is 0.

Condition (3): The upper limit of the number of binding sites with the sequence outside the related region is 2.

In the calculation of the local alignment score, <1> a complementary base pair is set to “X”=1 per pair, <2> a non-complementary base pair is set to “Y”=−3 per pair, and <3> a case where there is insertion or deletion is set to “Z”=−6 per one insertion or deletion between the sequences.

The threshold values of Examples 1 to 4 and Comparative Examples 2 to 4 were set as shown in Table 6.

In addition, the dimer formation rate of the same primer as the conditions (the parameters used for calculating the score, and the threshold value) for the local alignment score used in each of Examples and Comparative Examples was also calculated. One primer set in which a local alignment score between two primers for amplifying separately selected 91 target sites distributed in a range of 0 to 6 (that is, one pair was designed for each target site, and a total of 182 primers were prepared) was prepared and the preparation DNA (Human WGA Methylated DNA, Zymo Research Corporation) subjected to the bisulfite treatment was amplified by multiplex PCR. The sequence of the obtained amplification product was acquired by a next-generation sequencer (MiSeq, Illumina, Inc.). Here, the acquired sequence consists of a target amplification product containing a target site, a primer dimer, and other non-specific amplification products. All primer dimer sequences that can be generated from the prepared primer sequences were generated in the computer, and the generated primer dimer sequences were collated and counted with the sequences acquired by the next-generation sequencer to detect the actually generated primer dimer sequences and the number thereof. All combinations of two sequences selected from the prepared primer sequences were assigned to seven groups of 0 to 6 according to the local alignment score. A proportion of the number of actually generated primer dimers (10 or more sequences acquired by the next-generation sequencer) among the number of two sequences belonging to each group was calculated and defined as the dimer formation rate.

TABLE 6

| | Comparative | Example | Example | Example | Example | Comparative | Comparative |
|---|---|---|---|---|---|---|---|
| | Example 2 | 1 | 2 | 3 | 4 | Example 3 | Example 4 |
| X | 1 | 1 | 1 | 1 | 1 | 1 | 1 |
| Y | 3 | 3 | 3 | 3 | 3 | 3 | 3 |
| Z | 6 | 6 | 6 | 6 | 6 | 6 | 6 |
| Local alignment | 0 | 1 | 2 | 3 | 4 | 5 | 6 |
| score threshold | | | | | | | |
| value | | | | | | | |
| Primer design | 43% | 62% | 68% | 78% | 82% | 84% | 84% |
| success rate | | | | | | | |
| Dimer formation | 1% | 1% | 1% | 2% | 2% | 20% | 50% |
| rate | | | | | | | |

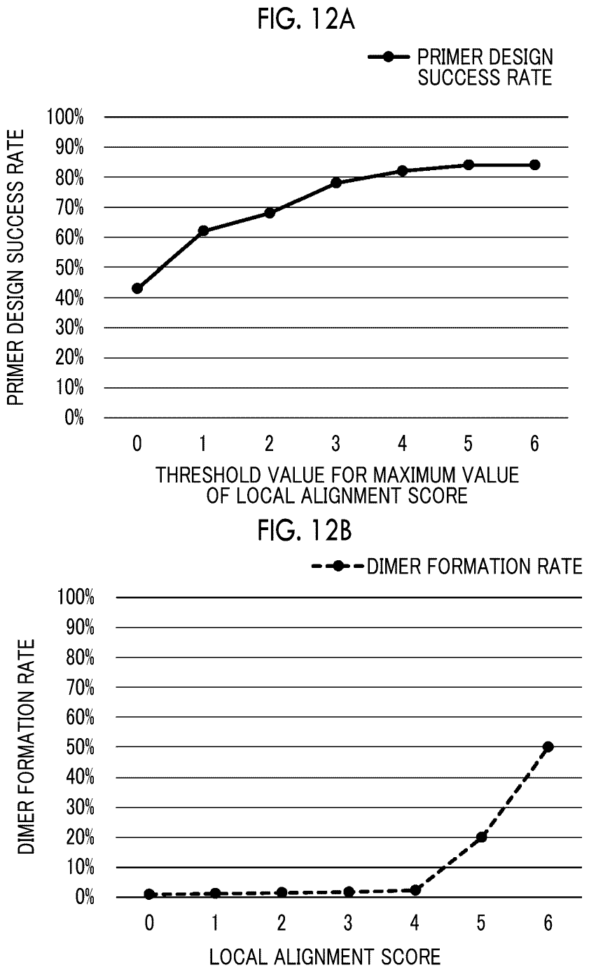

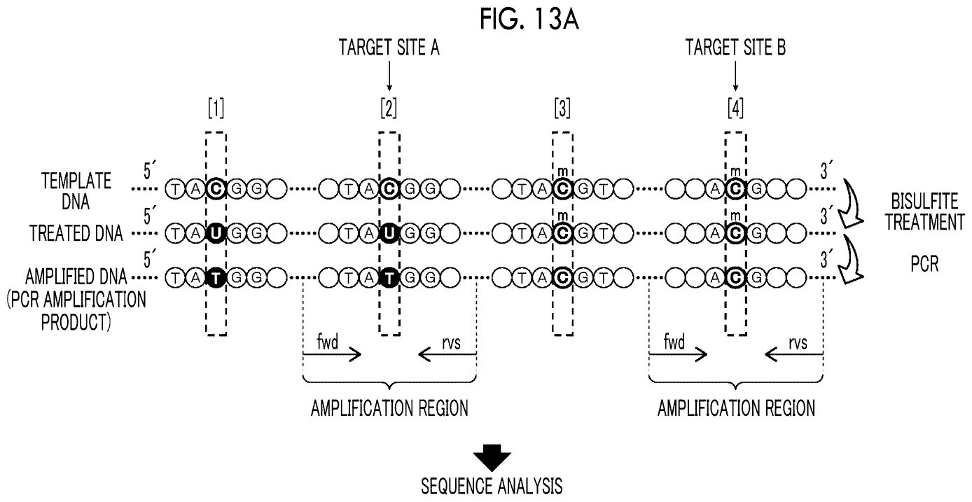

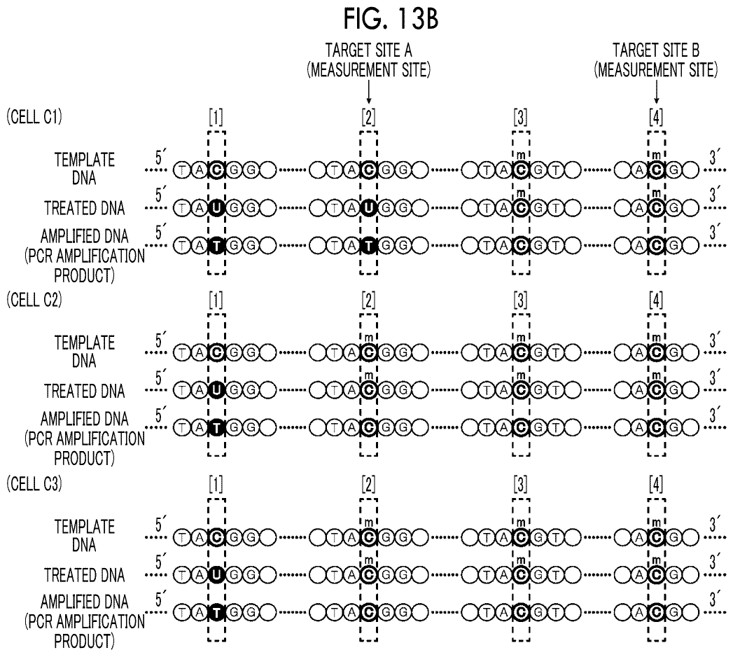

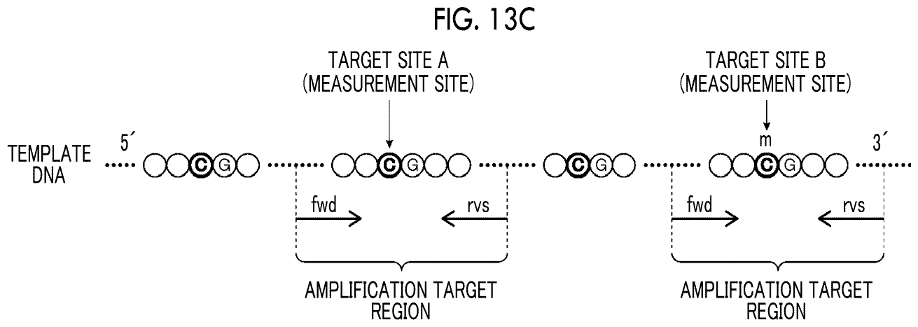

Table 7 shows whether the primer for each measurement site of Examples 1 to 4 and Comparative Examples 2 to 4 is successfully designed or failed to be designed and shows the primer design success rate calculated from the results of the success or failure of the primer design. In addition, Tables 8 to 10 show the primers that could be designed in Examples 2 to 4, and Tables 11 to 13 shows the primers that could be designed in Comparative Examples 2 to 4. FIG. 12A shows the primer design success rate for each threshold value set in the case of primer sequence determination based on each of Examples and Comparative Examples, and FIG. 12B shows the dimer formation rate for each threshold value designed in each of Examples and Comparative Examples.

TABLE 7

| | | | Success or failure of design | | | | | | |
|---|---|---|---|---|---|---|---|---|---|
| | | | Compar- | | | | | Compar- | Compar- |
| Measurement site | | | ative | | | | | ative | ative |
| | Chromo- | Coor- | Exam- | Exam- | Exam- | Exam- | Exam- | Exam- | Exam- |
| ID | some | dinate | ple 2 | ple 1 | ple 2 | ple 3 | ple 4 | ple 3 | ple 4 |
| 1 | 6 | 29870056 | — | — | — | — | — | — | — |
| 2 | 7 | 4389129 | X | X | X | X | X | X | X |
| 3 | 14 | 105391263 | X | X | X | X | X | X | X |
| 4 | 15 | 26302108 | — | — | — | — | — | — | — |
| 5 | 8 | 19313167 | X | X | X | X | X | X | X |
| 6 | 7 | 27561178 | — | X | X | X | X | X | X |
| 7 | 7 | 151553782 | — | — | X | X | X | X | X |
| 8 | 11 | 1862477 | X | X | X | X | X | X | X |
| 9 | 6 | 84221752 | — | — | — | — | — | X | X |
| 10 | 12 | 114677042 | X | X | X | X | X | X | X |
| 11 | 6 | 29589729 | — | — | — | — | — | — | — |
| 12 | 13 | 49076914 | — | X | X | X | X | X | X |
| 13 | 7 | 76109396 | X | X | X | X | X | X | X |
| 14 | 3 | 128186859 | X | X | X | X | X | X | X |
| 15 | 12 | 34756440 | X | X | — | X | X | X | X |
| 16 | 10 | 115991467 | X | X | X | X | X | X | X |
| 17 | 14 | 107095027 | — | — | X | X | X | X | X |
| 18 | 10 | 130268585 | X | X | X | X | X | X | X |
| 19 | 6 | 31515526 | — | — | — | — | — | — | — |
| 20 | 7 | 2414948 | X | — | X | X | X | X | X |
| 21 | 6 | 32030188 | — | — | — | — | — | — | — |
| 22 | 13 | 113992654 | X | X | X | X | X | X | X |
| 23 | 10 | 132099067 | — | X | X | X | X | X | X |
| 24 | 6 | 168618157 | — | X | X | X | X | X | X |
| 25 | 1 | 161916064 | X | — | X | X | X | X | X |
| 26 | 11 | 45354409 | X | X | X | X | X | X | X |
| 27 | 16 | 70516599 | X | X | X | X | X | X | X |
| 28 | 2 | 233096291 | X | X | X | X | X | X | X |
| 29 | 1 | 8601318 | X | X | X | X | X | X | X |
| 30 | 3 | 57125501 | X | X | X | X | X | X | X |
| 31 | 9 | 116298900 | X | X | X | X | X | X | X |
| 32 | 9 | 97317179 | X | X | X | X | X | X | X |
| 33 | 9 | 117692954 | — | — | — | — | — | — | — |
| 34 | 14 | 104742172 | — | — | — | — | — | — | — |
| 35 | 17 | 78058778 | X | X | X | X | X | X | X |
| 36 | 16 | 11482317 | X | X | X | X | X | X | X |
| 37 | 11 | 44291407 | X | X | X | X | X | X | X |
| 38 | 13 | 39564046 | X | X | X | X | X | X | X |
| 39 | 14 | 104824020 | — | — | — | — | X | X | X |
| 40 | 2 | 112822975 | — | — | X | X | X | X | X |
| 41 | 15 | 32162729 | — | — | — | — | — | — | — |
| 42 | 2 | 187826872 | — | X | — | X | X | X | X |
| 43 | 10 | 131460030 | — | X | — | X | X | X | X |
| 44 | 19 | 19106904 | — | — | — | X | X | X | X |
| 45 | 14 | 56856095 | — | — | — | X | X | X | X |
| 46 | 7 | 156755824 | — | — | — | — | X | X | X |
| 47 | 3 | 65652312 | X | X | X | X | X | X | X |
| 48 | 8 | 122680033 | — | — | — | — | — | — | — |
| 49 | 5 | 140090404 | — | X | X | X | X | X | X |
| 50 | 12 | 116715986 | — | — | X | X | X | X | X |

| | | | Success or failure of design | | | | | | |
|---|---|---|---|---|---|---|---|---|---|
| | | | Com- | | | | | Com- | Com- |
| | | | par- | | | | | par- | par- |
| Measurement site | | | ative | | | | | ative | ative |
| | Chromo- | Coor- | Exam- | Exam- | Exam- | Exam- | Exam- | Exam- | Exam- |
| ID | some | dinate | ple 2 | ple 1 | ple 2 | ple 3 | ple 4 | ple 3 | ple 4 |
| 51 | 19 | 54196147 | — | — | — | X | X | X | X |
| 52 | 3 | 128199781 | — | — | X | X | X | X | X |
| 53 | 1 | 46112691 | — | — | X | X | X | X | X |
| 54 | 5 | 147714437 | X | X | X | X | X | X | X |
| 55 | 1 | 3721794 | X | X | X | X | X | X | X |
| 56 | 7 | 18534872 | X | X | X | X | X | X | X |
| 57 | 8 | 72518106 | — | X | X | X | X | X | X |
| 58 | 1 | 12268883 | — | X | X | X | X | X | X |
| 59 | 2 | 99526035 | X | X | X | X | X | X | X |
| 60 | 14 | 101513595 | — | — | — | X | X | X | X |
| 61 | 6 | 64734868 | — | — | — | — | — | — | — |
| 62 | 17 | 40322138 | — | — | — | — | — | — | — |
| 63 | 5 | 138861855 | — | X | X | X | X | X | X |
| 64 | 3 | 178984973 | X | X | X | X | X | X | X |
| 65 | 3 | 100148679 | X | X | X | X | X | X | X |
| 66 | 11 | 20152992 | — | X | X | X | X | X | X |
| 67 | 14 | 23624363 | — | — | X | X | X | X | X |
| 68 | 6 | 2623483 | — | — | X | X | X | X | X |
| 69 | 1 | 44344466 | X | X | X | X | X | X | X |
| 70 | 6 | 39849807 | — | X | X | X | X | X | X |
| 71 | 12 | 51180192 | — | X | X | X | X | X | X |
| 72 | 17 | 43651976 | X | X | X | X | X | X | X |
| 73 | 10 | 104832357 | — | X | X | X | X | X | X |
| 74 | 1 | 220876396 | X | X | X | X | X | X | X |
| 75 | 12 | 63238340 | X | — | X | X | X | X | X |
| 76 | X | 146312617 | X | X | X | X | X | X | X |
| 77 | 14 | 76734327 | — | X | X | X | X | X | X |
| 78 | 13 | 19847419 | — | — | — | — | — | — | — |
| 79 | 8 | 7004738 | — | — | — | — | — | — | — |
| 80 | 4 | 106768095 | — | — | — | — | — | — | — |
| 81 | 12 | 67278182 | X | X | X | X | X | X | X |
| 82 | 7 | 157374793 | — | X | X | X | X | X | X |
| 83 | 1 | 8427556 | — | X | X | X | X | X | X |
| 84 | 7 | 121437819 | X | X | X | X | X | X | X |
| 85 | 5 | 79222121 | — | — | — | X | X | X | X |
| 86 | 1 | 212662017 | X | X | X | X | X | X | X |
| 87 | 10 | 3805441 | — | X | X | X | X | X | X |
| 88 | 14 | 75886161 | X | X | X | X | X | X | X |
| 89 | 17 | 39781108 | — | — | — | — | X | X | X |
| 90 | 13 | 113506845 | — | — | — | — | — | — | — |
| 91 | 2 | 175436504 | — | — | — | — | X | X | X |
| 92 | 9 | 130323725 | — | X | X | X | X | X | X |
| 93 | 1 | 19717337 | X | X | — | X | X | X | X |
| 94 | 13 | 96454018 | — | X | X | X | X | X | X |
| 95 | 1 | 206644843 | X | X | X | X | X | X | X |
| 96 | 6 | 30034500 | — | — | — | — | — | — | — |
| 97 | 11 | 130116833 | — | X | X | X | X | X | X |
| 98 | 17 | 76220898 | — | — | — | — | — | X | X |
| 99 | 2 | 113931518 | X | X | — | X | X | X | X |
| 100 | 1 | 243603467 | — | X | X | X | X | X | X |
| | | Design | 43% | 52% | 68% | 78% | 82% | 84% | 84% |
| | | access | | | | | | | |
| | | rate | | | | | | | |

TABLE 8
Primer designed in Example 2

| | Forward primer | | | Reverse primer | | |
|---|---|---|---|---|---|---|
| Mea- | | Base | | | Base | |
| sure- | | se- | | | se- | |
| ment | | quence | Se- | | quence | Se- |
| site | | (5′ → | quence | | (5′ → | quence |
| ID | Name | 3′) | number | Name | 3′) | number |
| 2 | 2F1 | TGGTAG | 133 | 2R1 | TAATCC | 201 |
| | | TGATTA | | | CACTTA | |
| | | GTTTAT | | | CAAAAA | |
| | | TTTTTG | | | ACAC | |
| 3 | 3F1 | TGTAGA | 134 | 3R1 | TTAATA | 202 |
| | | GAGGAG | | | TCTATC | |
| | | GAGGTG | | | CTAATT | |
| | | AG | | | CCAACC | |
| 5 | 5F1 | TTTTTG | 135 | 5R1 | TCAAAA | 203 |
| | | GGTTTG | | | CATTTC | |
| | | AAATGT | | | TAAAAC | |
| | | TA | | | TATTAA | |
| | | | | | TATC | |
| 6 | 6F1 | GGGTTG | 136 | 6R1 | TACTAA | 204 |
| | | AGGATT | | | TCTAAC | |
| | | AGTATT | | | AAAAAA | |
| | | GATT | | | CAAAAC | |
| | | | | | TTAAAC | |
| | | | | | A | |
| 7 | 7F1 | GGTTGA | 137 | 7R1 | TTAAAT | 205 |
| | | TGAGGT | | | CTAACA | |
| | | ATAGGT | | | CCCACA | |
| | | GA | | | CC | |
| 8 | 8F1 | AAGAAG | 138 | 8R1 | CAAATA | 206 |
| | | GAGGAT | | | TAAAAA | |
| | | ATAGAG | | | ATAATC | |
| | | AAGG | | | CCCA | |
| 10 | 10F1 | TTGTTT | 139 | 10R1 | CAAACA | 207 |
| | | TTTGTT | | | AAATTT | |
| | | GTGTGG | | | ACAACC | |
| | | AA | | | CA | |
| 12 | 12F1 | TTTTTA | 140 | 12R1 | ACCTCA | 208 |
| | | GGTTAT | | | CCCACT | |
| | | TTTTTA | | | TCTCCT | |
| | | AATGGA | | | AC | |
| 13 | 13F1 | TGTGAT | 141 | 13R1 | CACCCA | 209 |
| | | TTTAGT | | | ACTCAT | |
| | | ATTTGG | | | TTTTTT | |
| | | GAAG | | | AC | |
| 14 | 14F1 | TTTATG | 142 | 14R1 | AATACT | 210 |
| | | AAGGGA | | | CCCCAC | |
| | | GTTGTG | | | TAACTC | |
| | | GA | | | CC | |
| 16 | 16F1 | TTAGGT | 143 | 16R1 | CAAAAA | 211 |
| | | TGGTGG | | | ATCCCT | |
| | | TTTTTA | | | ATATAT | |
| | | TTT | | | TCCTTA | |
| 17 | 17F1 | TTTAGA | 144 | 17R1 | CCTCAT | 212 |
| | | TATAAA | | | ACTCTA | |
| | | TTTTTT | | | AAAACC | |
| | | TGTATG | | | CC | |
| | | GA | | | | |
| 18 | 18F1 | TTGGGG | 145 | 18R1 | CCCCAC | 213 |
| | | TAGAGT | | | TAATAC | |
| | | ATAGGT | | | TTCCTT | |
| | | TAGTT | | | ACC | |
| 20 | 20F1 | AGAGGT | 146 | 20R1 | CATACC | 214 |
| | | TGTTGT | | | TCCTAA | |
| | | TGTGTT | | | CATCCC | |
| | | TG | | | AC | |
| 22 | 22F1 | TGGTAG | 147 | 22R1 | CCTCTA | 215 |
| | | GTGTTT | | | ATCCCA | |
| | | TGGGTT | | | ATTCAA | |
| | | GA | | | TTA | |
| 23 | 23F1 | TGTTGT | 148 | 23R1 | AAAAAT | 216 |
| | | TTTGTT | | | AAACCT | |
| | | TGTATG | | | TACAAA | |
| | | GA | | | CTACAC | |
| | | | | | A | |
| 24 | 24F1 | GTTTTT | 149 | 24R1 | ACTCCC | 217 |
| | | TGTTGG | | | AAACAC | |
| | | TGGGAA | | | CCCTTT | |
| | | TA | | | TT | |
| 25 | 25F1 | TGATAA | 150 | 25R1 | CCATAT | 218 |
| | | AGATTT | | | TACCCC | |
| | | TGTAGG | | | AACTAC | |
| | | GGTTA | | | TCT | |
| 26 | 26F1 | GGAAGG | 151 | 26R1 | AAATAA | 219 |
| | | GTATTG | | | ATATTA | |
| | | GTGGGA | | | TTATAC | |
| | | TT | | | CCACTA | |
| | | | | | CACA | |
| 27 | 27F1 | AAGTGG | 152 | 27R1 | TAACCT | 220 |
| | | GTTTGG | | | AACCAC | |
| | | GAAGTA | | | AAACAA | |
| | | TG | | | CC | |
| 28 | 28F1 | TTTAGG | 153 | 28R1 | ACCTCA | 221 |
| | | GAGATA | | | AATAAC | |
| | | TATTTT | | | TTAAAA | |
| | | GGTTT | | | TTCACT | |
| 29 | 29F1 | TTTAAT | 154 | 29R1 | TCCCAT | 222 |
| | | GAATGG | | | TTTTCT | |
| | | ATATAA | | | AACATA | |
| | | GTGATT | | | TTTACT | |
| | | TA | | | | |
| 30 | 30F1 | TTGGGT | 155 | 30R1 | TTTCTC | 223 |
| | | GTGTAA | | | AACTTC | |
| | | GAATTT | | | ACACTT | |
| | | TT | | | AATTT | |
| 31 | 31F1 | AGTTGG | 156 | 31R1 | CAAATA | 224 |
| | | TTTTTG | | | CACACT | |
| | | AATTTA | | | AATCCC | |
| | | TTTTT | | | CA | |
| 32 | 32F1 | TTTTTT | 157 | 32R1 | CCAAAA | 225 |
| | | ATATTT | | | TCTCCT | |
| | | ATATAT | | | AACCTC | |
| | | AAGTGT | | | AATA | |
| | | TAGAAA | | | | |
| | | TG | | | | |
| 35 | 35F1 | GAGGAA | 158 | 35R1 | CCCTAC | 226 |
| | | GTAAGG | | | CTAAAA | |
| | | GTTTAT | | | CCTCAC | |
| | | TAATTT | | | CC | |
| 36 | 36F1 | ATTTTA | 159 | 36R1 | TCTTCC | 227 |
| | | TGGTTG | | | ATACTA | |
| | | GGGAAA | | | ATAACC | |
| | | TTG | | | TCACA | |
| 37 | 37F1 | GGGGTT | 160 | 37R1 | TTCCCC | 228 |
| | | AGGATG | | | CACAAA | |
| | | AGAGAA | | | AAACCC | |
| | | TG | | | TA | |
| 38 | 38F1 | TTGGTA | 161 | 38R1 | AAATTA | 229 |
| | | ATATAA | | | TTCAAA | |
| | | GGTATA | | | AAATAA | |
| | | GAGTAT | | | TTATAA | |
| | | AGGTTA | | | TAATAA | |
| | | | | | TATAC | |
| 40 | 40F1 | TAATTG | 162 | 40R1 | TAACTC | 230 |
| | | GGTAGG | | | CTAAAC | |
| | | GTGGGT | | | TTAAAT | |
| | | TA | | | AATCCT | |
| | | | | | CT | |
| 47 | 47F1 | GTTTTG | 163 | 47R1 | TCTCAA | 231 |
| | | AAGGGA | | | CATTAT | |
| | | AGATAG | | | TTCCTA | |
| | | GA | | | AAACTT | |
| | | | | | A | |
| 49 | 49F1 | AAAAAA | 164 | 49R1 | CAAAAT | 232 |
| | | ATTAAA | | | ATAAAT | |
| | | AAGAGT | | | TATATT | |
| | | AATAGG | | | TCAAAC | |
| | | AA | | | CTTA | |
| 50 | 50F1 | GTTTTG | 165 | 50R1 | AAACTC | 233 |
| | | GGGAAT | | | CTCTTC | |
| | | GTGTTT | | | CCAAAT | |
| | | TTA | | | ATAC | |
| 52 | 52F1 | GTAATT | 166 | 52R1 | TTAACA | 234 |
| | | GTTGGT | | | ACCCAA | |
| | | AGGTTG | | | CATTTC | |
| | | TTG | | | CC | |
| 53 | 53F1 | TTAAAT | 167 | 53R1 | ACTAAA | 235 |
| | | TTTTTT | | | AAATAA | |
| | | TTTTTA | | | AAAAAA | |
| | | GTTTTA | | | TAAATA | |
| | | ATTT | | | ATATTT | |
| | | | | | T | |
| 54 | 54F1 | TTTATG | 168 | 54R1 | CACCAC | 236 |
| | | AGATGA | | | TCTCCA | |
| | | TTAAAT | | | TATAAC | |
| | | GAAGAT | | | CTTA | |
| | | T | | | | |
| 55 | 55F1 | GGTTGT | 169 | 55R1 | CAAAAT | 237 |
| | | TGTAAT | | | CAACCA | |
| | | TGTTTG | | | CAACCT | |
| | | TTG | | | ACT | |
| 56 | 56F1 | GGTAAT | 170 | 56R1 | AAAAAA | 238 |
| | | AAAAAT | | | ATACAA | |
| | | AGAATA | | | AACTCT | |
| | | TTAGGA | | | ATATTA | |
| | | TTG | | | ATTCT | |
| 57 | 57F1 | GTTGTG | 171 | 57R1 | TCCAAC | 239 |
| | | GGGTTG | | | TACTTA | |
| | | TGAATT | | | TTCCCT | |
| | | TTT | | | CTTA | |
| 58 | 58F1 | GGTTTT | 172 | 58R1 | CAAAAA | 240 |
| | | AGTGAT | | | AAATTT | |
| | | TTTTTT | | | TCCACC | |
| | | TTTAGT | | | CTA | |
| | | T | | | | |
| 59 | 59F1 | GGGTGT | 173 | 59R1 | CTAAAA | 241 |
| | | AAATAA | | | AAAAAA | |
| | | ATTGAG | | | AATACC | |
| | | TTGTTA | | | ATTTAC | |
| | | | | | C | |
| 63 | 63F1 | GATATT | 174 | 63R1 | ACACTC | 242 |
| | | GGTTGT | | | AAAAAA | |
| | | TTTGGA | | | ACTACC | |
| | | TG | | | CTTA | |
| 64 | 64F1 | AAGAGG | 175 | 64R1 | CATACC | 243 |
| | | AAATGT | | | ATTAAC | |
| | | TTTGTT | | | TAAAAA | |
| | | TTG | | | ACCC | |
| 65 | 65F1 | ATGTGT | 176 | 65R1 | AAAAAT | 244 |
| | | TTTTTG | | | TTTCCT | |
| | | TTAAAT | | | ATAACT | |
| | | GGA | | | AATAAC | |
| | | | | | TTACA | |
| 66 | 66F1 | GAAAAG | 177 | 66R1 | AACTTA | 245 |
| | | AAAAAG | | | AACCCA | |
| | | AGAAAG | | | AAACTT | |
| | | TTTTT | | | TAAAAC | |
| | | | | | TAC | |
| 67 | 67F1 | GATAGT | 178 | 67R1 | CAAAAC | 246 |
| | | AATATT | | | ACCTCC | |
| | | TTTTTT | | | TCTCCT | |
| | | TTTTTA | | | TT | |
| | | GTTTTT | | | | |
| 68 | 68F1 | ATTAGT | 179 | 68R1 | TCTAAA | 247 |
| | | TGAGTT | | | CCCCTC | |
| | | TTTTTT | | | CTCATT | |
| | | TTTTTT | | | AC | |
| | | TA | | | | |
| 69 | 69F1 | TGTGGA | 180 | 69R1 | TCAAAA | 248 |
| | | AATATT | | | ATCTAC | |
| | | GATTTT | | | CTTCCA | |
| | | TGA | | | CC | |
| 70 | 70F1 | TTTTAT | 181 | 70R1 | CTAACC | 249 |
| | | ATGTTA | | | CCAAAA | |
| | | GGGAAA | | | ACAATA | |
| | | GTTTTT | | | CAC | |
| 71 | 71F1 | TTAGTA | 182 | 71R1 | AACCTA | 250 |
| | | GGAAAA | | | CAAAAA | |
| | | TTAATG | | | AATATA | |
| | | AATTGT | | | AACTAT | |
| | | TA | | | CTTT | |
| 72 | 72F1 | TTGAAT | 183 | 72R1 | AAATAT | 251 |
| | | GTTGTT | | | ATTCTA | |
| | | ATTTGG | | | TAATTC | |
| | | TATG | | | CCACAC | |
| | | | | | TTA | |
| 73 | 73F1 | TTATTT | 184 | 73R1 | AACTAA | 252 |
| | | GATTTT | | | CCACCC | |
| | | GTTTTG | | | TCTCCT | |
| | | GAG | | | AC | |
| 74 | 74F1 | GTTTTT | 185 | 74R1 | AACAAA | 253 |
| | | TAATTT | | | ACACTT | |
| | | TGAATT | | | AATCTC | |
| | | TTATTT | | | CTACA | |
| | | G | | | | |
| 75 | 75F1 | AGTTTT | 186 | 75R1 | CCTTTT | 254 |
| | | AATAGT | | | TATTTA | |
| | | TTTAAG | | | AAAATA | |
| | | TTTGGA | | | ATATTA | |
| | | TT | | | AACA | |
| 76 | 76F1 | GTATGG | 187 | 76R1 | TCCTAA | 255 |
| | | TATTTT | | | TAAACT | |
| | | TTGAAG | | | AAAAAT | |
| | | TGAAG | | | ATTAAA | |
| | | | | | ATTCTA | |
| 77 | 77F1 | ATGTTG | 188 | 77R1 | AAACAC | 256 |
| | | GTAGAG | | | AACAAA | |
| | | TGGGGT | | | CTAATT | |
| | | TGA | | | AACCA | |
| 81 | 81F1 | GGATTT | 189 | 81R1 | ATTCTC | 257 |
| | | GTAATT | | | TTTCCT | |
| | | GGTATA | | | TTAAAA | |
| | | GAAGG | | | TAAATA | |
| | | | | | TATC | |
| 82 | 82F1 | GAGGGG | 190 | 82R1 | CATCTC | 258 |
| | | ATGTTT | | | TTACTA | |
| | | TTTTGT | | | AAACTA | |
| | | TG | | | ACATCA | |
| | | | | | CA | |
| 83 | 83F1 | AAAGTT | 191 | 83R1 | CTACTA | 259 |
| | | TATTAT | | | AATATT | |
| | | ATGTAT | | | AATAAT | |
| | | TTTTTG | | | CAAAAA | |
| | | GAG | | | TAATTT | |
| | | | | | ATTA | |
| 84 | 84F1 | TTGTAG | 192 | 84R1 | AACTTC | 260 |
| | | TTGGTG | | | ACAAAA | |
| | | ATTGTT | | | ACATCT | |
| | | GA | | | CTCTA | |
| 86 | 86F1 | AATAAA | 193 | 86R1 | TCACAC | 261 |
| | | TTAAAG | | | CCTTAC | |
| | | AGTTAA | | | TAATTA | |
| | | GTATTA | | | CCC | |
| | | GAAATG | | | | |
| 87 | 87F1 | TTTTGT | 194 | 87R1 | CCTAAA | 262 |
| | | TTAATA | | | AAAATC | |
| | | AAGGTG | | | TTTCCA | |
| | | AAGG | | | ACC | |
| 88 | 88F1 | GGGGGT | 195 | 88R1 | CCCTAA | 263 |
| | | TTTTTT | | | ATCAAC | |
| | | TGGTTT | | | CAAAAT | |
| | | ATG | | | ATAC | |
| 92 | 92F1 | AGGATG | 196 | 92R1 | CACATA | 264 |
| | | AGAGTT | | | TTATCA | |
| | | TTGGTA | | | CCTCCC | |
| | | TTT | | | AC | |
| 94 | 94F1 | TTAGTA | 197 | 94R1 | TTTTCT | 265 |
| | | GGGGTT | | | CCCTTA | |
| | | TTAGAT | | | TAATTT | |
| | | TTTTT | | | TAACAC | |
| 95 | 95F1 | AATTAA | 198 | 95R1 | CACAAA | 266 |
| | | GGTTAA | | | ATCCAA | |
| | | GGGTTT | | | AAACAC | |
| | | TGA | | | ACC | |
| 97 | 97F1 | GAAAGG | 199 | 97R1 | CATACC | 267 |
| | | AGAGAG | | | AATCAT | |
| | | AATTTT | | | CCCCAT | |
| | | GTTA | | | CT | |
| 100 | 100F1 | TGAATT | 200 | 100R1 | TCATTT | 268 |
| | | TGTTGT | | | AAACCT | |
| | | TGATTT | | | CATAAC | |
| | | TG | | | CCTA | |

TABLE 9
Primer designed in Example 3

| | Forward primer | | | Reverse primer | | |
|---|---|---|---|---|---|---|
| Mea- | | Base | | | Base | |
| sure- | | se- | | | se- | |
| ment | | quence | Se- | | quence | Se- |
| site | | (5′ → | quence | | (5′ → | quence |
| ID | Name | 3′) | number | Name | 3′) | number |
| 2 | 2F1 | TGGTAG | 269 | 2R1 | TAATCC | 347 |
| | | TGATTA | | | CACTTA | |
| | | GTTTAT | | | CAAAAA | |
| | | TTTTTG | | | ACAC | |
| 3 | 3F1 | TGTAGA | 270 | 3R1 | TTAATA | 348 |
| | | GAGGAG | | | TCTATC | |
| | | GAGGTG | | | CTAATT | |
| | | AG | | | CCAACC | |
| 5 | 5F1 | TTTTTG | 271 | 5R1 | TCAAAA | 349 |
| | | GGTTTG | | | CATTTC | |
| | | AAATGT | | | TAAAAC | |
| | | TA | | | TATTAA | |
| | | | | | TATC | |
| 6 | 6F1 | GGGTTG | 272 | 6R1 | AAACAA | 350 |
| | | AGGATT | | | AACTTA | |
| | | AGTATT | | | AACAAT | |
| | | GAT | | | AATACT | |
| | | | | | TACTC | |
| 7 | 7F1 | GGTTGA | 273 | 7R1 | TTAAAT | 351 |
| | | TGAGGT | | | CTAACA | |
| | | ATAGGT | | | CCCACA | |
| | | GA | | | CC | |
| 8 | 8F1 | GAAGTA | 274 | 8R1 | AATATA | 352 |
| | | GGTTAA | | | AAAAAT | |
| | | GAAGGA | | | AATCCC | |
| | | GGAT | | | CAAAC | |
| 10 | 10F1 | TTGTTT | 275 | 10R1 | CAAACA | 353 |
| | | TTTGTT | | | AAATTT | |
| | | GTGTGG | | | ACAACC | |
| | | AA | | | CA | |
| 12 | 12F1 | TTTTTA | 276 | 12R1 | ACCTCA | 354 |
| | | GGTTAT | | | CCCACT | |
| | | TTTTTA | | | TCTCCT | |
| | | AATGGA | | | AC | |
| 13 | 13F1 | TGTGAT | 277 | 13R1 | CACCCA | 355 |
| | | TTTAGT | | | ACTCAT | |
| | | ATTTGG | | | TTTTTT | |
| | | GAAG | | | AC | |
| 14 | 14F1 | TTTTTA | 278 | 14R1 | ATACTC | 356 |
| | | TGAAGG | | | CCCACT | |
| | | GAGTTG | | | AACTCC | |
| | | TG | | | CC | |
| 15 | 15F1 | GTGTGG | 279 | 15R1 | ACCCAA | 357 |
| | | AAGGAA | | | AATCTA | |
| | | AAAAAA | | | CAAAAC | |
| | | AG | | | CC | |
| 16 | 16F1 | ATTTTT | 280 | 16R1 | AAATCC | 358 |
| | | TTATTA | | | CTATAT | |
| | | GGTTGG | | | ATTCCT | |
| | | TGG | | | TACCA | |
| 17 | 17F1 | TTTAGA | 281 | 17R1 | CCTCAT | 359 |
| | | TATAAA | | | ACTCTA | |
| | | TTTTTT | | | AAAACC | |
| | | TGTATG | | | CC | |
| | | GA | | | | |
| 18 | 18F1 | TTGGGG | 282 | 18R1 | TCCCCA | 360 |
| | | TAGAGT | | | CTAATA | |
| | | ATAGGT | | | CTTCCT | |
| | | TAGTT | | | TAC | |
| 20 | 20F1 | GGTTAT | 283 | 20R1 | ATACCT | 361 |
| | | TAGAGG | | | CCTAAC | |
| | | TTGTTG | | | ATCCCA | |
| | | TTGT | | | CT | |
| 22 | 22F1 | GGGTGT | 284 | 22R1 | TACCTC | 362 |
| | | GGTAGG | | | TAATCC | |
| | | TGTTTT | | | CAATTC | |
| | | GG | | | AA | |
| 23 | 23F1 | TGGATT | 285 | 23R1 | TATAAA | 363 |
| | | ATTAGG | | | CAACTT | |
| | | TTGAGT | | | AAAAAA | |
| | | TGTT | | | CAACCC | |
| | | | | | C | |
| 24 | 24F1 | TTTAGG | 286 | 24R1 | CCCCTT | 364 |
| | | TTTTTT | | | TTTCAT | |
| | | GTTGGT | | | CAAAAC | |
| | | GG | | | TT | |
| 25 | 25F1 | AGTATT | 287 | 25R1 | AAACTC | 365 |
| | | TTATGT | | | TACAAA | |
| | | TGTTTT | | | AACCAA | |
| | | AGTTGT | | | ATATAA | |
| | | TTT | | | TAA | |
| 26 | 26F1 | AGTAGG | 288 | 26R1 | ATTATT | 366 |
| | | AAGGGT | | | ATACCC | |
| | | ATTGGT | | | ACTACA | |
| | | GG | | | CAAATA | |
| | | | | | AA | |
| 27 | 27F1 | AAGTGG | 289 | 27R1 | TAACCT | 367 |
| | | GTTTGG | | | AACCAC | |
| | | GAAGTA | | | AAACAA | |
| | | TG | | | CC | |
| 28 | 28F1 | TTTTGG | 290 | 28R1 | ACCTCA | 368 |
| | | TTTTAA | | | AATAAC | |
| | | AAGAGA | | | TTAAAA | |
| | | GAAA | | | TTCACT | |
| 29 | 29F1 | TGATTT | 291 | 29R1 | AAAAAA | 369 |
| | | ATTTAT | | | AATATA | |
| | | TTTGTT | | | ACATCT | |
| | | TTAGGG | | | CCCA | |
| 30 | 30F1 | TAAGAG | 292 | 30R1 | TTCACA | 370 |
| | | GAGTTG | | | CTTAAT | |
| | | GGTGTG | | | TTAATT | |
| | | TA | | | ACCCA | |
| 31 | 31F1 | GTTGTG | 293 | 31R1 | CCCAAT | 371 |
| | | TTGGGA | | | AACATT | |
| | | GTTATT | | | AAAACA | |
| | | GT | | | ACC | |
| 32 | 32F1 | TTTTTT | 294 | 32R1 | CCAAAA | 372 |
| | | ATATTT | | | TCTCCT | |
| | | ATATAT | | | AACCTC | |
| | | AAGTGT | | | AA | |
| | | TAGAAA | | | | |
| | | TG | | | | |
| 35 | 35F1 | AGGAAG | 295 | 35R1 | CACCAA | 373 |
| | | TAAGGG | | | ACACCA | |
| | | TTTATT | | | CAATCA | |
| | | AATTTT | | | AC | |
| 36 | 36F1 | TATTTT | 296 | 36R1 | CTCCTT | 374 |
| | | ATGGTT | | | CTTCCA | |
| | | GGGGAA | | | TACTAA | |
| | | AT | | | TAACC | |
| 37 | 37F1 | GGGTTA | 297 | 37R1 | ACTTCC | 375 |
| | | GGATGA | | | CCCACA | |
| | | GAGAAT | | | AAAAAC | |
| | | GA | | | CC | |
| 38 | 38F1 | TGGTTT | 298 | 38R1 | AAATTA | 376 |
| | | TTTTGG | | | TTCAAA | |
| | | TAATAT | | | AAATAA | |
| | | AAGG | | | TTATAA | |
| | | | | | TAATAA | |
| | | | | | TATAC | |
| 40 | 40F1 | AATTTT | 299 | 40R1 | TAACTC | 377 |
| | | GTAATT | | | CTAAAC | |
| | | GGGTAG | | | TTAAAT | |
| | | GG | | | AATCCT | |
| | | | | | CT | |
| 42 | 42F1 | GGTGGG | 300 | 42R1 | TTTTTT | 378 |
| | | TTGAAA | | | TATAAT | |
| | | GGTTTT | | | TTTAAA | |
| | | TTAAG | | | AAATAA | |
| | | | | | CATC | |
| 43 | 43F1 | TTGGAG | 301 | 43R1 | AAACAA | 379 |
| | | TTTTTA | | | ATTACC | |
| | | GTTTTG | | | AATAAA | |
| | | AGTT | | | TTAAAA | |
| | | | | | ATA | |
| 44 | 44F1 | TTGTGT | 302 | 44R1 | CAACCC | 380 |
| | | GATAGA | | | ACCCAC | |
| | | GTTTAG | | | ACAAAT | |
| | | TTGG | | | TA | |
| 45 | 45F1 | TGTTGA | 303 | 45R1 | CTCAAA | 381 |
| | | ATTTGG | | | AAAATC | |
| | | TGTTTT | | | AAACTT | |
| | | TGTT | | | CAA | |
| 47 | 47F1 | AGTTTT | 304 | 47R1 | CAACAT | 382 |
| | | GAAGGG | | | TATTTC | |
| | | AAGATA | | | CTAAAA | |
| | | GG | | | CTTATA | |
| | | | | | AAT | |
| 49 | 49F1 | AAAAAA | 305 | 49R1 | ATATTT | 383 |
| | | ATTAAA | | | CAAACC | |
| | | AAGAGT | | | TTATCT | |
| | | AATAGG | | | TAAAAC | |
| | | AA | | | TT | |
| 50 | 50F1 | GGGAAT | 306 | 50R1 | AAACTC | 384 |
| | | GTGTTT | | | CTCTTC | |
| | | TTAGAG | | | CCAAAT | |
| | | GT | | | ATAC | |
| 51 | 51F1 | TTGTGA | 307 | 51R1 | AAAATC | 385 |
| | | ATATAG | | | CCCTTC | |
| | | GTGTGA | | | AATTCT | |
| | | GTTAAT | | | AC | |
| | | A | | | | |
| 52 | 52F1 | GGATGG | 308 | 52R1 | CCCTCC | 386 |
| | | GTGGAT | | | AAAAAA | |
| | | TAAATT | | | AAAAAT | |
| | | TT | | | ACTA | |
| 53 | 53F1 | TTTTGT | 309 | 53R1 | AAATAA | 387 |
| | | TAAATT | | | AAAAAA | |
| | | TTTTTT | | | TAAATA | |
| | | TTTTAG | | | ATATTT | |
| | | TT | | | TTCAA | |
| 54 | 54F1 | TGAGAT | 310 | 54R1 | TACCAA | 388 |
| | | GATTAA | | | CTACAC | |
| | | ATGAAG | | | CACTCT | |
| | | ATTAAA | | | CC | |
| 55 | 55F1 | GGTTGT | 311 | 55R1 | CAAAAT | 389 |
| | | TGTAAT | | | CAACCA | |
| | | TGTTTG | | | CAACCT | |
| | | TTGT | | | AC | |
| 56 | 56F1 | TTGTAG | 312 | 56R1 | AAACTT | 390 |
| | | TGTAGT | | | TTACTC | |
| | | TTGAGA | | | ATAATA | |
| | | AATAGG | | | TAATTT | |
| | | | | | CTACC | |
| 57 | 57F1 | TGTTTA | 313 | 57R1 | TTATAA | 391 |
| | | ATTGTT | | | AACTAT | |
| | | TGTTTT | | | AAACTC | |
| | | TTTTTA | | | CCTTTC | |
| | | G | | | A | |
| 58 | 58F1 | TGGAGG | 314 | 58R1 | CCCTCA | 392 |
| | | GTGGGA | | | ACCTCC | |
| | | GAGTTT | | | TAAATA | |
| | | AG | | | CAAAT | |
| 59 | 59F1 | TTTATG | 315 | 59R1 | AAAATA | 393 |
| | | AAATTT | | | CCATTT | |
| | | GTGGGT | | | ACCTAA | |
| | | GTA | | | CCAAT | |
| 60 | 60F1 | TTTGGT | 316 | 60R1 | CAAATC | 394 |
| | | GTATGT | | | TTTAAC | |
| | | ATTGTG | | | TCAAAA | |
| | | TATGT | | | TTAAAT | |
| | | | | | AATA | |
| 63 | 63F1 | GGATAT | 317 | 63R1 | CTCATT | 395 |
| | | TGGTTG | | | TTCAAA | |
| | | TTTTGG | | | CTACAC | |
| | | AT | | | TCAA | |
| 64 | 64F1 | GAGGAA | 318 | 64R1 | AAAAAC | 396 |
| | | ATGTTT | | | CCATTA | |
| | | TGTTTT | | | TTTCAA | |
| | | GG | | | CTT | |
| 65 | 65F1 | GTTTTT | 319 | 65R1 | AAAAAA | 397 |
| | | GGGGTT | | | ATTTTC | |
| | | ATAGTT | | | CTATAA | |
| | | GG | | | CTAATA | |
| | | | | | ACTT | |
| 66 | 66F1 | TTTGTT | 320 | 66R1 | CAAACT | 398 |
| | | TAGAGG | | | TCAATA | |
| | | TTTATG | | | CAATAA | |
| | | TTTGT | | | CCCA | |
| 67 | 67F1 | GTGGTT | 321 | 67R1 | TCCTCT | 399 |
| | | TTGAAA | | | CCTTTA | |
| | | TAGATT | | | AAAAAA | |
| | | TTGT | | | ATTC | |
| 68 | 68F1 | GGTGTT | 322 | 68R1 | TCCCTA | 400 |
| | | ATTTGA | | | TCTAAA | |
| | | GGTTAG | | | CCCCTC | |
| | | GAT | | | CT | |
| 69 | 69F1 | AGGAAG | 323 | 69R1 | AAATCT | 401 |
| | | ATATTG | | | ACCTTC | |
| | | TTTATG | | | CACCAA | |
| | | TGGA | | | AT | |
| 70 | 70F1 | TTATTT | 324 | 70R1 | AACTTT | 402 |
| | | TTTTAT | | | CTCAAA | |
| | | AGTTTT | | | AACATA | |
| | | ATAGGA | | | TTTCA | |
| | | TGG | | | | |
| 71 | 71F1 | AGGAAA | 325 | 71R1 | ATCTAC | 403 |
| | | ATTAAT | | | AACTCC | |
| | | GAATTG | | | CAAAAA | |
| | | TTAAAG | | | TTC | |
| 72 | 72F1 | TTGAAT | 326 | 72R1 | ACTTAA | 404 |
| | | GTTGTT | | | ACTCCT | |
| | | ATTTGG | | | CTCTAC | |
| | | TATG | | | TCATAA | |
| | | | | | AT | |
| 73 | 73F1 | TGTTAT | 327 | 73R1 | AACTAA | 405 |
| | | TTGATT | | | CCACCC | |
| | | TTGTTT | | | TCTCCT | |
| | | TGGA | | | AC | |
| 74 | 74F1 | TTGTAT | 328 | 74R1 | AAATTA | 406 |
| | | TTGTGT | | | AACTCT | |
| | | GATTTT | | | TAATAC | |
| | | AGATAA | | | ACTCCA | |
| | | G | | | TAA | |
| 75 | 75F1 | AGTTTT | 329 | 75R1 | TTATCC | 407 |
| | | AATAGT | | | TTTTTA | |
| | | TTTAAG | | | TTTAAA | |
| | | TTTGGA | | | AATAAT | |
| | | TT | | | ATTAAA | |
| 76 | 76F1 | GTATGG | 330 | 76R1 | TTCCTA | 408 |
| | | TATTTT | | | ATAAAC | |
| | | TTGAAG | | | TAAAAA | |
| | | TGAAG | | | TATTAA | |
| | | | | | AATTC | |
| 77 | 77F1 | AAGTTG | 331 | 77R1 | TCTTTT | 409 |
| | | ATTGGT | | | CTTTCT | |
| | | TAGAGT | | | ATCAAT | |
| | | TGG | | | ATTAAT | |
| | | | | | AAAATA | |
| 81 | 81F1 | GGATTT | 332 | 81R1 | ACAATT | 410 |
| | | GTAATT | | | CTCTTT | |
| | | GGTATA | | | CCTTTA | |
| | | GAAGG | | | AAATAA | |
| 82 | 82F1 | TTGGAT | 333 | 82R1 | TCTTAC | 411 |
| | | TTTAGA | | | TAAAAC | |
| | | TTATTA | | | TAACAT | |
| | | GGTTTT | | | CACAAA | |
| | | | | | ATAC | |
| 83 | 83F1 | GGGTTG | 334 | 83R1 | ATCAAA | 412 |
| | | GAATGT | | | AATAAT | |
| | | TTTGAA | | | TTATTA | |
| | | GG | | | AATATT | |
| | | | | | AAATAC | |
| | | | | | TT | |
| 84 | 84F1 | GTTGGT | 335 | 84R1 | AACTTC | 413 |
| | | GATTGT | | | ACAAAA | |
| | | TGAAAA | | | ACATCT | |
| | | TG | | | CTCT | |
| 85 | 85F1 | TTAGGA | 336 | 85R1 | AAATTA | 414 |
| | | TTTATG | | | ATATTA | |
| | | TGTTTT | | | TTTTTC | |
| | | ATAAAA | | | TAACCC | |
| | | TATAG | | | C | |
| 86 | 86F1 | AAATTA | 337 | 86R1 | TCACAC | 415 |
| | | AAGAGT | | | CCTTAC | |
| | | TAAGTA | | | TAATTA | |
| | | TTAGAA | | | CCC | |
| | | ATGAT | | | | |
| 87 | 87F1 | AAAATT | 338 | 87R1 | ACTCCC | 416 |
| | | TTTTTA | | | TCCTTA | |
| | | GTTTGG | | | TTCAAT | |
| | | GAAA | | | AAA | |
| 88 | 88F1 | ATTTTG | 339 | 88R1 | CTCAAC | 417 |
| | | GTTTTA | | | ACCCTA | |
| | | GGGGGT | | | CCCTAA | |
| | | GA | | | AT | |
| 92 | 92F1 | TTTTTT | 340 | 92R1 | CAAAAT | 418 |
| | | GGGTGT | | | ATAAAA | |
| | | TAAGTT | | | ATCAAA | |
| | | AGTT | | | TCCC | |
| 93 | 93F1 | GGTTTT | 341 | 93R1 | AACAAA | 419 |
| | | AGGTGA | | | AAAAAC | |
| | | TATTTG | | | TACTAA | |
| | | AATAAT | | | CATAAT | |
| | | AA | | | ACC | |
| 94 | 94F1 | GTAGGG | 342 | 94R1 | TTTTCT | 420 |
| | | GTTTTA | | | CCCTTA | |
| | | GATTTT | | | TAATTT | |
| | | TTTAG | | | TAACAC | |
| 95 | 95F1 | AGTTAA | 343 | 95R1 | AAAACA | 421 |
| | | GTTAGA | | | ACTCAA | |
| | | TGTGTT | | | AACTCT | |
| | | ATAATT | | | AAAATA | |
| | | AGAGTT | | | ATA | |
| 97 | 97F1 | AAAAAA | 344 | 97R1 | ACAAAC | 422 |
| | | TATTTT | | | TAAAAT | |
| | | TATTTG | | | AAATCT | |
| | | TGTAAT | | | ATAACA | |
| | | TATAAA | | | ATAATA | |
| | | | | | A | |
| 99 | 99F1 | TAGGAG | 345 | 99R1 | CTAAAC | 423 |
| | | GGGTGT | | | TCCCCA | |
| | | GTGTTG | | | AAAACA | |
| | | TG | | | CT | |
| 100 | 100F1 | TGAATT | 346 | 100R1 | CTCATT | 424 |
| | | TGTTGT | | | TAAACC | |
| | | TGATTT | | | TCATAA | |
| | | TGT | | | CCC | |

TABLE 10
Primer designed in Example 4

| | Forward primer | | | Reverse primer | | |
|---|---|---|---|---|---|---|
| Mea- | | Base | | | Base | |
| sure- | | se- | | | se- | |
| ment | | quence | Se- | | quence | Se- |
| site | | (5′ → | quence | | (5′ → | quence |
| ID | Name | 3′) | number | Name | 3′) | number |
| 2 | 2F3 | TGGTAG | 425 | 2R3 | TAATCC | 507 |
| | | TGATTA | | | CACTTA | |
| | | GTTTAT | | | CAAAAA | |
| | | TTTTTG | | | ACAC | |
| 3 | 3F3 | TGTAGA | 426 | 3R3 | TTAATA | 508 |
| | | GAGGAG | | | TCTATC | |
| | | GAGGTG | | | CTAATT | |
| | | AG | | | CCAACC | |
| 5 | 5F3 | TTTTTG | 427 | 5R3 | TCAAAA | 509 |
| | | GGTTTG | | | CATTTC | |
| | | AAATGT | | | TAAAAC | |
| | | TA | | | TATTAA | |
| | | | | | TATC | |
| 6 | 6F3 | GGGTTG | 428 | 6R3 | CAAAAC | 510 |
| | | AGGATT | | | TTAAAC | |
| | | AGTATT | | | AATAAT | |
| | | GAT | | | ACTTAC | |
| | | | | | TCA | |
| 7 | 7F3 | GGTTGA | 429 | 7R3 | TTAAAT | 511 |
| | | TGAGGT | | | CTAACA | |
| | | ATAGGT | | | CCCACA | |
| | | GA | | | CC | |
| 8 | 8F3 | GAAGTA | 430 | 8R3 | AATATA | 512 |
| | | GGTTAA | | | AAAAAT | |
| | | GAAGGA | | | AATCCC | |
| | | GGAT | | | CAAAC | |
| 10 | 10F3 | TTGTTT | 431 | 10R3 | CAAACA | 513 |
| | | TTTGTT | | | AAATTT | |
| | | GTGTGG | | | ACAACC | |
| | | AA | | | CA | |
| 12 | 12F3 | TTTTTA | 432 | 12R3 | ACCTCA | 514 |
| | | GGTTAT | | | CCCACT | |
| | | TTTTTA | | | TCTCCT | |
| | | AATGGA | | | AC | |
| 13 | 13F3 | TGTGAT | 433 | 13R3 | CACCCA | 515 |
| | | TTTAGT | | | ACTCAT | |
| | | ATTTGG | | | TTTTTT | |
| | | GAAG | | | AC | |
| 14 | 14F3 | GGGGAG | 434 | 14R3 | CCCACT | 516 |
| | | TTTTTT | | | AACTCC | |
| | | ATGAAG | | | CCAAAA | |
| | | GG | | | AC | |
| 15 | 15F3 | GTGTGG | 435 | 15R3 | ACCCAA | 517 |
| | | AAGGAA | | | AATCTA | |
| | | AAAAAA | | | CAAAAC | |
| | | AG | | | CC | |
| 16 | 16F3 | ATTTTT | 436 | 16R3 | AAATCC | 518 |
| | | TTATTA | | | CTATAT | |
| | | GGTTGG | | | ATTCCT | |
| | | TGG | | | TACCA | |
| 17 | 17F3 | TTTAGA | 437 | 17R3 | CCTCAT | 519 |
| | | TATAAA | | | ACTCTA | |
| | | TTTTTT | | | AAAACC | |
| | | TGTATG | | | CC | |
| | | GA | | | | |
| 18 | 18F3 | TTTATT | 438 | 18R3 | TCCCCA | 520 |
| | | GGGGTA | | | CTAATA | |
| | | GAGTAT | | | CTTCCT | |
| | | AGGTT | | | TAC | |
| 20 | 20F3 | GAGGTT | 439 | 20R3 | CATACC | 521 |
| | | GTTGTT | | | TCCTAA | |
| | | GTGTTT | | | CATCCC | |
| | | GT | | | AC | |
| 22 | 22F3 | GGGTGT | 440 | 22R3 | TACCTC | 522 |
| | | GGTAGG | | | TAATCC | |
| | | TGTTTT | | | CAATTC | |
| | | GG | | | AA | |
| 23 | 23F3 | TGTTGT | 441 | 23R3 | AAAAAT | 523 |
| | | TTTGTT | | | AAACCT | |
| | | TGTATG | | | TACAAA | |
| | | GA | | | CTACAC | |
| | | | | | A | |
| 24 | 24F3 | TTTAGG | 442 | 24R3 | CAAACA | 524 |
| | | TTTTTT | | | CCCCTT | |
| | | GTTGGT | | | TTTCAT | |
| | | GG | | | CA | |
| 25 | 25F3 | GAGTAT | 443 | 25R3 | AACTCT | 525 |
| | | TTTATG | | | ACAAAA | |
| | | TTGTTT | | | ACCAAA | |
| | | TAGTTG | | | TATAAT | |
| | | TTT | | | AAC | |
| 26 | 26F3 | AGTAGG | 444 | 26R3 | TTATAC | 526 |
| | | AAGGGT | | | CCACTA | |
| | | ATTGGT | | | CACAAA | |
| | | GG | | | TAAAAA | |
| 27 | 27F3 | GGAAGT | 445 | 27R3 | TAACCT | 527 |
| | | GGGTTT | | | AACCAC | |
| | | GGGAAG | | | AAACAA | |
| | | TA | | | CC | |
| 28 | 28F3 | TTTTGG | 446 | 28R3 | ACCTCA | 528 |
| | | TTTTAA | | | AATAAC | |
| | | AAGAGA | | | TTAAAA | |
| | | GAAA | | | TTCACT | |
| 29 | 29F3 | AAGAGT | 447 | 29R3 | TTTCTA | 529 |
| | | TTTTAA | | | ACATAT | |
| | | TGAATG | | | TTACTA | |
| | | GATATA | | | CTAAAA | |
| | | A | | | AATTTA | |
| | | | | | A | |
| 30 | 30F3 | AAGTGG | 448 | 30R3 | ACTTAA | 530 |
| | | TAAGAG | | | TTTAAT | |
| | | GAGTTG | | | TACCCA | |
| | | GG | | | AACAAT | |
| 31 | 31F3 | GGTTTT | 449 | 31R3 | CCCAAT | 531 |
| | | TGTTGT | | | AACATT | |
| | | GTTGGG | | | AAAACA | |
| | | AG | | | ACC | |
| 32 | 32F3 | TTTTTT | 450 | 32R3 | ATCCAA | 532 |
| | | ATATTT | | | AATCTC | |
| | | ATATAT | | | CTAACC | |
| | | AAGTGT | | | TC | |
| | | TAGAAA | | | | |
| | | TG | | | | |
| 35 | 35F3 | GAGAGG | 451 | 35R3 | CACCAA | 533 |
| | | AAGTAA | | | ACACCA | |
| | | GGGTTT | | | CAATCA | |
| | | ATTAA | | | AC | |
| 36 | 36F3 | TATTTT | 452 | 36R3 | AACAAC | 534 |
| | | ATGGTT | | | TCCTTC | |
| | | GGGGAA | | | TTCCAT | |
| | | AT | | | ACT | |
| 37 | 37F3 | GGAGAT | 453 | 37R3 | CACAAA | 535 |
| | | TGGGGT | | | AAACCC | |
| | | TAGGAT | | | TAAAAA | |
| | | GA | | | ACTAAA | |
| | | | | | AA | |
| 38 | 38F3 | TGGTTT | 454 | 38R3 | AAATTA | 536 |
| | | TTTTGG | | | TTCAAA | |
| | | TAATAT | | | AAATAA | |
| | | AAGG | | | TTATAA | |
| | | | | | TAATAA | |
| | | | | | TATAC | |
| 39 | 39F3 | AGTAGG | 455 | 39R3 | ATAACA | 537 |
| | | TTTTTA | | | AAACTC | |
| | | AAATAT | | | AAAACC | |
| | | GTGGTT | | | CC | |
| 40 | 40F3 | AATTTT | 456 | 40R3 | CACTTA | 538 |
| | | GTAATT | | | TTACCC | |
| | | GGGTAG | | | AAACTA | |
| | | GG | | | ATCTTT | |
| 42 | 42F3 | TTTTGT | 457 | 42R3 | AAAAAA | 539 |
| | | AGTTTT | | | ATCCCT | |
| | | GAGAGG | | | CAATAC | |
| | | TGA | | | AAC | |
| 43 | 43F3 | TTTATT | 458 | 43R3 | AAAACA | 540 |
| | | GGAGTT | | | AATTAC | |
| | | TTTAGT | | | CAATAA | |
| | | TTTGA | | | ATTAAA | |
| | | | | | A | |
| 44 | 44F3 | TTGTGT | 459 | 44R3 | CAACCC | 541 |
| | | GATAGA | | | ACCCAC | |
| | | GTTTAG | | | ACAAAT | |
| | | TTGG | | | TA | |
| 45 | 45F3 | TTTTGT | 460 | 45R3 | CTCAAA | 542 |
| | | GTGGAT | | | AAAATC | |
| | | AGTTGT | | | AAACTT | |
| | | TG | | | CAA | |
| 46 | 46F3 | TTGGGA | 461 | 46R3 | CCCACA | 543 |
| | | TAGTGT | | | AACTAC | |
| | | TTTGAG | | | TTCTAC | |
| | | TG | | | AAAT | |
| 47 | 47F3 | TTTTGA | 462 | 47R3 | ATTTCC | 544 |
| | | GAAGTT | | | TAAAAC | |
| | | TTGAAG | | | TTATAA | |
| | | GG | | | ATTTAT | |
| | | | | | AAAAA | |
| 49 | 49F3 | TTGTTT | 463 | 49R3 | TTTCAA | 545 |
| | | TTAAAA | | | ACCTTA | |
| | | AAATTA | | | TCTTAA | |
| | | AAAAGA | | | AACTTC | |
| | | G | | | | |
| 50 | 50F3 | TTGGGG | 464 | 50R3 | TAAACT | 546 |
| | | AATGTG | | | CCTCTT | |
| | | TTTTTA | | | CCCAAA | |
| | | GA | | | TAT | |
| 51 | 51F3 | GTTGTG | 465 | 51R3 | AAAATC | 547 |
| | | AATATA | | | CCCTTC | |
| | | GGTGTG | | | AATTCT | |
| | | AGTTAA | | | AC | |
| 52 | 52F3 | GTTTTT | 466 | 52R3 | AACCAA | 548 |
| | | TAGGAG | | | ACCCTT | |
| | | AGGGGG | | | AACAAC | |
| | | TG | | | CC | |
| 53 | 53F3 | TTATGT | 467 | 53R3 | AAAATT | 549 |
| | | ATTTTT | | | TCAAAA | |
| | | TTTTTA | | | AAATAC | |
| | | TTTAAA | | | TAAAAA | |
| | | AAATT | | | AT | |
| 54 | 54F3 | AATGAA | 468 | 54R3 | TACCAA | 550 |
| | | GATTAA | | | CTACAC | |
| | | AAAAAG | | | CACTCT | |
| | | TTAAGG | | | CC | |
| 55 | 55F3 | GGTTGT | 469 | 55R3 | CAAAAT | 551 |
| | | TGTAAT | | | CAACCA | |
| | | TGTTTG | | | CAACCT | |
| | | TTG | | | AC | |
| 56 | 56F3 | TTGTAG | 470 | 56R3 | TTTTAC | 552 |
| | | TGTAGT | | | TCATAA | |
| | | TTGAGA | | | TATAAT | |
| | | AATAGG | | | TTCTAC | |
| | | | | | CTCA | |
| 57 | 57F3 | AATTTT | 471 | 57R3 | AAAACT | 553 |
| | | ATATGT | | | ATAAAC | |
| | | GTTTAA | | | TCCCTT | |
| | | TTGTTT | | | TCATT | |
| | | GT | | | | |
| 58 | 58F3 | TATTGG | 472 | 58R3 | CCCTCA | 554 |
| | | AGGGTG | | | ACCTCC | |
| | | GGAGAG | | | TAAATA | |
| | | TT | | | CA | |
| 59 | 59F3 | GTTTAT | 473 | 59R3 | AAAATA | 555 |
| | | GAAATT | | | CCATTT | |
| | | TGTGGG | | | ACCTAA | |
| | | TG | | | CCAA | |
| 60 | 60F3 | GAGTGT | 474 | 60R3 | AACCAC | 556 |
| | | GTGATT | | | CACCTC | |
| | | GGGTTT | | | CAAATC | |
| | | GT | | | TT | |
| 63 | 63F3 | GGATAT | 475 | 63R3 | AACATC | 557 |
| | | TGGTTG | | | TCATTT | |
| | | TTTTGG | | | TCAAAC | |
| | | AT | | | TACAC | |
| 64 | 64F3 | TTTTTA | 476 | 64R3 | CCCATT | 558 |
| | | TAATTG | | | ATTTCA | |
| | | GTGAGG | | | ACTTAC | |
| | | GA | | | ACTCT | |
| 65 | 65F3 | GTTTTT | 477 | 65R3 | TTCCAT | 559 |
| | | GGGGTT | | | AACAAT | |
| | | ATAGTT | | | CACTCA | |
| | | GG | | | CTAA | |
| 66 | 66F3 | GAAAGA | 478 | 66R3 | AACTTA | 560 |
| | | AAAGAA | | | AACCCA | |
| | | AAAGAG | | | AAACTT | |
| | | AAAGT | | | TAAAAC | |
| | | | | | TAC | |
| 67 | 67F3 | TGTGGT | 479 | 67R3 | CCTCTC | 561 |
| | | TTTGAA | | | CTTTAA | |
| | | ATAGAT | | | AAAAAA | |
| | | TTTG | | | TTCC | |
| 68 | 68F3 | GGTGTT | 480 | 68R3 | TCCCTA | 562 |
| | | ATTTGA | | | TCTAAA | |
| | | GGTTAG | | | CCCCTC | |
| | | GAT | | | CT | |
| 69 | 69F3 | TGTGGA | 481 | 69R3 | AAATCT | 563 |
| | | AATATT | | | ACCTTC | |
| | | GATTTT | | | CACCAA | |
| | | TGA | | | AT | |
| 70 | 70F3 | TATAGG | 482 | 70R3 | CCTTCA | 564 |
| | | ATGGTA | | | CATACC | |
| | | GGGTTG | | | AAAAAA | |
| | | GG | | | AAC | |
| 71 | 71F3 | AGGAAA | 483 | 71R3 | ATCTAC | 565 |
| | | ATTAAT | | | AACTCC | |
| | | GAATTG | | | CAAAAA | |
| | | TTAAAG | | | TTC | |
| 72 | 72F3 | TGAATG | 484 | 72R3 | CCCCCT | 566 |
| | | TTGTTA | | | AAAATT | |
| | | TTTGGT | | | TACTAA | |
| | | ATGA | | | AAAA | |
| 73 | 73F3 | TTTGAT | 485 | 73R3 | AAACTA | 567 |
| | | TTTGTT | | | ACCACC | |
| | | TTGGAG | | | CTCTCC | |
| | | TG | | | TA | |
| 74 | 74F3 | TTTTAA | 486 | 74R3 | CTCTTA | 568 |
| | | TTTTGT | | | ATACAC | |
| | | ATTTGT | | | TCCATA | |
| | | GTGATT | | | ATTAAC | |
| | | | | | C | |
| 75 | 75F3 | AAAGTT | 487 | 75R3 | CCTTTT | 569 |
| | | TTAATA | | | TATTTA | |
| | | GTTTTA | | | AAAATA | |
| | | AGTTTG | | | ATATTA | |
| | | GA | | | AACAT | |
| 76 | 76F3 | GTATGG | 488 | 76R3 | TTCCTA | 570 |
| | | TATTTT | | | ATAAAC | |
| | | TTGAAG | | | TAAAAA | |
| | | TGAAG | | | TATTAA | |
| | | | | | AATTC | |
| 77 | 77F3 | AAGTTG | 489 | 77R3 | CCTCCT | 571 |
| | | ATTGGT | | | CTTTTC | |
| | | TAGAGT | | | TTTCTA | |
| | | TGG | | | TCA | |
| 81 | 81F3 | GGATTT | 490 | 81R3 | ATTACA | 572 |
| | | GTAATT | | | ATTCTC | |
| | | GGTATA | | | TTTCCT | |
| | | GAAGG | | | TTAAAA | |
| 82 | 82F3 | GAGGGG | 491 | 82R3 | CATCTC | 573 |
| | | ATGTTT | | | TTACTA | |
| | | TTTTGT | | | AAACTA | |
| | | TG | | | ACATCA | |
| | | | | | CA | |
| 83 | 83F3 | TTTAAA | 492 | 83R3 | AATTTA | 574 |
| | | GGAGGG | | | ATTAAA | |
| | | TTGGAA | | | TATTAA | |
| | | TG | | | ATACTT | |
| | | | | | AAAAAA | |
| | | | | | ATTA | |
| 84 | 84F3 | AGTTGG | 493 | 84R3 | AACTTC | 575 |
| | | TGATTG | | | ACAAAA | |
| | | TTGAAA | | | ACATCT | |
| | | AT | | | CTCT | |
| 85 | 85F3 | GGTTAG | 494 | 85R3 | AAATTA | 576 |
| | | GATTTA | | | ATATTA | |
| | | TGTGTT | | | TTTTTC | |
| | | TTATAA | | | TAACCC | |
| | | AAT | | | C | |
| 86 | 86F3 | TTAAAG | 495 | 86R3 | TCACAC | 577 |
| | | AGTTAA | | | CCTTAC | |
| | | GTATTA | | | TAATTA | |
| | | GAAATG | | | CCC | |
| | | ATGT | | | | |
| 87 | 87F3 | TAAAGG | 496 | 87R3 | AAAAAA | 578 |
| | | TGAAGG | | | TCTTTC | |
| | | GTGTGG | | | CAACCT | |
| | | GG | | | AAA | |
| 88 | 88F3 | TTTGGT | 497 | 88R3 | TCTCAA | 579 |
| | | TTATGG | | | CACCCT | |
| | | GGATTT | | | ACCCTA | |
| | | AT | | | AA | |
| 89 | 89F3 | GTTAGG | 498 | 89R3 | CAACTA | 580 |
| | | TTGGGG | | | TACTTT | |
| | | TGGTGG | | | CCCATA | |
| | | TT | | | ACCTAA | |
| 91 | 91F3 | GAGGTG | 499 | 91R3 | AAACCC | 581 |
| | | GGGGTT | | | CAAAAC | |
| | | TTTTAT | | | TCCCAC | |
| | | TG | | | AAC | |
| 92 | 92F3 | TATTTT | 500 | 92R3 | AAAATA | 582 |
| | | TTTGGG | | | TAAAAA | |
| | | TGTTAA | | | TCAAAT | |
| | | GTTAG | | | CCCC | |
| 93 | 93F3 | GGTTTT | 501 | 93R3 | CATAAA | 583 |
| | | AGGTGA | | | AAAAAA | |
| | | TATTTG | | | CAAAAA | |
| | | AATAAT | | | AAACTA | |
| | | | | | CT | |
| 94 | 94F3 | AGTATT | 502 | 94R3 | CAACAA | 584 |
| | | GGTGTA | | | AAACTC | |
| | | TATGAG | | | CAAACT | |
| | | AAGGA | | | TC | |
| 95 | 95F3 | AGAGTT | 503 | 95R3 | CAAAAC | 585 |
| | | AAGTTA | | | TCTAAA | |
| | | GATGTG | | | ATAATA | |
| | | TTATAA | | | AACAAT | |
| | | TTAGAG | | | AAAAT | |
| 97 | 97F3 | AGATTA | 504 | 97R3 | CTATCT | 586 |
| | | AAAAAT | | | AAAAAT | |
| | | ATTTTT | | | ACAAAC | |
| | | ATTTGT | | | TAAAAT | |
| | | GTAA | | | AAATCT | |
| 99 | 99F3 | GGGAGT | 505 | 99R3 | CTAAAC | 587 |
| | | AGGAGG | | | TCCCCA | |
| | | GGTGTG | | | AAAACA | |
| | | TG | | | CT | |
| 100 | 100F3 | TGAATT | 506 | 100R3 | CTCATT | 588 |
| | | TGTTGT | | | TAAACC | |
| | | TGATTT | | | TCATAA | |
| | | TGT | | | CCC | |

TABLE 11
Primer designed in Comparative Example 2

| | Forward primer | | | Reverse primer | | |
|---|---|---|---|---|---|---|
| Mea- | | Base | | | Base | |
| sure- | | se- | | | se- | |
| ment | | quence | Se- | | quence | Se- |
| site | | (5′ → | quence | | (5′ → | quence |
| ID | Name | 3′) | number | Name | 3′) | number |
| 2 | 2F1 | TTTTTT | 589 | 2R1 | AATCCC | 632 |
| | | TTATAG | | | ACTTAC | |
| | | TTTTTG | | | AAAAAA | |
| | | GTAGTG | | | CA | |
| | | A | | | | |
| 3 | 3F1 | GAGGAG | 590 | 3R1 | ACCTTA | 633 |
| | | GAGGTG | | | ATATCT | |
| | | AGTTGT | | | ATCCTA | |
| | | AG | | | ATTCCA | |
| 5 | 5F1 | AATAAT | 591 | 5R1 | TCTAAA | 634 |
| | | TTTTTT | | | ACTATT | |
| | | TTTGGG | | | AATATC | |
| | | TTTGA | | | TCTAAA | |
| | | | | | AAACTA | |
| | | | | | A | |
| 8 | 8F1 | AAGAAG | 592 | 8R1 | CAAATA | 635 |
| | | GAGGAT | | | TAAAAA | |
| | | ATAGAG | | | ATAATC | |
| | | AAGG | | | CCCA | |
| 10 | 10F1 | AAAGGG | 593 | 10R1 | CTCCAC | 636 |
| | | GTAAAT | | | TAAATA | |
| | | AGAATT | | | ACTATC | |
| | | TGTAG | | | TCTTAC | |
| | | | | | TATATA | |
| | | | | | A | |
| 13 | 13F1 | TTTTAA | 594 | 13R1 | CTCAAA | 637 |
| | | GGTGTT | | | ATCCCA | |
| | | AGGGGA | | | ACCTCA | |
| | | AG | | | AAA | |
| 14 | 14F1 | GGGAGT | 595 | 14R1 | CCCCAC | 638 |
| | | TTTTTA | | | TAACTC | |
| | | TGAAGG | | | CCCAAA | |
| | | GA | | | AA | |
| 15 | 15F1 | GTGTGG | 596 | 15R1 | CAAAAA | 639 |
| | | AAGGAA | | | AACCCA | |
| | | AAAAAA | | | AAATCT | |
| | | AG | | | ACAAAA | |
| 16 | 16F1 | GTTTGT | 597 | 16R1 | TTACCA | 640 |
| | | TTGTTA | | | ATATTC | |
| | | TTTTTT | | | TCATTA | |
| | | TATTAG | | | ATTTAA | |
| | | G | | | TATAA | |
| 18 | 18F1 | TTTATT | 598 | 18R1 | AATCAA | 641 |
| | | GATGTT | | | CACCCA | |
| | | TTTTTG | | | CTAAAA | |
| | | TTAGG | | | CA | |
| 20 | 20F1 | TTGGTA | 599 | 20R1 | CCAAAA | 642 |
| | | TTTTAT | | | ACTATT | |
| | | TTTTGA | | | ACTATA | |
| | | GAGG | | | CTTATT | |
| | | | | | TCCA | |
| 22 | 22F1 | TGGTAG | 600 | 22R1 | AATCCC | 643 |
| | | GTGTTT | | | AATTCA | |
| | | TGGGTT | | | ATTAAA | |
| | | GA | | | AAA | |
| 25 | 25F1 | TTTTGT | 601 | 25R1 | AAAATA | 644 |
| | | AGGGGT | | | CTCCAT | |
| | | TAGGTG | | | ATTACC | |
| | | TAG | | | CCA | |
| 26 | 26F1 | TTATTA | 602 | 26R1 | CCACTA | 645 |
| | | TTTATT | | | CACAAA | |
| | | TTTTGG | | | TAAAAA | |
| | | GTGAAG | | | AATAAA | |
| 27 | 27F1 | GGGTTT | 603 | 27R1 | AACCTA | 646 |
| | | GGGAAG | | | ACCACA | |
| | | TATGGA | | | AACAAC | |
| | | AG | | | CA | |
| 28 | 28F1 | TTTTGG | 604 | 28R1 | CTATAC | 647 |
| | | TTTTAA | | | CTACAT | |
| | | AAGAGA | | | ATACAT | |
| | | GAAA | | | ACCTCA | |
| | | | | | AATAA | |
| 29 | 29F1 | AGGGTT | 605 | 29R1 | TTTTTC | 648 |
| | | ATATTT | | | TCTTTT | |
| | | TAATAT | | | TCCCAA | |
| | | GTAGAA | | | AA | |
| | | AAA | | | | |
| 30 | 30F1 | AGAGGA | 606 | 30R1 | TTCACA | 649 |
| | | GTTGGG | | | CTTAAT | |
| | | TGTGTA | | | TTAATT | |
| | | AG | | | ACCCA | |
| 31 | 31F1 | GGTTTT | 607 | 31R1 | CCAATA | 650 |
| | | TGTTGT | | | ACATTA | |
| | | GTTGGG | | | AAACAA | |
| | | AG | | | CCA | |
| 32 | 32F1 | TTAGGG | 608 | 32R1 | CCACAC | 651 |
| | | TTTTTT | | | ATAAAT | |
| | | AATTTT | | | ACCAAA | |
| | | AGTATA | | | AATAA | |
| | | TAAAG | | | | |
| 35 | 35F1 | AGGGTT | 609 | 35R1 | CTCTAC | 652 |
| | | TATTAA | | | CCCACC | |
| | | TTTTTT | | | AAACAC | |
| | | TAATAA | | | CA | |
| | | GTAG | | | | |
| 36 | 36F1 | GGGTTT | 610 | 36R1 | ACTAAA | 653 |
| | | TAAGTA | | | ACACAA | |
| | | GGGAGG | | | AACACT | |
| | | TAG | | | AAAACA | |
| 37 | 37F1 | AGAAAA | 611 | 37R1 | AACTAA | 654 |
| | | TTTTGG | | | AACCAA | |
| | | GAGGTT | | | AATAAA | |
| | | GA | | | AAAATA | |
| | | | | | AA | |
| 38 | 38F1 | GGTAAT | 612 | 38R1 | AAATTT | 655 |
| | | ATAAGG | | | AAAAAA | |
| | | TATAGA | | | TTATTC | |
| | | GTATAG | | | AAAAAA | |
| | | GTTAGG | | | TAA | |
| 47 | 47F1 | TTGGAA | 613 | 47R1 | CCTAAA | 656 |
| | | TTTATA | | | AAAAAA | |
| | | GGTTTG | | | ATAAAA | |
| | | TAAAG | | | ATAACA | |
| | | | | | CA | |
| 54 | 54F1 | AATGAA | 614 | 54R1 | CAACTA | 657 |
| | | GATTAA | | | CACCAC | |
| | | AAAAAG | | | TCTCCA | |
| | | TTAAGG | | | TATAA | |
| 55 | 55F1 | GTTTGT | 615 | 55R1 | CCTACT | 658 |
| | | TGTTTT | | | AATCTT | |
| | | GTAGAA | | | ACTCAA | |
| | | AAATAA | | | CAAACA | |
| 56 | 56F1 | GTTTGA | 616 | 56R1 | AAATAC | 659 |
| | | GAAATA | | | AAAACT | |
| | | GGTAAT | | | CTATAT | |
| | | AAAAAT | | | TAATTC | |
| | | AGA | | | TAAAAT | |
| | | | | | AA | |
| 59 | 59F1 | TTGTGG | 617 | 59R1 | AAAATA | 660 |
| | | GTGTAA | | | TACTAA | |
| | | ATAAAT | | | AAAAAA | |
| | | TGA | | | AAAATA | |
| | | | | | CCA | |
| 64 | 64F1 | AATTGG | 618 | 64R1 | CAATTC | 661 |
| | | AATATG | | | AAAATT | |
| | | TTATTA | | | TATAAA | |
| | | ATTAGA | | | AAAAAA | |
| | | AAA | | | AA | |
| 65 | 65F1 | TTGGGG | 619 | 65R1 | CAATCA | 662 |
| | | TTATAG | | | CTCACT | |
| | | TTGGAG | | | AAACAA | |
| | | AG | | | AACA | |
| 69 | 69F1 | GGAAAT | 620 | 69R1 | CTTCCA | 663 |
| | | ATTGAT | | | CCAAAT | |
| | | TTTTGA | | | ATTCAA | |
| | | TAGAAG | | | AA | |
| 72 | 72F1 | TTTTTT | 621 | 72R1 | CCATTT | 664 |
| | | GAGATT | | | AATATA | |
| | | TGTTAA | | | AATCAC | |
| | | GAAAG | | | ATAACC | |
| | | | | | A | |
| 74 | 74F1 | AGGGGT | 622 | 74R1 | AAATTT | 665 |
| | | AGTTGT | | | CATTTA | |
| | | AGAGGT | | | CAAAAT | |
| | | AGA | | | AAATAA | |
| | | | | | CA | |
| 75 | 75F1 | TTATTT | 623 | 75R1 | CCTTTT | 666 |
| | | AATTTT | | | TATTTA | |
| | | ATATTT | | | AAAATA | |
| | | TGAAGG | | | ATATTA | |
| | | AGA | | | AACA | |
| 76 | 76F1 | AGTGTT | 624 | 76R1 | CATACA | 667 |
| | | GGGATT | | | TAACAC | |
| | | TTGATT | | | TTCTTA | |
| | | GA | | | AAATAA | |
| | | | | | AACA | |
| 81 | 81F1 | GGATTT | 625 | 81R1 | TTATCC | 668 |
| | | GTAATT | | | TAAAAA | |
| | | GGTATA | | | AATTAT | |
| | | GAAGG | | | AAAAAA | |
| | | | | | TAATAA | |
| 84 | 84F1 | TGTGTA | 626 | 84R1 | TCAAAA | 669 |
| | | GGTTTT | | | AAATCA | |
| | | TTGGTA | | | CTATAT | |
| | | GG | | | AAACCA | |
| 86 | 86F1 | AAGAGT | 627 | 86R1 | CACACC | 670 |
| | | TAAGTA | | | CTTACT | |
| | | TTAGAA | | | AATTAC | |
| | | ATGATG | | | CCA | |
| | | TAAG | | | | |
| 88 | 88F1 | TTTTAG | 628 | 88R1 | CTCAAA | 671 |
| | | TATTTT | | | AAATAA | |
| | | GTTTTA | | | ATTTCC | |
| | | AGTTAG | | | AAAA | |
| | | TTAAAG | | | | |
| 93 | 93F1 | GGTTTT | 629 | 93R1 | ATCAAA | 672 |
| | | AGGTGA | | | ATCATA | |
| | | TATTTG | | | AAAAAA | |
| | | AATAAT | | | AACAAA | |
| | | AA | | | A | |
| 95 | 95F1 | ATTTTG | 630 | 95R1 | ACAAAA | 673 |
| | | GGATAA | | | TTAAAC | |
| | | TAGGTA | | | CAAATA | |
| | | GTGA | | | TACCA | |
| 99 | 99F1 | GTGTGT | 631 | 99R1 | ACCTAA | 674 |
| | | GTTGTG | | | ACTCCC | |
| | | GTGAGG | | | CAAAAA | |
| | | AG | | | CA | |

TABLE 12
Primer designed in Comparative Example 3

| | Forward primer | | | Reverse primer | | |
|---|---|---|---|---|---|---|
| Mea- | | Base | | | Base | |
| sure- | | se- | | | se- | |
| ment | | quence | Se- | | quence | Se- |
| site | | (5′ → | quence | | (5′ → | quence |
| ID | Name | 3′) | number | Name | 3′) | number |
| 2 | 2F4 | TGGTAG | 675 | 2R4 | TAATCC | 759 |
| | | TGATTA | | | CACTTA | |
| | | GTTTAT | | | CAAAAA | |
| | | TTTTTG | | | ACA | |
| 3 | 3F4 | TGTAGA | 676 | 3R4 | TTAATA | 760 |
| | | GAGGAG | | | TCTATC | |
| | | GAGGTG | | | CTAATT | |
| | | AG | | | CCAACC | |
| 5 | 5F4 | TTTTTG | 677 | 5R4 | TCAAAA | 761 |
| | | GGTTTG | | | CATTTC | |
| | | AAATGT | | | TAAAAC | |
| | | TA | | | TATTAA | |
| | | | | | TATC | |
| 6 | 6F4 | GGGTTG | 678 | 6R4 | CAAAAC | 762 |
| | | AGGATT | | | TTAAAC | |
| | | AGTATT | | | AATAAT | |
| | | GAT | | | ACTTAC | |
| | | | | | TCA | |
| 7 | 7F4 | GGTTGA | 679 | 7R4 | TTAAAT | 763 |
| | | TGAGGT | | | CTAACA | |
| | | ATAGGT | | | CCCACA | |
| | | GA | | | CC | |
| 8 | 8F4 | GAAGTA | 680 | 8R4 | AATATA | 764 |
| | | GGTTAA | | | AAAAAT | |
| | | GAAGGA | | | AATCCC | |
| | | GGAT | | | CAAAC | |
| 9 | 9F4 | AGGATG | 681 | 9R4 | AAAAAA | 765 |
| | | GGGATT | | | CCAACC | |
| | | TTAGGT | | | TTTTCC | |
| | | TG | | | CT | |
| 10 | 10F4 | TTGTTT | 682 | 10R4 | CAAACA | 766 |
| | | TTTGTT | | | AAATTT | |
| | | GTGTGG | | | ACAACC | |
| | | AA | | | CA | |
| 12 | 12F4 | TTTTTA | 683 | 12R4 | ACCTCA | 767 |
| | | GGTTAT | | | CCCACT | |
| | | TTTTTA | | | TCTCCT | |
| | | AATGGA | | | AC | |
| 13 | 13F4 | GTTTTT | 684 | 13R4 | CTCAAA | 768 |
| | | AAGGTG | | | ATCCCA | |
| | | TTAGGG | | | ACCTCA | |
| | | GA | | | AAA | |
| 14 | 14F4 | GGGGAG | 685 | 14R4 | CCCACT | 769 |
| | | TTTTTT | | | AACTCC | |
| | | ATGAAG | | | CCAAAA | |
| | | GG | | | AC | |
| 15 | 15F4 | GTGTGG | 686 | 15R4 | ACCCAA | 770 |
| | | AAGGAA | | | AATCTA | |
| | | AAAAAA | | | CAAAAC | |
| | | AG | | | CC | |
| 16 | 16F4 | ATTTTT | 687 | 16R4 | AAATCC | 771 |
| | | TTATTA | | | CTATAT | |
| | | GGTTGG | | | ATTCCT | |
| | | TGG | | | TACCA | |
| 17 | 17F4 | TTTAGA | 688 | 17R4 | CCTCAT | 772 |
| | | TATAAA | | | ACTCTA | |
| | | TTTTTT | | | AAAACC | |
| | | TGTATG | | | CC | |
| | | GA | | | | |
| 18 | 18F4 | TTTATT | 689 | 18R4 | TCCCCA | 773 |
| | | GGGGTA | | | CTAATA | |
| | | GAGTAT | | | CTTCCT | |
| | | AGGTT | | | TAC | |
| 20 | 20F4 | AGAGGT | 690 | 20R4 | CATACC | 774 |
| | | TGTTGT | | | TCCTAA | |
| | | TGTGTT | | | CATCCC | |
| | | TG | | | AC | |
| 22 | 22F4 | GGGTGT | 691 | 22R4 | TACCTC | 775 |
| | | GGTAGG | | | TAATCC | |
| | | TGTTTT | | | CAATTC | |
| | | GG | | | AA | |
| 23 | 23F4 | TGTTGT | 692 | 23R4 | AAAAAT | 776 |
| | | TTTGTT | | | AAACCT | |
| | | TGTATG | | | TACAAA | |
| | | GA | | | CTACAC | |
| | | | | | A | |
| 24 | 24F4 | TTTAGG | 693 | 24R4 | CAAACA | 777 |
| | | TTTTTT | | | CCCCTT | |
| | | GTTGGT | | | TTTCAT | |
| | | GG | | | CA | |
| 25 | 25F4 | GAGTAT | 694 | 25R4 | AACTCT | 778 |
| | | TTTATG | | | ACAAAA | |
| | | TTGTTT | | | ACCAAA | |
| | | TAGTTG | | | TATAAT | |
| | | TTT | | | AAC | |
| 26 | 26F4 | AGTAGG | 695 | 26R4 | ACCCAC | 779 |
| | | AAGGGT | | | TACACA | |
| | | ATTGGT | | | AATAAA | |
| | | GG | | | AAAAT | |
| 27 | 27F4 | GGAAGT | 696 | 27R4 | TAACCT | 780 |
| | | GGGTTT | | | AACCAC | |
| | | GGGAAG | | | AAACAA | |
| | | TA | | | CC | |
| 28 | 28F4 | TTTTGG | 697 | 28R4 | ACCTCA | 781 |
| | | TTTTAA | | | AATAAC | |
| | | AAGAGA | | | TTAAAA | |
| | | GAAA | | | TTCACT | |
| 29 | 29F4 | TTTTTA | 698 | 29R4 | CCATTT | 782 |
| | | ATGAAT | | | TTCTAA | |
| | | GGATAT | | | CATATT | |
| | | AAGTGA | | | TACTAC | |
| | | | | | TAAA | |
| 30 | 30F4 | AAGTGG | 699 | 30R4 | ACTTAA | 783 |
| | | TAAGAG | | | TTTAAT | |
| | | GAGTTG | | | TACCCA | |
| | | GG | | | AACAAT | |
| 31 | 31F4 | AGGTTT | 700 | 31R4 | CCAATA | 784 |
| | | TTGTTG | | | ACATTA | |
| | | TGTTGG | | | AAACAA | |
| | | GA | | | CCA | |
| 32 | 32F4 | TTTTTT | 701 | 32R4 | ATCCAA | 785 |
| | | ATATTT | | | AATCTC | |
| | | ATATAT | | | CTAACC | |
| | | AAGTGT | | | TC | |
| | | TAGAAA | | | | |
| | | TG | | | | |
| 35 | 35F4 | GAGAGG | 702 | 35R4 | CCACCA | 786 |
| | | AAGTAA | | | AACACC | |
| | | GGGTTT | | | ACAATC | |
| | | ATTAA | | | AA | |
| 36 | 36F4 | TATTTT | 703 | 36R4 | AAAAAA | 787 |
| | | ATGGTT | | | CAACTC | |
| | | GGGGAA | | | CTTCTT | |
| | | AT | | | CC | |
| 37 | 37F4 | GGAGAT | 704 | 37R4 | CACAAA | 788 |
| | | TGGGGT | | | AAACCC | |
| | | TAGGA | | | TAAAAA | |
| | | TGA | | | ACTAAA | |
| | | | | | AA | |
| 38 | 38F4 | TGGTTT | 705 | 38R4 | AAATTA | 789 |
| | | TTTTGG | | | TTCAAA | |
| | | TAATAT | | | AAATAA | |
| | | AAGG | | | TTATAA | |
| | | | | | TAATAA | |
| | | | | | TATAC | |
| 39 | 39F4 | AGTAGG | 706 | 39R4 | ATAACA | 790 |
| | | TTTTTA | | | AAACTC | |
| | | AAATAT | | | AAAACC | |
| | | GTGGTT | | | CC | |
| 40 | 40F4 | AATTTT | 707 | 40R4 | CACTTA | 791 |
| | | GTAATT | | | TTACCC | |
| | | GGGTAG | | | AAACTA | |
| | | GG | | | ATCTTT | |
| 42 | 42F4 | TTTTGT | 708 | 42R4 | AAAAAA | 792 |
| | | AGTTTT | | | ATCCCT | |
| | | GAGAGG | | | CAATAC | |
| | | TGA | | | AAC | |
| 43 | 43F4 | TTGGAG | 709 | 43R4 | AAAACA | 793 |
| | | TTTTTA | | | AATTAC | |
| | | GTTTTG | | | CAATAA | |
| | | AGTT | | | ATTAAA | |
| | | | | | A | |
| 44 | 44F4 | TTGTGT | 710 | 44R4 | CAACCC | 794 |
| | | GATAGA | | | ACCCAC | |
| | | GTTTAG | | | ACAAAT | |
| | | TTGG | | | TA | |
| 45 | 45F4 | TTTTGT | 711 | 45R4 | CTCAAA | 795 |
| | | GTGGAT | | | AAAATC | |
| | | AGTTGT | | | AAACTT | |
| | | TG | | | CAA | |
| 46 | 46F4 | TTGGGA | 712 | 46R4 | CCCACA | 796 |
| | | TAGTGT | | | AACTAC | |
| | | TTTGAG | | | TTCTAC | |
| | | TG | | | AAA | |
| 47 | 47F4 | TTTTGA | 713 | 47R4 | ATTTCC | 797 |
| | | GAAGTT | | | TAAAAC | |
| | | TTGAAG | | | TTATAA | |
| | | GG | | | ATTTAT | |
| | | | | | AAAAA | |
| 49 | 49F4 | TTGTTT | 714 | 49R4 | TTTCAA | 798 |
| | | TTAAAA | | | ACCTTA | |
| | | AAATTA | | | TCTTAA | |
| | | AAAAGA | | | AACTTC | |
| | | G | | | | |
| 50 | 50F4 | GAGTGT | 715 | 50R4 | CCCTTT | 799 |
| | | TTTGGG | | | ATACTT | |
| | | GAATGT | | | TAATTT | |
| | | GT | | | TCTCC | |
| 51 | 51F4 | GTTGTG | 716 | 51R4 | AAAATC | 800 |
| | | AATATA | | | CCCTTC | |
| | | GGTGTG | | | AATTCT | |
| | | AGTTAA | | | AC | |
| 52 | 52F4 | AATTGT | 717 | 52R4 | AACCAA | 801 |
| | | TGGTAG | | | ACCCTT | |
| | | GTTGTT | | | AACAAC | |
| | | GG | | | CC | |
| 53 | 53F4 | TAGATT | 718 | 53R4 | AAAAAA | 802 |
| | | TTTTTT | | | AATAAA | |
| | | GTTAAA | | | TAATAT | |
| | | TTTTTT | | | TTTTCA | |
| | | TT | | | AAA | |
| 54 | 54F4 | TGAGAT | 719 | 54R4 | TACCAA | 803 |
| | | GATTAA | | | CTACAC | |
| | | ATGAAG | | | CACTCT | |
| | | ATTAAA | | | CC | |
| 55 | 55F4 | GGTTGT | 720 | 55R4 | CAAAAT | 804 |
| | | TGTAAT | | | CAACCA | |
| | | TGTTTG | | | CAACCT | |
| | | TTG | | | AC | |
| 56 | 56F4 | TTGTAG | 721 | 56R4 | TTTTAC | 805 |
| | | TGTAGT | | | TCATAA | |
| | | TTGAGA | | | TATAAT | |
| | | AATAGG | | | TTCTAC | |
| | | | | | CTCA | |
| 57 | 57F4 | TGTGTT | 722 | 57R4 | AAAACT | 806 |
| | | TAATTG | | | ATAAAC | |
| | | TTTGTT | | | TCCCTT | |
| | | TTTTT | | | TCATT | |
| 58 | 58F4 | TATTGG | 723 | 58R4 | CCCTCA | 807 |
| | | AGGGTG | | | ACCTCC | |
| | | GGAGAG | | | TAAATA | |
| | | TT | | | CA | |
| 59 | 59F4 | TTGTTT | 724 | 59R4 | AAAAAT | 808 |
| | | ATGAAA | | | ACCATT | |
| | | TTTGTG | | | TACCTA | |
| | | GG | | | ACCA | |
| 60 | 60F4 | GAGTGT | 725 | 60R4 | AACCAC | 809 |
| | | GTGATT | | | CACCTC | |
| | | GGGTTT | | | CAAATC | |
| | | GT | | | TT | |
| 63 | 63F4 | GGATAT | 726 | 63R4 | ACACTC | 810 |
| | | TGGTTG | | | AAAAAA | |
| | | TTTTGG | | | ACTACC | |
| | | AT | | | CTT | |
| 64 | 64F4 | TTTTTA | 727 | 64R4 | CCCATT | 811 |
| | | TAATTG | | | ATTTCA | |
| | | GTGAGG | | | ACTTAC | |
| | | GA | | | ACTC | |
| 65 | 65F4 | GGATGA | 728 | 65R4 | TCACTC | 812 |
| | | GTAGTT | | | ACTAAA | |
| | | TTTGGG | | | CAAAAC | |
| | | GT | | | AAAA | |
| 66 | 66F4 | GAAAGA | 729 | 66R4 | AACTTA | 813 |
| | | AAAGAA | | | AACCCA | |
| | | AAAGAG | | | AAACTT | |
| | | AAAGT | | | TAAAAC | |
| | | | | | TAC | |
| 67 | 67F4 | TGTGGT | 730 | 67R4 | CCTCTC | 814 |
| | | TTTGAA | | | CTTTAA | |
| | | ATAGAT | | | AAAAAA | |
| | | TTTG | | | TTCC | |
| 68 | 68F4 | TTTAAA | 731 | 68R4 | TCCCTA | 815 |
| | | GGGTGT | | | TCTAAA | |
| | | TATTTG | | | CCCCTC | |
| | | AGG | | | CT | |
| 69 | 69F4 | AGGAAG | 732 | 69R4 | AAAATC | 816 |
| | | ATATTG | | | TACCTT | |
| | | TTTATG | | | CCACCA | |
| | | TGGA | | | AA | |
| 70 | 70F4 | TATAGG | 733 | 70R4 | CATACC | 817 |
| | | ATGGTA | | | AAAAAA | |
| | | GGGTTG | | | AACTTT | |
| | | GG | | | CTCA | |
| 71 | 71F4 | AGGAAA | 734 | 71R4 | ATCTAC | 818 |
| | | ATTAAT | | | AACTCC | |
| | | GAATTG | | | CAAAAA | |
| | | TTAAAG | | | TTC | |
| 72 | 72F4 | TTGAAT | 735 | 72R4 | CCCCTA | 819 |
| | | GTTGTT | | | AAATTT | |
| | | ATTTGG | | | ACTAAA | |
| | | TATG | | | AAAATT | |
| | | | | | A | |
| 73 | 73F4 | TTTGAT | 736 | 73R4 | AAACTA | 820 |
| | | TTTGTT | | | ACCACC | |
| | | TTGGAG | | | CTCTCC | |
| | | TG | | | TA | |
| 74 | 74F4 | TTTTAA | 737 | 74R4 | CTCTTA | 821 |
| | | TTTTGT | | | ATACAC | |
| | | ATTTGT | | | TCCATA | |
| | | GTGATT | | | ATTAAC | |
| | | | | | C | |
| 75 | 75F4 | AAAGTT | 738 | 75R4 | CCTTTT | 822 |
| | | TTAATA | | | TATTTA | |
| | | GTTTTA | | | AAAATA | |
| | | AGTTTG | | | ATATTA | |
| | | GA | | | AACA | |
| 76 | 76F4 | TGTATG | 739 | 76R4 | TTCCTA | 823 |
| | | GTATTT | | | ATAAAC | |
| | | TTTGAA | | | TAAAAA | |
| | | GTGA | | | TATTAA | |
| | | | | | AATTC | |
| 77 | 77F4 | TTGATT | 740 | 77R4 | CCTCCT | 824 |
| | | GGTTAG | | | CTTTTC | |
| | | AGTTGG | | | TTTCTA | |
| | | TT | | | TCA | |
| 81 | 81F4 | GGATTT | 741 | 81R4 | ATTACA | 825 |
| | | GTAATT | | | ATTCTC | |
| | | GGTATA | | | TTTCCT | |
| | | GAAGG | | | TTAAAA | |
| 82 | 82F4 | GAGGGG | 742 | 82R4 | CATCTC | 826 |
| | | ATGTTT | | | TTACTA | |
| | | TTTTGT | | | AAACTA | |
| | | TG | | | ACATCA | |
| | | | | | CA | |
| 83 | 83F4 | TTTAAA | 743 | 83R4 | TTTATT | 827 |
| | | GGAGGG | | | AAATAT | |
| | | TTGGAA | | | TAAATA | |
| | | TG | | | CTTAAA | |
| | | | | | AAAATT | |
| | | | | | AAA | |
| 84 | 84F4 | TTGTAG | 744 | 84R4 | AACTTC | 828 |
| | | TTGGTG | | | ACAAAA | |
| | | ATTGTT | | | ACATCT | |
| | | GA | | | CTCT | |
| 85 | 85F4 | GGTTAG | 745 | 85R4 | AAATTA | 829 |
| | | GATTTA | | | ATATTA | |
| | | TGTGTT | | | TTTTTC | |
| | | TTATAA | | | TAACCC | |
| | | AAT | | | C | |
| 86 | 86F4 | ATTAAA | 746 | 86R4 | TCACAC | 830 |
| | | GAGTTA | | | CCTTAC | |
| | | AGTATT | | | TAATTA | |
| | | AGAAAT | | | CCC | |
| | | GATG | | | | |
| 87 | 87F4 | TAAAGG | 747 | 87R4 | TCCTAA | 831 |
| | | TGAAGG | | | AAAAAT | |
| | | GTGTGG | | | CTTTCC | |
| | | GG | | | AAC | |
| 88 | 88F4 | AGTGTG | 748 | 88R4 | TCAACC | 832 |
| | | GGGGTT | | | AAAATA | |
| | | TTTTTT | | | TACCTT | |
| | | GG | | | CTAAA | |
| 89 | 89F4 | GTTAGG | 749 | 89R4 | CCAACT | 833 |
| | | TTGGGG | | | ATACTT | |
| | | TGGTGG | | | TCCCAT | |
| | | TT | | | AACCT | |
| 91 | 91F4 | GGAGGT | 750 | 91R4 | AAACCC | 834 |
| | | GGGGGT | | | CAAAAC | |
| | | TTTTTA | | | TCCCAC | |
| | | TT | | | AAC | |
| 92 | 92F4 | TTTTTT | 751 | 92R4 | CAAAAT | 835 |
| | | TGGGTG | | | ATAAAA | |
| | | TTAAGT | | | ATCAAA | |
| | | TAGT | | | TCCC | |
| 93 | 93F4 | GGTTTT | 752 | 93R4 | ATCAAA | 836 |
| | | AGGTGA | | | ATCATA | |
| | | TATTTG | | | AAAAAA | |
| | | AATAAT | | | AACAAA | |
| | | | | | A | |
| 94 | 94F4 | TTTTAG | 753 | 94R4 | TTCTCC | 837 |
| | | TAGGGG | | | CTTATA | |
| | | TTTTAG | | | ATTTTA | |
| | | ATTTT | | | ACACA | |
| 95 | 95F4 | AGAGTT | 754 | 95R4 | CAACTC | 838 |
| | | AAGTTA | | | AAAACT | |
| | | GATGTG | | | CTAAAA | |
| | | TTATAA | | | TAATAA | |
| | | TTAGAG | | | ACA | |
| 97 | 97F4 | AGATTA | 755 | 97R4 | CTATCT | 839 |
| | | AAAAAT | | | AAAAAT | |
| | | ATTTTT | | | ACAAAC | |
| | | ATTTGT | | | TAAAAT | |
| | | GTAA | | | AAATCT | |
| 98 | 98F4 | TGATAT | 756 | 98R4 | CCCTAA | 840 |
| | | AAATAG | | | CCTACC | |
| | | GTTTGG | | | AACAAC | |
| | | GGT | | | CA | |
| 99 | 99F4 | GGGAGT | 757 | 99R4 | CCTAAA | 841 |
| | | AGGAGG | | | CTCCCC | |
| | | GGTGTG | | | AAAAAC | |
| | | TG | | | AC | |
| 100 | 100F4 | TGAATT | 758 | 100R4 | CTCATT | 842 |
| | | TGTTGT | | | TAAACC | |
| | | TGATTT | | | TCATAA | |
| | | TG | | | CCC | |

TABLE 13
Primer designed in Comparative Example 4

| | Forward primer | | | Reverse primer | | |
|---|---|---|---|---|---|---|
| Mea- | | Base | | | Base | |
| sure- | | se- | | | se | |
| ment | | quence | Se- | | quence | Se- |
| site | | (5′ → | quence | | (5′ → | quence |
| ID | Name | 3′) | number | Name | 3′) | number |
| 2 | 2F1 | TGGTAG | 843 | 2R1 | TAATCC | 927 |
| | | TGATTA | | | CACTTA | |
| | | GTTTAT | | | CAAAAA | |
| | | TTTTTG | | | ACA | |
| 3 | 3F1 | TGTAGA | 844 | 3R1 | TTAATA | 928 |
| | | GAGGAG | | | TCTATC | |
| | | GAGGT | | | CTAATT | |
| | | GAG | | | CCAACC | |
| 5 | 5F1 | TTTTTG | 845 | 5R1 | AATCAA | 929 |
| | | GGTTTG | | | AACATT | |
| | | AAATGT | | | TCTAAA | |
| | | TA | | | ACTATT | |
| | | | | | AAT | |
| 6 | 6F1 | GGGTTG | 846 | 6R1 | CAAAAC | 930 |
| | | AGGATT | | | TTAAAC | |
| | | AGTATT | | | AATAAT | |
| | | GAT | | | ACTTAC | |
| | | | | | TCA | |
| 7 | 7F1 | GGTTGA | 847 | 7R1 | TTAAAT | 931 |
| | | TGAGGT | | | CTAACA | |
| | | ATAGGT | | | CCCACA | |
| | | GA | | | CC | |
| 8 | 8F1 | GAAGTA | 848 | 8R1 | AATATA | 932 |
| | | GGTTAA | | | AAAAAT | |
| | | GAAGGA | | | AATCCC | |
| | | GGAT | | | CAAAC | |
| 9 | 9F1 | AGGATG | 849 | 9R1 | AAAAAA | 933 |
| | | GGGATT | | | CCAACC | |
| | | TTAGGT | | | TTTTCC | |
| | | TG | | | CT | |
| 10 | 10F1 | TTGTTT | 850 | 10R1 | CAAACA | 934 |
| | | TTTGTT | | | AAATTT | |
| | | GTGTGG | | | ACAACC | |
| | | AA | | | CA | |
| 12 | 12F1 | TTTTTA | 851 | 12R1 | ACCTCA | 935 |
| | | GGTTAT | | | CCCACT | |
| | | TTTTTA | | | TCTCCT | |
| | | AATGGA | | | AC | |
| 13 | 13F1 | GTTTTT | 852 | 13R1 | CTCAAA | 936 |
| | | AAGGTG | | | ATCCCA | |
| | | TTAGGG | | | ACCTCA | |
| | | GA | | | AAA | |
| 14 | 14F1 | GGGGAG | 853 | 14R1 | CCCACT | 937 |
| | | TTTTTT | | | AACTCC | |
| | | ATGAAG | | | CCAAAA | |
| | | GG | | | AC | |
| 15 | 15F1 | GTGTGG | 854 | 15R1 | ACCCAA | 938 |
| | | AAGGAA | | | AATCTA | |
| | | AAAAAA | | | CAAAAC | |
| | | AG | | | CC | |
| 16 | 16F1 | ATTTTT | 855 | 16R1 | AAATCC | 939 |
| | | TTATTA | | | CTATAT | |
| | | GGTTGG | | | ATTCCT | |
| | | TGG | | | TACCA | |
| 17 | 17F1 | TTTAGA | 856 | 17R1 | CCTCAT | 940 |
| | | TATAAA | | | ACTCTA | |
| | | TTTTTT | | | AAAACC | |
| | | TGTATG | | | CC | |
| | | GA | | | | |
| 18 | 18F1 | TTTATT | 857 | 18R1 | TCCCCA | 941 |
| | | GGGGTA | | | CTAATA | |
| | | GAGTAT | | | CTTCCT | |
| | | AGGTT | | | TAC | |
| 20 | 20F1 | AGAGGT | 858 | 20R1 | CATACC | 942 |
| | | TGTTGT | | | TCCTAA | |
| | | TGTGTT | | | CATCCC | |
| | | TG | | | AC | |
| 22 | 22F1 | GGGTGT | 859 | 22R1 | TACCTC | 943 |
| | | GGTAGG | | | TAATCC | |
| | | TGTTTT | | | CAATTC | |
| | | GG | | | AA | |
| 23 | 23F1 | TGTTGT | 860 | 23R1 | AAAAAT | 944 |
| | | TTTGTT | | | AAACCT | |
| | | TGTATG | | | TACAAA | |
| | | GA | | | CTACAC | |
| | | | | | A | |
| 24 | 24F1 | TTTAGG | 861 | 24R1 | CAAACA | 945 |
| | | TTTTTT | | | CCCCTT | |
| | | GTTGGT | | | TTTCAT | |
| | | GG | | | CA | |
| 25 | 25F1 | GAGTAT | 862 | 25R1 | AACTCT | 946 |
| | | TTTATG | | | ACAAAA | |
| | | TTGTTT | | | ACCAAA | |
| | | TAGTTG | | | TATAAT | |
| | | TTT | | | AAC | |
| 26 | 26F1 | AGTAGG | 863 | 26R1 | ACCCAC | 947 |
| | | AAGGGT | | | TACACA | |
| | | ATTGGT | | | AATAAA | |
| | | GG | | | AAAAT | |
| 27 | 27F1 | GGAAGT | 864 | 27R1 | TAACCT | 948 |
| | | GGGTTT | | | AACCAC | |
| | | GGGAAG | | | AAACAA | |
| | | TA | | | CC | |
| 28 | 28F1 | TTTTGG | 865 | 28R1 | ACCTCA | 949 |
| | | TTTTAA | | | AATAAC | |
| | | AAGAGA | | | TTAAAA | |
| | | GAAA | | | TTCACT | |
| 29 | 29F1 | AAAGAG | 866 | 29R1 | TTCTAA | 950 |
| | | TTTTTA | | | CATATT | |
| | | ATGAAT | | | TACTAC | |
| | | GGATAT | | | TAAAAA | |
| | | | | | ATTTAA | |
| | | | | | A | |
| 30 | 30F1 | AAGTGG | 867 | 30R1 | CACTTA | 951 |
| | | TAAGAG | | | ATTTAA | |
| | | GAGTTG | | | TTACCC | |
| | | GG | | | AAACA | |
| 31 | 31F1 | AGGTTT | 868 | 31R1 | CCAATA | 952 |
| | | TTGTTG | | | ACATTA | |
| | | TGTTGG | | | AAACAA | |
| | | GA | | | CCA | |
| 32 | 32F1 | TTTTTT | 869 | 32R1 | ATCCAA | 953 |
| | | ATATTT | | | AATCTC | |
| | | ATATAT | | | CTAACC | |
| | | AAGTGT | | | TC | |
| | | TAGAAA | | | | |
| | | TG | | | | |
| 35 | 35F1 | GAGAGG | 870 | 35R1 | CCACCA | 954 |
| | | AAGTAA | | | AACACC | |
| | | GGGTTT | | | ACAATC | |
| | | ATTAA | | | AA | |
| 36 | 36F1 | TATTTT | 871 | 36R1 | AAAAAA | 955 |
| | | ATGGTT | | | CAACTC | |
| | | GGGGAA | | | CTTCTT | |
| | | AT | | | CC | |
| 37 | 37F1 | GGAGAT | 872 | 37R1 | CACAAA | 956 |
| | | TGGGGT | | | AAACCC | |
| | | TAGGAT | | | TAAAAA | |
| | | GA | | | ACTAAA | |
| | | | | | AA | |
| 38 | 38F1 | TGGTTT | 873 | 38R1 | AAATTA | 957 |
| | | TTTTGG | | | TTCAAA | |
| | | TAATAT | | | AAATAA | |
| | | AAGG | | | TTATAA | |
| | | | | | TAATAA | |
| | | | | | TATAC | |
| 39 | 39F1 | AGTAGG | 874 | 39R1 | ATAACA | 958 |
| | | TTTTTA | | | AAACTC | |
| | | AAATAT | | | AAAACC | |
| | | GTGGTT | | | CC | |
| 40 | 40F1 | AATTTT | 875 | 40R1 | CACTTA | 959 |
| | | GTAATT | | | TTACCC | |
| | | GGGTAG | | | AAACTA | |
| | | GG | | | ATCTTT | |
| 42 | 42F1 | TTTTGT | 876 | 42R1 | AAAAAA | 960 |
| | | AGTTTT | | | ATCCCT | |
| | | GAGAGG | | | CAATAC | |
| | | TGA | | | AAC | |
| 43 | 43F1 | TTGGAG | 877 | 43R1 | AAAACA | 961 |
| | | TTTTTA | | | AATTAC | |
| | | GTTTTG | | | CAATAA | |
| | | AGTT | | | ATTAAA | |
| | | | | | A | |
| 44 | 44F1 | TTGTGT | 878 | 44R1 | CAACCC | 962 |
| | | GATAGA | | | ACCCAC | |
| | | GTTTAG | | | ACAAAT | |
| | | TTGG | | | TA | |
| 45 | 45F1 | TTTTGT | 879 | 45R1 | CTCAAA | 963 |
| | | GTGGAT | | | AAAATC | |
| | | AGTTGT | | | AAACTT | |
| | | TG | | | CAA | |
| 46 | 46F1 | TTGGGA | 880 | 46R1 | CCCACA | 964 |
| | | TAGTGT | | | AACTAC | |
| | | TTTGAG | | | TTCTAC | |
| | | TG | | | AAA | |
| 47 | 47F1 | TTTTGA | 881 | 47R1 | ATTTCC | 965 |
| | | GAAGTT | | | TAAAAC | |
| | | TTGAAG | | | TTATAA | |
| | | GG | | | ATTTAT | |
| | | | | | AAAAA | |
| 49 | 49F1 | TTGTTT | 882 | 49R1 | TTTCAA | 966 |
| | | TTAAAA | | | ACCTTA | |
| | | AAATTA | | | TCTTAA | |
| | | AAAAGA | | | AACTTC | |
| | | G | | | | |
| 50 | 50F1 | GAGTGT | 883 | 50R1 | CCCTTT | 967 |
| | | TTTGGG | | | ATACTT | |
| | | GAATGT | | | TAATTT | |
| | | GT | | | TCTCC | |
| 51 | 51F1 | GTTGTG | 884 | 51R1 | AAAATC | 968 |
| | | AATATA | | | CCCTTC | |
| | | GGTGTG | | | AATTCT | |
| | | AGTTAA | | | AC | |
| 52 | 52F1 | AATTGT | 885 | 52R1 | AACCAA | 969 |
| | | TGGTAG | | | ACCCTT | |
| | | GTTGTT | | | AACAAC | |
| | | GG | | | CC | |
| 53 | 53F1 | TAGATT | 886 | 53R1 | AAAAAA | 970 |
| | | TTTTTT | | | AATAAA | |
| | | GTTAAA | | | TAATAT | |
| | | TTTTTT | | | TTTTCA | |
| | | TT | | | AAA | |
| 54 | 54F1 | TGAGAT | 887 | 54R1 | TACCAA | 971 |
| | | GATTAA | | | CTACAC | |
| | | ATGAAG | | | CACTCT | |
| | | ATTAAA | | | CC | |
| 55 | 55F1 | GGTTGT | 888 | 55R1 | CACAAA | 972 |
| | | TGTAAT | | | ATCAAC | |
| | | TGTTTG | | | CACAAC | |
| | | TTG | | | CT | |
| 56 | 56F1 | TTGTAG | 889 | 56R1 | TTTTAC | 973 |
| | | TGTAGT | | | TCATAA | |
| | | TTGAGA | | | TATAAT | |
| | | AATAGG | | | TTCTAC | |
| | | | | | CTCA | |
| 57 | 57F1 | TGTGTT | 890 | 57R1 | AAAACT | 974 |
| | | TAATTG | | | ATAAAC | |
| | | TTTGTT | | | TCCCTT | |
| | | TTTTT | | | TCATT | |
| 58 | 58F1 | TATTGG | 891 | 58R1 | CCCTCA | 975 |
| | | AGGGTG | | | ACCTCC | |
| | | GGAGAG | | | TAAATA | |
| | | TT | | | CA | |
| 59 | 59F1 | TTGTTT | 892 | 59R1 | AAAAAT | 976 |
| | | ATGAAA | | | ACCATT | |
| | | TTTGTG | | | TACCTA | |
| | | GG | | | ACCA | |
| 60 | 60F1 | GAGTGT | 893 | 60R1 | AACCAC | 977 |
| | | GTGATT | | | CACCTC | |
| | | GGGTTT | | | CAAATC | |
| | | GT | | | TT | |
| 63 | 63F1 | GGATAT | 894 | 63R1 | ACACTC | 978 |
| | | TGGTTG | | | AAAAAA | |
| | | TTTTGG | | | ACTACC | |
| | | AT | | | CTT | |
| 64 | 64F1 | GGGGAT | 895 | 64R1 | TTTCAA | 979 |
| | | TTTTTA | | | CTTACA | |
| | | TAATTG | | | CTCTAA | |
| | | GT | | | CAAACA | |
| 65 | 65F1 | GGATGA | 896 | 65R1 | TCACTC | 980 |
| | | GTAGTT | | | ACTAAA | |
| | | TTTGGG | | | CAAAAC | |
| | | GT | | | AAAA | |
| 66 | 66F1 | GAAAGA | 897 | 66R1 | AACTTA | 981 |
| | | AAAGAA | | | AACCCA | |
| | | AAAGAG | | | AAACTT | |
| | | AAAGT | | | TAAAAC | |
| | | | | | TAC | |
| 67 | 67F1 | TGTGGT | 898 | 67R1 | CCTCTC | 982 |
| | | TTTGAA | | | CTTTAA | |
| | | ATAGAT | | | AAAAAA | |
| | | TTTG | | | TTCC | |
| 68 | 68F1 | AAAGGG | 899 | 68R1 | TCCCTA | 983 |
| | | TGTTAT | | | TCTAAA | |
| | | TTGAGG | | | CCCCTC | |
| | | TT | | | CT | |
| 69 | 69F1 | TGTGGA | 900 | 69R1 | TCAAAA | 984 |
| | | AATATT | | | ATCTAC | |
| | | GATTTT | | | CTTCCA | |
| | | TGA | | | CC | |
| 70 | 70F1 | TTATAG | 901 | 70R1 | TCACAT | 985 |
| | | GATGGT | | | ACCAAA | |
| | | AGGGTT | | | AAAAAC | |
| | | GG | | | TTTC | |
| 71 | 71F1 | AGGAAA | 902 | 71R1 | ATCTAC | 986 |
| | | ATTAAT | | | AACTCC | |
| | | GAATTG | | | CAAAAA | |
| | | TTAAAG | | | TTC | |
| 72 | 72F1 | TTGAAT | 903 | 72R1 | CCCCTA | 987 |
| | | GTTGTT | | | AAATTT | |
| | | ATTTGG | | | ACTAAA | |
| | | TATG | | | AAAATT | |
| 73 | 73F1 | TTTGAT | 904 | 73R1 | AAACTA | 988 |
| | | TTTGTT | | | ACCACC | |
| | | TTGGAG | | | CTCTCC | |
| | | TG | | | TA | |
| 74 | 74F1 | TTTTAA | 905 | 74R1 | CTCTTA | 989 |
| | | TTTTGT | | | ATACAC | |
| | | ATTTGT | | | TCCATA | |
| | | GTGATT | | | ATTAAC | |
| | | | | | C | |
| 75 | 75F1 | AAAGTT | 906 | 75R1 | CCTTTT | 990 |
| | | TTAATA | | | TATTTA | |
| | | GTTTTA | | | AAAATA | |
| | | AGTTTG | | | ATATTA | |
| | | GA | | | AACA | |
| 76 | 76F1 | TGTATG | 907 | 76R1 | TTCCTA | 991 |
| | | GTATTT | | | ATAAAC | |
| | | TTTGAA | | | TAAAAA | |
| | | GTGA | | | TATTAA | |
| | | | | | AATTC | |
| 77 | 77F1 | TTGATT | 908 | 77R1 | CCTCCT | 992 |
| | | GGTTAG | | | CTTTTC | |
| | | AGTTGG | | | TTTCTA | |
| | | TT | | | TCA | |
| 81 | 81F1 | GGATTT | 909 | 81R1 | ATTACA | 993 |
| | | GTAATT | | | ATTCTC | |
| | | GGTATA | | | TTTCCT | |
| | | GAAGG | | | TTAAAA | |
| 82 | 82F1 | GAGGGG | 910 | 82R1 | CATCTC | 994 |
| | | ATGTTT | | | TTACTA | |
| | | TTTTGT | | | AAACTA | |
| | | TG | | | ACATCA | |
| | | | | | CA | |
| 83 | 83F1 | TTTAAA | 911 | 83R1 | TTTATT | 995 |
| | | GGAGGG | | | AAATAT | |
| | | TTGGAA | | | TAAATA | |
| | | TG | | | CTTAAA | |
| | | | | | AAAATT | |
| | | | | | AAA | |
| 84 | 84F1 | TTGTAG | 912 | 84R1 | AACTTC | 996 |
| | | TTGGTG | | | ACAAAA | |
| | | ATTGTT | | | ACATCT | |
| | | GA | | | CTCT | |
| 85 | 85F1 | AGGTTA | 913 | 85R1 | AAATTA | 997 |
| | | GGATTT | | | ATATTA | |
| | | ATGTGT | | | TTTTTC | |
| | | TTTATA | | | TAACCC | |
| | | A | | | C | |
| 86 | 86F1 | ATTAAA | 914 | 86R1 | TCACAC | 998 |
| | | GAGTTA | | | CCTTAC | |
| | | AGTATT | | | TAATTA | |
| | | AGAAAT | | | CCC | |
| | | GATG | | | | |
| 87 | 87F1 | TAATAA | 915 | 87R1 | TCCTAA | 999 |
| | | AGGTGA | | | AAAAAT | |
| | | AGGGTG | | | CTTTCC | |
| | | TG | | | AAC | |
| 88 | 88F1 | AGTGTG | 916 | 88R1 | AAATCA | 1000 |
| | | GGGGTT | | | ACCAAA | |
| | | TTTTTT | | | ATATAC | |
| | | GG | | | CTTCT | |
| 89 | 89F1 | GTTAGG | 917 | 89R1 | CCAACT | 1001 |
| | | TTGGGG | | | ATACTT | |
| | | TGGTGG | | | TCCCAT | |
| | | TT | | | AACCT | |
| 91 | 91F1 | TTGGAG | 918 | 91R1 | AAACCC | 1002 |
| | | GTGGGG | | | CAAAAC | |
| | | GTTTTT | | | TCCCAC | |
| | | TA | | | AAC | |
| 92 | 92F1 | TTTTTT | 919 | 92R1 | CAAAAT | 1003 |
| | | TGGGTG | | | ATAAAA | |
| | | TTAAGT | | | ATCAAA | |
| | | TAGT | | | TCCC | |
| 93 | 93F1 | GGTTTT | 920 | 93R1 | ATCAAA | 1004 |
| | | AGGTGA | | | ATCATA | |
| | | TATTTG | | | AAAAAA | |
| | | AATAAT | | | AACAAA | |
| | | | | | A | |
| 94 | 94F1 | TTTTAG | 921 | 94R1 | TTCTCC | 1005 |
| | | TAGGGG | | | CTTATA | |
| | | TTTTAG | | | ATTTTA | |
| | | ATTTT | | | ACACA | |
| 95 | 95F1 | AGAGTT | 922 | 95R1 | AACAAC | 1006 |
| | | AAGTTA | | | TCAAAA | |
| | | GATGTG | | | CTCTAA | |
| | | TTATAA | | | AATAAT | |
| | | TTAGAG | | | AAA | |
| 97 | 97F1 | AGATTA | 923 | 97R1 | CTATCT | 1007 |
| | | AAAAAT | | | AAAAAT | |
| | | ATTTTT | | | ACAAAC | |
| | | ATTTGT | | | TAAAAT | |
| | | GTAA | | | AAATCT | |
| 98 | 98F1 | TGATAT | 924 | 98R1 | CCCTAA | 1008 |
| | | AAATAG | | | CCTACC | |
| | | GTTTGG | | | AACAAC | |
| | | GGT | | | CA | |
| 99 | 99F1 | GGGAGT | 925 | 99R1 | CCTAAA | 1009 |
| | | AGGAGG | | | CTCCCC | |
| | | GGTGTG | | | AAAAAC | |
| | | TG | | | AC | |
| 100 | 100F1 | TGAATT | 926 | 100R1 | CTCATT | 1010 |
| | | TGTTGT | | | TAAACC | |
| | | TGATTT | | | TCATAA | |
| | | TG | | | CCC | |

As shown in Table 7, FIG. 12A, and FIG. 12B, it can be seen that the maximum value of the local alignment score is determined as threshold values of an integer of 1 to 4, and the adopted primer sequence pairs (Examples 1 to 4) have a very low dimer formation of 2% or less while acquiring a high primer design success rate. On the other hand, it can be seen that, in the adopted primer sequence pairs (Comparative Examples 2 to 4) in which the maximum value of the local alignment score is determined with the threshold values of 0, 5, and 6, even in a case where the dimer formation rate is low, the primer design success rate is low, or even in a case where the primer design success rate is high, the dimer formation rate is high.

The primer design success rate (84%) of Comparative Example 3 slightly exceeds the primer design success rate (82%) of Example 4. However, in Example 4, that is, in a case where multiplex PCR is performed using the primer designed and manufactured according to the present invention, the dimer formation rate is suppressed to 2% or less, whereas in Comparative Example 3, that is, in a case where the maximum value of the local alignment score is determined with the threshold value 5 outside the numerical range according to the present invention, dimers are formed in about 20% of the adopted primer sequence pairs. Therefore, in a case where the primer sequence pair designed and manufactured in Comparative Example 3 is used in multiplex PCR, problems such as inability to amplify a desired target site, and generation of a large amount of primer dimers to inhibit the amplified sequence of the other target site occur, and there is a high possibility of failure.

### EXPLANATION OF REFERENCES

10, 10A: primer design device
12: input unit
14: storage unit
16: output unit
18: primer design processing unit
20: base sequence data acquisition unit
22: target site information acquisition unit
24: base conversion unit
26: complementary strand generation unit
28: partial sequence cutting unit
30: primer candidate sequence selection unit
32: primer sequence determination unit
34: control unit
36: communication interface
38: communication network
40: server
42: search server

The primer designed according to the present invention can be used for measuring the DNA methylation degree of a biological sample in the fields of drug discovery, diagnosis, and other bioindustries.

[Sequence list] International application F00852W1JP23021016_13.xml based on International Patent Cooperation Treaty

What is claimed is:

1. A primer design method for amplicon methylation sequence analysis, which is a method for designing a primer for amplicon methylation sequence analysis, the method utilizing a bisulfite reaction or an enzyme reaction and a multiplex PCR for measuring a methylation degree of at least one double-stranded genomic DNA and being used for simultaneously amplifying a plurality of regions each including two or more target sites where the methylation degree is measured, the design method comprising:
a complementary strand generation step of generating a complementary strand with respect to a template strand of the DNA;
a partial sequence cutting step of selecting one target site from the two or more target sites and, from each of the strands, cutting out one or more partial sequences having a predetermined length from a base sequence located on a 5′ terminal side of the selected target site;
a primer candidate sequence selection step of selecting the one or more cut-out partial sequences as one or more primer candidate sequences;
a primer sequence determination step of adopting and determining a forward primer sequence and a reverse primer sequence for amplifying a region including the selected predetermined target site from the one or more primer candidate sequences; and
a repeating step of repeating the partial sequence cutting step, the primer candidate sequence selection step, and the primer sequence determination step until all of the two or more target sites are selected in the partial sequence cutting step,
wherein (I) in a case where one or more primer sequences of a different target site have not yet been determined, the primer sequence determination step includes
[1] selecting one or more primer candidate sequence pairs related to the predetermined target site from the one or more primer candidate sequences,
[2] selecting one primer candidate sequence pair from the one or more primer candidate sequence pairs of the predetermined target site, and calculating a local alignment score between sequences of the selected primer candidate sequence pair, and
[3] adopting and determining the primer candidate sequence pair for which the local alignment score being equal to or less than a predetermined threshold value is calculated as the forward primer sequence and the reverse primer sequence for amplifying the region including the predetermined target site, (II) in a case where one or more primer sequences of the different target site have already been determined, the primer sequence determination step includes
[1] selecting one or more primer candidate sequence pairs related to the predetermined target site from the one or more primer candidate sequences,
[2] selecting one primer candidate sequence pair from the one or more primer candidate sequence pairs of the predetermined target site, and calculating a local alignment score between each of candidate sequences of the selected primer candidate sequence pair and each of the already determined primer sequences of the different target site, and a local alignment score between the sequences of the selected primer candidate sequence pair, and
[3] detecting a maximum value from all the calculated local alignment scores, and adopting and determining a primer candidate sequence pair for which a local alignment score having the maximum value being equal to or less than a predetermined threshold value is calculated, as the forward primer sequence and the reverse primer sequence for amplifying the region including the predetermined target site,
in the step [3] of the (I) and the (II), in a case where the primer candidate sequence pair is not adopted as the forward primer sequence and the reverse primer sequence for amplifying the region including the predetermined target site, one different pair is selected from the one or more primer candidate sequence pairs selected in the step [1] of the (I) and the (II), and the steps [2] and [3] are repeated until at least one primer candidate sequence pair is adopted,
in a case where <1> a complementary base pair is set to “X” per pair, <2> a non-complementary base pair is set to “Y” per pair, and <3> a case where there is insertion or deletion is set to “Z” per one insertion or deletion between the primer candidate sequences, the local alignment score is calculated using “X” of 1, “Y” of −4 to −2, and “Z” of −6 to −3, and
the predetermined threshold value is 1 to 4.

2. The primer design method for amplicon methylation sequence analysis according to claim 1,
wherein in the primer sequence determination step, (I) in the case where the number of the target sites is two or more and one or more primer sequences of a different target site have not yet been determined,
in the step [2], all pairs are selected from the one or more primer candidate sequence pairs of the predetermined target site, and for each pair, a local alignment score between sequences of the selected primer candidate sequence pair is calculated, and
in the step [3], one or more primer candidate sequence pairs for which the local alignment score being equal to or less than the predetermined threshold value is calculated are selected, and a primer candidate sequence pair having a smallest value of the maximum value of the local alignment score is further detected from all the selected pairs, and is adopted and determined as the forward primer sequence and the reverse primer sequence for amplifying the region including the predetermined target site, and (II) in the case where one or more primer sequences of the different target site have already been determined,
in the step [2], all pairs are selected from the one or more primer candidate sequence pairs of the predetermined target site, and for each pair, a local alignment score between each of candidate sequences of the selected primer candidate sequence pair and each of the already determined primer sequences of the different target site, and a local alignment score between the sequences of the selected primer candidate sequence pair are calculated, and
in the step [3], for each pair, a maximum value is detected from all the calculated local alignment scores, a primer candidate sequence pair for which a local alignment score having the maximum value being equal to or less than a predetermined threshold value is calculated is selected, and a primer candidate sequence pair having a smallest value of the maximum value of the local alignment score is further detected from all the selected pairs, and is adopted and determined as the forward primer sequence and the reverse primer sequence for amplifying the region including the predetermined target site.

3. The primer design method for amplicon methylation sequence analysis according to claim 1, the design method further comprising:
a base sequence data acquisition step of acquiring base sequence data of the double-stranded genomic DNA;
a target site information acquisition step of acquiring the two or more target sites and position information of the target sites; and
a base conversion step of converting “C” which is methylatable in the double-stranded genomic DNA into “Y” and converting the other “C” into “T” in the base sequence data,
wherein in the complementary strand generation step, a complementary strand is generated for each template strand of the double-stranded genomic DNA after the base conversion,
in the partial sequence cutting step, one target site is selected from the two or more target sites, and from each of the strands, one or more partial sequences having a predetermined length are cut out from a base sequence located on a 5′ terminal side of the “Y” obtained by conversion of the selected target site or “R” complementary to the “Y”, based on the position information of the selected target site,
in the primer candidate sequence selection step, a partial sequence satisfying a predetermined selection condition is selected from the one or more partial sequences cut out from each of the strands, as the primer candidate sequence,
the methylatable “C” is “C” in a CG sequence, and
the predetermined selection condition includes
(1) a Tm value is within a predetermined range,
(2) the number of YG sequences or CR sequences included in the partial sequence is equal to or less than predetermined number, and
(3) an upper limit of the number of binding sites with a sequence outside a related region on the double-stranded genomic DNA after the base conversion is equal to or less than a predetermined number that is equal to or more than 1,
[provided that “C”, “G”, “Y”, and “R” are base codes established by IUPAC, “C” represents cytosine, “G” represents guanine, “Y” represents thymine or cytosine, and “R” represents adenine or guanine].

4. The primer design method according to claim 3,
wherein the methylatable “C” further includes “C” in a CHG sequence, and
the predetermined selection condition further includes
(4) the number of YHG sequences or CDR sequences included in the partial sequence is equal to or less than a predetermined number,
[provided that “C”, “G”, “Y”, “H”, “R”, and “D” are base codes established by IUPAC, “C” represents cytosine, “G” represents guanine, “Y” represents thymine or cytosine, “H” represents adenine, cytosine, or thymine, “D” represents thymine, guanine, or adenine, and “R” represents adenine or guanine].

5. The primer design method according to claim 3,
wherein the methylatable “C” further includes “C” in a CHH sequence, and
the predetermined selection condition further includes
(5) the number of YHH sequences or DDR sequences included in the partial sequence is equal to or less than a predetermined number,
[provided that “Y”, “H”, “R”, and “D” are base codes established by IUPAC, “Y” represents thymine or cytosine, “H” represents adenine, cytosine, or thymine, “D” represents thymine, guanine, or adenine, and “R” represents adenine or guanine].

6. The primer design method according to claim 3,
wherein in the primer candidate sequence selection step, the double-stranded genomic DNA after the base conversion is divided into a first template strand and a second template strand, a complementary strand of the first template strand is a first complementary strand, a complementary strand of the second template strand is a second complementary strand, and
the primer candidate sequence selection step is a step of selecting a partial sequence satisfying a predetermined selection condition as a forward primer candidate sequence of the first template strand from one or more partial sequences cut out from the first template strand, selecting a partial sequence satisfying the predetermined selection condition as a reverse primer candidate sequence of the first template strand from one or more partial sequences cut out from the first complementary strand, selecting a partial sequence satisfying the predetermined selection condition as a forward primer candidate sequence of the second template strand from one or more partial sequences cut out from the second template strand, and selecting a partial sequence satisfying the predetermined selection condition as a reverse primer candidate sequence of the second template strand from one or more partial sequences cut out from the second complementary strand.

7. The primer design method according to claim 3,
wherein the primer sequence determination step is a step of calculating a length of a PCR amplification product predicted to be amplified by PCR for all combinations of the one or more forward primer candidate sequences of the first template strand and the one or more reverse primer candidate sequences of the first template strand selected in the primer candidate sequence selection step, adopting a combination of primer candidate sequences for which the calculated length of the PCR amplification product is within a predetermined range as a forward primer sequence and a reverse primer sequence of the first template strand for amplifying a region including the target site selected in the partial sequence cutting step, calculating a length of a PCR amplification product predicted to be amplified by PCR for all combinations of the one or more forward primer candidate sequences of the second template strand and the one or more reverse primer candidate sequences of the second template strand selected in the primer candidate sequence selection step, and adopting and determining a combination of primer candidate sequences for which the calculated length of the PCR amplification product is within a predetermined range as a forward primer sequence and a reverse primer sequence of the second template strand for amplifying the region including the target site selected in the partial sequence cutting step.

8. The primer design method according to claim 1,
wherein, in advance, a correspondence relationship between at least the number of the target sites, the predetermined threshold value, and a primer design success rate is measured using the primer design method according to claim 1, and the correspondence relationship is stored in a storage unit,
in a case where a user sets at least the primer design success rate desired by the user and the number of the target sites via an input unit and gives an instruction to execute primer design, the predetermined threshold value corresponding to the primer design success rate and the number of the target sites, which are equal to or greater than set values and have a small difference, is read out from the correspondence relationship stored in the storage unit, and
a primer sequence for amplifying a region including the predetermined target site is adopted and determined from the one or more primer candidate sequences based on the read-out predetermined threshold value.

9. A manufacturing method for a primer comprising:
a primer design step; and
a synthesis step of synthesizing a primer based on a primer sequence designed in the primer design step,
wherein the primer design step is performed by the primer design method according to claim 1.

10. A primer design device for amplicon methylation sequence analysis, which is a device for designing a primer for amplicon methylation sequence analysis, the device utilizing a bisulfite reaction or an enzyme reaction and a multiplex PCR for measuring a methylation degree of at least one double-stranded DNA and being used for simultaneously amplifying a plurality of regions each including two or more target sites where the methylation degree is measured, the design device comprising:
a complementary strand generation unit that generates a complementary strand with respect to a template strand of the DNA;
a partial sequence cutting unit that selects one target site from the two or more target sites and, from each of the strands, cuts out one or more partial sequences having a predetermined length from a base sequence located on a 5′ terminal side of the selected target site;
a primer candidate sequence selection unit that selects the one or more cut-out partial sequences as one or more primer candidate sequences;
a primer sequence determination unit that adopts and determines a forward primer sequence and a reverse primer sequence for amplifying a region including the selected predetermined target site from the one or more primer candidate sequences; and
a control unit that performs control configured to repeat each processing in the partial sequence cutting unit, the primer candidate sequence selection unit, and the primer sequence determination unit until all of the two or more target sites are selected in the partial sequence cutting unit,
wherein (I) in a case where one or more primer sequences of a different target site have not yet been determined, the primer sequence determination unit performs the following steps,
[1] selecting one or more primer candidate sequence pairs related to the predetermined target site from the one or more primer candidate sequences,
[2] selecting one primer candidate sequence pair from the one or more primer candidate sequence pairs of the predetermined target site, and calculating a local alignment score between sequences of the selected primer candidate sequence pair, and
[3] adopting and determining the primer candidate sequence pair for which the local alignment score being equal to or less than a predetermined threshold value is calculated as the forward primer sequence and the reverse primer sequence for amplifying the region including the predetermined target site, (II) in a case where one or more primer sequences of the different target site have already been determined, the primer sequence determination unit performs the following steps,
[1] selecting one or more primer candidate sequence pairs related to the predetermined target site from the one or more primer candidate sequences,
[2] selecting one primer candidate sequence pair from the one or more primer candidate sequence pairs of the predetermined target site, and for each pair, calculating a local alignment score between each of candidate sequences of the selected primer candidate sequence pair and each of the already determined primer sequences of the different target site, and a local alignment score between the sequences of the selected primer candidate sequence pair, and
[3] detecting a maximum value from all the calculated local alignment scores, and adopting and determining a primer candidate sequence pair for which a local alignment score having the maximum value being equal to or less than a predetermined threshold value is calculated, as the forward primer sequence and the reverse primer sequence for amplifying the region including the predetermined target site,
in the step [3] of the (I) and the (II), in a case where the primer candidate sequence pair is not adopted as the forward primer sequence and the reverse primer sequence for amplifying the region including the predetermined target site, one different pair is selected from the one or more primer candidate sequence pairs selected in the step [1] of the (I) and the (II), and the steps [2] and [3] are repeated until at least one primer candidate sequence pair is adopted,
in a case where <1> a complementary base pair is set to “X” per pair, <2> a non-complementary base pair is set to “Y” per pair, and <3> a case where there is insertion or deletion is set to “Z” per one insertion or deletion between the primer candidate sequences, the local alignment score is calculated using “X” of 1, “Y” of −4 to −2, and “Z” of −6 to −3, and
the predetermined threshold value is 1 to 4.

11. The primer design device for amplicon methylation sequence analysis according to claim 10,
wherein in the primer sequence determination unit, (I) in the case where one or more primer sequences of a different target site have not yet been determined,
in the step [2], all pairs are selected from the one or more primer candidate sequence pairs of the predetermined target site, and for each pair, a local alignment score between sequences of the selected primer candidate sequence pair is calculated, and
in the step [3], one or more primer candidate sequence pairs for which the local alignment score being equal to or less than a predetermined threshold value is calculated are selected, and a primer candidate sequence pair having a smallest value of the maximum value of the local alignment score is further detected from all the selected pairs, and is adopted and determined as the forward primer sequence and the reverse primer sequence for amplifying a region including the predetermined target site, and
(II) in the case where one or more primer sequences of the different target site have already been determined,
in the step [2], all pairs are selected from the one or more primer candidate sequence pairs of the predetermined target site, and for each pair, a local alignment score between each of candidate sequences of the selected primer candidate sequence pair and each of the already determined primer sequences of the different target site, and a local alignment score between the sequences of the selected primer candidate sequence pair are calculated, and
in the step [3], for each pair, a maximum value is detected from all the calculated local alignment scores, a primer candidate sequence pair for which a local alignment score having the maximum value being equal to or less than a predetermined threshold value is calculated is selected, and a primer candidate sequence pair having a smallest value of the maximum value of the local alignment score is further detected from all the selected pairs, and is adopted and determined as the forward primer sequence and the reverse primer sequence for amplifying the region including the predetermined target site.

12. The primer design device for amplicon methylation sequence analysis according to claim 10, the design device further comprising:
a base sequence data acquisition unit that acquires base sequence data of the double-stranded genomic DNA;
a target site information acquisition unit that acquires the two or more target sites and position information of the target sites; and
a base conversion unit that converts “C” which is methylatable in the double-stranded genomic DNA into “Y” and converts the other “C” into “T” in the base sequence data,
wherein in the complementary strand generation unit, a complementary strand is generated for each template strand of the double-stranded genomic DNA after the base conversion,
in the partial sequence cutting unit, one target site is selected from the two or more target sites, and from each of the strands, one or more partial sequences having a predetermined length are cut out from a base sequence located on a 5′ terminal side of the “Y” obtained by conversion of the selected target site or “R” complementary to the “Y”, based on the position information of the selected target site,
in the primer candidate sequence selection unit, a partial sequence satisfying a predetermined selection condition is selected from the one or more partial sequences cut out from each of the strands, as the primer candidate sequence,
the methylatable “C” is “C” in a CG sequence, and
the predetermined selection condition includes
(1) Tm is within a predetermined range,
(2) the number of YG sequences or CR sequences included in the partial sequence is equal to or less than predetermined number, and
(3) an upper limit of the number of binding sites with a sequence outside a related region on the double-stranded genomic DNA after the base conversion is equal to or less than a predetermined number that is equal to or more than 1,
[provided that “C”, “G”, “Y”, and “R” are base codes established by IUPAC, “C” represents cytosine, “G” represents guanine, “Y” represents thymine or cytosine, and “R” represents adenine or guanine].

13. The primer design device according to claim 12,
wherein the methylatable “C” further includes “C” in a CHG sequence, and
the predetermined selection condition further includes
(4) the number of YHG sequences or CDR sequences included in the partial sequence is equal to or less than a predetermined number,
[provided that “C”, “G”, “Y”, “H”, “R”, and “D” are base codes established by IUPAC, “C” represents cytosine, “G” represents guanine, “Y” represents thymine or cytosine, “H” represents adenine, cytosine, or thymine, “D” represents thymine, guanine, or adenine, and “R” represents adenine or guanine].

14. The primer design device according to claim 12,
wherein the methylatable “C” further includes “C” in a CHH sequence, and
the predetermined selection condition further includes
(5) the number of YHH sequences or DDR sequences included in the partial sequence is equal to or less than a predetermined number,
[provided that “Y”, “H”, “R”, and “D” are base codes established by IUPAC, “Y” represents thymine or cytosine, “H” represents adenine, cytosine, or thymine, “D” represents thymine, guanine, or adenine, and “R” represents adenine or guanine].

15. The primer design device according to claim 12,
wherein in the primer candidate sequence selection unit, the double-stranded genomic DNA after the base conversion is divided into a first template strand and a second template strand, a complementary strand of the first template strand is a first complementary strand, a complementary strand of the second template strand is a second complementary strand, and
the primer candidate sequence selection unit is a unit that selects a partial sequence satisfying a predetermined selection condition as a forward primer candidate sequence of the first template strand from one or more partial sequences cut out from the first template strand, selects a partial sequence satisfying the predetermined selection condition as a reverse primer candidate sequence of the first template strand from one or more partial sequences cut out from the first complementary strand, selects a partial sequence satisfying the predetermined selection condition as a forward primer candidate sequence of the second template strand from one or more partial sequences cut out from the second template strand, and selects a partial sequence satisfying the predetermined selection condition as a reverse primer candidate sequence of the second template strand from one or more partial sequences cut out from the second complementary strand.

16. The primer design device according to claim 15,
wherein the primer sequence determination unit is a unit that calculates a length of a PCR amplification product predicted to be amplified by PCR for all combinations of the one or more forward primer candidate sequences of the first template strand and the one or more reverse primer candidate sequences of the first template strand selected in the primer candidate sequence selection unit, adopts a combination of primer candidate sequences for which the calculated length of the PCR amplification product is within a predetermined range as a forward primer sequence and a reverse primer sequence of the first template strand for amplifying a region including the target site selected in the partial sequence cutting unit, calculates a length of a PCR amplification product predicted to be amplified by PCR for all combinations of the one or more forward primer candidate sequences of the second template strand and the one or more reverse primer candidate sequences of the second template strand selected in the primer candidate sequence selection unit, and adopts and determines a combination of primer candidate sequences for which the calculated length of the PCR amplification product is within a predetermined range as a forward primer sequence and a reverse primer sequence of the second template strand for amplifying the region including a target site selected in the partial sequence cutting unit.

17. The primer design device for amplicon methylation sequence analysis according to claim 10, further comprising:
a storage unit that measures a correspondence relationship between at least the number of the target sites, the predetermined threshold value, and a primer design success rate in advance using the primer design device according to claim 10, and stores the correspondence relationship; and
an input unit through which a user inputs an instruction,
wherein, in the primer sequence determination unit, in a case where the user sets at least the primer design success rate desired by the user and the number of the target sites via the input unit and gives an instruction to execute primer design, the predetermined threshold value corresponding to the primer design success rate and the number of the target sites, which are equal to or greater than set values and have a small difference, is read out from the correspondence relationship stored in the storage unit, and a primer sequence for amplifying a region including the predetermined target site is adopted and determined from the one or more primer candidate sequences based on the read-out predetermined threshold value.

18. The primer design device according to claim 12, the design device further comprising:
a communication interface,
wherein the design device is capable of being connected to a server via an external communication network by the communication interface and is capable of operating at least one unit selected from the group consisting of the base sequence data acquisition unit, the target site information acquisition unit, the base conversion unit, the complementary strand generation unit, the partial sequence cutting unit, the primer candidate sequence selection unit, and the primer sequence determination unit by programs in the server.

19. A program for designing a primer, the program being configured to execute the primer design method according to claim 1 on a computer.

20. A computer-readable recording medium,
wherein the program for designing a primer according to claim 19 is recorded.


SEQUENCE LISTING (1010 sequences)

SEQ ID NO: 1 - DNA, 25 bp, synthetic construct
  source 1..25: mol_type = other DNA
ttttttttatagtttttggtagtga

SEQ ID NO: 2 - DNA, 20 bp, synthetic construct
  source 1..20: mol_type = other DNA
tgtagagaggaggaggtgag

SEQ ID NO: 3 - DNA, 25 bp, synthetic construct
  source 1..25: mol_type = other DNA
ggtttgaaatgttatttttaataag

SEQ ID NO: 4 - DNA, 25 bp, synthetic construct
  source 1..25: mol_type = other DNA
tagttgttgatttgataggaggtag

SEQ ID NO: 5 - DNA, 23 bp, synthetic construct
  source 1..23: mol_type = other DNA
ggttaagaaggaggatatagaga

SEQ ID NO: 6 - DNA, 20 bp, synthetic construct
  source 1..20: mol_type = other DNA
ttgttttttgttgtgtggaa

SEQ ID NO: 7 - DNA, 24 bp, synthetic construct
  source 1..24: mol_type = other DNA
ggttattttttaaatggatagtga

SEQ ID NO: 8 - DNA, 20 bp, synthetic construct
  source 1..20: mol_type = other DNA
tttttaaggtgttaggggaa

SEQ ID NO: 9 - DNA, 20 bp, synthetic construct
  source 1..20: mol_type = other DNA
ggagttttttatgaagggag

SEQ ID NO: 10 - DNA, 20 bp, synthetic construct
  source 1..20: mol_type = other DNA
gtgtggaaggaaaaaaaaag

SEQ ID NO: 11 - DNA, 26 bp, synthetic construct
  source 1..26: mol_type = other DNA
ttgtttgtttgttatttttttattag

SEQ ID NO: 12 - DNA, 23 bp, synthetic construct
  source 1..23: mol_type = other DNA
ggggtagagtataggttagttga

SEQ ID NO: 13 - DNA, 20 bp, synthetic construct
  source 1..20: mol_type = other DNA
tggtaggtgttttgggttga

SEQ ID NO: 14 - DNA, 23 bp, synthetic construct
  source 1..23: mol_type = other DNA
gtttgtatggattattaggttga

SEQ ID NO: 15 - DNA, 27 bp, synthetic construct
  source 1..27: mol_type = other DNA
ggatttttttttagttttttaaataag

SEQ ID NO: 16 - DNA, 23 bp, synthetic construct
  source 1..23: mol_type = other DNA
ttattatttattttttgggtgaa

SEQ ID NO: 17 - DNA, 24 bp, synthetic construct
  source 1..24: mol_type = other DNA
ggaggttagtttggttataggtag

SEQ ID NO: 18 - DNA, 22 bp, synthetic construct
  source 1..22: mol_type = other DNA
ttttggttttaaaagagagaaa

SEQ ID NO: 19 - DNA, 28 bp, synthetic construct
  source 1..28: mol_type = other DNA
ttagggttatattttaatatgtagaaaa

SEQ ID NO: 20 - DNA, 25 bp, synthetic construct
  source 1..25: mol_type = other DNA
ttatatttttaagtggtaagaggag

SEQ ID NO: 21 - DNA, 20 bp, synthetic construct
  source 1..20: mol_type = other DNA
ggtttttgttgtgttgggag

SEQ ID NO: 22 - DNA, 32 bp, synthetic construct
  source 1..32: mol_type = other DNA
tttttatatttatatataagtgttagaaatga

SEQ ID NO: 23 - DNA, 27 bp, synthetic construct
  source 1..27: mol_type = other DNA
taagggtttattaatttttttaataag

SEQ ID NO: 24 - DNA, 20 bp, synthetic construct
  source 1..20: mol_type = other DNA
ttatggttggggaaattgag

SEQ ID NO: 25 - DNA, 20 bp, synthetic construct
  source 1..20: mol_type = other DNA
agattggggttaggatgaga

SEQ ID NO: 26 - DNA, 27 bp, synthetic construct
  source 1..27: mol_type = other DNA
gtttttttggtaatataaggtatagag

SEQ ID NO: 27 - DNA, 21 bp, synthetic construct
  source 1..21: mol_type = other DNA
ttttgtagttttgagaggtga

SEQ ID NO: 28 - DNA, 34 bp, synthetic construct
  source 1..34: mol_type = other DNA
aaattattagtaaattaaaaatattaaaataaaa

SEQ ID NO: 29 - DNA, 20 bp, synthetic construct
  source 1..20: mol_type = other DNA
ttgagaagttttgaagggaa

SEQ ID NO: 30 - DNA, 25 bp, synthetic construct
  source 1..25: mol_type = other DNA
ttgtttttaaaaaaattaaaaagag

SEQ ID NO: 31 - DNA, 24 bp, synthetic construct
  source 1..24: mol_type = other DNA
tgagatgattaaatgaagattaaa

SEQ ID NO: 32 - DNA, 24 bp, synthetic construct
  source 1..24: mol_type = other DNA
gtttgttgttttgtagaaaaataa

SEQ ID NO: 33 - DNA, 28 bp, synthetic construct
  source 1..28: mol_type = other DNA
tagtttgagaaataggtaataaaaatag

SEQ ID NO: 34 - DNA, 28 bp, synthetic construct
  source 1..28: mol_type = other DNA
ttatttttgttaatttaagtaaggtaga

SEQ ID NO: 35 - DNA, 23 bp, synthetic construct
  source 1..23: mol_type = other DNA
gggttttttattttggaattaag

SEQ ID NO: 36 - DNA, 21 bp, synthetic construct
  source 1..21: mol_type = other DNA
ttgtgggtgtaaataaattga

SEQ ID NO: 37 - DNA, 20 bp, synthetic construct
  source 1..20: mol_type = other DNA
ttggttgttttggatgatga

SEQ ID NO: 38 - DNA, 24 bp, synthetic construct
  source 1..24: mol_type = other DNA
gtttaaggtttataagaagaggaa

SEQ ID NO: 39 - DNA, 20 bp, synthetic construct
  source 1..20: mol_type = other DNA
ttggggttatagttggagag

SEQ ID NO: 40 - DNA, 24 bp, synthetic construct
  source 1..24: mol_type = other DNA
tgttttagaaagaaaagaaaaaga

SEQ ID NO: 41 - DNA, 24 bp, synthetic construct
  source 1..24: mol_type = other DNA
ggaaatattgatttttgatagaag

SEQ ID NO: 42 - DNA, 20 bp, synthetic construct
  source 1..20: mol_type = other DNA
taggatggtagggttgggag

SEQ ID NO: 43 - DNA, 27 bp, synthetic construct
  source 1..27: mol_type = other DNA
aaaattaatgaattgttaaagtttaag

SEQ ID NO: 44 - DNA, 23 bp, synthetic construct
  source 1..23: mol_type = other DNA
ttttttgagatttgttaagaaag

SEQ ID NO: 45 - DNA, 20 bp, synthetic construct
  source 1..20: mol_type = other DNA
ttgattttgttttggagtga

SEQ ID NO: 46 - DNA, 27 bp, synthetic construct
  source 1..27: mol_type = other DNA
aattttgaattttatttgtaaagtaga

SEQ ID NO: 47 - DNA, 23 bp, synthetic construct
  source 1..23: mol_type = other DNA
ttgtttatttttagtggattgag

SEQ ID NO: 48 - DNA, 21 bp, synthetic construct
  source 1..21: mol_type = other DNA
atgttggtagagtggggttga

SEQ ID NO: 49 - DNA, 24 bp, synthetic construct
  source 1..24: mol_type = other DNA
gaggatttgtaattggtatagaag

SEQ ID NO: 50 - DNA, 20 bp, synthetic construct
  source 1..20: mol_type = other DNA
gtgggtgatttgatgggtga

SEQ ID NO: 51 - DNA, 20 bp, synthetic construct
  source 1..20: mol_type = other DNA
atgttttgaaggagggttga

SEQ ID NO: 52 - DNA, 20 bp, synthetic construct
  source 1..20: mol_type = other DNA
ttggtgattgttgaaaatga

SEQ ID NO: 53 - DNA, 30 bp, synthetic construct
  source 1..30: mol_type = other DNA
ataaattaaagagttaagtattagaaatga

SEQ ID NO: 54 - DNA, 23 bp, synthetic construct
  source 1..23: mol_type = other DNA
ttttttttgtttaataaaggtga

SEQ ID NO: 55 - DNA, 30 bp, synthetic construct
  source 1..30: mol_type = other DNA
tgtatgttttagtattttgttttaagttag

SEQ ID NO: 56 - DNA, 25 bp, synthetic construct
  source 1..25: mol_type = other DNA
tgggtgttaagttagtttaataaag

SEQ ID NO: 57 - DNA, 20 bp, synthetic construct
  source 1..20: mol_type = other DNA
ttatagggaagggtagggag

SEQ ID NO: 58 - DNA, 26 bp, synthetic construct
  source 1..26: mol_type = other DNA
tgaattttagtattggtgtatatgag

SEQ ID NO: 59 - DNA, 30 bp, synthetic construct
  source 1..30: mol_type = other DNA
agagttaagttagatgtgttataattagag

SEQ ID NO: 60 - DNA, 25 bp, synthetic construct
  source 1..25: mol_type = other DNA
atttaggaatgtagattaaagtgaa

SEQ ID NO: 61 - DNA, 20 bp, synthetic construct
  source 1..20: mol_type = other DNA
gtgtgtgttgtggtgaggag

SEQ ID NO: 62 - DNA, 27 bp, synthetic construct
  source 1..27: mol_type = other DNA
gagttagtgtttttattataggagaga

SEQ ID NO: 63 - DNA, 20 bp, synthetic construct
  source 1..20: mol_type = other DNA
aatcccacttacaaaaaaca

SEQ ID NO: 64 - DNA, 24 bp, synthetic construct
  source 1..24: mol_type = other DNA
ttaatatctatcctaattccaacc

SEQ ID NO: 65 - DNA, 22 bp, synthetic construct
  source 1..22: mol_type = other DNA
tcacaatcaaaacatttctaaa

SEQ ID NO: 66 - DNA, 23 bp, synthetic construct
  source 1..23: mol_type = other DNA
aaaaaccaatactaacctaatcc

SEQ ID NO: 67 - DNA, 22 bp, synthetic construct
  source 1..22: mol_type = other DNA
caaatataaaaaataatcccca

SEQ ID NO: 68 - DNA, 20 bp, synthetic construct
  source 1..20: mol_type = other DNA
caaacaaaatttacaaccca

SEQ ID NO: 69 - DNA, 20 bp, synthetic construct
  source 1..20: mol_type = other DNA
cctcacccacttctcctaca

SEQ ID NO: 70 - DNA, 21 bp, synthetic construct
  source 1..21: mol_type = other DNA
ctcaaaatcccaacctcaaaa

SEQ ID NO: 71 - DNA, 20 bp, synthetic construct
  source 1..20: mol_type = other DNA
tccccactaactccccaaaa

SEQ ID NO: 72 - DNA, 20 bp, synthetic construct
  source 1..20: mol_type = other DNA
acccaaaatctacaaaaccc

SEQ ID NO: 73 - DNA, 29 bp, synthetic construct
  source 1..29: mol_type = other DNA
ttaccaatattctcattaatttaatataa

SEQ ID NO: 74 - DNA, 21 bp, synthetic construct
  source 1..21: mol_type = other DNA
tccccactaatacttccttac

SEQ ID NO: 75 - DNA, 22 bp, synthetic construct
  source 1..22: mol_type = other DNA
tctaatcccaattcaattaaaa

SEQ ID NO: 76 - DNA, 25 bp, synthetic construct
  source 1..25: mol_type = other DNA
aaaaataaaccttacaaactacaca

SEQ ID NO: 77 - DNA, 24 bp, synthetic construct
  source 1..24: mol_type = other DNA
tccaaatctcctactaataaaaac

SEQ ID NO: 78 - DNA, 24 bp, synthetic construct
  source 1..24: mol_type = other DNA
ccactacacaaataaaaaaataaa

SEQ ID NO: 79 - DNA, 27 bp, synthetic construct
  source 1..27: mol_type = other DNA
aaacataaaaaatctaatctattcaaa

SEQ ID NO: 80 - DNA, 25 bp, synthetic construct
  source 1..25: mol_type = other DNA
attcactactatctaataaaaccca

SEQ ID NO: 81 - DNA, 20 bp, synthetic construct
  source 1..20: mol_type = other DNA
tttttttctctttttcccaa

SEQ ID NO: 82 - DNA, 24 bp, synthetic construct
  source 1..24: mol_type = other DNA
aattacccaaacaatattaatacc

SEQ ID NO: 83 - DNA, 21 bp, synthetic construct
  source 1..21: mol_type = other DNA
ccaataacattaaaacaacca

SEQ ID NO: 84 - DNA, 25 bp, synthetic construct
  source 1..25: mol_type = other DNA
tctcctaacctcaatatataaaaca

SEQ ID NO: 85 - DNA, 20 bp, synthetic construct
  source 1..20: mol_type = other DNA
aaactctaccccaccaaaca

SEQ ID NO: 86 - DNA, 23 bp, synthetic construct
  source 1..23: mol_type = other DNA
ctccttcttccatactaataacc

SEQ ID NO: 87 - DNA, 22 bp, synthetic construct
  source 1..22: mol_type = other DNA
cccacaaaaaaccctaaaaaac

SEQ ID NO: 88 - DNA, 35 bp, synthetic construct
  source 1..35: mol_type = other DNA
aaattattcaaaaaataattataataataatatac

SEQ ID NO: 89 - DNA, 21 bp, synthetic construct
  source 1..21: mol_type = other DNA
aaaaaaatccctcaatacaac

SEQ ID NO: 90 - DNA, 23 bp, synthetic construct
  source 1..23: mol_type = other DNA
ctaaaatttccaattttaaatcc

SEQ ID NO: 91 - DNA, 30 bp, synthetic construct
  source 1..30: mol_type = other DNA
ttatttcctaaaacttataaatttataaaa

SEQ ID NO: 92 - DNA, 28 bp, synthetic construct
  source 1..28: mol_type = other DNA
aattatatttcaaaccttatcttaaaac

SEQ ID NO: 93 - DNA, 24 bp, synthetic construct
  source 1..24: mol_type = other DNA
cttacacacctaaactaattacca

SEQ ID NO: 94 - DNA, 24 bp, synthetic construct
  source 1..24: mol_type = other DNA
cctactaatcttactcaacaaaca

SEQ ID NO: 95 - DNA, 32 bp, synthetic construct
  source 1..32: mol_type = other DNA
attaattctaaaataatactaaaaacttttac

SEQ ID NO: 96 - DNA, 23 bp, synthetic construct
  source 1..23: mol_type = other DNA
cctttcatttaaaatatttccaa

SEQ ID NO: 97 - DNA, 20 bp, synthetic construct
  source 1..20: mol_type = other DNA
ccctcaacctcctaaataca

SEQ ID NO: 98 - DNA, 22 bp, synthetic construct
  source 1..22: mol_type = other DNA
aaaaataccatttacctaacca

SEQ ID NO: 99 - DNA, 23 bp, synthetic construct
  source 1..23: mol_type = other DNA
aacatctcattttcaaactacac

SEQ ID NO: 100 - DNA, 22 bp, synthetic construct
  source 1..22: mol_type = other DNA
cataccattaactaaaaaaccc

SEQ ID NO: 101 - DNA, 22 bp, synthetic construct
  source 1..22: mol_type = other DNA
tccataacaatcactcactaaa

SEQ ID NO: 102 - DNA, 28 bp, synthetic construct
  source 1..28: mol_type = other DNA
aacttaaacccaaaactttaaaactaca

SEQ ID NO: 103 - DNA, 20 bp, synthetic construct
  source 1..20: mol_type = other DNA
ccttccaccaaatattcaaa

SEQ ID NO: 104 - DNA, 20 bp, synthetic construct
  source 1..20: mol_type = other DNA
tccttcacataccaaaaaaa

SEQ ID NO: 105 - DNA, 22 bp, synthetic construct
  source 1..22: mol_type = other DNA
tccttaaaaaaaaacctacaaa

SEQ ID NO: 106 - DNA, 25 bp, synthetic construct
  source 1..25: mol_type = other DNA
ccatttaatataaatcacataacca

SEQ ID NO: 107 - DNA, 20 bp, synthetic construct
  source 1..20: mol_type = other DNA
actaaccaccctctcctaca

SEQ ID NO: 108 - DNA, 23 bp, synthetic construct
  source 1..23: mol_type = other DNA
aacaaaacacttaatctcctaca

SEQ ID NO: 109 - DNA, 25 bp, synthetic construct
  source 1..25: mol_type = other DNA
ttcttaaaataaaacactacacaca

SEQ ID NO: 110 - DNA, 22 bp, synthetic construct
  source 1..22: mol_type = other DNA
cacaacaaactaattaaccaaa

SEQ ID NO: 111 - DNA, 24 bp, synthetic construct
  source 1..24: mol_type = other DNA
acaattctctttcctttaaaataa

SEQ ID NO: 112 - DNA, 25 bp, synthetic construct
  source 1..25: mol_type = other DNA
aaataccctcctattattttaaaac

SEQ ID NO: 113 - DNA, 34 bp, synthetic construct
  source 1..34: mol_type = other DNA
aataatcaaaaataatttattaaatattaaatac

SEQ ID NO: 114 - DNA, 25 bp, synthetic construct
  source 1..25: mol_type = other DNA
cacaaaaacatctctctatatacaa

SEQ ID NO: 115 - DNA, 22 bp, synthetic construct
  source 1..22: mol_type = other DNA
catcacacccttactaattacc

SEQ ID NO: 116 - DNA, 22 bp, synthetic construct
  source 1..22: mol_type = other DNA
ctttccaacctaaaaaataaca

SEQ ID NO: 117 - DNA, 22 bp, synthetic construct
  source 1..22: mol_type = other DNA
atctcaaaaaataaatttccaa

SEQ ID NO: 118 - DNA, 22 bp, synthetic construct
  source 1..22: mol_type = other DNA
caaaatataaaaatcaaatccc

SEQ ID NO: 119 - DNA, 25 bp, synthetic construct
  source 1..25: mol_type = other DNA
tcaaataacacctaaataataatcc

SEQ ID NO: 120 - DNA, 20 bp, synthetic construct
  source 1..20: mol_type = other DNA
actccaaacttccccaacaa

SEQ ID NO: 121 - DNA, 27 bp, synthetic construct
  source 1..27: mol_type = other DNA
acaactcaaaactctaaaataataaac

SEQ ID NO: 122 - DNA, 23 bp, synthetic construct
  source 1..23: mol_type = other DNA
tttatacaatcacaaacatacca

SEQ ID NO: 123 - DNA, 20 bp, synthetic construct
  source 1..20: mol_type = other DNA
cttaacctaaactccccaaa

SEQ ID NO: 124 - DNA, 24 bp, synthetic construct
  source 1..24: mol_type = other DNA
atttaaacctcataaccctataaa

SEQ ID NO: 125 - DNA, 25 bp, synthetic construct
  source 1..25: mol_type = other DNA
gtttgaaatgttatttttaataagg

SEQ ID NO: 126 - DNA, 22 bp, synthetic construct
  source 1..22: mol_type = other DNA
tgtatagatggggaaatagagg

SEQ ID NO: 127 - DNA, 20 bp, synthetic construct
  source 1..20: mol_type = other DNA
ttggggttaggatgagagaa

SEQ ID NO: 128 - DNA, 20 bp, synthetic construct
  source 1..20: mol_type = other DNA
ataaaggtgaagggtgtggg

SEQ ID NO: 129 - DNA, 32 bp, synthetic construct
  source 1..32: mol_type = other DNA
tctaaaactattaatatctctaaaaaactaaa

SEQ ID NO: 130 - DNA, 27 bp, synthetic construct
  source 1..27: mol_type = other DNA
aacaaaaaaaacactaataaaactaaa

SEQ ID NO: 131 - DNA, 25 bp, synthetic construct
  source 1..25: mol_type = other DNA
ccacaaaaaaccctaaaaaactaaa

SEQ ID NO: 132 - DNA, 22 bp, synthetic construct
  source 1..22: mol_type = other DNA
tttccaacctaaaaaataacaa

SEQ ID NO: 133 - DNA, 24 bp, synthetic construct
  source 1..24: mol_type = other DNA
tggtagtgattagtttattttttg

SEQ ID NO: 134 - DNA, 20 bp, synthetic construct
  source 1..20: mol_type = other DNA
tgtagagaggaggaggtgag

SEQ ID NO: 135 - DNA, 20 bp, synthetic construct
  source 1..20: mol_type = other DNA
tttttgggtttgaaatgtta

SEQ ID NO: 136 - DNA, 22 bp, synthetic construct
  source 1..22: mol_type = other DNA
gggttgaggattagtattgatt

SEQ ID NO: 137 - DNA, 20 bp, synthetic construct
  source 1..20: mol_type = other DNA
ggttgatgaggtataggtga

SEQ ID NO: 138 - DNA, 22 bp, synthetic construct
  source 1..22: mol_type = other DNA
aagaaggaggatatagagaagg

SEQ ID NO: 139 - DNA, 20 bp, synthetic construct
  source 1..20: mol_type = other DNA
ttgttttttgttgtgtggaa

SEQ ID NO: 140 - DNA, 24 bp, synthetic construct
  source 1..24: mol_type = other DNA
tttttaggttattttttaaatgga

SEQ ID NO: 141 - DNA, 22 bp, synthetic construct
  source 1..22: mol_type = other DNA
tgtgattttagtatttgggaag

SEQ ID NO: 142 - DNA, 20 bp, synthetic construct
  source 1..20: mol_type = other DNA
tttatgaagggagttgtgga

SEQ ID NO: 143 - DNA, 21 bp, synthetic construct
  source 1..21: mol_type = other DNA
ttaggttggtggtttttattt

SEQ ID NO: 144 - DNA, 26 bp, synthetic construct
  source 1..26: mol_type = other DNA
tttagatataaatttttttgtatgga

SEQ ID NO: 145 - DNA, 23 bp, synthetic construct
  source 1..23: mol_type = other DNA
ttggggtagagtataggttagtt

SEQ ID NO: 146 - DNA, 20 bp, synthetic construct
  source 1..20: mol_type = other DNA
agaggttgttgttgtgtttg

SEQ ID NO: 147 - DNA, 20 bp, synthetic construct
  source 1..20: mol_type = other DNA
tggtaggtgttttgggttga

SEQ ID NO: 148 - DNA, 20 bp, synthetic construct
  source 1..20: mol_type = other DNA
tgttgttttgtttgtatgga

SEQ ID NO: 149 - DNA, 20 bp, synthetic construct
  source 1..20: mol_type = other DNA
gttttttgttggtgggaata

SEQ ID NO: 150 - DNA, 23 bp, synthetic construct
  source 1..23: mol_type = other DNA
tgataaagattttgtaggggtta

SEQ ID NO: 151 - DNA, 20 bp, synthetic construct
  source 1..20: mol_type = other DNA
ggaagggtattggtgggatt

SEQ ID NO: 152 - DNA, 20 bp, synthetic construct
  source 1..20: mol_type = other DNA
aagtgggtttgggaagtatg

SEQ ID NO: 153 - DNA, 23 bp, synthetic construct
  source 1..23: mol_type = other DNA
tttagggagatatattttggttt

SEQ ID NO: 154 - DNA, 26 bp, synthetic construct
  source 1..26: mol_type = other DNA
tttaatgaatggatataagtgattta

SEQ ID NO: 155 - DNA, 20 bp, synthetic construct
  source 1..20: mol_type = other DNA
ttgggtgtgtaagaattttt

SEQ ID NO: 156 - DNA, 23 bp, synthetic construct
  source 1..23: mol_type = other DNA
agttggtttttgaatttattttt

SEQ ID NO: 157 - DNA, 32 bp, synthetic construct
  source 1..32: mol_type = other DNA
ttttttatatttatatataagtgttagaaatg

SEQ ID NO: 158 - DNA, 24 bp, synthetic construct
  source 1..24: mol_type = other DNA
gaggaagtaagggtttattaattt

SEQ ID NO: 159 - DNA, 21 bp, synthetic construct
  source 1..21: mol_type = other DNA
attttatggttggggaaattg

SEQ ID NO: 160 - DNA, 20 bp, synthetic construct
  source 1..20: mol_type = other DNA
ggggttaggatgagagaatg

SEQ ID NO: 161 - DNA, 30 bp, synthetic construct
  source 1..30: mol_type = other DNA
ttggtaatataaggtatagagtataggtta

SEQ ID NO: 162 - DNA, 20 bp, synthetic construct
  source 1..20: mol_type = other DNA
taattgggtagggtgggtta

SEQ ID NO: 163 - DNA, 20 bp, synthetic construct
  source 1..20: mol_type = other DNA
gttttgaagggaagatagga

SEQ ID NO: 164 - DNA, 26 bp, synthetic construct
  source 1..26: mol_type = other DNA
aaaaaaattaaaaagagtaataggaa

SEQ ID NO: 165 - DNA, 21 bp, synthetic construct
  source 1..21: mol_type = other DNA
gttttggggaatgtgttttta

SEQ ID NO: 166 - DNA, 21 bp, synthetic construct
  source 1..21: mol_type = other DNA
gtaattgttggtaggttgttg

SEQ ID NO: 167 - DNA, 28 bp, synthetic construct
  source 1..28: mol_type = other DNA
ttaaattttttttttttagttttaattt

SEQ ID NO: 168 - DNA, 25 bp, synthetic construct
  source 1..25: mol_type = other DNA
tttatgagatgattaaatgaagatt

SEQ ID NO: 169 - DNA, 21 bp, synthetic construct
  source 1..21: mol_type = other DNA
ggttgttgtaattgtttgttg

SEQ ID NO: 170 - DNA, 27 bp, synthetic construct
  source 1..27: mol_type = other DNA
ggtaataaaaatagaatattaggattg

SEQ ID NO: 171 - DNA, 21 bp, synthetic construct
  source 1..21: mol_type = other DNA
gttgtggggttgtgaattttt

SEQ ID NO: 172 - DNA, 25 bp, synthetic construct
  source 1..25: mol_type = other DNA
ggttttagtgattttttttttagtt

SEQ ID NO: 173 - DNA, 24 bp, synthetic construct
  source 1..24: mol_type = other DNA
gggtgtaaataaattgagttgtta

SEQ ID NO: 174 - DNA, 20 bp, synthetic construct
  source 1..20: mol_type = other DNA
gatattggttgttttggatg

SEQ ID NO: 175 - DNA, 21 bp, synthetic construct
  source 1..21: mol_type = other DNA
aagaggaaatgttttgttttg

SEQ ID NO: 176 - DNA, 21 bp, synthetic construct
  source 1..21: mol_type = other DNA
atgtgttttttgttaaatgga

SEQ ID NO: 177 - DNA, 23 bp, synthetic construct
  source 1..23: mol_type = other DNA
gaaaagaaaaagagaaagttttt

SEQ ID NO: 178 - DNA, 30 bp, synthetic construct
  source 1..30: mol_type = other DNA
gatagtaatatttttttttttttagttttt

SEQ ID NO: 179 - DNA, 26 bp, synthetic construct
  source 1..26: mol_type = other DNA
attagttgagttttttttttttttta

SEQ ID NO: 180 - DNA, 21 bp, synthetic construct
  source 1..21: mol_type = other DNA
tgtggaaatattgatttttga

SEQ ID NO: 181 - DNA, 24 bp, synthetic construct
  source 1..24: mol_type = other DNA
ttttatatgttagggaaagttttt

SEQ ID NO: 182 - DNA, 26 bp, synthetic construct
  source 1..26: mol_type = other DNA
ttagtaggaaaattaatgaattgtta

SEQ ID NO: 183 - DNA, 22 bp, synthetic construct
  source 1..22: mol_type = other DNA
ttgaatgttgttatttggtatg

SEQ ID NO: 184 - DNA, 21 bp, synthetic construct
  source 1..21: mol_type = other DNA
ttatttgattttgttttggag

SEQ ID NO: 185 - DNA, 25 bp, synthetic construct
  source 1..25: mol_type = other DNA
gttttttaattttgaattttatttg

SEQ ID NO: 186 - DNA, 26 bp, synthetic construct
  source 1..26: mol_type = other DNA
agttttaatagttttaagtttggatt

SEQ ID NO: 187 - DNA, 23 bp, synthetic construct
  source 1..23: mol_type = other DNA
gtatggtattttttgaagtgaag

SEQ ID NO: 188 - DNA, 21 bp, synthetic construct
  source 1..21: mol_type = other DNA
atgttggtagagtggggttga

SEQ ID NO: 189 - DNA, 23 bp, synthetic construct
  source 1..23: mol_type = other DNA
ggatttgtaattggtatagaagg

SEQ ID NO: 190 - DNA, 20 bp, synthetic construct
  source 1..20: mol_type = other DNA
gaggggatgtttttttgttg

SEQ ID NO: 191 - DNA, 27 bp, synthetic construct
  source 1..27: mol_type = other DNA
aaagtttattatatgtattttttggag

SEQ ID NO: 192 - DNA, 20 bp, synthetic construct
  source 1..20: mol_type = other DNA
ttgtagttggtgattgttga

SEQ ID NO: 193 - DNA, 30 bp, synthetic construct
  source 1..30: mol_type = other DNA
aataaattaaagagttaagtattagaaatg

SEQ ID NO: 194 - DNA, 22 bp, synthetic construct
  source 1..22: mol_type = other DNA
ttttgtttaataaaggtgaagg

SEQ ID NO: 195 - DNA, 21 bp, synthetic construct
  source 1..21: mol_type = other DNA
gggggttttttttggtttatg

SEQ ID NO: 196 - DNA, 21 bp, synthetic construct
  source 1..21: mol_type = other DNA
aggatgagagttttggtattt

SEQ ID NO: 197 - DNA, 23 bp, synthetic construct
  source 1..23: mol_type = other DNA
ttagtaggggttttagatttttt

SEQ ID NO: 198 - DNA, 21 bp, synthetic construct
  source 1..21: mol_type = other DNA
aattaaggttaagggttttga

SEQ ID NO: 199 - DNA, 22 bp, synthetic construct
  source 1..22: mol_type = other DNA
gaaaggagagagaattttgtta

SEQ ID NO: 200 - DNA, 20 bp, synthetic construct
  source 1..20: mol_type = other DNA
tgaatttgttgttgattttg

SEQ ID NO: 201 - DNA, 22 bp, synthetic construct
  source 1..22: mol_type = other DNA
taatcccacttacaaaaaacac

SEQ ID NO: 202 - RNA, 24 nt, synthetic construct
  source 1..24: mol_type = other RNA
ttaatatctatcctaattccaacc

SEQ ID NO: 203 - DNA, 28 bp, synthetic construct
  source 1..28: mol_type = other DNA
tcaaaacatttctaaaactattaatatc

SEQ ID NO: 204 - DNA, 31 bp, synthetic construct
  source 1..31: mol_type = other DNA
tactaatctaacaaaaaacaaaacttaaaca

SEQ ID NO: 205 - DNA, 20 bp, synthetic construct
  source 1..20: mol_type = other DNA
ttaaatctaacacccacacc

SEQ ID NO: 206 - DNA, 22 bp, synthetic construct
  source 1..22: mol_type = other DNA
caaatataaaaaataatcccca

SEQ ID NO: 207 - DNA, 20 bp, synthetic construct
  source 1..20: mol_type = other DNA
caaacaaaatttacaaccca

SEQ ID NO: 208 - DNA, 20 bp, synthetic construct
  source 1..20: mol_type = other DNA
acctcacccacttctcctac

SEQ ID NO: 209 - DNA, 20 bp, synthetic construct
  source 1..20: mol_type = other DNA
cacccaactcatttttttac

SEQ ID NO: 210 - DNA, 20 bp, synthetic construct
  source 1..20: mol_type = other DNA
aatactccccactaactccc

SEQ ID NO: 211 - DNA, 24 bp, synthetic construct
  source 1..24: mol_type = other DNA
caaaaaatccctatatattcctta

SEQ ID NO: 212 - DNA, 20 bp, synthetic construct
  source 1..20: mol_type = other DNA
cctcatactctaaaaacccc

SEQ ID NO: 213 - DNA, 21 bp, synthetic construct
  source 1..21: mol_type = other DNA
ccccactaatacttccttacc

SEQ ID NO: 214 - DNA, 20 bp, synthetic construct
  source 1..20: mol_type = other DNA
catacctcctaacatcccac

SEQ ID NO: 215 - DNA, 21 bp, synthetic construct
  source 1..21: mol_type = other DNA
cctctaatcccaattcaatta

SEQ ID NO: 216 - DNA, 25 bp, synthetic construct
  source 1..25: mol_type = other DNA
aaaaataaaccttacaaactacaca

SEQ ID NO: 217 - DNA, 20 bp, synthetic construct
  source 1..20: mol_type = other DNA
actcccaaacaccccttttt

SEQ ID NO: 218 - DNA, 21 bp, synthetic construct
  source 1..21: mol_type = other DNA
ccatattaccccaactactct

SEQ ID NO: 219 - DNA, 28 bp, synthetic construct
  source 1..28: mol_type = other DNA
aaataaatattattatacccactacaca

SEQ ID NO: 220 - DNA, 20 bp, synthetic construct
  source 1..20: mol_type = other DNA
taacctaaccacaaacaacc

SEQ ID NO: 221 - DNA, 24 bp, synthetic construct
  source 1..24: mol_type = other DNA
acctcaaataacttaaaattcact

SEQ ID NO: 222 - DNA, 24 bp, synthetic construct
  source 1..24: mol_type = other DNA
tcccatttttctaacatatttact

SEQ ID NO: 223 - DNA, 23 bp, synthetic construct
  source 1..23: mol_type = other DNA
tttctcaacttcacacttaattt

SEQ ID NO: 224 - DNA, 20 bp, synthetic construct
  source 1..20: mol_type = other DNA
caaatacacactaatcccca

SEQ ID NO: 225 - DNA, 22 bp, synthetic construct
  source 1..22: mol_type = other DNA
ccaaaatctcctaacctcaata

SEQ ID NO: 226 - DNA, 20 bp, synthetic construct
  source 1..20: mol_type = other DNA
ccctacctaaaacctcaccc

SEQ ID NO: 227 - DNA, 23 bp, synthetic construct
  source 1..23: mol_type = other DNA
tcttccatactaataacctcaca

SEQ ID NO: 228 - DNA, 20 bp, synthetic construct
  source 1..20: mol_type = other DNA
ttcccccacaaaaaacccta

SEQ ID NO: 229 - DNA, 35 bp, synthetic construct; identical to SEQ ID NO: 88
  source 1..35: mol_type = other DNA

SEQ ID NO: 230 - DNA, 26 bp, synthetic construct
  source 1..26: mol_type = other DNA
taactcctaaacttaaataatcctct

SEQ ID NO: 231 - DNA, 25 bp, synthetic construct
  source 1..25: mol_type = other DNA
tctcaacattatttcctaaaactta

SEQ ID NO: 232 - DNA, 28 bp, synthetic construct
  source 1..28: mol_type = other DNA
caaaatataaattatatttcaaacctta

SEQ ID NO: 233 - DNA, 22 bp, synthetic construct
  source 1..22: mol_type = other DNA
aaactcctcttcccaaatatac

SEQ ID NO: 234 - DNA, 20 bp, synthetic construct
  source 1..20: mol_type = other DNA
ttaacaacccaacatttccc

SEQ ID NO: 235 - DNA, 31 bp, synthetic construct
  source 1..31: mol_type = other DNA
actaaaaaataaaaaaaataaataatatttt

SEQ ID NO: 236 - DNA, 22 bp, synthetic construct
  source 1..22: mol_type = other DNA
caccactctccatataacctta

SEQ ID NO: 237 - DNA, 21 bp, synthetic construct
  source 1..21: mol_type = other DNA
caaaatcaaccacaacctact

SEQ ID NO: 238 - DNA, 29 bp, synthetic construct
  source 1..29: mol_type = other DNA
aaaaaaatacaaaactctatattaattct

SEQ ID NO: 239 - DNA, 22 bp, synthetic construct
  source 1..22: mol_type = other DNA
tccaactacttattccctctta

SEQ ID NO: 240 - DNA, 21 bp, synthetic construct
  source 1..21: mol_type = other DNA
caaaaaaaattttccacccta

SEQ ID NO: 241 - DNA, 25 bp, synthetic construct
  source 1..25: mol_type = other DNA
ctaaaaaaaaaaaataccatttacc

SEQ ID NO: 242 - DNA, 22 bp, synthetic construct
  source 1..22: mol_type = other DNA
acactcaaaaaaactaccctta

SEQ ID NO: 243 - DNA, 22 bp, synthetic construct
  source 1..22: mol_type = other DNA
cataccattaactaaaaaaccc

SEQ ID NO: 244 - DNA, 29 bp, synthetic construct
  source 1..29: mol_type = other DNA
aaaaattttcctataactaataacttaca

SEQ ID NO: 245 - DNA, 27 bp, synthetic construct
  source 1..27: mol_type = other DNA
aacttaaacccaaaactttaaaactac

SEQ ID NO: 246 - DNA, 20 bp, synthetic construct
  source 1..20: mol_type = other DNA
caaaacacctcctctccttt

SEQ ID NO: 247 - DNA, 20 bp, synthetic construct
  source 1..20: mol_type = other DNA
tctaaacccctcctcattac

SEQ ID NO: 248 - DNA, 20 bp, synthetic construct
  source 1..20: mol_type = other DNA
tcaaaaatctaccttccacc

SEQ ID NO: 249 - DNA, 21 bp, synthetic construct
  source 1..21: mol_type = other DNA
ctaaccccaaaaacaatacac

SEQ ID NO: 250 - DNA, 28 bp, synthetic construct
  source 1..28: mol_type = other DNA
aacctacaaaaaaatataaactatcttt

SEQ ID NO: 251 - DNA, 27 bp, synthetic construct
  source 1..27: mol_type = other DNA
aaatatattctataattcccacactta

SEQ ID NO: 252 - DNA, 20 bp, synthetic construct
  source 1..20: mol_type = other DNA
aactaaccaccctctcctac

SEQ ID NO: 253 - DNA, 23 bp, synthetic construct
  source 1..23: mol_type = other DNA
aacaaaacacttaatctcctaca

SEQ ID NO: 254 - DNA, 28 bp, synthetic construct
  source 1..28: mol_type = other DNA
cctttttatttaaaaataatattaaaca

SEQ ID NO: 255 - DNA, 30 bp, synthetic construct
  source 1..30: mol_type = other DNA
tcctaataaactaaaaatattaaaattcta

SEQ ID NO: 256 - DNA, 23 bp, synthetic construct
  source 1..23: mol_type = other DNA
aaacacaacaaactaattaacca

SEQ ID NO: 257 - DNA, 28 bp, synthetic construct
  source 1..28: mol_type = other DNA
attctctttcctttaaaataaatatatc

SEQ ID NO: 258 - DNA, 26 bp, synthetic construct
  source 1..26: mol_type = other DNA
catctcttactaaaactaacatcaca

SEQ ID NO: 259 - DNA, 34 bp, synthetic construct
  source 1..34: mol_type = other DNA
ctactaaatattaataatcaaaaataatttatta

SEQ ID NO: 260 - DNA, 23 bp, synthetic construct
  source 1..23: mol_type = other DNA
aacttcacaaaaacatctctcta

SEQ ID NO: 261 - DNA, 21 bp, synthetic construct
  source 1..21: mol_type = other DNA
tcacacccttactaattaccc

SEQ ID NO: 262 - DNA, 21 bp, synthetic construct
  source 1..21: mol_type = other DNA
cctaaaaaaatctttccaacc

SEQ ID NO: 263 - DNA, 22 bp, synthetic construct
  source 1..22: mol_type = other DNA
ccctaaatcaaccaaaatatac

SEQ ID NO: 264 - DNA, 20 bp, synthetic construct
  source 1..20: mol_type = other DNA
cacatattatcacctcccac

SEQ ID NO: 265 - DNA, 24 bp, synthetic construct
  source 1..24: mol_type = other DNA
ttttctcccttataattttaacac

SEQ ID NO: 266 - DNA, 21 bp, synthetic construct
  source 1..21: mol_type = other DNA
cacaaaatccaaaaacacacc

SEQ ID NO: 267 - DNA, 20 bp, synthetic construct
  source 1..20: mol_type = other DNA
cataccaatcatccccatct

SEQ ID NO: 268 - DNA, 22 bp, synthetic construct
  source 1..22: mol_type = other DNA
tcatttaaacctcataacccta

SEQ ID NO: 269 - DNA, 24 bp, synthetic construct
  source 1..24: mol_type = other DNA
tggtagtgattagtttattttttg

SEQ ID NO: 270 - DNA, 20 bp, synthetic construct
  source 1..20: mol_type = other DNA
tgtagagaggaggaggtgag

SEQ ID NO: 271 - DNA, 20 bp, synthetic construct
  source 1..20: mol_type = other DNA
tttttgggtttgaaatgtta

SEQ ID NO: 272 - DNA, 21 bp, synthetic construct
  source 1..21: mol_type = other DNA
gggttgaggattagtattgat

SEQ ID NO: 273 - DNA, 20 bp, synthetic construct
  source 1..20: mol_type = other DNA
ggttgatgaggtataggtga

SEQ ID NO: 274 - DNA, 22 bp, synthetic construct
  source 1..22: mol_type = other DNA
gaagtaggttaagaaggaggat

SEQ ID NO: 275 - DNA, 20 bp, synthetic construct
  source 1..20: mol_type = other DNA
ttgttttttgttgtgtggaa

SEQ ID NO: 276 - DNA, 24 bp, synthetic construct
  source 1..24: mol_type = other DNA
tttttaggttattttttaaatgga

SEQ ID NO: 277 - DNA, 22 bp, synthetic construct
  source 1..22: mol_type = other DNA
tgtgattttagtatttgggaag

SEQ ID NO: 278 - DNA, 20 bp, synthetic construct
  source 1..20: mol_type = other DNA
tttttatgaagggagttgtg

SEQ ID NO: 279 - DNA, 20 bp, synthetic construct
  source 1..20: mol_type = other DNA
gtgtggaaggaaaaaaaaag

SEQ ID NO: 280 - DNA, 21 bp, synthetic construct
  source 1..21: mol_type = other DNA
atttttttattaggttggtgg

SEQ ID NO: 281 - DNA, 26 bp, synthetic construct
  source 1..26: mol_type = other DNA
tttagatataaatttttttgtatgga

SEQ ID NO: 282 - DNA, 23 bp, synthetic construct
  source 1..23: mol_type = other DNA
ttggggtagagtataggttagtt

SEQ ID NO: 283 - DNA, 22 bp, synthetic construct
  source 1..22: mol_type = other DNA
ggttattagaggttgttgttgt

SEQ ID NO: 284 - DNA, 20 bp, synthetic construct
  source 1..20: mol_type = other DNA
gggtgtggtaggtgttttgg

SEQ ID NO: 285 - DNA, 22 bp, synthetic construct
  source 1..22: mol_type = other DNA
tggattattaggttgagttgtt

SEQ ID NO: 286 - DNA, 20 bp, synthetic construct
  source 1..20: mol_type = other DNA
tttaggttttttgttggtgg

SEQ ID NO: 287 - DNA, 27 bp, synthetic construct
  source 1..27: mol_type = other DNA
agtattttatgttgttttagttgtttt

SEQ ID NO: 288 - DNA, 20 bp, synthetic construct
  source 1..20: mol_type = other DNA
agtaggaagggtattggtgg

SEQ ID NO: 289 - DNA, 20 bp, synthetic construct
  source 1..20: mol_type = other DNA
aagtgggtttgggaagtatg

SEQ ID NO: 290 - DNA, 22 bp, synthetic construct
  source 1..22: mol_type = other DNA
ttttggttttaaaagagagaaa

SEQ ID NO: 291 - DNA, 24 bp, synthetic construct
  source 1..24: mol_type = other DNA
tgatttatttattttgttttaggg

SEQ ID NO: 292 - DNA, 20 bp, synthetic construct
  source 1..20: mol_type = other DNA
taagaggagttgggtgtgta

SEQ ID NO: 293 - DNA, 20 bp, synthetic construct
  source 1..20: mol_type = other DNA
gttgtgttgggagttattgt

SEQ ID NO: 294 - DNA, 32 bp, synthetic construct; identical to SEQ ID NO: 157
  source 1..32: mol_type = other DNA

SEQ ID NO: 295 - DNA, 24 bp, synthetic construct
  source 1..24: mol_type = other DNA
aggaagtaagggtttattaatttt

SEQ ID NO: 296 - DNA, 20 bp, synthetic construct
  source 1..20: mol_type = other DNA
tattttatggttggggaaat

SEQ ID NO: 297 - DNA, 20 bp, synthetic construct
  source 1..20: mol_type = other DNA
gggttaggatgagagaatga

SEQ ID NO: 298 - DNA, 22 bp, synthetic construct
  source 1..22: mol_type = other DNA
tggtttttttggtaatataagg

SEQ ID NO: 299 - DNA, 20 bp, synthetic construct
  source 1..20: mol_type = other DNA
aattttgtaattgggtaggg

SEQ ID NO: 300 - DNA, 23 bp, synthetic construct
  source 1..23: mol_type = other DNA
ggtgggttgaaaggttttttaag

SEQ ID NO: 301 - DNA, 22 bp, synthetic construct
  source 1..22: mol_type = other DNA
ttggagtttttagttttgagtt

SEQ ID NO: 302 - DNA, 22 bp, synthetic construct
  source 1..22: mol_type = other DNA
ttgtgtgatagagtttagttgg

SEQ ID NO: 303 - DNA, 22 bp, synthetic construct
  source 1..22: mol_type = other DNA
tgttgaatttggtgtttttgtt

SEQ ID NO: 304 - DNA, 20 bp, synthetic construct
  source 1..20: mol_type = other DNA
agttttgaagggaagatagg

SEQ ID NO: 305 - DNA, 26 bp, synthetic construct
  source 1..26: mol_type = other DNA
aaaaaaattaaaaagagtaataggaa

SEQ ID NO: 306 - DNA, 20 bp, synthetic construct
  source 1..20: mol_type = other DNA
gggaatgtgtttttagaggt

SEQ ID NO: 307 - DNA, 25 bp, synthetic construct
  source 1..25: mol_type = other DNA
ttgtgaatataggtgtgagttaata

SEQ ID NO: 308 - DNA, 20 bp, synthetic construct
  source 1..20: mol_type = other DNA
ggatgggtggattaaatttt

SEQ ID NO: 309 - DNA, 26 bp, synthetic construct
  source 1..26: mol_type = other DNA
ttttgttaaattttttttttttagtt

SEQ ID NO: 310 - DNA, 24 bp, synthetic construct
  source 1..24: mol_type = other DNA
tgagatgattaaatgaagattaaa

SEQ ID NO: 311 - DNA, 22 bp, synthetic construct
  source 1..22: mol_type = other DNA
ggttgttgtaattgtttgttgt

SEQ ID NO: 312 - DNA, 24 bp, synthetic construct
  source 1..24: mol_type = other DNA
ttgtagtgtagtttgagaaatagg

SEQ ID NO: 313 - DNA, 25 bp, synthetic construct
  source 1..25: mol_type = other DNA
tgtttaattgtttgtttttttttag

SEQ ID NO: 314 - DNA, 20 bp, synthetic construct
  source 1..20: mol_type = other DNA
tggagggtgggagagtttag

SEQ ID NO: 315 - DNA, 21 bp, synthetic construct
  source 1..21: mol_type = other DNA
tttatgaaatttgtgggtgta

SEQ ID NO: 316 - DNA, 23 bp, synthetic construct
  source 1..23: mol_type = other DNA
tttggtgtatgtattgtgtatgt

SEQ ID NO: 317 - DNA, 20 bp, synthetic construct
  source 1..20: mol_type = other DNA
ggatattggttgttttggat

SEQ ID NO: 318 - DNA, 20 bp, synthetic construct
  source 1..20: mol_type = other DNA
gaggaaatgttttgttttgg

SEQ ID NO: 319 - DNA, 20 bp, synthetic construct
  source 1..20: mol_type = other DNA
gtttttggggttatagttgg

SEQ ID NO: 320 - DNA, 23 bp, synthetic construct
  source 1..23: mol_type = other DNA
tttgtttagaggtttatgtttgt

SEQ ID NO: 321 - DNA, 22 bp, synthetic construct
  source 1..22: mol_type = other DNA
gtggttttgaaatagattttgt

SEQ ID NO: 322 - DNA, 21 bp, synthetic construct
  source 1..21: mol_type = other DNA
ggtgttatttgaggttaggat

SEQ ID NO: 323 - DNA, 22 bp, synthetic construct
  source 1..22: mol_type = other DNA
aggaagatattgtttatgtgga

SEQ ID NO: 324 - DNA, 27 bp, synthetic construct
  source 1..27: mol_type = other DNA
ttatttttttatagttttataggatgg

SEQ ID NO: 325 - DNA, 24 bp, synthetic construct
  source 1..24: mol_type = other DNA
aggaaaattaatgaattgttaaag

SEQ ID NO: 326 - DNA, 22 bp, synthetic construct
  source 1..22: mol_type = other DNA
ttgaatgttgttatttggtatg

SEQ ID NO: 327 - DNA, 22 bp, synthetic construct
  source 1..22: mol_type = other DNA
tgttatttgattttgttttgga

SEQ ID NO: 328 - DNA, 25 bp, synthetic construct
  source 1..25: mol_type = other DNA
ttgtatttgtgtgattttagataag

SEQ ID NO: 329 - DNA, 26 bp, synthetic construct
  source 1..26: mol_type = other DNA
agttttaatagttttaagtttggatt

SEQ ID NO: 330 - DNA, 23 bp, synthetic construct
  source 1..23: mol_type = other DNA
gtatggtattttttgaagtgaag

SEQ ID NO: 331 - DNA, 21 bp, synthetic construct
  source 1..21: mol_type = other DNA
aagttgattggttagagttgg

SEQ ID NO: 332 - DNA, 23 bp, synthetic construct
  source 1..23: mol_type = other DNA
ggatttgtaattggtatagaagg

SEQ ID NO: 333 - DNA, 24 bp, synthetic construct
  source 1..24: mol_type = other DNA
ttggattttagattattaggtttt

SEQ ID NO: 334 - DNA, 20 bp, synthetic construct
  source 1..20: mol_type = other DNA
gggttggaatgttttgaagg

SEQ ID NO: 335 - DNA, 20 bp, synthetic construct
  source 1..20: mol_type = other DNA
gttggtgattgttgaaaatg

SEQ ID NO: 336 - DNA, 29 bp, synthetic construct
  source 1..29: mol_type = other DNA
ttaggatttatgtgttttataaaatatag

SEQ ID NO: 337 - DNA, 29 bp, synthetic construct
  source 1..29: mol_type = other DNA
aaattaaagagttaagtattagaaatgat

SEQ ID NO: 338 - DNA, 22 bp, synthetic construct
  source 1..22: mol_type = other DNA
aaaatttttttagtttgggaaa

SEQ ID NO: 339 - DNA, 20 bp, synthetic construct
  source 1..20: mol_type = other DNA
attttggttttagggggtga

SEQ ID NO: 340 - DNA, 22 bp, synthetic construct
  source 1..22: mol_type = other DNA
ttttttgggtgttaagttagtt

SEQ ID NO: 341 - DNA, 26 bp, synthetic construct
  source 1..26: mol_type = other DNA
ggttttaggtgatatttgaataataa

SEQ ID NO: 342 - DNA, 23 bp, synthetic construct
  source 1..23: mol_type = other DNA
gtaggggttttagatttttttag

SEQ ID NO: 343 - DNA, 30 bp, synthetic construct
  source 1..30: mol_type = other DNA
agttaagttagatgtgttataattagagtt

SEQ ID NO: 344 - DNA, 30 bp, synthetic construct
  source 1..30: mol_type = other DNA
aaaaaatatttttatttgtgtaattataaa

SEQ ID NO: 345 - DNA, 20 bp, synthetic construct
  source 1..20: mol_type = other DNA
taggaggggtgtgtgttgtg

SEQ ID NO: 346 - DNA, 21 bp, synthetic construct
  source 1..21: mol_type = other DNA
tgaatttgttgttgattttgt

SEQ ID NO: 347 - DNA, 22 bp, synthetic construct
  source 1..22: mol_type = other DNA
taatcccacttacaaaaaacac

SEQ ID NO: 348 - DNA, 24 bp, synthetic construct
  source 1..24: mol_type = other DNA
ttaatatctatcctaattccaacc

SEQ ID NO: 349 - DNA, 28 bp, synthetic construct
  source 1..28: mol_type = other DNA
tcaaaacatttctaaaactattaatatc

SEQ ID NO: 350 - DNA, 29 bp, synthetic construct
  source 1..29: mol_type = other DNA
aaacaaaacttaaacaataatacttactc

SEQ ID NO: 351 - DNA, 20 bp, synthetic construct
  source 1..20: mol_type = other DNA
ttaaatctaacacccacacc

SEQ ID NO: 352 - DNA, 23 bp, synthetic construct
  source 1..23: mol_type = other DNA
aatataaaaaataatccccaaac

SEQ ID NO: 353 - DNA, 20 bp, synthetic construct
  source 1..20: mol_type = other DNA
caaacaaaatttacaaccca

SEQ ID NO: 354 - DNA, 20 bp, synthetic construct
  source 1..20: mol_type = other DNA
acctcacccacttctcctac

SEQ ID NO: 355 - DNA, 20 bp, synthetic construct
  source 1..20: mol_type = other DNA
cacccaactcatttttttac

SEQ ID NO: 356 - DNA, 20 bp, synthetic construct
  source 1..20: mol_type = other DNA
atactccccactaactcccc

SEQ ID NO: 357 - DNA, 20 bp, synthetic construct
  source 1..20: mol_type = other DNA
acccaaaatctacaaaaccc

SEQ ID NO: 358 - DNA, 23 bp, synthetic construct
  source 1..23: mol_type = other DNA
aaatccctatatattccttacca

SEQ ID NO: 359 - DNA, 20 bp, synthetic construct
  source 1..20: mol_type = other DNA
cctcatactctaaaaacccc

SEQ ID NO: 360 - DNA, 21 bp, synthetic construct
  source 1..21: mol_type = other DNA
tccccactaatacttccttac

SEQ ID NO: 361 - DNA, 20 bp, synthetic construct
  source 1..20: mol_type = other DNA
atacctcctaacatcccact

SEQ ID NO: 362 - DNA, 20 bp, synthetic construct
  source 1..20: mol_type = other DNA
tacctctaatcccaattcaa

SEQ ID NO: 363 - DNA, 25 bp, synthetic construct
  source 1..25: mol_type = other DNA
tataaacaacttaaaaaacaacccc

SEQ ID NO: 364 - DNA, 20 bp, synthetic construct
  source 1..20: mol_type = other DNA
cccctttttcatcaaaactt

SEQ ID NO: 365 - DNA, 27 bp, synthetic construct
  source 1..27: mol_type = other DNA
aaactctacaaaaaccaaatataataa

SEQ ID NO: 366 - DNA, 26 bp, synthetic construct
  source 1..26: mol_type = other DNA
attattatacccactacacaaataaa

SEQ ID NO: 367 - DNA, 20 bp, synthetic construct
  source 1..20: mol_type = other DNA
taacctaaccacaaacaacc

SEQ ID NO: 368 - DNA, 24 bp, synthetic construct
  source 1..24: mol_type = other DNA
acctcaaataacttaaaattcact

SEQ ID NO: 369 - DNA, 22 bp, synthetic construct
  source 1..22: mol_type = other DNA
aaaaaaaatataacatctccca

SEQ ID NO: 370 - DNA, 23 bp, synthetic construct
  source 1..23: mol_type = other DNA
ttcacacttaatttaattaccca

SEQ ID NO: 371 - DNA, 21 bp, synthetic construct
  source 1..21: mol_type = other DNA
cccaataacattaaaacaacc

SEQ ID NO: 372 - DNA, 20 bp, synthetic construct
  source 1..20: mol_type = other DNA
ccaaaatctcctaacctcaa

SEQ ID NO: 373 - DNA, 20 bp, synthetic construct
  source 1..20: mol_type = other DNA
caccaaacaccacaatcaac

SEQ ID NO: 374 - DNA, 23 bp, synthetic construct
  source 1..23: mol_type = other DNA
ctccttcttccatactaataacc

SEQ ID NO: 375 - DNA, 20 bp, synthetic construct
  source 1..20: mol_type = other DNA
acttcccccacaaaaaaccc

SEQ ID NO: 376 - DNA, 35 bp, synthetic construct; identical to SEQ ID NO: 88
  source 1..35: mol_type = other DNA

SEQ ID NO: 377 - DNA, 26 bp, synthetic construct
  source 1..26: mol_type = other DNA
taactcctaaacttaaataatcctct

SEQ ID NO: 378 - DNA, 28 bp, synthetic construct
  source 1..28: mol_type = other DNA
tttttttataattttaaaaaataacatc

SEQ ID NO: 379 - DNA, 27 bp, synthetic construct
  source 1..27: mol_type = other DNA
aaacaaattaccaataaattaaaaata

SEQ ID NO: 380 - DNA, 20 bp, synthetic construct
  source 1..20: mol_type = other DNA
caacccacccacacaaatta

SEQ ID NO: 381 - DNA, 21 bp, synthetic construct
  source 1..21: mol_type = other DNA
ctcaaaaaaatcaaacttcaa

SEQ ID NO: 382 - DNA, 27 bp, synthetic construct
  source 1..27: mol_type = other DNA
caacattatttcctaaaacttataaat

SEQ ID NO: 383 - DNA, 26 bp, synthetic construct
  source 1..26: mol_type = other DNA
atatttcaaaccttatcttaaaactt

SEQ ID NO: 384 - DNA, 22 bp, synthetic construct
  source 1..22: mol_type = other DNA
aaactcctcttcccaaatatac

SEQ ID NO: 385 - DNA, 20 bp, synthetic construct
  source 1..20: mol_type = other DNA
aaaatccccttcaattctac

SEQ ID NO: 386 - DNA, 22 bp, synthetic construct
  source 1..22: mol_type = other DNA
ccctccaaaaaaaaaaatacta

SEQ ID NO: 387 - DNA, 29 bp, synthetic construct
  source 1..29: mol_type = other DNA
aaataaaaaaaataaataatatttttcaa

SEQ ID NO: 388 - DNA, 20 bp, synthetic construct
  source 1..20: mol_type = other DNA
taccaactacaccactctcc

SEQ ID NO: 389 - DNA, 20 bp, synthetic construct
  source 1..20: mol_type = other DNA
caaaatcaaccacaacctac

SEQ ID NO: 390 - DNA, 29 bp, synthetic construct
  source 1..29: mol_type = other DNA
aaacttttactcataatataatttctacc

SEQ ID NO: 391 - DNA, 25 bp, synthetic construct
  source 1..25: mol_type = other DNA
ttataaaactataaactccctttca

SEQ ID NO: 392 - DNA, 23 bp, synthetic construct
  source 1..23: mol_type = other DNA
ccctcaacctcctaaatacaaat

SEQ ID NO: 393 - DNA, 23 bp, synthetic construct
  source 1..23: mol_type = other DNA
aaaataccatttacctaaccaat

SEQ ID NO: 394 - DNA, 28 bp, synthetic construct
  source 1..28: mol_type = other DNA
caaatctttaactcaaaattaaataata

SEQ ID NO: 395 - DNA, 22 bp, synthetic construct
  source 1..22: mol_type = other DNA
ctcattttcaaactacactcaa

SEQ ID NO: 396 - DNA, 21 bp, synthetic construct
  source 1..21: mol_type = other DNA
aaaaacccattatttcaactt

SEQ ID NO: 397 - DNA, 28 bp, synthetic construct
  source 1..28: mol_type = other DNA
aaaaaaattttcctataactaataactt

SEQ ID NO: 398 - DNA, 22 bp, synthetic construct
  source 1..22: mol_type = other DNA
caaacttcaatacaataaccca

SEQ ID NO: 399 - DNA, 22 bp, synthetic construct
  source 1..22: mol_type = other DNA
tcctctcctttaaaaaaaattc

SEQ ID NO: 400 - DNA, 20 bp, synthetic construct
  source 1..20: mol_type = other DNA
tccctatctaaacccctcct

SEQ ID NO: 401 - DNA, 20 bp, synthetic construct
  source 1..20: mol_type = other DNA
aaatctaccttccaccaaat

SEQ ID NO: 402 - DNA, 23 bp, synthetic construct
  source 1..23: mol_type = other DNA
aactttctcaaaaacatatttca

SEQ ID NO: 403 - DNA, 21 bp, synthetic construct
  source 1..21: mol_type = other DNA
atctacaactcccaaaaattc

SEQ ID NO: 404 - DNA, 26 bp, synthetic construct
  source 1..26: mol_type = other DNA
acttaaactcctctctactcataaat

SEQ ID NO: 405 - DNA, 20 bp, synthetic construct
  source 1..20: mol_type = other DNA
aactaaccaccctctcctac

SEQ ID NO: 406 - DNA, 27 bp, synthetic construct
  source 1..27: mol_type = other DNA
aaattaaactcttaatacactccataa

SEQ ID NO: 407 - DNA, 30 bp, synthetic construct
  source 1..30: mol_type = other DNA
ttatcctttttatttaaaaataatattaaa

SEQ ID NO: 408 - DNA, 29 bp, synthetic construct
  source 1..29: mol_type = other DNA
ttcctaataaactaaaaatattaaaattc

SEQ ID NO: 409 - DNA, 30 bp, synthetic construct
  source 1..30: mol_type = other DNA
tcttttctttctatcaatattaataaaata

SEQ ID NO: 410 - DNA, 24 bp, synthetic construct
  source 1..24: mol_type = other DNA
acaattctctttcctttaaaataa

SEQ ID NO: 411 - DNA, 28 bp, synthetic construct
  source 1..28: mol_type = other DNA
tcttactaaaactaacatcacaaaatac

SEQ ID NO: 412 - DNA, 32 bp, synthetic construct
  source 1..32: mol_type = other DNA
atcaaaaataatttattaaatattaaatactt

SEQ ID NO: 413 - DNA, 22 bp, synthetic construct
  source 1..22: mol_type = other DNA
aacttcacaaaaacatctctct

SEQ ID NO: 414 - DNA, 25 bp, synthetic construct
  source 1..25: mol_type = other DNA
aaattaatattatttttctaacccc

SEQ ID NO: 415 - DNA, 21 bp, synthetic construct
  source 1..21: mol_type = other DNA
tcacacccttactaattaccc

SEQ ID NO: 416 - DNA, 21 bp, synthetic construct
  source 1..21: mol_type = other DNA
actccctccttattcaataaa

SEQ ID NO: 417 - DNA, 20 bp, synthetic construct
  source 1..20: mol_type = other DNA
ctcaacaccctaccctaaat

SEQ ID NO: 418 - DNA, 22 bp, synthetic construct
  source 1..22: mol_type = other DNA
caaaatataaaaatcaaatccc

SEQ ID NO: 419 - DNA, 27 bp, synthetic construct
  source 1..27: mol_type = other DNA
aacaaaaaaaactactaacataatacc

SEQ ID NO: 420 - DNA, 24 bp, synthetic construct
  source 1..24: mol_type = other DNA
ttttctcccttataattttaacac

SEQ ID NO: 421 - DNA, 27 bp, synthetic construct
  source 1..27: mol_type = other DNA
aaaacaactcaaaactctaaaataata

SEQ ID NO: 422 - DNA, 31 bp, synthetic construct
  source 1..31: mol_type = other DNA
acaaactaaaataaatctataacaataataa

SEQ ID NO: 423 - DNA, 20 bp, synthetic construct
  source 1..20: mol_type = other DNA
ctaaactccccaaaaacact

SEQ ID NO: 424 - DNA, 21 bp, synthetic construct
  source 1..21: mol_type = other DNA
ctcatttaaacctcataaccc

SEQ ID NO: 425 - DNA, 24 bp, synthetic construct
  source 1..24: mol_type = other DNA
tggtagtgattagtttattttttg

SEQ ID NO: 426 - DNA, 20 bp, synthetic construct
  source 1..20: mol_type = other DNA
tgtagagaggaggaggtgag

SEQ ID NO: 427 - DNA, 20 bp, synthetic construct
  source 1..20: mol_type = other DNA
tttttgggtttgaaatgtta

SEQ ID NO: 428 - DNA, 21 bp, synthetic construct
  source 1..21: mol_type = other DNA
gggttgaggattagtattgat

SEQ ID NO: 429 - DNA, 20 bp, synthetic construct
  source 1..20: mol_type = other DNA
ggttgatgaggtataggtga

SEQ ID NO: 430 - DNA, 22 bp, synthetic construct
  source 1..22: mol_type = other DNA
gaagtaggttaagaaggaggat

SEQ ID NO: 431 - DNA, 20 bp, synthetic construct
  source 1..20: mol_type = other DNA
ttgttttttgttgtgtggaa

SEQ ID NO: 432 - DNA, 24 bp, synthetic construct
  source 1..24: mol_type = other DNA
tttttaggttattttttaaatgga

SEQ ID NO: 433 - DNA, 22 bp, synthetic construct
  source 1..22: mol_type = other DNA
tgtgattttagtatttgggaag

SEQ ID NO: 434 - DNA, 20 bp, synthetic construct
  source 1..20: mol_type = other DNA
ggggagttttttatgaaggg

SEQ ID NO: 435 - DNA, 20 bp, synthetic construct
  source 1..20: mol_type = other DNA
gtgtggaaggaaaaaaaaag

SEQ ID NO: 436 - DNA, 21 bp, synthetic construct
  source 1..21: mol_type = other DNA
atttttttattaggttggtgg

SEQ ID NO: 437 - DNA, 26 bp, synthetic construct
  source 1..26: mol_type = other DNA
tttagatataaatttttttgtatgga

SEQ ID NO: 438 - DNA, 23 bp, synthetic construct
  source 1..23: mol_type = other DNA
tttattggggtagagtataggtt

SEQ ID NO: 439 - DNA, 20 bp, synthetic construct
  source 1..20: mol_type = other DNA
gaggttgttgttgtgtttgt

SEQ ID NO: 440 - DNA, 20 bp, synthetic construct
  source 1..20: mol_type = other DNA
gggtgtggtaggtgttttgg

SEQ ID NO: 441 - DNA, 20 bp, synthetic construct
  source 1..20: mol_type = other DNA
tgttgttttgtttgtatgga

SEQ ID NO: 442 - DNA, 20 bp, synthetic construct
  source 1..20: mol_type = other DNA
tttaggttttttgttggtgg

SEQ ID NO: 443 - DNA, 27 bp, synthetic construct
  source 1..27: mol_type = other DNA
gagtattttatgttgttttagttgttt

SEQ ID NO: 444 - DNA, 20 bp, synthetic construct
  source 1..20: mol_type = other DNA
agtaggaagggtattggtgg

SEQ ID NO: 445 - DNA, 20 bp, synthetic construct
  source 1..20: mol_type = other DNA
ggaagtgggtttgggaagta

SEQ ID NO: 446 - DNA, 22 bp, synthetic construct
  source 1..22: mol_type = other DNA
ttttggttttaaaagagagaaa

SEQ ID NO: 447 - DNA, 25 bp, synthetic construct
  source 1..25: mol_type = other DNA
aagagtttttaatgaatggatataa

SEQ ID NO: 448 - DNA, 20 bp, synthetic construct
  source 1..20: mol_type = other DNA
aagtggtaagaggagttggg

SEQ ID NO: 449 - DNA, 20 bp, synthetic construct
  source 1..20: mol_type = other DNA
ggtttttgttgtgttgggag

SEQ ID NO: 450 - DNA, 32 bp, synthetic construct; identical to SEQ ID NO: 157
  source 1..32: mol_type = other DNA

SEQ ID NO: 451 - DNA, 23 bp, synthetic construct
  source 1..23: mol_type = other DNA
gagaggaagtaagggtttattaa

SEQ ID NO: 452 - DNA, 20 bp, synthetic construct
  source 1..20: mol_type = other DNA
tattttatggttggggaaat

SEQ ID NO: 453 - DNA, 20 bp, synthetic construct
  source 1..20: mol_type = other DNA
ggagattggggttaggatga

SEQ ID NO: 454 - DNA, 22 bp, synthetic construct
  source 1..22: mol_type = other DNA
tggtttttttggtaatataagg

SEQ ID NO: 455 - DNA, 24 bp, synthetic construct
  source 1..24: mol_type = other DNA
agtaggtttttaaaatatgtggtt

SEQ ID NO: 456 - DNA, 20 bp, synthetic construct
  source 1..20: mol_type = other DNA
aattttgtaattgggtaggg

SEQ ID NO: 457 - DNA, 21 bp, synthetic construct
  source 1..21: mol_type = other DNA
ttttgtagttttgagaggtga

SEQ ID NO: 458 - DNA, 23 bp, synthetic construct
  source 1..23: mol_type = other DNA
tttattggagtttttagttttga

SEQ ID NO: 459 - DNA, 22 bp, synthetic construct
  source 1..22: mol_type = other DNA
ttgtgtgatagagtttagttgg

SEQ ID NO: 460 - DNA, 20 bp, synthetic construct
  source 1..20: mol_type = other DNA
ttttgtgtggatagttgttg

SEQ ID NO: 461 - DNA, 20 bp, synthetic construct
  source 1..20: mol_type = other DNA
ttgggatagtgttttgagtg

SEQ ID NO: 462 - DNA, 20 bp, synthetic construct
  source 1..20: mol_type = other DNA
ttttgagaagttttgaaggg

SEQ ID NO: 463 - DNA, 25 bp, synthetic construct
  source 1..25: mol_type = other DNA
ttgtttttaaaaaaattaaaaagag

SEQ ID NO: 464 - DNA, 20 bp, synthetic construct
  source 1..20: mol_type = other DNA
ttggggaatgtgtttttaga

SEQ ID NO: 465 - DNA, 24 bp, synthetic construct
  source 1..24: mol_type = other DNA
gttgtgaatataggtgtgagttaa

SEQ ID NO: 466 - DNA, 20 bp, synthetic construct
  source 1..20: mol_type = other DNA
gttttttaggagagggggtg

SEQ ID NO: 467 - DNA, 29 bp, synthetic construct
  source 1..29: mol_type = other DNA
ttatgtattttttttttatttaaaaaatt

SEQ ID NO: 468 - DNA, 24 bp, synthetic construct
  source 1..24: mol_type = other DNA
aatgaagattaaaaaaagttaagg

SEQ ID NO: 469 - DNA, 21 bp, synthetic construct
  source 1..21: mol_type = other DNA
ggttgttgtaattgtttgttg

SEQ ID NO: 470 - DNA, 24 bp, synthetic construct
  source 1..24: mol_type = other DNA
ttgtagtgtagtttgagaaatagg

SEQ ID NO: 471 - DNA, 26 bp, synthetic construct
  source 1..26: mol_type = other DNA
aattttatatgtgtttaattgtttgt

SEQ ID NO: 472 - DNA, 20 bp, synthetic construct
  source 1..20: mol_type = other DNA
tattggagggtgggagagtt

SEQ ID NO: 473 - DNA, 20 bp, synthetic construct
  source 1..20: mol_type = other DNA
gtttatgaaatttgtgggtg

SEQ ID NO: 474 - DNA, 20 bp, synthetic construct
  source 1..20: mol_type = other DNA
gagtgtgtgattgggtttgt

SEQ ID NO: 475 - DNA, 20 bp, synthetic construct
  source 1..20: mol_type = other DNA
ggatattggttgttttggat

SEQ ID NO: 476 - DNA, 20 bp, synthetic construct
  source 1..20: mol_type = other DNA
tttttataattggtgaggga

SEQ ID NO: 477 - DNA, 20 bp, synthetic construct
  source 1..20: mol_type = other DNA
gtttttggggttatagttgg

SEQ ID NO: 478 - DNA, 23 bp, synthetic construct
  source 1..23: mol_type = other DNA
gaaagaaaagaaaaagagaaagt

SEQ ID NO: 479 - DNA, 22 bp, synthetic construct
  source 1..22: mol_type = other DNA
tgtggttttgaaatagattttg

SEQ ID NO: 480 - DNA, 21 bp, synthetic construct
  source 1..21: mol_type = other DNA
ggtgttatttgaggttaggat

SEQ ID NO: 481 - DNA, 21 bp, synthetic construct
  source 1..21: mol_type = other DNA
tgtggaaatattgatttttga

SEQ ID NO: 482 - DNA, 20 bp, synthetic construct
  source 1..20: mol_type = other DNA
tataggatggtagggttggg

SEQ ID NO: 483 - DNA, 24 bp, synthetic construct
  source 1..24: mol_type = other DNA
aggaaaattaatgaattgttaaag

SEQ ID NO: 484 - DNA, 22 bp, synthetic construct
  source 1..22: mol_type = other DNA
tgaatgttgttatttggtatga

SEQ ID NO: 485 - DNA, 20 bp, synthetic construct
  source 1..20: mol_type = other DNA
tttgattttgttttggagtg

SEQ ID NO: 486 - DNA, 24 bp, synthetic construct
  source 1..24: mol_type = other DNA
ttttaattttgtatttgtgtgatt

SEQ ID NO: 487 - DNA, 26 bp, synthetic construct
  source 1..26: mol_type = other DNA
aaagttttaatagttttaagtttgga

SEQ ID NO: 488 - DNA, 23 bp, synthetic construct
  source 1..23: mol_type = other DNA
gtatggtattttttgaagtgaag

SEQ ID NO: 489 - DNA, 21 bp, synthetic construct
  source 1..21: mol_type = other DNA
aagttgattggttagagttgg

SEQ ID NO: 490 - DNA, 23 bp, synthetic construct
  source 1..23: mol_type = other DNA
ggatttgtaattggtatagaagg

SEQ ID NO: 491 - DNA, 20 bp, synthetic construct
  source 1..20: mol_type = other DNA
gaggggatgtttttttgttg

SEQ ID NO: 492 - DNA, 20 bp, synthetic construct
  source 1..20: mol_type = other DNA
tttaaaggagggttggaatg

SEQ ID NO: 493 - DNA, 20 bp, synthetic construct
  source 1..20: mol_type = other DNA
agttggtgattgttgaaaat

SEQ ID NO: 494 - DNA, 27 bp, synthetic construct
  source 1..27: mol_type = other DNA
ggttaggatttatgtgttttataaaat

SEQ ID NO: 495 - DNA, 28 bp, synthetic construct
  source 1..28: mol_type = other DNA
ttaaagagttaagtattagaaatgatgt

SEQ ID NO: 496 - DNA, 20 bp, synthetic construct
  source 1..20: mol_type = other DNA
taaaggtgaagggtgtgggg

SEQ ID NO: 497 - DNA, 20 bp, synthetic construct
  source 1..20: mol_type = other DNA
tttggtttatggggatttat

SEQ ID NO: 498 - DNA, 20 bp, synthetic construct
  source 1..20: mol_type = other DNA
gttaggttggggtggtggtt

SEQ ID NO: 499 - DNA, 20 bp, synthetic construct
  source 1..20: mol_type = other DNA
gaggtgggggttttttattg

SEQ ID NO: 500 - DNA, 23 bp, synthetic construct
  source 1..23: mol_type = other DNA
tatttttttgggtgttaagttag

SEQ ID NO: 501 - DNA, 24 bp, synthetic construct
  source 1..24: mol_type = other DNA
ggttttaggtgatatttgaataat

SEQ ID NO: 502 - DNA, 23 bp, synthetic construct
  source 1..23: mol_type = other DNA
agtattggtgtatatgagaagga

SEQ ID NO: 503 - DNA, 30 bp, synthetic construct; identical to SEQ ID NO: 59
  source 1..30: mol_type = other DNA

SEQ ID NO: 504 - DNA, 28 bp, synthetic construct
  source 1..28: mol_type = other DNA
agattaaaaaatatttttatttgtgtaa

SEQ ID NO: 505 - DNA, 20 bp, synthetic construct
  source 1..20: mol_type = other DNA
gggagtaggaggggtgtgtg

SEQ ID NO: 506 - DNA, 21 bp, synthetic construct
  source 1..21: mol_type = other DNA
tgaatttgttgttgattttgt

SEQ ID NO: 507 - DNA, 22 bp, synthetic construct
  source 1..22: mol_type = other DNA
taatcccacttacaaaaaacac

SEQ ID NO: 508 - DNA, 24 bp, synthetic construct
  source 1..24: mol_type = other DNA
ttaatatctatcctaattccaacc

SEQ ID NO: 509 - DNA, 28 bp, synthetic construct
  source 1..28: mol_type = other DNA
tcaaaacatttctaaaactattaatatc

SEQ ID NO: 510 - DNA, 27 bp, synthetic construct
  source 1..27: mol_type = other DNA
caaaacttaaacaataatacttactca

SEQ ID NO: 511 - DNA, 20 bp, synthetic construct
  source 1..20: mol_type = other DNA
ttaaatctaacacccacacc

SEQ ID NO: 512 - DNA, 23 bp, synthetic construct
  source 1..23: mol_type = other DNA
aatataaaaaataatccccaaac

SEQ ID NO: 513 - DNA, 20 bp, synthetic construct
  source 1..20: mol_type = other DNA
caaacaaaatttacaaccca

SEQ ID NO: 514 - DNA, 20 bp, synthetic construct
  source 1..20: mol_type = other DNA
acctcacccacttctcctac

SEQ ID NO: 515 - DNA, 20 bp, synthetic construct
  source 1..20: mol_type = other DNA
cacccaactcatttttttac

SEQ ID NO: 516 - DNA, 20 bp, synthetic construct
  source 1..20: mol_type = other DNA
cccactaactccccaaaaac

SEQ ID NO: 517 - DNA, 20 bp, synthetic construct
  source 1..20: mol_type = other DNA
acccaaaatctacaaaaccc

SEQ ID NO: 518 - DNA, 23 bp, synthetic construct
  source 1..23: mol_type = other DNA
aaatccctatatattccttacca

SEQ ID NO: 519 - DNA, 20 bp, synthetic construct
  source 1..20: mol_type = other DNA
cctcatactctaaaaacccc

SEQ ID NO: 520 - DNA, 21 bp, synthetic construct
  source 1..21: mol_type = other DNA
tccccactaatacttccttac

SEQ ID NO: 521 - DNA, 20 bp, synthetic construct
  source 1..20: mol_type = other DNA
catacctcctaacatcccac

SEQ ID NO: 522 - DNA, 20 bp, synthetic construct
  source 1..20: mol_type = other DNA
tacctctaatcccaattcaa

SEQ ID NO: 523 - DNA, 25 bp, synthetic construct
  source 1..25: mol_type = other DNA
aaaaataaaccttacaaactacaca

SEQ ID NO: 524 - DNA, 20 bp, synthetic construct
  source 1..20: mol_type = other DNA
caaacacccctttttcatca

SEQ ID NO: 525 - DNA, 27 bp, synthetic construct
  source 1..27: mol_type = other DNA
aactctacaaaaaccaaatataataac

SEQ ID NO: 526 - DNA, 24 bp, synthetic construct
  source 1..24: mol_type = other DNA
ttatacccactacacaaataaaaa

SEQ ID NO: 527 - DNA, 20 bp, synthetic construct
  source 1..20: mol_type = other DNA
taacctaaccacaaacaacc

SEQ ID NO: 528 - DNA, 24 bp, synthetic construct
  source 1..24: mol_type = other DNA
acctcaaataacttaaaattcact

SEQ ID NO: 529 - DNA, 31 bp, synthetic construct
  source 1..31: mol_type = other DNA
tttctaacatatttactactaaaaaatttaa

SEQ ID NO: 530 - DNA, 24 bp, synthetic construct
  source 1..24: mol_type = other DNA
acttaatttaattacccaaacaat

SEQ ID NO: 531 - DNA, 21 bp, synthetic construct
  source 1..21: mol_type = other DNA
cccaataacattaaaacaacc

SEQ ID NO: 532 - DNA, 20 bp, synthetic construct
  source 1..20: mol_type = other DNA
atccaaaatctcctaacctc

SEQ ID NO: 533 - DNA, 20 bp, synthetic construct
  source 1..20: mol_type = other DNA
caccaaacaccacaatcaac

SEQ ID NO: 534 - DNA, 21 bp, synthetic construct
  source 1..21: mol_type = other DNA
aacaactccttcttccatact

SEQ ID NO: 535 - DNA, 26 bp, synthetic construct
  source 1..26: mol_type = other DNA
cacaaaaaaccctaaaaaactaaaaa

SEQ ID NO: 536 - DNA, 35 bp, synthetic construct; identical to SEQ ID NO: 88
  source 1..35: mol_type = other DNA

SEQ ID NO: 537 - DNA, 20 bp, synthetic construct
  source 1..20: mol_type = other DNA
ataacaaaactcaaaacccc

SEQ ID NO: 538 - DNA, 24 bp, synthetic construct
  source 1..24: mol_type = other DNA
cacttattacccaaactaatcttt

SEQ ID NO: 539 - DNA, 21 bp, synthetic construct
  source 1..21: mol_type = other DNA
aaaaaaatccctcaatacaac

SEQ ID NO: 540 - DNA, 25 bp, synthetic construct
  source 1..25: mol_type = other DNA
aaaacaaattaccaataaattaaaa

SEQ ID NO: 541 - DNA, 20 bp, synthetic construct
  source 1..20: mol_type = other DNA
caacccacccacacaaatta

SEQ ID NO: 542 - DNA, 21 bp, synthetic construct
  source 1..21: mol_type = other DNA
ctcaaaaaaatcaaacttcaa

SEQ ID NO: 543 - DNA, 22 bp, synthetic construct
  source 1..22: mol_type = other DNA
cccacaaactacttctacaaat

SEQ ID NO: 544 - DNA, 29 bp, synthetic construct
  source 1..29: mol_type = other DNA
atttcctaaaacttataaatttataaaaa

SEQ ID NO: 545 - DNA, 24 bp, synthetic construct
  source 1..24: mol_type = other DNA
tttcaaaccttatcttaaaacttc

SEQ ID NO: 546 - DNA, 21 bp, synthetic construct
  source 1..21: mol_type = other DNA
taaactcctcttcccaaatat

SEQ ID NO: 547 - DNA, 20 bp, synthetic construct
  source 1..20: mol_type = other DNA
aaaatccccttcaattctac

SEQ ID NO: 548 - DNA, 20 bp, synthetic construct
  source 1..20: mol_type = other DNA
aaccaaacccttaacaaccc

SEQ ID NO: 549 - DNA, 26 bp, synthetic construct
  source 1..26: mol_type = other DNA
aaaatttcaaaaaaatactaaaaaat

SEQ ID NO: 550 - DNA, 20 bp, synthetic construct
  source 1..20: mol_type = other DNA
taccaactacaccactctcc

SEQ ID NO: 551 - DNA, 20 bp, synthetic construct
  source 1..20: mol_type = other DNA
caaaatcaaccacaacctac

SEQ ID NO: 552 - DNA, 28 bp, synthetic construct
  source 1..28: mol_type = other DNA
ttttactcataatataatttctacctca

SEQ ID NO: 553 - DNA, 23 bp, synthetic construct
  source 1..23: mol_type = other DNA
aaaactataaactccctttcatt

SEQ ID NO: 554 - DNA, 20 bp, synthetic construct
  source 1..20: mol_type = other DNA
ccctcaacctcctaaataca

SEQ ID NO: 555 - DNA, 22 bp, synthetic construct
  source 1..22: mol_type = other DNA
aaaataccatttacctaaccaa

SEQ ID NO: 556 - DNA, 20 bp, synthetic construct
  source 1..20: mol_type = other DNA
aaccaccacctccaaatctt

SEQ ID NO: 557 - DNA, 23 bp, synthetic construct
  source 1..23: mol_type = other DNA
aacatctcattttcaaactacac

SEQ ID NO: 558 - DNA, 23 bp, synthetic construct
  source 1..23: mol_type = other DNA
cccattatttcaacttacactct

SEQ ID NO: 559 - DNA, 22 bp, synthetic construct
  source 1..22: mol_type = other DNA
ttccataacaatcactcactaa

SEQ ID NO: 560 - DNA, 27 bp, synthetic construct
  source 1..27: mol_type = other DNA
aacttaaacccaaaactttaaaactac

SEQ ID NO: 561 - DNA, 22 bp, synthetic construct
  source 1..22: mol_type = other DNA
cctctcctttaaaaaaaattcc

SEQ ID NO: 562 - DNA, 20 bp, synthetic construct
  source 1..20: mol_type = other DNA
tccctatctaaacccctcct

SEQ ID NO: 563 - DNA, 20 bp, synthetic construct
  source 1..20: mol_type = other DNA
aaatctaccttccaccaaat

SEQ ID NO: 564 - DNA, 21 bp, synthetic construct
  source 1..21: mol_type = other DNA
ccttcacataccaaaaaaaac

SEQ ID NO: 565 - DNA, 21 bp, synthetic construct
  source 1..21: mol_type = other DNA
atctacaactcccaaaaattc

SEQ ID NO: 566 - DNA, 22 bp, synthetic construct
  source 1..22: mol_type = other DNA
ccccctaaaatttactaaaaaa

SEQ ID NO: 567 - DNA, 20 bp, synthetic construct
  source 1..20: mol_type = other DNA
aaactaaccaccctctccta

SEQ ID NO: 568 - DNA, 25 bp, synthetic construct
  source 1..25: mol_type = other DNA
ctcttaatacactccataattaacc

SEQ ID NO: 569 - DNA, 29 bp, synthetic construct
  source 1..29: mol_type = other DNA
cctttttatttaaaaataatattaaacat

SEQ ID NO: 570 - DNA, 29 bp, synthetic construct
  source 1..29: mol_type = other DNA
ttcctaataaactaaaaatattaaaattc

SEQ ID NO: 571 - DNA, 21 bp, synthetic construct
  source 1..21: mol_type = other DNA
cctcctcttttctttctatca

SEQ ID NO: 572 - DNA, 24 bp, synthetic construct
  source 1..24: mol_type = other DNA
attacaattctctttcctttaaaa

SEQ ID NO: 573 - DNA, 26 bp, synthetic construct
  source 1..26: mol_type = other DNA
catctcttactaaaactaacatcaca

SEQ ID NO: 574 - DNA, 34 bp, synthetic construct
  source 1..34: mol_type = other DNA
aatttattaaatattaaatacttaaaaaaattaa

SEQ ID NO: 575 - DNA, 22 bp, synthetic construct
  source 1..22: mol_type = other DNA
aacttcacaaaaacatctctct

SEQ ID NO: 576 - DNA, 25 bp, synthetic construct
  source 1..25: mol_type = other DNA
aaattaatattatttttctaacccc

SEQ ID NO: 577 - DNA, 21 bp, synthetic construct
  source 1..21: mol_type = other DNA
tcacacccttactaattaccc

SEQ ID NO: 578 - DNA, 21 bp, synthetic construct
  source 1..21: mol_type = other DNA
aaaaaatctttccaacctaaa

SEQ ID NO: 579 - DNA, 20 bp, synthetic construct
  source 1..20: mol_type = other DNA
tctcaacaccctaccctaaa

SEQ ID NO: 580 - DNA, 24 bp, synthetic construct
  source 1..24: mol_type = other DNA
caactatactttcccataacctaa

SEQ ID NO: 581 - DNA, 21 bp, synthetic construct
  source 1..21: mol_type = other DNA
aaaccccaaaactcccacaac

SEQ ID NO: 582 - DNA, 22 bp, synthetic construct
  source 1..22: mol_type = other DNA
aaaatataaaaatcaaatcccc

SEQ ID NO: 583 - DNA, 26 bp, synthetic construct
  source 1..26: mol_type = other DNA
cataaaaaaaaacaaaaaaaactact

SEQ ID NO: 584 - DNA, 20 bp, synthetic construct
  source 1..20: mol_type = other DNA
caacaaaaactccaaacttc

SEQ ID NO: 585 - DNA, 29 bp, synthetic construct
  source 1..29: mol_type = other DNA
caaaactctaaaataataaacaataaaat

SEQ ID NO: 586 - DNA, 30 bp, synthetic construct
  source 1..30: mol_type = other DNA
ctatctaaaaatacaaactaaaataaatct

SEQ ID NO: 587 - DNA, 20 bp, synthetic construct
  source 1..20: mol_type = other DNA
ctaaactccccaaaaacact

SEQ ID NO: 588 - DNA, 21 bp, synthetic construct
  source 1..21: mol_type = other DNA
ctcatttaaacctcataaccc

SEQ ID NO: 589 - DNA, 25 bp, synthetic construct
  source 1..25: mol_type = other DNA
ttttttttatagtttttggtagtga

SEQ ID NO: 590 - DNA, 20 bp, synthetic construct
  source 1..20: mol_type = other DNA
gaggaggaggtgagttgtag

SEQ ID NO: 591 - DNA, 23 bp, synthetic construct
  source 1..23: mol_type = other DNA
aataattttttttttgggtttga

SEQ ID NO: 592 - DNA, 22 bp, synthetic construct
  source 1..22: mol_type = other DNA
aagaaggaggatatagagaagg

SEQ ID NO: 593 - DNA, 23 bp, synthetic construct
  source 1..23: mol_type = other DNA
aaaggggtaaatagaatttgtag

SEQ ID NO: 594 - DNA, 20 bp, synthetic construct
  source 1..20: mol_type = other DNA
ttttaaggtgttaggggaag

SEQ ID NO: 595 - DNA, 20 bp, synthetic construct
  source 1..20: mol_type = other DNA
gggagttttttatgaaggga

SEQ ID NO: 596 - DNA, 20 bp, synthetic construct
  source 1..20: mol_type = other DNA
gtgtggaaggaaaaaaaaag

SEQ ID NO: 597 - DNA, 25 bp, synthetic construct
  source 1..25: mol_type = other DNA
gtttgtttgttatttttttattagg

SEQ ID NO: 598 - DNA, 23 bp, synthetic construct
  source 1..23: mol_type = other DNA
tttattgatgtttttttgttagg

SEQ ID NO: 599 - DNA, 22 bp, synthetic construct
  source 1..22: mol_type = other DNA
ttggtattttatttttgagagg

SEQ ID NO: 600 - DNA, 20 bp, synthetic construct
  source 1..20: mol_type = other DNA
tggtaggtgttttgggttga

SEQ ID NO: 601 - DNA, 21 bp, synthetic construct
  source 1..21: mol_type = other DNA
ttttgtaggggttaggtgtag

SEQ ID NO: 602 - DNA, 24 bp, synthetic construct
  source 1..24: mol_type = other DNA
ttattatttattttttgggtgaag

SEQ ID NO: 603 - DNA, 20 bp, synthetic construct
  source 1..20: mol_type = other DNA
gggtttgggaagtatggaag

SEQ ID NO: 604 - DNA, 22 bp, synthetic construct
  source 1..22: mol_type = other DNA
ttttggttttaaaagagagaaa

SEQ ID NO: 605 - DNA, 27 bp, synthetic construct
  source 1..27: mol_type = other DNA
agggttatattttaatatgtagaaaaa

SEQ ID NO: 606 - DNA, 20 bp, synthetic construct
  source 1..20: mol_type = other DNA
agaggagttgggtgtgtaag

SEQ ID NO: 607 - DNA, 20 bp, synthetic construct
  source 1..20: mol_type = other DNA
ggtttttgttgtgttgggag

SEQ ID NO: 608 - DNA, 29 bp, synthetic construct
  source 1..29: mol_type = other DNA
ttagggttttttaattttagtatataaag

SEQ ID NO: 609 - DNA, 28 bp, synthetic construct
  source 1..28: mol_type = other DNA
agggtttattaatttttttaataagtag

SEQ ID NO: 610 - DNA, 21 bp, synthetic construct
  source 1..21: mol_type = other DNA
gggttttaagtagggaggtag

SEQ ID NO: 611 - DNA, 20 bp, synthetic construct
  source 1..20: mol_type = other DNA
agaaaattttgggaggttga

SEQ ID NO: 612 - DNA, 30 bp, synthetic construct
  source 1..30: mol_type = other DNA
ggtaatataaggtatagagtataggttagg

SEQ ID NO: 613 - DNA, 23 bp, synthetic construct
  source 1..23: mol_type = other DNA
ttggaatttataggtttgtaaag

SEQ ID NO: 614 - DNA, 24 bp, synthetic construct
  source 1..24: mol_type = other DNA
aatgaagattaaaaaaagttaagg

SEQ ID NO: 615 - DNA, 24 bp, synthetic construct
  source 1..24: mol_type = other DNA
gtttgttgttttgtagaaaaataa

SEQ ID NO: 616 - DNA, 27 bp, synthetic construct
  source 1..27: mol_type = other DNA
gtttgagaaataggtaataaaaataga

SEQ ID NO: 617 - DNA, 21 bp, synthetic construct
  source 1..21: mol_type = other DNA
ttgtgggtgtaaataaattga

SEQ ID NO: 618 - DNA, 27 bp, synthetic construct
  source 1..27: mol_type = other DNA
aattggaatatgttattaattagaaaa

SEQ ID NO: 619 - DNA, 20 bp, synthetic construct
  source 1..20: mol_type = other DNA
ttggggttatagttggagag

SEQ ID NO: 620 - DNA, 24 bp, synthetic construct
  source 1..24: mol_type = other DNA
ggaaatattgatttttgatagaag

SEQ ID NO: 621 - DNA, 23 bp, synthetic construct
  source 1..23: mol_type = other DNA
ttttttgagatttgttaagaaag

SEQ ID NO: 622 - DNA, 21 bp, synthetic construct
  source 1..21: mol_type = other DNA
aggggtagttgtagaggtaga

SEQ ID NO: 623 - DNA, 27 bp, synthetic construct
  source 1..27: mol_type = other DNA
ttatttaattttatattttgaaggaga

SEQ ID NO: 624 - DNA, 20 bp, synthetic construct
  source 1..20: mol_type = other DNA
agtgttgggattttgattga

SEQ ID NO: 625 - DNA, 23 bp, synthetic construct
  source 1..23: mol_type = other DNA
ggatttgtaattggtatagaagg

SEQ ID NO: 626 - DNA, 20 bp, synthetic construct
  source 1..20: mol_type = other DNA
tgtgtaggttttttggtagg

SEQ ID NO: 627 - DNA, 28 bp, synthetic construct
  source 1..28: mol_type = other DNA
aagagttaagtattagaaatgatgtaag

SEQ ID NO: 628 - DNA, 30 bp, synthetic construct
  source 1..30: mol_type = other DNA
ttttagtattttgttttaagttagttaaag

SEQ ID NO: 629 - DNA, 26 bp, synthetic construct
  source 1..26: mol_type = other DNA
ggttttaggtgatatttgaataataa

SEQ ID NO: 630 - DNA, 22 bp, synthetic construct
  source 1..22: mol_type = other DNA
attttgggataataggtagtga

SEQ ID NO: 631 - DNA, 20 bp, synthetic construct
  source 1..20: mol_type = other DNA
gtgtgtgttgtggtgaggag

SEQ ID NO: 632 - DNA, 20 bp, synthetic construct
  source 1..20: mol_type = other DNA
aatcccacttacaaaaaaca

SEQ ID NO: 633 - DNA, 24 bp, synthetic construct
  source 1..24: mol_type = other DNA
accttaatatctatcctaattcca

SEQ ID NO: 634 - DNA, 31 bp, synthetic construct
  source 1..31: mol_type = other DNA
tctaaaactattaatatctctaaaaaactaa

SEQ ID NO: 635 - DNA, 22 bp, synthetic construct
  source 1..22: mol_type = other DNA
caaatataaaaaataatcccca

SEQ ID NO: 636 - DNA, 31 bp, synthetic construct
  source 1..31: mol_type = other DNA
ctccactaaataactatctcttactatataa

SEQ ID NO: 637 - DNA, 21 bp, synthetic construct
  source 1..21: mol_type = other DNA
ctcaaaatcccaacctcaaaa

SEQ ID NO: 638 - DNA, 20 bp, synthetic construct
  source 1..20: mol_type = other DNA
ccccactaactccccaaaaa

SEQ ID NO: 639 - DNA, 24 bp, synthetic construct
  source 1..24: mol_type = other DNA
caaaaaaacccaaaatctacaaaa

SEQ ID NO: 640 - DNA, 29 bp, synthetic construct
  source 1..29: mol_type = other DNA
ttaccaatattctcattaatttaatataa

SEQ ID NO: 641 - DNA, 20 bp, synthetic construct
  source 1..20: mol_type = other DNA
aatcaacacccactaaaaca

SEQ ID NO: 642 - DNA, 28 bp, synthetic construct
  source 1..28: mol_type = other DNA
ccaaaaactattactatacttatttcca

SEQ ID NO: 643 - DNA, 21 bp, synthetic construct
  source 1..21: mol_type = other DNA
aatcccaattcaattaaaaaa

SEQ ID NO: 644 - DNA, 21 bp, synthetic construct
  source 1..21: mol_type = other DNA
aaaatactccatattacccca

SEQ ID NO: 645 - DNA, 24 bp, synthetic construct
  source 1..24: mol_type = other DNA
ccactacacaaataaaaaaataaa

SEQ ID NO: 646 - DNA, 20 bp, synthetic construct
  source 1..20: mol_type = other DNA
aacctaaccacaaacaacca

SEQ ID NO: 647 - DNA, 29 bp, synthetic construct
  source 1..29: mol_type = other DNA
ctatacctacatatacatacctcaaataa

SEQ ID NO: 648 - DNA, 20 bp, synthetic construct
  source 1..20: mol_type = other DNA
tttttctctttttcccaaaa

SEQ ID NO: 649 - DNA, 23 bp, synthetic construct
  source 1..23: mol_type = other DNA
ttcacacttaatttaattaccca

SEQ ID NO: 650 - DNA, 21 bp, synthetic construct
  source 1..21: mol_type = other DNA
ccaataacattaaaacaacca

SEQ ID NO: 651 - DNA, 23 bp, synthetic construct
  source 1..23: mol_type = other DNA
ccacacataaataccaaaaataa

SEQ ID NO: 652 - DNA, 20 bp, synthetic construct
  source 1..20: mol_type = other DNA
ctctaccccaccaaacacca

SEQ ID NO: 653 - DNA, 24 bp, synthetic construct
  source 1..24: mol_type = other DNA
actaaaacacaaaacactaaaaca

SEQ ID NO: 654 - DNA, 26 bp, synthetic construct
  source 1..26: mol_type = other DNA
aactaaaaccaaaataaaaaaataaa

SEQ ID NO: 655 - DNA, 27 bp, synthetic construct
  source 1..27: mol_type = other DNA
aaatttaaaaaattattcaaaaaataa

SEQ ID NO: 656 - DNA, 26 bp, synthetic construct
  source 1..26: mol_type = other DNA
cctaaaaaaaaaataaaaataacaca

SEQ ID NO: 657 - DNA, 23 bp, synthetic construct
  source 1..23: mol_type = other DNA
caactacaccactctccatataa

SEQ ID NO: 658 - DNA, 24 bp, synthetic construct
  source 1..24: mol_type = other DNA
cctactaatcttactcaacaaaca

SEQ ID NO: 659 - DNA, 32 bp, synthetic construct
  source 1..32: mol_type = other DNA
aaatacaaaactctatattaattctaaaataa

SEQ ID NO: 660 - DNA, 27 bp, synthetic construct
  source 1..27: mol_type = other DNA
aaaatatactaaaaaaaaaaaatacca

SEQ ID NO: 661 - DNA, 26 bp, synthetic construct
  source 1..26: mol_type = other DNA
caattcaaaatttataaaaaaaaaaa

SEQ ID NO: 662 - DNA, 22 bp, synthetic construct
  source 1..22: mol_type = other DNA
caatcactcactaaacaaaaca

SEQ ID NO: 663 - DNA, 20 bp, synthetic construct
  source 1..20: mol_type = other DNA
cttccaccaaatattcaaaa

SEQ ID NO: 664 - DNA, 25 bp, synthetic construct
  source 1..25: mol_type = other DNA
ccatttaatataaatcacataacca

SEQ ID NO: 665 - DNA, 26 bp, synthetic construct
  source 1..26: mol_type = other DNA
aaatttcatttacaaaataaataaca

SEQ ID NO: 666 - DNA, 28 bp, synthetic construct
  source 1..28: mol_type = other DNA
cctttttatttaaaaataatattaaaca

SEQ ID NO: 667 - DNA, 28 bp, synthetic construct
  source 1..28: mol_type = other DNA
catacataacacttcttaaaataaaaca

SEQ ID NO: 668 - DNA, 30 bp, synthetic construct
  source 1..30: mol_type = other DNA
ttatcctaaaaaaattataaaaaataataa

SEQ ID NO: 669 - DNA, 24 bp, synthetic construct
  source 1..24: mol_type = other DNA
tcaaaaaaatcactatataaacca

SEQ ID NO: 670 - DNA, 21 bp, synthetic construct
  source 1..21: mol_type = other DNA
cacacccttactaattaccca

SEQ ID NO: 671 - DNA, 22 bp, synthetic construct
  source 1..22: mol_type = other DNA
ctcaaaaaataaatttccaaaa

SEQ ID NO: 672 - DNA, 25 bp, synthetic construct
  source 1..25: mol_type = other DNA
atcaaaatcataaaaaaaaacaaaa

SEQ ID NO: 673 - DNA, 23 bp, synthetic construct
  source 1..23: mol_type = other DNA
acaaaattaaaccaaatatacca

SEQ ID NO: 674 - DNA, 20 bp, synthetic construct
  source 1..20: mol_type = other DNA
acctaaactccccaaaaaca

SEQ ID NO: 675 - DNA, 24 bp, synthetic construct
  source 1..24: mol_type = other DNA
tggtagtgattagtttattttttg

SEQ ID NO: 676 - DNA, 20 bp, synthetic construct
  source 1..20: mol_type = other DNA
tgtagagaggaggaggtgag

SEQ ID NO: 677 - DNA, 20 bp, synthetic construct
  source 1..20: mol_type = other DNA
tttttgggtttgaaatgtta

SEQ ID NO: 678 - DNA, 21 bp, synthetic construct
  source 1..21: mol_type = other DNA
gggttgaggattagtattgat

SEQ ID NO: 679 - DNA, 20 bp, synthetic construct
  source 1..20: mol_type = other DNA
ggttgatgaggtataggtga

SEQ ID NO: 680 - DNA, 22 bp, synthetic construct
  source 1..22: mol_type = other DNA
gaagtaggttaagaaggaggat

SEQ ID NO: 681 - DNA, 20 bp, synthetic construct
  source 1..20: mol_type = other DNA
aggatggggattttaggttg

SEQ ID NO: 682 - DNA, 20 bp, synthetic construct
  source 1..20: mol_type = other DNA
ttgttttttgttgtgtggaa

SEQ ID NO: 683 - DNA, 24 bp, synthetic construct
  source 1..24: mol_type = other DNA
tttttaggttattttttaaatgga

SEQ ID NO: 684 - DNA, 20 bp, synthetic construct
  source 1..20: mol_type = other DNA
gtttttaaggtgttagggga

SEQ ID NO: 685 - DNA, 20 bp, synthetic construct
  source 1..20: mol_type = other DNA
ggggagttttttatgaaggg

SEQ ID NO: 686 - DNA, 20 bp, synthetic construct
  source 1..20: mol_type = other DNA
gtgtggaaggaaaaaaaaag

SEQ ID NO: 687 - DNA, 21 bp, synthetic construct
  source 1..21: mol_type = other DNA
atttttttattaggttggtgg

SEQ ID NO: 688 - DNA, 26 bp, synthetic construct
  source 1..26: mol_type = other DNA
tttagatataaatttttttgtatgga

SEQ ID NO: 689 - DNA, 23 bp, synthetic construct
  source 1..23: mol_type = other DNA
tttattggggtagagtataggtt

SEQ ID NO: 690 - DNA, 20 bp, synthetic construct
  source 1..20: mol_type = other DNA
agaggttgttgttgtgtttg

SEQ ID NO: 691 - DNA, 20 bp, synthetic construct
  source 1..20: mol_type = other DNA
gggtgtggtaggtgttttgg

SEQ ID NO: 692 - DNA, 20 bp, synthetic construct
  source 1..20: mol_type = other DNA
tgttgttttgtttgtatgga

SEQ ID NO: 693 - DNA, 20 bp, synthetic construct
  source 1..20: mol_type = other DNA
tttaggttttttgttggtgg

SEQ ID NO: 694 - DNA, 27 bp, synthetic construct
  source 1..27: mol_type = other DNA
gagtattttatgttgttttagttgttt

SEQ ID NO: 695 - DNA, 20 bp, synthetic construct
  source 1..20: mol_type = other DNA
agtaggaagggtattggtgg

SEQ ID NO: 696 - DNA, 20 bp, synthetic construct
  source 1..20: mol_type = other DNA
ggaagtgggtttgggaagta

SEQ ID NO: 697 - DNA, 22 bp, synthetic construct
  source 1..22: mol_type = other DNA
ttttggttttaaaagagagaaa

SEQ ID NO: 698 - DNA, 24 bp, synthetic construct
  source 1..24: mol_type = other DNA
tttttaatgaatggatataagtga

SEQ ID NO: 699 - DNA, 20 bp, synthetic construct
  source 1..20: mol_type = other DNA
aagtggtaagaggagttggg

SEQ ID NO: 700 - DNA, 20 bp, synthetic construct
  source 1..20: mol_type = other DNA
aggtttttgttgtgttggga

SEQ ID NO: 701 - DNA, 32 bp, synthetic construct; identical to SEQ ID NO: 157
  source 1..32: mol_type = other DNA

SEQ ID NO: 702 - DNA, 23 bp, synthetic construct
  source 1..23: mol_type = other DNA
gagaggaagtaagggtttattaa

SEQ ID NO: 703 - DNA, 20 bp, synthetic construct
  source 1..20: mol_type = other DNA
tattttatggttggggaaat

SEQ ID NO: 704 - DNA, 20 bp, synthetic construct
  source 1..20: mol_type = other DNA
ggagattggggttaggatga

SEQ ID NO: 705 - DNA, 22 bp, synthetic construct
  source 1..22: mol_type = other DNA
tggtttttttggtaatataagg

SEQ ID NO: 706 - DNA, 24 bp, synthetic construct
  source 1..24: mol_type = other DNA
agtaggtttttaaaatatgtggtt

SEQ ID NO: 707 - DNA, 20 bp, synthetic construct
  source 1..20: mol_type = other DNA
aattttgtaattgggtaggg

SEQ ID NO: 708 - DNA, 21 bp, synthetic construct
  source 1..21: mol_type = other DNA
ttttgtagttttgagaggtga

SEQ ID NO: 709 - DNA, 22 bp, synthetic construct
  source 1..22: mol_type = other DNA
ttggagtttttagttttgagtt

SEQ ID NO: 710 - DNA, 22 bp, synthetic construct
  source 1..22: mol_type = other DNA
ttgtgtgatagagtttagttgg

SEQ ID NO: 711 - DNA, 20 bp, synthetic construct
  source 1..20: mol_type = other DNA
ttttgtgtggatagttgttg

SEQ ID NO: 712 - DNA, 20 bp, synthetic construct
  source 1..20: mol_type = other DNA
ttgggatagtgttttgagtg

SEQ ID NO: 713 - DNA, 20 bp, synthetic construct
  source 1..20: mol_type = other DNA
ttttgagaagttttgaaggg

SEQ ID NO: 714 - DNA, 25 bp, synthetic construct
  source 1..25: mol_type = other DNA
ttgtttttaaaaaaattaaaaagag

SEQ ID NO: 715 - DNA, 20 bp, synthetic construct
  source 1..20: mol_type = other DNA
gagtgttttggggaatgtgt

SEQ ID NO: 716 - DNA, 24 bp, synthetic construct
  source 1..24: mol_type = other DNA
gttgtgaatataggtgtgagttaa

SEQ ID NO: 717 - DNA, 20 bp, synthetic construct
  source 1..20: mol_type = other DNA
aattgttggtaggttgttgg

SEQ ID NO: 718 - DNA, 26 bp, synthetic construct
  source 1..26: mol_type = other DNA
tagattttttttgttaaatttttttt

SEQ ID NO: 719 - DNA, 24 bp, synthetic construct
  source 1..24: mol_type = other DNA
tgagatgattaaatgaagattaaa

SEQ ID NO: 720 - DNA, 21 bp, synthetic construct
  source 1..21: mol_type = other DNA
ggttgttgtaattgtttgttg

SEQ ID NO: 721 - DNA, 24 bp, synthetic construct
  source 1..24: mol_type = other DNA
ttgtagtgtagtttgagaaatagg

SEQ ID NO: 722 - DNA, 23 bp, synthetic construct
  source 1..23: mol_type = other DNA
tgtgtttaattgtttgttttttt

SEQ ID NO: 723 - DNA, 20 bp, synthetic construct
  source 1..20: mol_type = other DNA
tattggagggtgggagagtt

SEQ ID NO: 724 - DNA, 20 bp, synthetic construct
  source 1..20: mol_type = other DNA
ttgtttatgaaatttgtggg

SEQ ID NO: 725 - DNA, 20 bp, synthetic construct
  source 1..20: mol_type = other DNA
gagtgtgtgattgggtttgt

SEQ ID NO: 726 - DNA, 20 bp, synthetic construct
  source 1..20: mol_type = other DNA
ggatattggttgttttggat

SEQ ID NO: 727 - DNA, 20 bp, synthetic construct
  source 1..20: mol_type = other DNA
tttttataattggtgaggga

SEQ ID NO: 728 - DNA, 20 bp, synthetic construct
  source 1..20: mol_type = other DNA
ggatgagtagtttttggggt

SEQ ID NO: 729 - DNA, 23 bp, synthetic construct
  source 1..23: mol_type = other DNA
gaaagaaaagaaaaagagaaagt

SEQ ID NO: 730 - DNA, 22 bp, synthetic construct
  source 1..22: mol_type = other DNA
tgtggttttgaaatagattttg

SEQ ID NO: 731 - DNA, 21 bp, synthetic construct
  source 1..21: mol_type = other DNA
tttaaagggtgttatttgagg

SEQ ID NO: 732 - DNA, 22 bp, synthetic construct
  source 1..22: mol_type = other DNA
aggaagatattgtttatgtgga

SEQ ID NO: 733 - DNA, 20 bp, synthetic construct
  source 1..20: mol_type = other DNA
tataggatggtagggttggg

SEQ ID NO: 734 - DNA, 24 bp, synthetic construct
  source 1..24: mol_type = other DNA
aggaaaattaatgaattgttaaag

SEQ ID NO: 735 - DNA, 22 bp, synthetic construct
  source 1..22: mol_type = other DNA
ttgaatgttgttatttggtatg

SEQ ID NO: 736 - DNA, 20 bp, synthetic construct
  source 1..20: mol_type = other DNA
tttgattttgttttggagtg

SEQ ID NO: 737 - DNA, 24 bp, synthetic construct
  source 1..24: mol_type = other DNA
ttttaattttgtatttgtgtgatt

SEQ ID NO: 738 - DNA, 26 bp, synthetic construct
  source 1..26: mol_type = other DNA
aaagttttaatagttttaagtttgga

SEQ ID NO: 739 - DNA, 22 bp, synthetic construct
  source 1..22: mol_type = other DNA
tgtatggtattttttgaagtga

SEQ ID NO: 740 - DNA, 20 bp, synthetic construct
  source 1..20: mol_type = other DNA
ttgattggttagagttggtt

SEQ ID NO: 741 - DNA, 23 bp, synthetic construct
  source 1..23: mol_type = other DNA
ggatttgtaattggtatagaagg

SEQ ID NO: 742 - DNA, 20 bp, synthetic construct
  source 1..20: mol_type = other DNA
gaggggatgtttttttgttg

SEQ ID NO: 743 - DNA, 20 bp, synthetic construct
  source 1..20: mol_type = other DNA
tttaaaggagggttggaatg

SEQ ID NO: 744 - DNA, 20 bp, synthetic construct
  source 1..20: mol_type = other DNA
ttgtagttggtgattgttga

SEQ ID NO: 745 - DNA, 27 bp, synthetic construct
  source 1..27: mol_type = other DNA
ggttaggatttatgtgttttataaaat

SEQ ID NO: 746 - DNA, 28 bp, synthetic construct
  source 1..28: mol_type = other DNA
attaaagagttaagtattagaaatgatg

SEQ ID NO: 747 - DNA, 20 bp, synthetic construct
  source 1..20: mol_type = other DNA
taaaggtgaagggtgtgggg

SEQ ID NO: 748 - DNA, 20 bp, synthetic construct
  source 1..20: mol_type = other DNA
agtgtgggggttttttttgg

SEQ ID NO: 749 - DNA, 20 bp, synthetic construct
  source 1..20: mol_type = other DNA
gttaggttggggtggtggtt

SEQ ID NO: 750 - DNA, 20 bp, synthetic construct
  source 1..20: mol_type = other DNA
ggaggtgggggttttttatt

SEQ ID NO: 751 - DNA, 22 bp, synthetic construct
  source 1..22: mol_type = other DNA
tttttttgggtgttaagttagt

SEQ ID NO: 752 - DNA, 24 bp, synthetic construct
  source 1..24: mol_type = other DNA
ggttttaggtgatatttgaataat

SEQ ID NO: 753 - DNA, 23 bp, synthetic construct
  source 1..23: mol_type = other DNA
ttttagtaggggttttagatttt

SEQ ID NO: 754 - DNA, 30 bp, synthetic construct; identical to SEQ ID NO: 59
  source 1..30: mol_type = other DNA

SEQ ID NO: 755 - DNA, 28 bp, synthetic construct
  source 1..28: mol_type = other DNA
agattaaaaaatatttttatttgtgtaa

SEQ ID NO: 756 - DNA, 21 bp, synthetic construct
  source 1..21: mol_type = other DNA
tgatataaataggtttggggt

SEQ ID NO: 757 - DNA, 20 bp, synthetic construct
  source 1..20: mol_type = other DNA
gggagtaggaggggtgtgtg

SEQ ID NO: 758 - DNA, 20 bp, synthetic construct
  source 1..20: mol_type = other DNA
tgaatttgttgttgattttg

SEQ ID NO: 759 - DNA, 21 bp, synthetic construct
  source 1..21: mol_type = other DNA
taatcccacttacaaaaaaca

SEQ ID NO: 760 - DNA, 24 bp, synthetic construct
  source 1..24: mol_type = other DNA
ttaatatctatcctaattccaacc

SEQ ID NO: 761 - DNA, 28 bp, synthetic construct
  source 1..28: mol_type = other DNA
tcaaaacatttctaaaactattaatatc

SEQ ID NO: 762 - DNA, 27 bp, synthetic construct
  source 1..27: mol_type = other DNA
caaaacttaaacaataatacttactca

SEQ ID NO: 763 - DNA, 20 bp, synthetic construct
  source 1..20: mol_type = other DNA
ttaaatctaacacccacacc

SEQ ID NO: 764 - DNA, 23 bp, synthetic construct
  source 1..23: mol_type = other DNA
aatataaaaaataatccccaaac

SEQ ID NO: 765 - DNA, 20 bp, synthetic construct
  source 1..20: mol_type = other DNA
aaaaaaccaaccttttccct

SEQ ID NO: 766 - DNA, 20 bp, synthetic construct
  source 1..20: mol_type = other DNA
caaacaaaatttacaaccca

SEQ ID NO: 767 - DNA, 20 bp, synthetic construct
  source 1..20: mol_type = other DNA
acctcacccacttctcctac

SEQ ID NO: 768 - DNA, 21 bp, synthetic construct
  source 1..21: mol_type = other DNA
ctcaaaatcccaacctcaaaa

SEQ ID NO: 769 - DNA, 20 bp, synthetic construct
  source 1..20: mol_type = other DNA
cccactaactccccaaaaac

SEQ ID NO: 770 - DNA, 20 bp, synthetic construct
  source 1..20: mol_type = other DNA
acccaaaatctacaaaaccc

SEQ ID NO: 771 - DNA, 23 bp, synthetic construct
  source 1..23: mol_type = other DNA
aaatccctatatattccttacca

SEQ ID NO: 772 - DNA, 20 bp, synthetic construct
  source 1..20: mol_type = other DNA
cctcatactctaaaaacccc

SEQ ID NO: 773 - DNA, 21 bp, synthetic construct
  source 1..21: mol_type = other DNA
tccccactaatacttccttac

SEQ ID NO: 774 - DNA, 20 bp, synthetic construct
  source 1..20: mol_type = other DNA
catacctcctaacatcccac

SEQ ID NO: 775 - DNA, 20 bp, synthetic construct
  source 1..20: mol_type = other DNA
tacctctaatcccaattcaa

SEQ ID NO: 776 - DNA, 25 bp, synthetic construct
  source 1..25: mol_type = other DNA
aaaaataaaccttacaaactacaca

SEQ ID NO: 777 - DNA, 20 bp, synthetic construct
  source 1..20: mol_type = other DNA
caaacacccctttttcatca

SEQ ID NO: 778 - DNA, 27 bp, synthetic construct
  source 1..27: mol_type = other DNA
aactctacaaaaaccaaatataataac

SEQ ID NO: 779 - DNA, 23 bp, synthetic construct
  source 1..23: mol_type = other DNA
acccactacacaaataaaaaaat

SEQ ID NO: 780 - DNA, 20 bp, synthetic construct
  source 1..20: mol_type = other DNA
taacctaaccacaaacaacc

SEQ ID NO: 781 - DNA, 24 bp, synthetic construct
  source 1..24: mol_type = other DNA
acctcaaataacttaaaattcact

SEQ ID NO: 782 - DNA, 28 bp, synthetic construct
  source 1..28: mol_type = other DNA
ccatttttctaacatatttactactaaa

SEQ ID NO: 783 - DNA, 24 bp, synthetic construct
  source 1..24: mol_type = other DNA
acttaatttaattacccaaacaat

SEQ ID NO: 784 - DNA, 21 bp, synthetic construct
  source 1..21: mol_type = other DNA
ccaataacattaaaacaacca

SEQ ID NO: 785 - DNA, 20 bp, synthetic construct
  source 1..20: mol_type = other DNA
atccaaaatctcctaacctc

SEQ ID NO: 786 - DNA, 20 bp, synthetic construct
  source 1..20: mol_type = other DNA
ccaccaaacaccacaatcaa

SEQ ID NO: 787 - DNA, 20 bp, synthetic construct
  source 1..20: mol_type = other DNA
aaaaaacaactccttcttcc

SEQ ID NO: 788 - DNA, 26 bp, synthetic construct
  source 1..26: mol_type = other DNA
cacaaaaaaccctaaaaaactaaaaa

SEQ ID NO: 789 - DNA, 35 bp, synthetic construct; identical to SEQ ID NO: 88
  source 1..35: mol_type = other DNA

SEQ ID NO: 790 - DNA, 20 bp, synthetic construct
  source 1..20: mol_type = other DNA
ataacaaaactcaaaacccc

SEQ ID NO: 791 - DNA, 24 bp, synthetic construct
  source 1..24: mol_type = other DNA
cacttattacccaaactaatcttt

SEQ ID NO: 792 - DNA, 21 bp, synthetic construct
  source 1..21: mol_type = other DNA
aaaaaaatccctcaatacaac

SEQ ID NO: 793 - DNA, 25 bp, synthetic construct
  source 1..25: mol_type = other DNA
aaaacaaattaccaataaattaaaa

SEQ ID NO: 794 - DNA, 20 bp, synthetic construct
  source 1..20: mol_type = other DNA
caacccacccacacaaatta

SEQ ID NO: 795 - DNA, 21 bp, synthetic construct
  source 1..21: mol_type = other DNA
ctcaaaaaaatcaaacttcaa

SEQ ID NO: 796 - DNA, 21 bp, synthetic construct
  source 1..21: mol_type = other DNA
cccacaaactacttctacaaa

SEQ ID NO: 797 - DNA, 29 bp, synthetic construct
  source 1..29: mol_type = other DNA
atttcctaaaacttataaatttataaaaa

SEQ ID NO: 798 - DNA, 24 bp, synthetic construct
  source 1..24: mol_type = other DNA
tttcaaaccttatcttaaaacttc

SEQ ID NO: 799 - DNA, 23 bp, synthetic construct
  source 1..23: mol_type = other DNA
ccctttatactttaattttctcc

SEQ ID NO: 800 - DNA, 20 bp, synthetic construct
  source 1..20: mol_type = other DNA
aaaatccccttcaattctac

SEQ ID NO: 801 - DNA, 20 bp, synthetic construct
  source 1..20: mol_type = other DNA
aaccaaacccttaacaaccc

SEQ ID NO: 802 - DNA, 27 bp, synthetic construct
  source 1..27: mol_type = other DNA
aaaaaaaataaataatatttttcaaaa

SEQ ID NO: 803 - DNA, 20 bp, synthetic construct
  source 1..20: mol_type = other DNA
taccaactacaccactctcc

SEQ ID NO: 804 - DNA, 20 bp, synthetic construct
  source 1..20: mol_type = other DNA
caaaatcaaccacaacctac

SEQ ID NO: 805 - DNA, 28 bp, synthetic construct
  source 1..28: mol_type = other DNA
ttttactcataatataatttctacctca

SEQ ID NO: 806 - DNA, 23 bp, synthetic construct
  source 1..23: mol_type = other DNA
aaaactataaactccctttcatt

SEQ ID NO: 807 - DNA, 20 bp, synthetic construct
  source 1..20: mol_type = other DNA
ccctcaacctcctaaataca

SEQ ID NO: 808 - DNA, 22 bp, synthetic construct
  source 1..22: mol_type = other DNA
aaaaataccatttacctaacca

SEQ ID NO: 809 - DNA, 20 bp, synthetic construct
  source 1..20: mol_type = other DNA
aaccaccacctccaaatctt

SEQ ID NO: 810 - DNA, 21 bp, synthetic construct
  source 1..21: mol_type = other DNA
acactcaaaaaaactaccctt

SEQ ID NO: 811 - DNA, 22 bp, synthetic construct
  source 1..22: mol_type = other DNA
cccattatttcaacttacactc

SEQ ID NO: 812 - DNA, 22 bp, synthetic construct
  source 1..22: mol_type = other DNA
tcactcactaaacaaaacaaaa

SEQ ID NO: 813 - DNA, 27 bp, synthetic construct
  source 1..27: mol_type = other DNA
aacttaaacccaaaactttaaaactac

SEQ ID NO: 814 - DNA, 22 bp, synthetic construct
  source 1..22: mol_type = other DNA
cctctcctttaaaaaaaattcc

SEQ ID NO: 815 - DNA, 20 bp, synthetic construct
  source 1..20: mol_type = other DNA
tccctatctaaacccctcct

SEQ ID NO: 816 - DNA, 20 bp, synthetic construct
  source 1..20: mol_type = other DNA
aaaatctaccttccaccaaa

SEQ ID NO: 817 - DNA, 22 bp, synthetic construct
  source 1..22: mol_type = other DNA
cataccaaaaaaaactttctca

SEQ ID NO: 818 - DNA, 21 bp, synthetic construct
  source 1..21: mol_type = other DNA
atctacaactcccaaaaattc

SEQ ID NO: 819 - DNA, 25 bp, synthetic construct
  source 1..25: mol_type = other DNA
cccctaaaatttactaaaaaaatta

SEQ ID NO: 820 - DNA, 20 bp, synthetic construct
  source 1..20: mol_type = other DNA
aaactaaccaccctctccta

SEQ ID NO: 821 - DNA, 25 bp, synthetic construct
  source 1..25: mol_type = other DNA
ctcttaatacactccataattaacc

SEQ ID NO: 822 - DNA, 28 bp, synthetic construct
  source 1..28: mol_type = other DNA
cctttttatttaaaaataatattaaaca

SEQ ID NO: 823 - DNA, 29 bp, synthetic construct
  source 1..29: mol_type = other DNA
ttcctaataaactaaaaatattaaaattc

SEQ ID NO: 824 - DNA, 21 bp, synthetic construct
  source 1..21: mol_type = other DNA
cctcctcttttctttctatca

SEQ ID NO: 825 - DNA, 24 bp, synthetic construct
  source 1..24: mol_type = other DNA
attacaattctctttcctttaaaa

SEQ ID NO: 826 - DNA, 26 bp, synthetic construct
  source 1..26: mol_type = other DNA
catctcttactaaaactaacatcaca

SEQ ID NO: 827 - DNA, 33 bp, synthetic construct
  source 1..33: mol_type = other DNA
tttattaaatattaaatacttaaaaaaattaaa

SEQ ID NO: 828 - DNA, 22 bp, synthetic construct
  source 1..22: mol_type = other DNA
aacttcacaaaaacatctctct

SEQ ID NO: 829 - DNA, 25 bp, synthetic construct
  source 1..25: mol_type = other DNA
aaattaatattatttttctaacccc

SEQ ID NO: 830 - DNA, 21 bp, synthetic construct
  source 1..21: mol_type = other DNA
tcacacccttactaattaccc

SEQ ID NO: 831 - DNA, 21 bp, synthetic construct
  source 1..21: mol_type = other DNA
tcctaaaaaaatctttccaac

SEQ ID NO: 832 - DNA, 23 bp, synthetic construct
  source 1..23: mol_type = other DNA
tcaaccaaaatataccttctaaa

SEQ ID NO: 833 - DNA, 23 bp, synthetic construct
  source 1..23: mol_type = other DNA
ccaactatactttcccataacct

SEQ ID NO: 834 - DNA, 21 bp, synthetic construct
  source 1..21: mol_type = other DNA
aaaccccaaaactcccacaac

SEQ ID NO: 835 - DNA, 22 bp, synthetic construct
  source 1..22: mol_type = other DNA
caaaatataaaaatcaaatccc

SEQ ID NO: 836 - DNA, 25 bp, synthetic construct
  source 1..25: mol_type = other DNA
atcaaaatcataaaaaaaaacaaaa

SEQ ID NO: 837 - DNA, 23 bp, synthetic construct
  source 1..23: mol_type = other DNA
ttctcccttataattttaacaca

SEQ ID NO: 838 - DNA, 27 bp, synthetic construct
  source 1..27: mol_type = other DNA
caactcaaaactctaaaataataaaca

SEQ ID NO: 839 - DNA, 30 bp, synthetic construct; identical to SEQ ID NO: 586
  source 1..30: mol_type = other DNA

SEQ ID NO: 840 - DNA, 20 bp, synthetic construct
  source 1..20: mol_type = other DNA
ccctaacctaccaacaacca

SEQ ID NO: 841 - DNA, 20 bp, synthetic construct
  source 1..20: mol_type = other DNA
cctaaactccccaaaaacac

SEQ ID NO: 842 - DNA, 21 bp, synthetic construct
  source 1..21: mol_type = other DNA
ctcatttaaacctcataaccc

SEQ ID NO: 843 - DNA, 24 bp, synthetic construct
  source 1..24: mol_type = other DNA
tggtagtgattagtttattttttg

SEQ ID NO: 844 - DNA, 20 bp, synthetic construct
  source 1..20: mol_type = other DNA
tgtagagaggaggaggtgag

SEQ ID NO: 845 - DNA, 20 bp, synthetic construct
  source 1..20: mol_type = other DNA
tttttgggtttgaaatgtta

SEQ ID NO: 846 - DNA, 21 bp, synthetic construct
  source 1..21: mol_type = other DNA
gggttgaggattagtattgat

SEQ ID NO: 847 - DNA, 20 bp, synthetic construct
  source 1..20: mol_type = other DNA
ggttgatgaggtataggtga

SEQ ID NO: 848 - DNA, 22 bp, synthetic construct
  source 1..22: mol_type = other DNA
gaagtaggttaagaaggaggat

SEQ ID NO: 849 - DNA, 20 bp, synthetic construct
  source 1..20: mol_type = other DNA
aggatggggattttaggttg

SEQ ID NO: 850 - DNA, 20 bp, synthetic construct
  source 1..20: mol_type = other DNA
ttgttttttgttgtgtggaa

SEQ ID NO: 851 - DNA, 24 bp, synthetic construct
  source 1..24: mol_type = other DNA
tttttaggttattttttaaatgga

SEQ ID NO: 852 - DNA, 20 bp, synthetic construct
  source 1..20: mol_type = other DNA
gtttttaaggtgttagggga

SEQ ID NO: 853 - DNA, 20 bp, synthetic construct
  source 1..20: mol_type = other DNA
ggggagttttttatgaaggg

SEQ ID NO: 854 - DNA, 20 bp, synthetic construct
  source 1..20: mol_type = other DNA
gtgtggaaggaaaaaaaaag

SEQ ID NO: 855 - DNA, 21 bp, synthetic construct
  source 1..21: mol_type = other DNA
atttttttattaggttggtgg

SEQ ID NO: 856 - DNA, 26 bp, synthetic construct
  source 1..26: mol_type = other DNA
tttagatataaatttttttgtatgga

SEQ ID NO: 857 - DNA, 23 bp, synthetic construct
  source 1..23: mol_type = other DNA
tttattggggtagagtataggtt

SEQ ID NO: 858 - DNA, 20 bp, synthetic construct
  source 1..20: mol_type = other DNA
agaggttgttgttgtgtttg

SEQ ID NO: 859 - DNA, 20 bp, synthetic construct
  source 1..20: mol_type = other DNA
gggtgtggtaggtgttttgg

SEQ ID NO: 860 - DNA, 20 bp, synthetic construct
  source 1..20: mol_type = other DNA
tgttgttttgtttgtatgga

SEQ ID NO: 861 - DNA, 20 bp, synthetic construct
  source 1..20: mol_type = other DNA
tttaggttttttgttggtgg

SEQ ID NO: 862 - DNA, 27 bp, synthetic construct
  source 1..27: mol_type = other DNA
gagtattttatgttgttttagttgttt

SEQ ID NO: 863 - DNA, 20 bp, synthetic construct
  source 1..20: mol_type = other DNA
agtaggaagggtattggtgg

SEQ ID NO: 864 - DNA, 20 bp, synthetic construct
  source 1..20: mol_type = other DNA
ggaagtgggtttgggaagta

SEQ ID NO: 865 - DNA, 22 bp, synthetic construct
  source 1..22: mol_type = other DNA
ttttggttttaaaagagagaaa

SEQ ID NO: 866 - DNA, 24 bp, synthetic construct
  source 1..24: mol_type = other DNA
aaagagtttttaatgaatggatat

SEQ ID NO: 867 - DNA, 20 bp, synthetic construct
  source 1..20: mol_type = other DNA
aagtggtaagaggagttggg

SEQ ID NO: 868 - DNA, 20 bp, synthetic construct
  source 1..20: mol_type = other DNA
aggtttttgttgtgttggga

SEQ ID NO: 869 - DNA, 32 bp, synthetic construct; identical to SEQ ID NO: 157
  source 1..32: mol_type = other DNA

SEQ ID NO: 870 - DNA, 23 bp, synthetic construct
  source 1..23: mol_type = other DNA
gagaggaagtaagggtttattaa

SEQ ID NO: 871 - DNA, 20 bp, synthetic construct
  source 1..20: mol_type = other DNA
tattttatggttggggaaat

SEQ ID NO: 872 - DNA, 20 bp, synthetic construct
  source 1..20: mol_type = other DNA
ggagattggggttaggatga

SEQ ID NO: 873 - DNA, 22 bp, synthetic construct
  source 1..22: mol_type = other DNA
tggtttttttggtaatataagg

SEQ ID NO: 874 - DNA, 24 bp, synthetic construct
  source 1..24: mol_type = other DNA
agtaggtttttaaaatatgtggtt

SEQ ID NO: 875 - DNA, 20 bp, synthetic construct
  source 1..20: mol_type = other DNA
aattttgtaattgggtaggg

SEQ ID NO: 876 - DNA, 21 bp, synthetic construct
  source 1..21: mol_type = other DNA
ttttgtagttttgagaggtga

SEQ ID NO: 877 - DNA, 22 bp, synthetic construct
  source 1..22: mol_type = other DNA
ttggagtttttagttttgagtt

SEQ ID NO: 878 - DNA, 22 bp, synthetic construct
  source 1..22: mol_type = other DNA
ttgtgtgatagagtttagttgg

SEQ ID NO: 879 - DNA, 20 bp, synthetic construct
  source 1..20: mol_type = other DNA
ttttgtgtggatagttgttg

SEQ ID NO: 880 - DNA, 20 bp, synthetic construct
  source 1..20: mol_type = other DNA
ttgggatagtgttttgagtg

SEQ ID NO: 881 - DNA, 20 bp, synthetic construct
  source 1..20: mol_type = other DNA
ttttgagaagttttgaaggg

SEQ ID NO: 882 - DNA, 25 bp, synthetic construct
  source 1..25: mol_type = other DNA
ttgtttttaaaaaaattaaaaagag

SEQ ID NO: 883 - DNA, 20 bp, synthetic construct
  source 1..20: mol_type = other DNA
gagtgttttggggaatgtgt

SEQ ID NO: 884 - DNA, 24 bp, synthetic construct
  source 1..24: mol_type = other DNA
gttgtgaatataggtgtgagttaa

SEQ ID NO: 885 - DNA, 20 bp, synthetic construct
  source 1..20: mol_type = other DNA
aattgttggtaggttgttgg

SEQ ID NO: 886 - DNA, 26 bp, synthetic construct
  source 1..26: mol_type = other DNA
tagattttttttgttaaatttttttt

SEQ ID NO: 887 - DNA, 24 bp, synthetic construct
  source 1..24: mol_type = other DNA
tgagatgattaaatgaagattaaa

SEQ ID NO: 888 - DNA, 21 bp, synthetic construct
  source 1..21: mol_type = other DNA
ggttgttgtaattgtttgttg

SEQ ID NO: 889 - DNA, 24 bp, synthetic construct
  source 1..24: mol_type = other DNA
ttgtagtgtagtttgagaaatagg

SEQ ID NO: 890 - DNA, 23 bp, synthetic construct
  source 1..23: mol_type = other DNA
tgtgtttaattgtttgttttttt

SEQ ID NO: 891 - DNA, 20 bp, synthetic construct
  source 1..20: mol_type = other DNA
tattggagggtgggagagtt

SEQ ID NO: 892 - DNA, 20 bp, synthetic construct
  source 1..20: mol_type = other DNA
ttgtttatgaaatttgtggg

SEQ ID NO: 893 - DNA, 20 bp, synthetic construct
  source 1..20: mol_type = other DNA
gagtgtgtgattgggtttgt

SEQ ID NO: 894 - DNA, 20 bp, synthetic construct
  source 1..20: mol_type = other DNA
ggatattggttgttttggat

SEQ ID NO: 895 - DNA, 20 bp, synthetic construct
  source 1..20: mol_type = other DNA
ggggattttttataattggt

SEQ ID NO: 896 - DNA, 20 bp, synthetic construct
  source 1..20: mol_type = other DNA
ggatgagtagtttttggggt

SEQ ID NO: 897 - DNA, 23 bp, synthetic construct
  source 1..23: mol_type = other DNA
gaaagaaaagaaaaagagaaagt

SEQ ID NO: 898 - DNA, 22 bp, synthetic construct
  source 1..22: mol_type = other DNA
tgtggttttgaaatagattttg

SEQ ID NO: 899 - DNA, 20 bp, synthetic construct
  source 1..20: mol_type = other DNA
aaagggtgttatttgaggtt

SEQ ID NO: 900 - DNA, 21 bp, synthetic construct
  source 1..21: mol_type = other DNA
tgtggaaatattgatttttga

SEQ ID NO: 901 - DNA, 20 bp, synthetic construct
  source 1..20: mol_type = other DNA
ttataggatggtagggttgg

SEQ ID NO: 902 - DNA, 24 bp, synthetic construct
  source 1..24: mol_type = other DNA
aggaaaattaatgaattgttaaag

SEQ ID NO: 903 - DNA, 22 bp, synthetic construct
  source 1..22: mol_type = other DNA
ttgaatgttgttatttggtatg

SEQ ID NO: 904 - DNA, 20 bp, synthetic construct
  source 1..20: mol_type = other DNA
tttgattttgttttggagtg

SEQ ID NO: 905 - DNA, 24 bp, synthetic construct
  source 1..24: mol_type = other DNA
ttttaattttgtatttgtgtgatt

SEQ ID NO: 906 - DNA, 26 bp, synthetic construct
  source 1..26: mol_type = other DNA
aaagttttaatagttttaagtttgga

SEQ ID NO: 907 - DNA, 22 bp, synthetic construct
  source 1..22: mol_type = other DNA
tgtatggtattttttgaagtga

SEQ ID NO: 908 - DNA, 20 bp, synthetic construct
  source 1..20: mol_type = other DNA
ttgattggttagagttggtt

SEQ ID NO: 909 - DNA, 23 bp, synthetic construct
  source 1..23: mol_type = other DNA
ggatttgtaattggtatagaagg

SEQ ID NO: 910 - DNA, 20 bp, synthetic construct
  source 1..20: mol_type = other DNA
gaggggatgtttttttgttg

SEQ ID NO: 911 - DNA, 20 bp, synthetic construct
  source 1..20: mol_type = other DNA
tttaaaggagggttggaatg

SEQ ID NO: 912 - DNA, 20 bp, synthetic construct
  source 1..20: mol_type = other DNA
ttgtagttggtgattgttga

SEQ ID NO: 913 - DNA, 25 bp, synthetic construct
  source 1..25: mol_type = other DNA
aggttaggatttatgtgttttataa

SEQ ID NO: 914 - DNA, 28 bp, synthetic construct
  source 1..28: mol_type = other DNA
attaaagagttaagtattagaaatgatg

SEQ ID NO: 915 - DNA, 20 bp, synthetic construct
  source 1..20: mol_type = other DNA
taataaaggtgaagggtgtg

SEQ ID NO: 916 - DNA, 20 bp, synthetic construct
  source 1..20: mol_type = other DNA
agtgtgggggttttttttgg

SEQ ID NO: 917 - DNA, 20 bp, synthetic construct
  source 1..20: mol_type = other DNA
gttaggttggggtggtggtt

SEQ ID NO: 918 - DNA, 20 bp, synthetic construct
  source 1..20: mol_type = other DNA
ttggaggtgggggtttttta

SEQ ID NO: 919 - DNA, 22 bp, synthetic construct
  source 1..22: mol_type = other DNA
tttttttgggtgttaagttagt

SEQ ID NO: 920 - DNA, 24 bp, synthetic construct
  source 1..24: mol_type = other DNA
ggttttaggtgatatttgaataat

SEQ ID NO: 921 - DNA, 23 bp, synthetic construct
  source 1..23: mol_type = other DNA
ttttagtaggggttttagatttt

SEQ ID NO: 922 - DNA, 30 bp, synthetic construct; identical to SEQ ID NO: 59
  source 1..30: mol_type = other DNA

SEQ ID NO: 923 - DNA, 28 bp, synthetic construct
  source 1..28: mol_type = other DNA
agattaaaaaatatttttatttgtgtaa

SEQ ID NO: 924 - DNA, 21 bp, synthetic construct
  source 1..21: mol_type = other DNA
tgatataaataggtttggggt

SEQ ID NO: 925 - DNA, 20 bp, synthetic construct
  source 1..20: mol_type = other DNA
gggagtaggaggggtgtgtg

SEQ ID NO: 926 - DNA, 20 bp, synthetic construct
  source 1..20: mol_type = other DNA
tgaatttgttgttgattttg

SEQ ID NO: 927 - DNA, 21 bp, synthetic construct
  source 1..21: mol_type = other DNA
taatcccacttacaaaaaaca

SEQ ID NO: 928 - DNA, 24 bp, synthetic construct
  source 1..24: mol_type = other DNA
ttaatatctatcctaattccaacc

SEQ ID NO: 929 - DNA, 27 bp, synthetic construct
  source 1..27: mol_type = other DNA
aatcaaaacatttctaaaactattaat

SEQ ID NO: 930 - DNA, 27 bp, synthetic construct
  source 1..27: mol_type = other DNA
caaaacttaaacaataatacttactca

SEQ ID NO: 931 - DNA, 20 bp, synthetic construct
  source 1..20: mol_type = other DNA
ttaaatctaacacccacacc

SEQ ID NO: 932 - DNA, 23 bp, synthetic construct
  source 1..23: mol_type = other DNA
aatataaaaaataatccccaaac

SEQ ID NO: 933 - DNA, 20 bp, synthetic construct
  source 1..20: mol_type = other DNA
aaaaaaccaaccttttccct

SEQ ID NO: 934 - DNA, 20 bp, synthetic construct
  source 1..20: mol_type = other DNA
caaacaaaatttacaaccca

SEQ ID NO: 935 - DNA, 20 bp, synthetic construct
  source 1..20: mol_type = other DNA
acctcacccacttctcctac

SEQ ID NO: 936 - DNA, 21 bp, synthetic construct
  source 1..21: mol_type = other DNA
ctcaaaatcccaacctcaaaa

SEQ ID NO: 937 - DNA, 20 bp, synthetic construct
  source 1..20: mol_type = other DNA
cccactaactccccaaaaac

SEQ ID NO: 938 - DNA, 20 bp, synthetic construct
  source 1..20: mol_type = other DNA
acccaaaatctacaaaaccc

SEQ ID NO: 939 - DNA, 23 bp, synthetic construct
  source 1..23: mol_type = other DNA
aaatccctatatattccttacca

SEQ ID NO: 940 - DNA, 20 bp, synthetic construct
  source 1..20: mol_type = other DNA
cctcatactctaaaaacccc

SEQ ID NO: 941 - DNA, 21 bp, synthetic construct
  source 1..21: mol_type = other DNA
tccccactaatacttccttac

SEQ ID NO: 942 - DNA, 20 bp, synthetic construct
  source 1..20: mol_type = other DNA
catacctcctaacatcccac

SEQ ID NO: 943 - DNA, 20 bp, synthetic construct
  source 1..20: mol_type = other DNA
tacctctaatcccaattcaa

SEQ ID NO: 944 - DNA, 25 bp, synthetic construct
  source 1..25: mol_type = other DNA
aaaaataaaccttacaaactacaca

SEQ ID NO: 945 - DNA, 20 bp, synthetic construct
  source 1..20: mol_type = other DNA
caaacacccctttttcatca

SEQ ID NO: 946 - DNA, 27 bp, synthetic construct
  source 1..27: mol_type = other DNA
aactctacaaaaaccaaatataataac

SEQ ID NO: 947 - DNA, 23 bp, synthetic construct
  source 1..23: mol_type = other DNA
acccactacacaaataaaaaaat

SEQ ID NO: 948 - DNA, 20 bp, synthetic construct
  source 1..20: mol_type = other DNA
taacctaaccacaaacaacc

SEQ ID NO: 949 - DNA, 24 bp, synthetic construct
  source 1..24: mol_type = other DNA
acctcaaataacttaaaattcact

SEQ ID NO: 950 - DNA, 31 bp, synthetic construct
  source 1..31: mol_type = other DNA
ttctaacatatttactactaaaaaatttaaa

SEQ ID NO: 951 - DNA, 23 bp, synthetic construct
  source 1..23: mol_type = other DNA
cacttaatttaattacccaaaca

SEQ ID NO: 952 - DNA, 21 bp, synthetic construct
  source 1..21: mol_type = other DNA
ccaataacattaaaacaacca

SEQ ID NO: 953 - DNA, 20 bp, synthetic construct
  source 1..20: mol_type = other DNA
atccaaaatctcctaacctc

SEQ ID NO: 954 - DNA, 20 bp, synthetic construct
  source 1..20: mol_type = other DNA
ccaccaaacaccacaatcaa

SEQ ID NO: 955 - DNA, 20 bp, synthetic construct
  source 1..20: mol_type = other DNA
aaaaaacaactccttcttcc

SEQ ID NO: 956 - DNA, 26 bp, synthetic construct
  source 1..26: mol_type = other DNA
cacaaaaaaccctaaaaaactaaaaa

SEQ ID NO: 957 - DNA, 35 bp, synthetic construct; identical to SEQ ID NO: 88
  source 1..35: mol_type = other DNA

SEQ ID NO: 958 - DNA, 20 bp, synthetic construct
  source 1..20: mol_type = other DNA
ataacaaaactcaaaacccc

SEQ ID NO: 959 - DNA, 24 bp, synthetic construct
  source 1..24: mol_type = other DNA
cacttattacccaaactaatcttt

SEQ ID NO: 960 - DNA, 21 bp, synthetic construct
  source 1..21: mol_type = other DNA
aaaaaaatccctcaatacaac

SEQ ID NO: 961 - DNA, 25 bp, synthetic construct
  source 1..25: mol_type = other DNA
aaaacaaattaccaataaattaaaa

SEQ ID NO: 962 - DNA, 20 bp, synthetic construct
  source 1..20: mol_type = other DNA
caacccacccacacaaatta

SEQ ID NO: 963 - DNA, 21 bp, synthetic construct
  source 1..21: mol_type = other DNA
ctcaaaaaaatcaaacttcaa

SEQ ID NO: 964 - DNA, 21 bp, synthetic construct
  source 1..21: mol_type = other DNA
cccacaaactacttctacaaa

SEQ ID NO: 965 - DNA, 29 bp, synthetic construct
  source 1..29: mol_type = other DNA
atttcctaaaacttataaatttataaaaa

SEQ ID NO: 966 - DNA, 24 bp, synthetic construct
  source 1..24: mol_type = other DNA
tttcaaaccttatcttaaaacttc

SEQ ID NO: 967 - DNA, 23 bp, synthetic construct
  source 1..23: mol_type = other DNA
ccctttatactttaattttctcc

SEQ ID NO: 968 - DNA, 20 bp, synthetic construct
  source 1..20: mol_type = other DNA
aaaatccccttcaattctac

SEQ ID NO: 969 - DNA, 20 bp, synthetic construct
  source 1..20: mol_type = other DNA
aaccaaacccttaacaaccc

SEQ ID NO: 970 - DNA, 27 bp, synthetic construct
  source 1..27: mol_type = other DNA
aaaaaaaataaataatatttttcaaaa

SEQ ID NO: 971 - DNA, 20 bp, synthetic construct
  source 1..20: mol_type = other DNA
taccaactacaccactctcc

SEQ ID NO: 972 - DNA, 20 bp, synthetic construct
  source 1..20: mol_type = other DNA
cacaaaatcaaccacaacct

SEQ ID NO: 973 - DNA, 28 bp, synthetic construct
  source 1..28: mol_type = other DNA
ttttactcataatataatttctacctca

SEQ ID NO: 974 - DNA, 23 bp, synthetic construct
  source 1..23: mol_type = other DNA
aaaactataaactccctttcatt

SEQ ID NO: 975 - DNA, 20 bp, synthetic construct
  source 1..20: mol_type = other DNA
ccctcaacctcctaaataca

SEQ ID NO: 976 - DNA, 22 bp, synthetic construct
  source 1..22: mol_type = other DNA
aaaaataccatttacctaacca

SEQ ID NO: 977 - DNA, 20 bp, synthetic construct
  source 1..20: mol_type = other DNA
aaccaccacctccaaatctt

SEQ ID NO: 978 - DNA, 21 bp, synthetic construct
  source 1..21: mol_type = other DNA
acactcaaaaaaactaccctt

SEQ ID NO: 979 - DNA, 24 bp, synthetic construct
  source 1..24: mol_type = other DNA
tttcaacttacactctaacaaaca

SEQ ID NO: 980 - DNA, 22 bp, synthetic construct
  source 1..22: mol_type = other DNA
tcactcactaaacaaaacaaaa

SEQ ID NO: 981 - DNA, 27 bp, synthetic construct
  source 1..27: mol_type = other DNA
aacttaaacccaaaactttaaaactac

SEQ ID NO: 982 - DNA, 22 bp, synthetic construct
  source 1..22: mol_type = other DNA
cctctcctttaaaaaaaattcc

SEQ ID NO: 983 - DNA, 20 bp, synthetic construct
  source 1..20: mol_type = other DNA
tccctatctaaacccctcct

SEQ ID NO: 984 - DNA, 20 bp, synthetic construct
  source 1..20: mol_type = other DNA
tcaaaaatctaccttccacc

SEQ ID NO: 985 - DNA, 22 bp, synthetic construct
  source 1..22: mol_type = other DNA
tcacataccaaaaaaaactttc

SEQ ID NO: 986 - DNA, 21 bp, synthetic construct
  source 1..21: mol_type = other DNA
atctacaactcccaaaaattc

SEQ ID NO: 987 - DNA, 24 bp, synthetic construct
  source 1..24: mol_type = other DNA
cccctaaaatttactaaaaaaatt

SEQ ID NO: 988 - DNA, 20 bp, synthetic construct
  source 1..20: mol_type = other DNA
aaactaaccaccctctccta

SEQ ID NO: 989 - DNA, 25 bp, synthetic construct
  source 1..25: mol_type = other DNA
ctcttaatacactccataattaacc

SEQ ID NO: 990 - DNA, 28 bp, synthetic construct
  source 1..28: mol_type = other DNA
cctttttatttaaaaataatattaaaca

SEQ ID NO: 991 - DNA, 29 bp, synthetic construct
  source 1..29: mol_type = other DNA
ttcctaataaactaaaaatattaaaattc

SEQ ID NO: 992 - DNA, 21 bp, synthetic construct
  source 1..21: mol_type = other DNA
cctcctcttttctttctatca

SEQ ID NO: 993 - DNA, 24 bp, synthetic construct
  source 1..24: mol_type = other DNA
attacaattctctttcctttaaaa

SEQ ID NO: 994 - DNA, 26 bp, synthetic construct
  source 1..26: mol_type = other DNA
catctcttactaaaactaacatcaca

SEQ ID NO: 995 - DNA, 33 bp, synthetic construct; identical to SEQ ID NO: 827
  source 1..33: mol_type = other DNA

SEQ ID NO: 996 - DNA, 22 bp, synthetic construct
  source 1..22: mol_type = other DNA
aacttcacaaaaacatctctct

SEQ ID NO: 997 - DNA, 25 bp, synthetic construct
  source 1..25: mol_type = other DNA
aaattaatattatttttctaacccc

SEQ ID NO: 998 - DNA, 21 bp, synthetic construct
  source 1..21: mol_type = other DNA
tcacacccttactaattaccc

SEQ ID NO: 999 - DNA, 21 bp, synthetic construct
  source 1..21: mol_type = other DNA
tcctaaaaaaatctttccaac

SEQ ID NO: 1000 - DNA, 23 bp, synthetic construct
  source 1..23: mol_type = other DNA
aaatcaaccaaaatataccttct

SEQ ID NO: 1001 - DNA, 23 bp, synthetic construct
  source 1..23: mol_type = other DNA
ccaactatactttcccataacct

SEQ ID NO: 1002 - DNA, 21 bp, synthetic construct
  source 1..21: mol_type = other DNA
aaaccccaaaactcccacaac

SEQ ID NO: 1003 - DNA, 22 bp, synthetic construct
  source 1..22: mol_type = other DNA
caaaatataaaaatcaaatccc

SEQ ID NO: 1004 - DNA, 25 bp, synthetic construct
  source 1..25: mol_type = other DNA
atcaaaatcataaaaaaaaacaaaa

SEQ ID NO: 1005 - DNA, 23 bp, synthetic construct
  source 1..23: mol_type = other DNA
ttctcccttataattttaacaca

SEQ ID NO: 1006 - DNA, 27 bp, synthetic construct
  source 1..27: mol_type = other DNA
aacaactcaaaactctaaaataataaa

SEQ ID NO: 1007 - DNA, 30 bp, synthetic construct; identical to SEQ ID NO: 586
  source 1..30: mol_type = other DNA

SEQ ID NO: 1008 - DNA, 20 bp, synthetic construct
  source 1..20: mol_type = other DNA
ccctaacctaccaacaacca

SEQ ID NO: 1009 - DNA, 20 bp, synthetic construct
  source 1..20: mol_type = other DNA
cctaaactccccaaaaacac

SEQ ID NO: 1010 - DNA, 21 bp, synthetic construct
  source 1..21: mol_type = other DNA
ctcatttaaacctcataaccc